(19) United States
(12) Patent Application Publication
(10) Pub. No.: US 2024/0376475 A1
(43) Pub. Date: Nov. 14, 2024

(54) COMPOSITIONS AND METHODS FOR INHIBITING EXPRESSION OF ANGIOPOIETIN-LIKE 3 (ANGPTL3) PROTEIN

(71) Applicant: Shanghai Argo Biopharmaceutical Co., Ltd., Shanghai (CN)

(72) Inventors: Dongxu Shu, Ningbo (CN); Pengcheng Patrick Shao, Warrington Township, PA (US)

(73) Assignee: Shanghai Argo Biopharmaceutical Co., Ltd., Shanghai (CN)

(21) Appl. No.: 18/554,692

(22) PCT Filed: Sep. 22, 2022
(86) PCT No.: PCT/CN2022/120421

§ 371 Date: Oct. 10, 2023

(30) Foreign Application Priority Data
Sep. 23, 2021 (WO) PCT/CN2021/119734

(51) Int. Cl. C12N 15/113; A61K 9/00; A61K 31/713; A61K 45/06; A61P 3/06

(52) U.S. Cl. CPC C12N 15/1136; A61K 9/0019; A61K 31/713; A61K 45/06; A61P 3/06; C12N 2310/14; C12N 2310/315; C12N 2310/321; C12N 2310/322; C12N 2320/32

(57) ABSTRACT

Compositions and methods useful to reduce expression of Angiopoietin-like 3 (ANGPTL3) gene and for treatment of ANGPTL3-associated diseases and conditions are provided. Provided are ANGPTL3 dsRNA agents, ANGPTL3 antisense polynucleotide agents, compositions comprising ANGPTL3 dsRNA agents, and, compositions comprising ANGPTL3 antisense polynucleotide agents that can be used to reduce ANGPTL3 expression in cells and subjects.

### FIELD OF THE INVENTION

The invention relates, in part, to compositions and methods that can be used to inhibit angiopoietin-like 3 (ANGPTL3) protein expression.

### BACKGROUND

Angiopoietin-like protein 3 (ANGPTL3) is a secreted protein that is mainly expressed in hepatocytes (Conklin et al. Identification of a mammalian angiopoietin-related protein expressed specifically in liver. Genomics 1999, 62:477-482). It is an inhibitor of lipoprotein lipase (LPL) and endothelial lipase (EL). Acting through inhibition of LPL and EL, ANGPTL3 reduces hydrolysis of triglycerides (TG), particularly in muscle and fat tissue (Kersten S. Physiological regulation of lipoprotein lipase. Biochem Biophys Acta 2014; 1841:919-933. Shimamura et al. Angiopoietin-like protein3 regulates plasma HDL cholesterol through suppression of endothelial lipase. Arterioscler. Thromb. Vasc. Biol. 2007; 27:366-372). Thus, inhibition of ANGPTL3 disinhibits LPL and EL activity, which results reduction of TG and high-density lipoprotein cholesterol (HDL-C). Inhibition of ANGPTL3 also leads to reduction of low-density lipoprotein cholesterol (LDL-C), possibly through EL-mediated processing of VLDL (Adam, et al. Angiopoietin-like protein3 governs LDL-cholesterol levels through endothelial lipase-dependent VLDL clearance. J Lipid Res 2020; 61:1271-1286). It is also noteworthy that current LDL-C lowering therapies, such as statins and PCSK9 inhibitors are LDL-R dependent, and are not effective for patients with low or no residue LDL-R activity. LDL-C lowering through inhibition of ANGPTL3 is LDL-R independent, which could be an effective therapeutic approach to manage lipids for patients with low or no LDL-R activity.

Hyperlipidemia is strongly associated with diseases including high blood pressure, atherosclerosis, heart diseases, diabetes, nonalcoholic steatohepatitis (NASH). Study have shown beneficial effect of loss function mutation of ANGPTL3 in human. Homozygous loss of ANGPTL3 function causes familial combined hypolipidemia characterized by low plasma levels of triglycerides, high-density lipoprotein (HDL) cholesterol, and LDL-C and a decreased risk of coronary artery disease (Romeo et el., Rare loss-of-function mutations in ANGPTL family members contribute to plasma triglyceride levels in humans., J. Clin. Invest., 2009, 119:70-79; Musunuru, et al., Exome sequencing, ANGPTL3 mutations, and familial combined hypolipidemia., N Engl J Med, 2010, 363:2220-2227). ANGPTL3 has emerged as a promising drug target for treating diseases caused by hyperlipidemia with therapeutic modalities including antibody, antisense oligonucleotide (ASO) and siRNA in development. siRNA, particularly GalNAc-conjugated siRNA has been shown to be safe, effective and with long during of activity. Thus, there is a need for new ANGPTL3 siRNA agents for treating various diseases and conditions.

### SUMMARY OF THE INVENTION

According to an aspect of the invention, a double-stranded ribonucleic acid (dsRNA) agent for inhibiting expression of Angiopoietin-like 3 (ANGPTL3) is provided, the dsRNA agent including a sense strand and an antisense strand, nucleotide positions 2 to 18 in the antisense strand including a region of complementarity to an ANGPTL3 RNA transcript, wherein the region of complementarity includes at least 15 contiguous nucleotides that differ by 0, 1, 2, or 3 nucleotides from one of the antisense sequences listed in one of Tables 1-5, and optionally including a targeting ligand. In some embodiments, the region of complementarity to an ANGPTL3 RNA transcript includes at least 15, 16, 17, 18, or 19 contiguous nucleotides that differ by no more than 3 nucleotides from one of the antisense sequences listed in one of Tables 1-5. In certain embodiments, the antisense strand of dsRNA is at least substantially complementary to any one of a target region of SEQ ID NO: 235 and is provided in any one of Tables 1-5. In some embodiments, the antisense strand of dsRNA is fully complementary to any one of a target region of SEQ ID NO: 235 and is provided in any one of Tables 1-5. In some embodiments, the dsRNA agent includes a sense strand sequence set forth in any one of Tables 1-5, wherein the sense strand sequence is at least substantially complementary to the antisense strand sequence in the dsRNA agent. In certain embodiments, the dsRNA agent includes a sense strand sequence set forth in any one of Tables 1-5, wherein the sense strand sequence is fully complementary to the antisense strand sequence in the dsRNA agent. In some embodiments, the dsRNA agent includes an antisense strand sequence set forth in any one of Tables 1-5. In some embodiments, the dsRNA agent includes the sequences set forth as a duplex sequence in any of Tables 1-5. In some embodiments, the antisense strand of dsRNA consists of a nucleotide sequence II: 5′-ziuagaguauaaccuuccz₂-3′, wherein z₁ is selected from c, g, a or u, z₂ is a nucleotide sequence IV. In certain embodiments, z₁ is u. In certain embodiments, the nucleotide sequence IV is 0-15 nucleotides in length. In certain embodiments, the nucleotide sequence IV is selected from a, au, aa, ac, ag, auu, aua, auc, aug, auug, auuu, auua, auuc, auuuu, auuuug, auucuu, auucga, auuuuga, auuuugag, auuuugaga or auuuugagacuucca. In certain embodiments, the nucleotide sequence IV is 1, 2, 3 or 4 nucleotides in length. In certain embodiments, the nucleotide sequence IV is selected from a, au, aa, ac, ag, auu, aua, auc, aug, auug, auuu, auua or auuc. In certain embodiments, the antisense strand of dsRNA consists of a nucleotide sequence II′: 5′-ziuagaguauaaccuuccaz₂′-3′, wherein z₁ is selected from c, g, a or u, z₂′ is a nucleotide sequence IV′. In certain embodiments, z₁ is u. In certain embodiments, the nucleotide sequence IV′ is 0-15 nucleotides in length. In certain embodiments, the nucleotide sequence IV′ is 1, 2, 3 or 4 nucleotides in length. In certain embodiments, the nucleotide sequence IV′ is selected from u, a, c, g, uu, ua, uc, ug, uug, uuu, uua or uuc. In some embodiments, the sense strand of dsRNA consists of a nucleotide sequence III: 5′-z₃ggaagguuauacucuaz₄-3′, wherein z₃ is a nucleotide sequence V, z₄ is selected from c, g, a or u. In certain embodiments, z₄ is a. In certain embodiments, the nucleotide sequence V is 0-15 nucleotides in length. In certain embodiments, the nucleotide sequence V is selected from u, au, uu, gu, cu, aau, uau, gau, cau, gaau, caau, aaau, uaau, aaaau, caaaau, ucaaaau, cucaaaau, ucucaaaau or uggaagucucaaaau. In certain embodiments, the nucleotide sequence V is 1, 2, 3 or 4 nucleotides in length. In certain embodiments, the nucleotide sequence V is selected from u, au, uu, gu, cu, aau, uau, gau, cau, gaau, caau, aaau or uaau. In certain embodiments, the sense strand of dsRNA consists of a nucleotide sequence III′: 5′-z₃′uggaagguuauacucuaz4-3′, wherein z₃′ is a nucleotide sequence V′, z₄ is selected from c, g, a or u. In certain embodiments, z₄ is a. In certain embodiments, the nucleotide sequence V′ is 1, 2, 3 or 4 nucleotides in length. In certain embodiments, the nucleotide sequence V′ is selected from a, u, g, c, aa, ua, ga, ca, gaa, caa, aaa or uaa. In some embodiments, z₁ is a nucleotide complementary to z₄. In some embodiments, z₂ is a nucleotide sequence complementary to z₃.

In some embodiments, z₂, is a nucleotide sequence complementary to z₃. In some embodiments, the dsRNA agent includes a sense strand and an antisense strand, wherein the antisense strand of dsRNA consists of the nucleotide sequence II or II′ as described above, wherein the sense strand is no more than 30 nucleotides in length comprising a region of complementarity to the antisense strand including at least 15, 16, 17, 18, or 19 nucleotides. In some embodiments, the dsRNA agent includes a sense strand and an antisense strand, wherein the sense strand of dsRNA consists of the nucleotide sequence III and the antisense strand of dsRNA consists of the nucleotide sequence II, wherein the nucleotide sequence II and III are as described above. In some embodiments, the dsRNA agent includes a sense strand and an antisense strand, wherein the sense strand of dsRNA consists of the nucleotide sequence III′ and the antisense strand of dsRNA consists of the nucleotide sequence II′, wherein the nucleotide sequence II′ and III′ are as described above.

In some embodiments, the dsRNAs include a sense strand and an antisense strand, the antisense strand comprising a region of complementarity which includes at least 15 contiguous nucleotides that differ by 0, 1, 2, or 3 nucleotides from any one of the nucleotide sequences selected from the group consisting of

(SEQ ID NO: 139)
5′-uacaugaaaaacuugagaguc-3′
(SEQ ID NO: 158)
5′-uuagaguauaaccuuccauuc-3′
(SEQ ID NO: 120)
5′-uguuuuugaaauaugucauuc-3′
(SEQ ID NO: 123)
5′-uucauaaaaagacugaucaac-3′
(SEQ ID NO: 125)
5′-uuaguuuauauguaguucuuc-3′
(SEQ ID NO: 129)
5′-uguugaguucaagugacauac-3′
(SEQ ID NO: 147)
5′-uuuuugugauccaucuauucc-3′
(SEQ ID NO: 149)
5′-uauugaaguuuugugauccac-3′
(SEQ ID NO: 155)
5′-ugauuucccaaguaaaaagac-3′
(SEQ ID NO: 156)
5′-uuuuucuccacacucaucauc-3′

In certain embodiments, the sense and antisense strands comprise nucleotide sequences selected from the group consisting of

(SEQ ID NO: 22)
5′-gacucucaaguuuuucaugua-3′
(SEQ ID NO: 139)
5′-uacaugaaaaacuugagaguc-3′
(SEQ ID NO: 41)
5′-gaauggaagguuauacucuaa-3′
(SEQ ID NO: 158)
5′-uuagaguauaaccuuccauuc-3′
(SEQ ID NO: 3)
5′-gaaugacauauuucaaaaaca-3′
(SEQ ID NO: 120)
5′-uguuuuugaaauaugucauuc-3′
(SEQ ID NO: 6)
5′-guugaucagucuuuuuaugaa-3′
(SEQ ID NO: 123)
5′-uucauaaaaagacugaucaac-3′
(SEQ ID NO: 8)
5′-gaagaacuacauauaaacuaa-3′
(SEQ ID NO: 125)
5′-uuaguuuauauguaguucuuc-3′
(SEQ ID NO: 12)
5′-guaugucacuugaacucaaca-3′
(SEQ ID NO: 129)
5′-uguugaguucaagugacauac-3′
(SEQ ID NO: 30)
5′-ggaauagauggaucacaaaaa-3′
(SEQ ID NO: 147)
5′-uuuuugugauccaucuauucc-3′
(SEQ ID NO: 32)
5′-guggaucacaaaacuucaaua-3′
(SEQ ID NO: 149)
5′-uauugaaguuuugugauccac-3′
(SEQ ID NO: 38)
5′-gucuuuuuacuugggaaauca-3′
(SEQ ID NO: 155)
5′-ugauuucccaaguaaaaagac-3′
(SEQ ID NO: 39)
5′-gaugaugaguguggagaaaaa-3′
(SEQ ID NO: 156)
5′-uuuuucuccacacucaucauc-3′

In some embodiments, the dsRNA agent includes at least one modified nucleotide. In certain embodiments, all or substantially all of the nucleotides of the antisense strand are modified nucleotides. In some embodiments, the at least one modified nucleotide comprises: a 2′-O-methyl nucleotide, 2′-Fluoro nucleotide, 2′-deoxy nucleotide, 2′3′-seco nucleotide mimic, locked nucleotide, unlocked nucleic acid nucleotide (UNA), glycol nucleic acid nucleotide (GNA), 2′-F-Arabino nucleotide, 2′-methoyxyethyl nucleotide, abasic nucleotide, ribitol, inverted nucleotide, inverted abasic nucleotide, inverted 2′-Ome nucleotide, inverted 2′-deoxy nucleotide, 2′-amino-modified nucleotide, 2′-alkyl-modified nucleotide, mopholino nucleotide, and 3′-OMe nucleotide, a nucleotide including a 5′-phosphorothioate group, or a terminal nucleotide linked to a cholesteryl derivative or dodecanoic acid bisdecylamide group, a 2′-amino-modified nucleotide, a phosphoramidite, or a non-natural base including nucleotide. In some embodiments, the dsRNA agent includes an E-vinylphosphonate nucleotide at the 5′ end of the guide strand. In certain embodiments, the dsRNA agent includes at least one phosphorothioate internucleoside linkage. In certain embodiments, the sense strand includes at least one phosphorothioate internucleoside linkage. In some embodiments, the antisense strand includes at least one phosphorothioate internucleoside linkage. In some embodiments, the sense strand includes 1, 2, 3, 4, 5, or 6, phosphorothioate internucleoside linkages. In some embodiments, the antisense strand includes 1, 2, 3, 4, 5, or 6, phosphorothioate internucleoside linkages. In certain embodiments, all or substantially all of the nucleotides of the sense strand and the antisense strand are modified nucleotides. In some embodiments, the modified sense strand is a modified sense strand sequence set forth in one of Tables 2-5. In some embodiments, the modified antisense strand is a modified antisense strand sequence set forth in one of Tables 2-5. In certain embodiments, the sense strand is complementary or substantially complementary to the antisense strand, and the region of complementarity is between 16 and 23 nucleotides in length. In some embodiments, the region of complementarity is 19-21 nucleotides in length. In certain embodiments, the region of complementarity is 14, 15, 16, 17, 18, 19, 20, 21, 22, 23, 24, 25, 26, 27, 28, 29, or 30 nucleotides in length. In some embodiments, each strand is no more than 30 nucleotides in length. In some embodiments, each strand is no more than 25 nucleotides in length. In some embodiments, each strand is no more than 23 nucleotides in length. In some embodiments, each strand is no more than 21 nucleotides in length. In certain embodiments, the dsRNA agent includes at least one modified nucleotide and further includes one or more targeting groups or linking groups. In some embodiments, the one or more targeting groups or linking groups are conjugated to the sense strand. In some embodiments, the targeting group or linking group includes N-acetyl-galactosamine (GalNAc). In some embodiments, the targeting group has a structure:

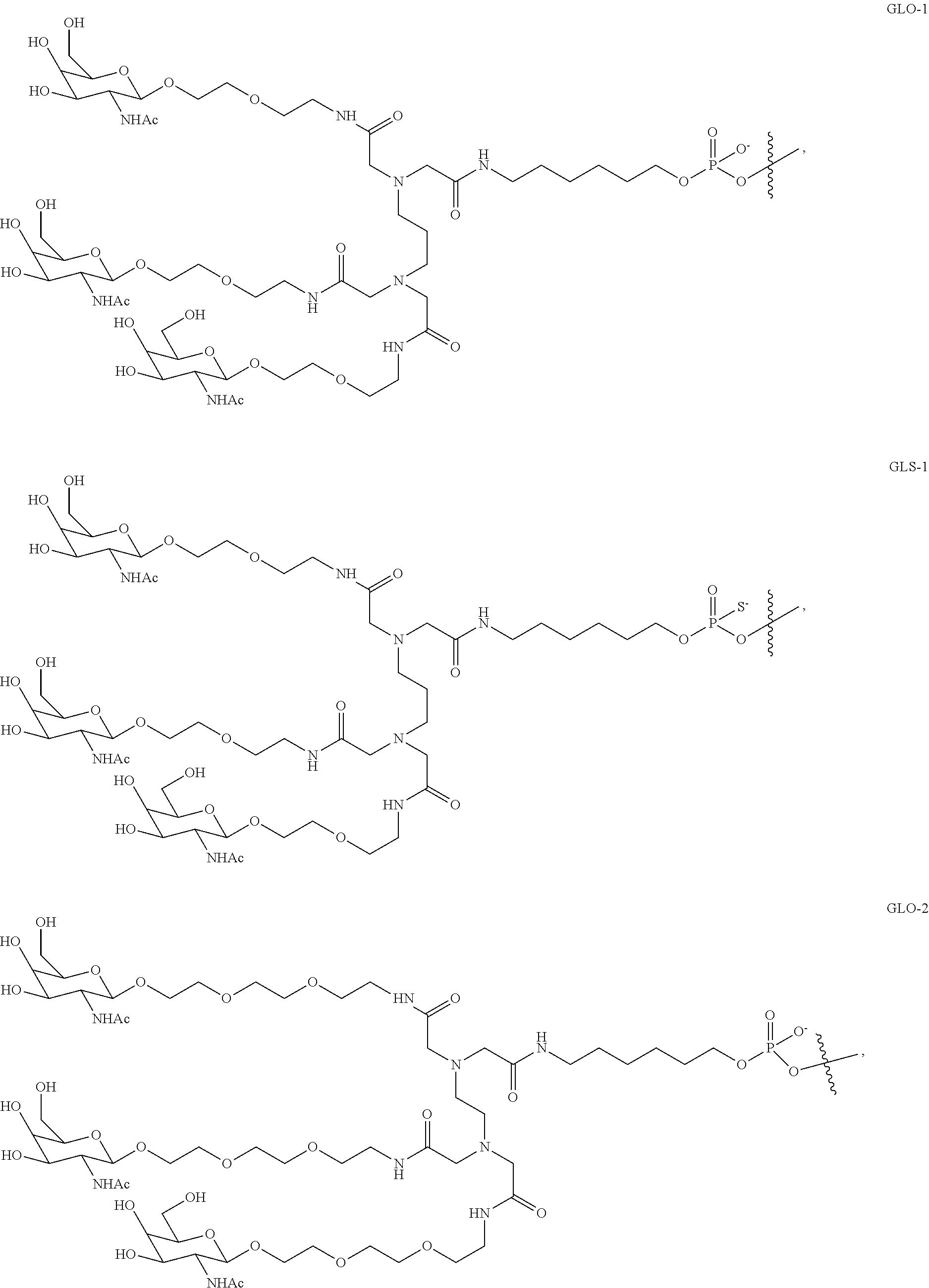 [SMILES: *OP(=O)([O-])OCCCCCCNC(=O)CN(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([S-])OCCCCCCNC(=O)CN(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([O-])OCCCCCCNC(=O)CN(CCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O]

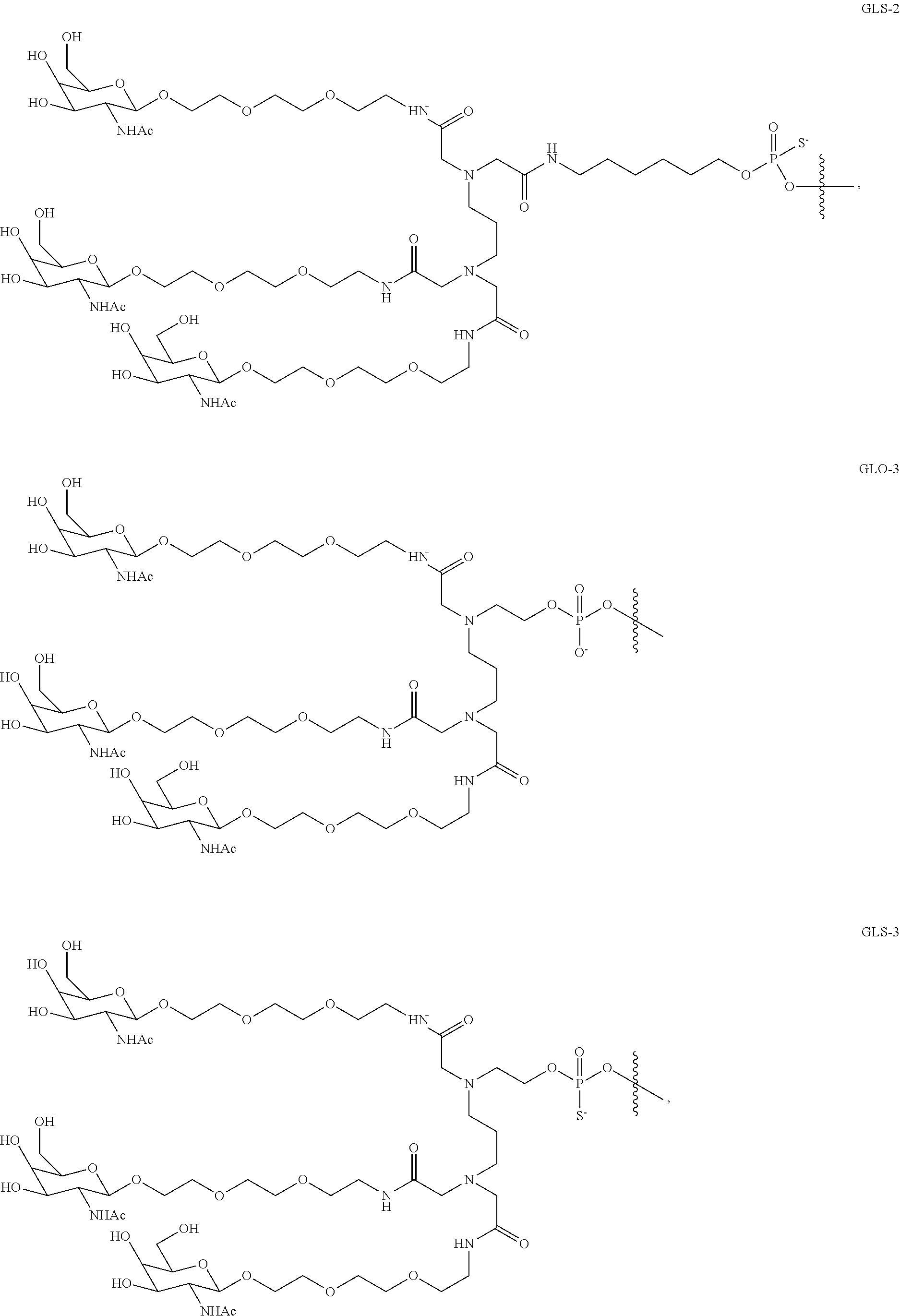 [SMILES: *OP(=O)([S-])OCCCCCCNC(=O)CN(CCCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O ; *OP(=O)([O-])OCCN(CCCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O ; *OP(=O)([S-])OCCN(CCCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O]

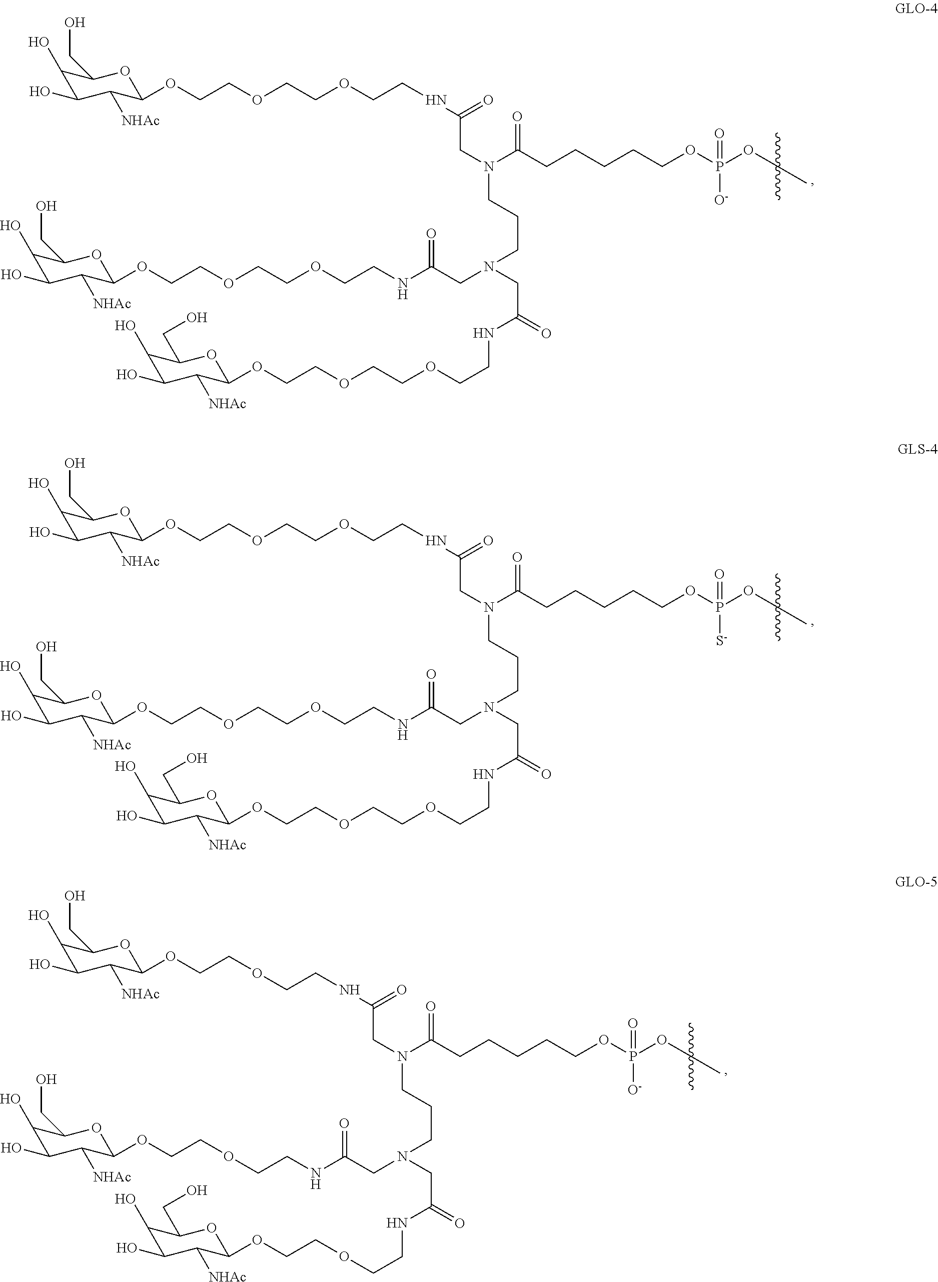 [SMILES: *OP(=O)([O-])OCCCCCC(=O)N(CCCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O ; *OP(=O)([S-])OCCCCCC(=O)N(CCCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O ; *OP(=O)([O-])OCCCCCC(=O)N(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O]

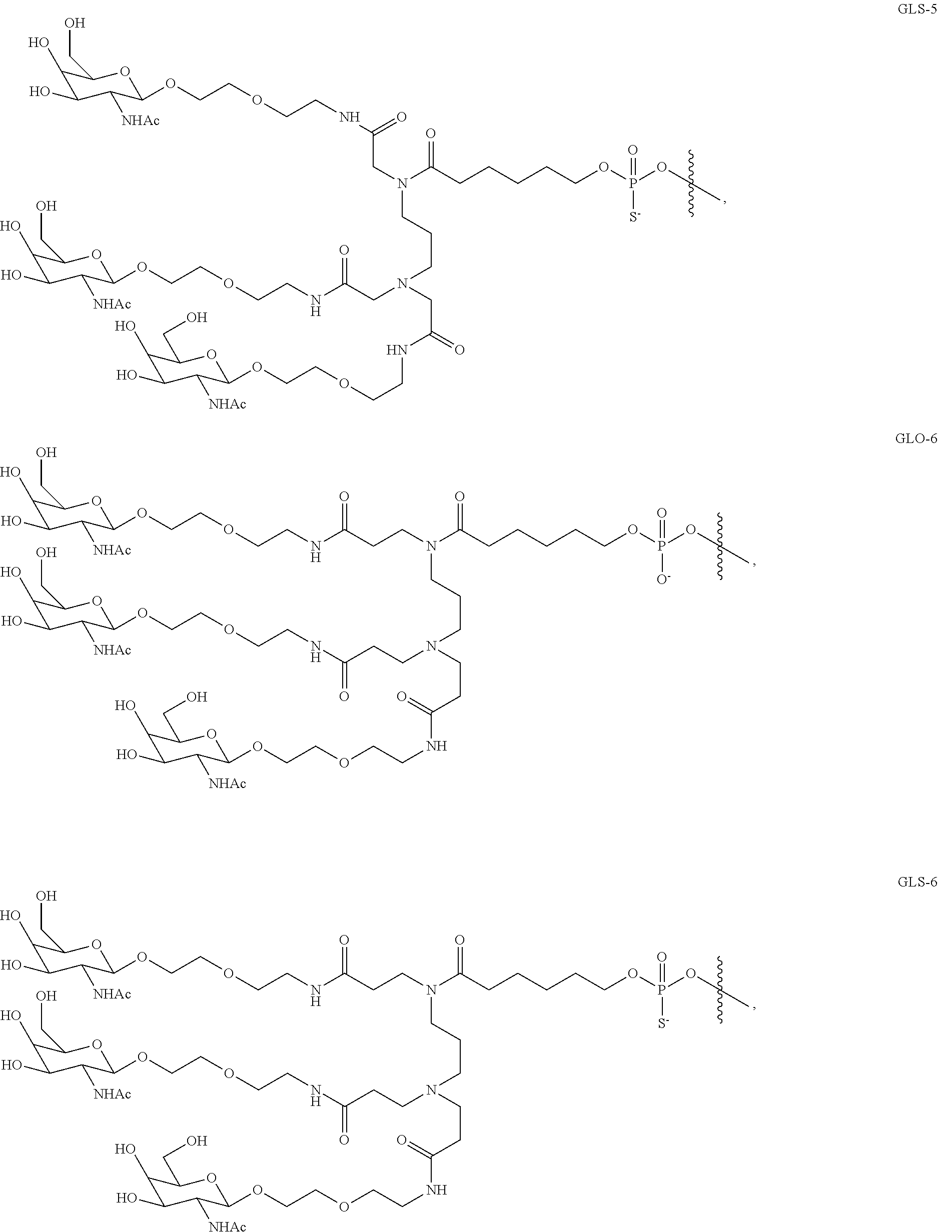 [SMILES: *OP(=O)([S-])OCCCCCC(=O)N(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([O-])OCCCCCC(=O)N(CCCN(CCC(=O)NCCOCCOC1OC(CO)C(O)C(O)C1NC(C)=O)CCC(=O)NCCOCCOC1OC(CO)C(O)C(O)C1NC(C)=O)CCC(=O)NCCOCCOC1OC(CO)C(O)C(O)C1NC(C)=O ; *OP(=O)([S-])OCCCCCC(=O)N(CCCN(CCC(=O)NCCOCCOC1OC(CO)C(O)C(O)C1NC(C)=O)CCC(=O)NCCOCCOC1OC(CO)C(O)C(O)C1NC(C)=O)CCC(=O)NCCOCCOC1OC(CO)C(O)C(O)C1NC(C)=O]

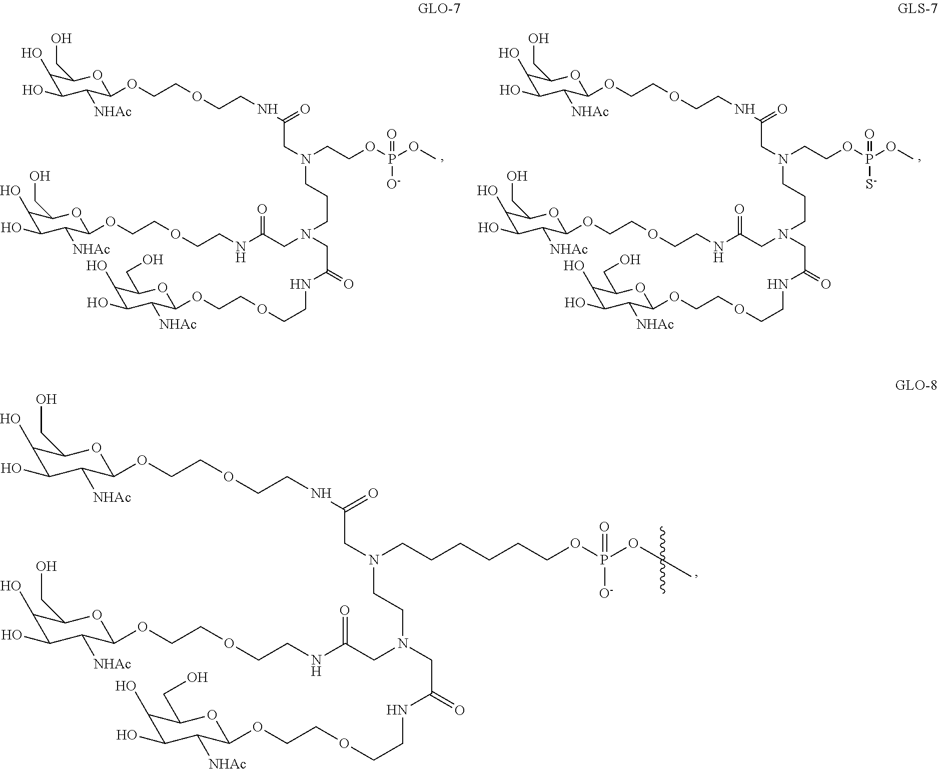 [SMILES: COP(=O)([O-])OCCN(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; COP(=O)([S-])OCCN(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([O-])OCCCCCCN(CCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O]

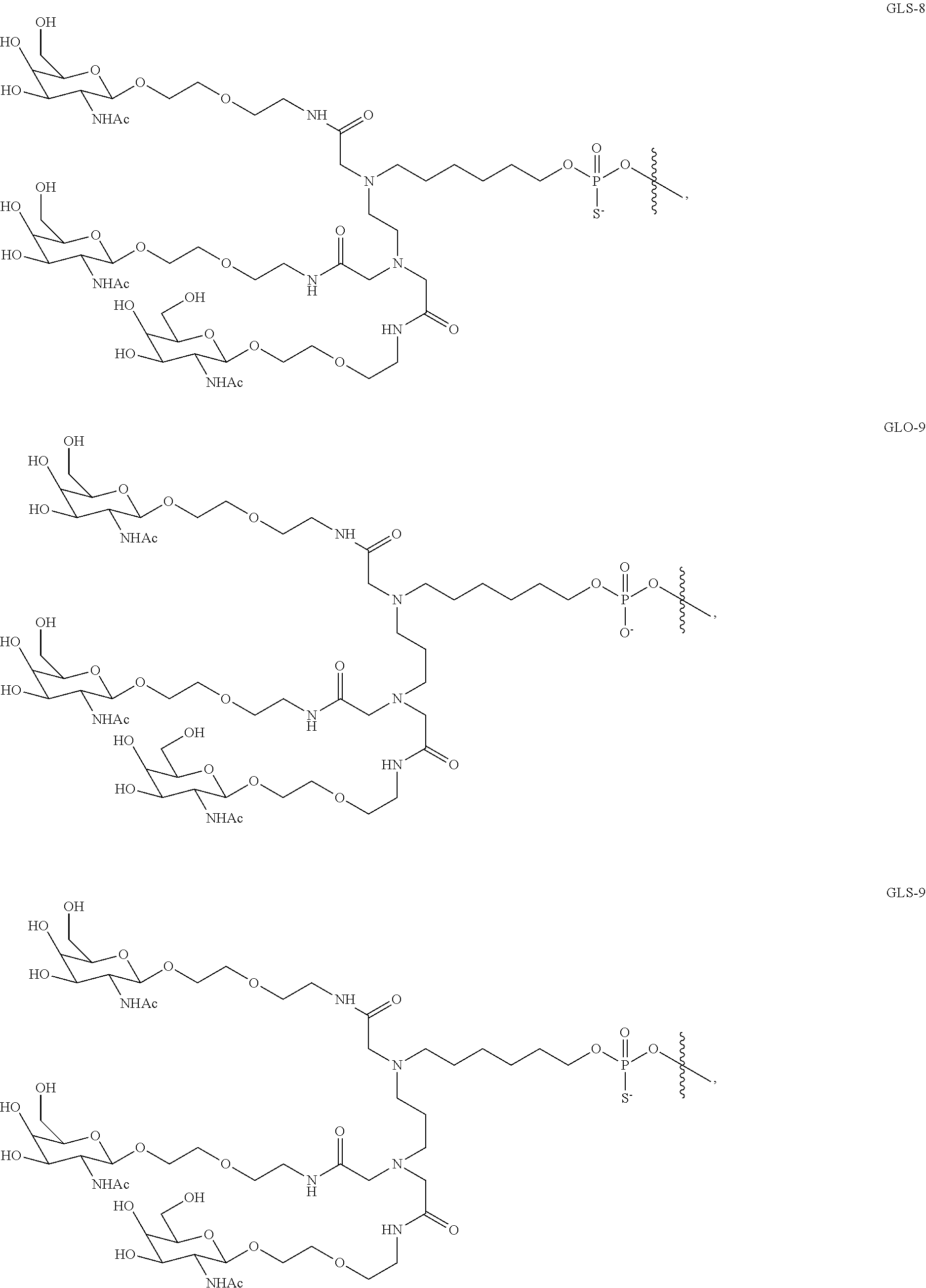 [SMILES: *OP(=O)([S-])OCCCCCCN(CCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([O-])OCCCCCCN(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([S-])OCCCCCCN(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O]

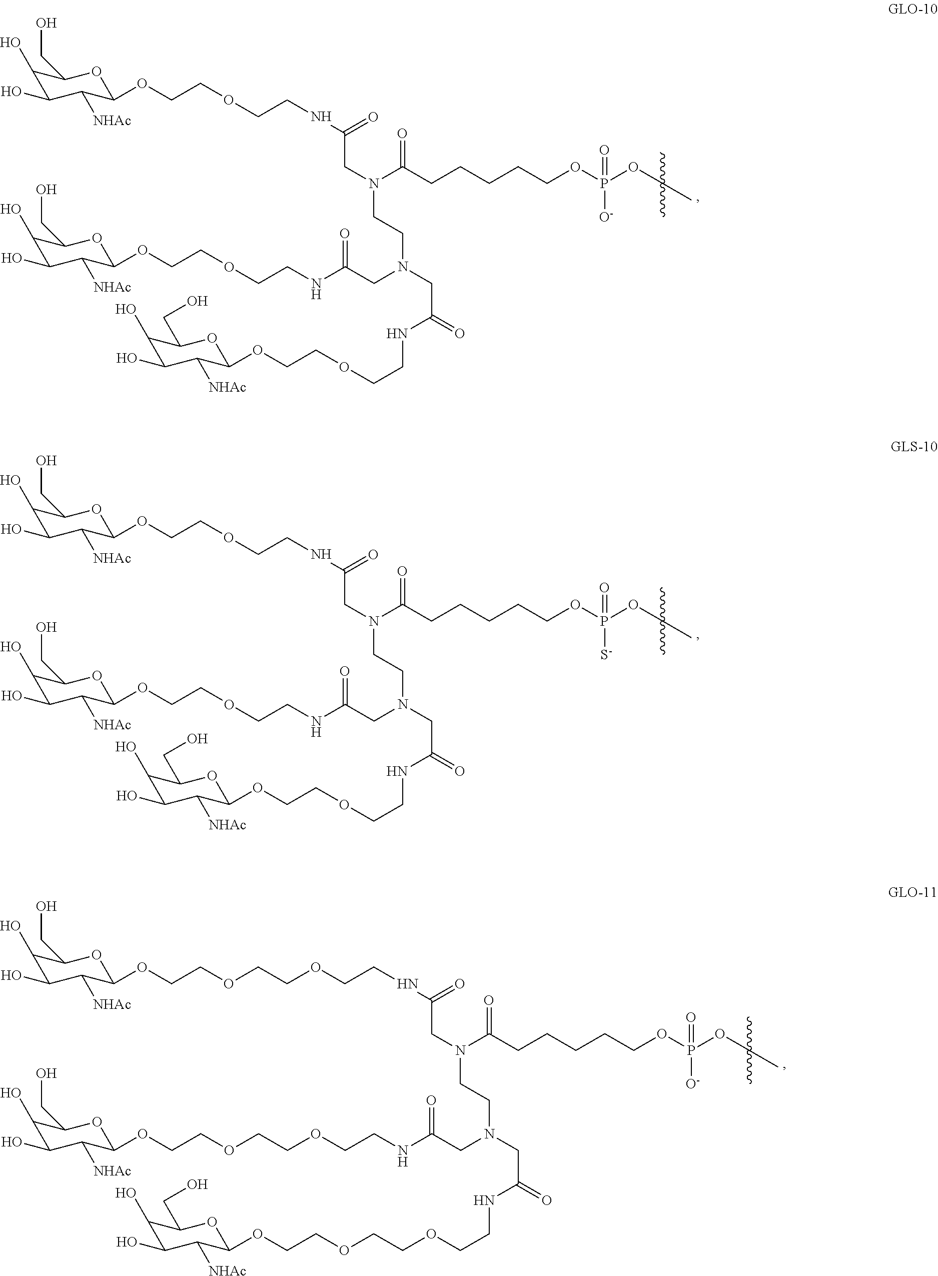 [SMILES: *OP(=O)([O-])OCCCCCC(=O)N(CCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([S-])OCCCCCC(=O)N(CCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([O-])OCCCCCC(=O)N(CCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O]

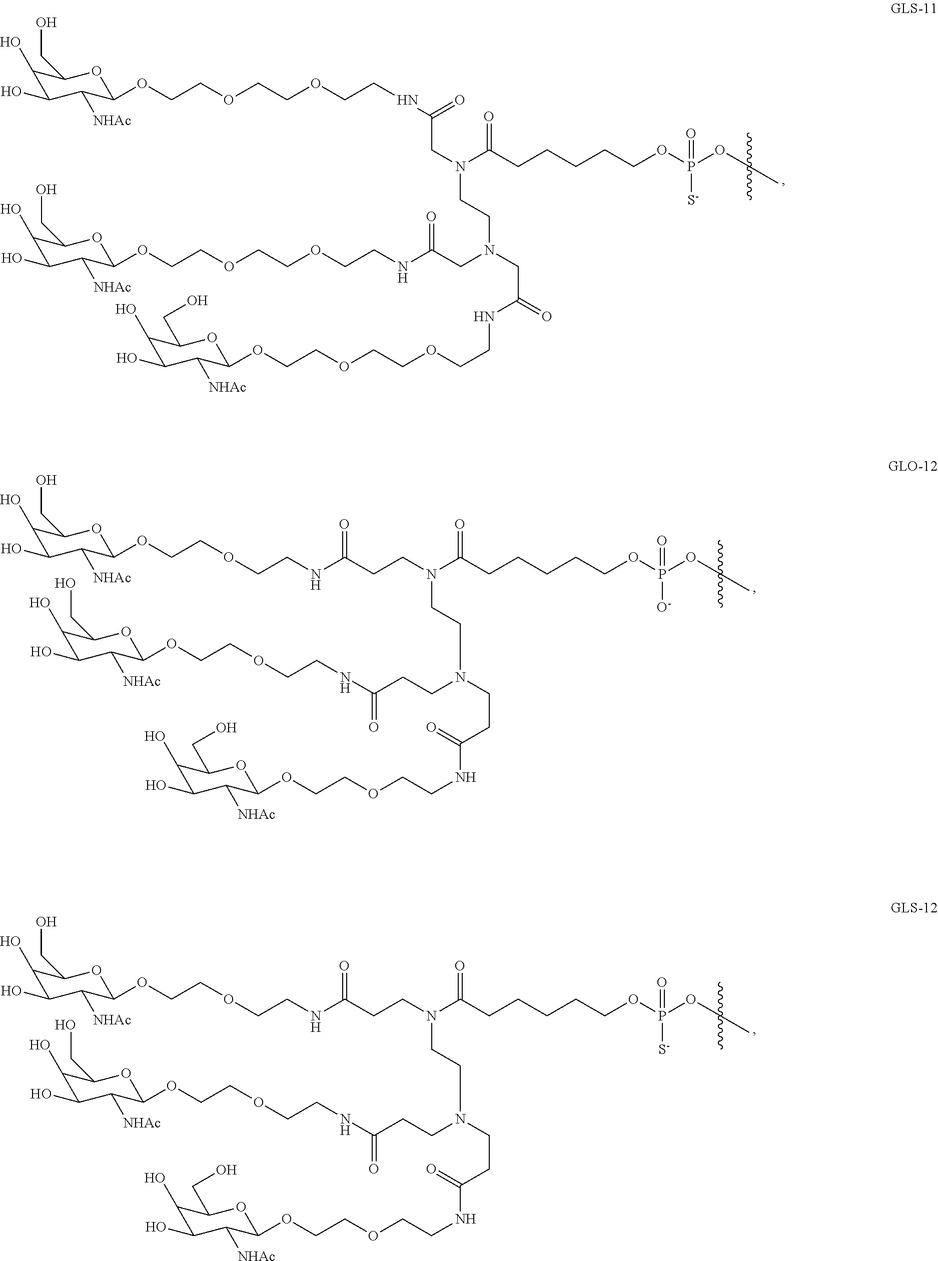 [SMILES: *OP(=O)([S-])OCCCCCC(=O)N(CCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O ; *OP(=O)([O-])OCCCCCC(=O)N(CCC(=O)NCCOCCOC1OC(CO)C(O)C(O)C1NC(C)=O)CCN(CCC(=O)NCCOCCOC1OC(CO)C(O)C(O)C1NC(C)=O)CCC(=O)NCCOCCOC1OC(CO)C(O)C(O)C1NC(C)=O ; *OP(=O)([S-])OCCCCCC(=O)N(CCC(=O)NCCOCCOC1OC(CO)C(O)C(O)C1NC(C)=O)CCN(CCC(=O)NCCOCCOC1OC(CO)C(O)C(O)C1NC(C)=O)CCC(=O)NCCOCCOC1OC(CO)C(O)C(O)C1NC(C)=O]

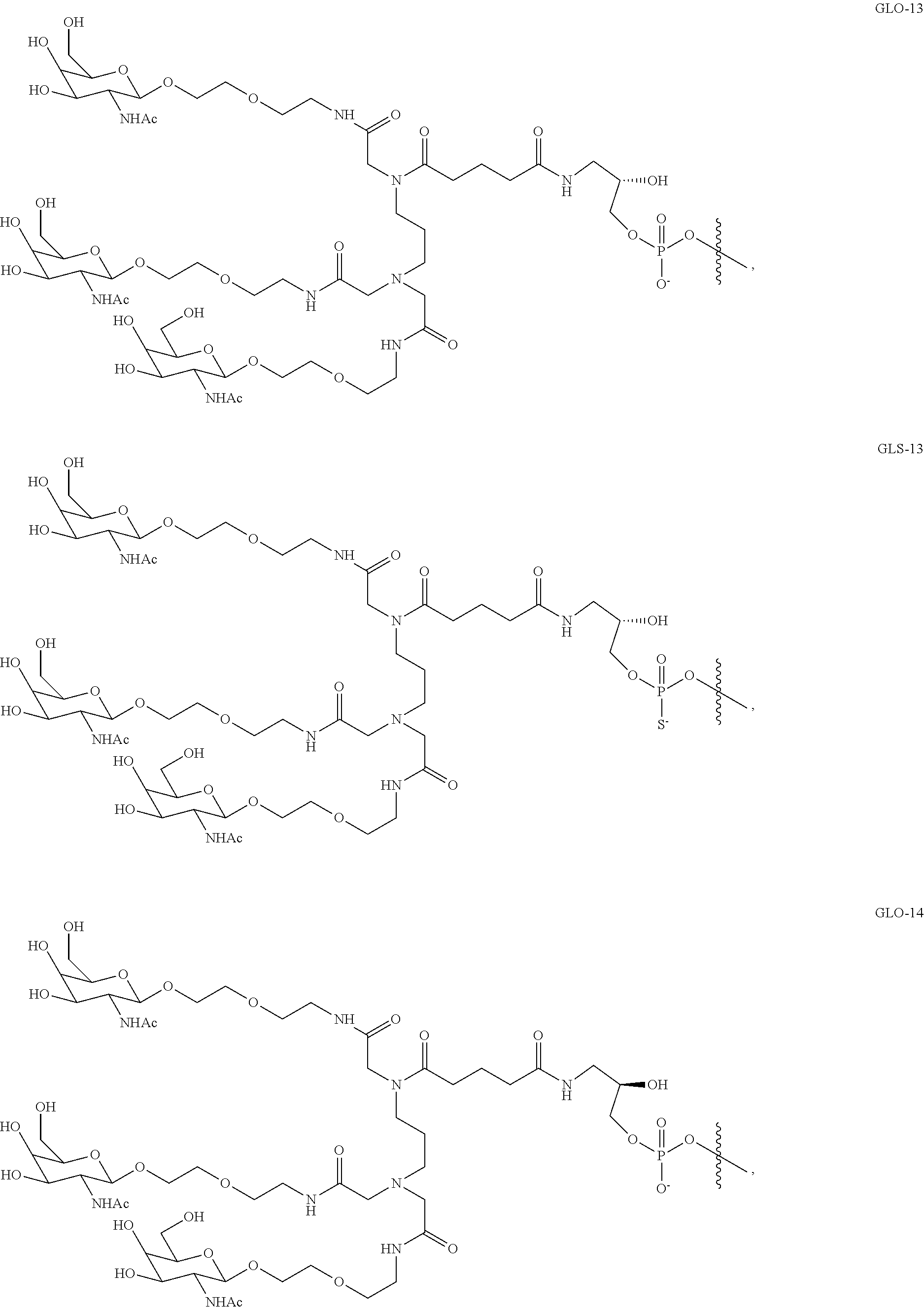 [SMILES: *OP(=O)([O-])OC[C@@H](O)CNC(=O)CCCC(=O)N(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([S-])OC[C@@H](O)CNC(=O)CCCC(=O)N(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([O-])OC[C@H](O)CNC(=O)CCCC(=O)N(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O]

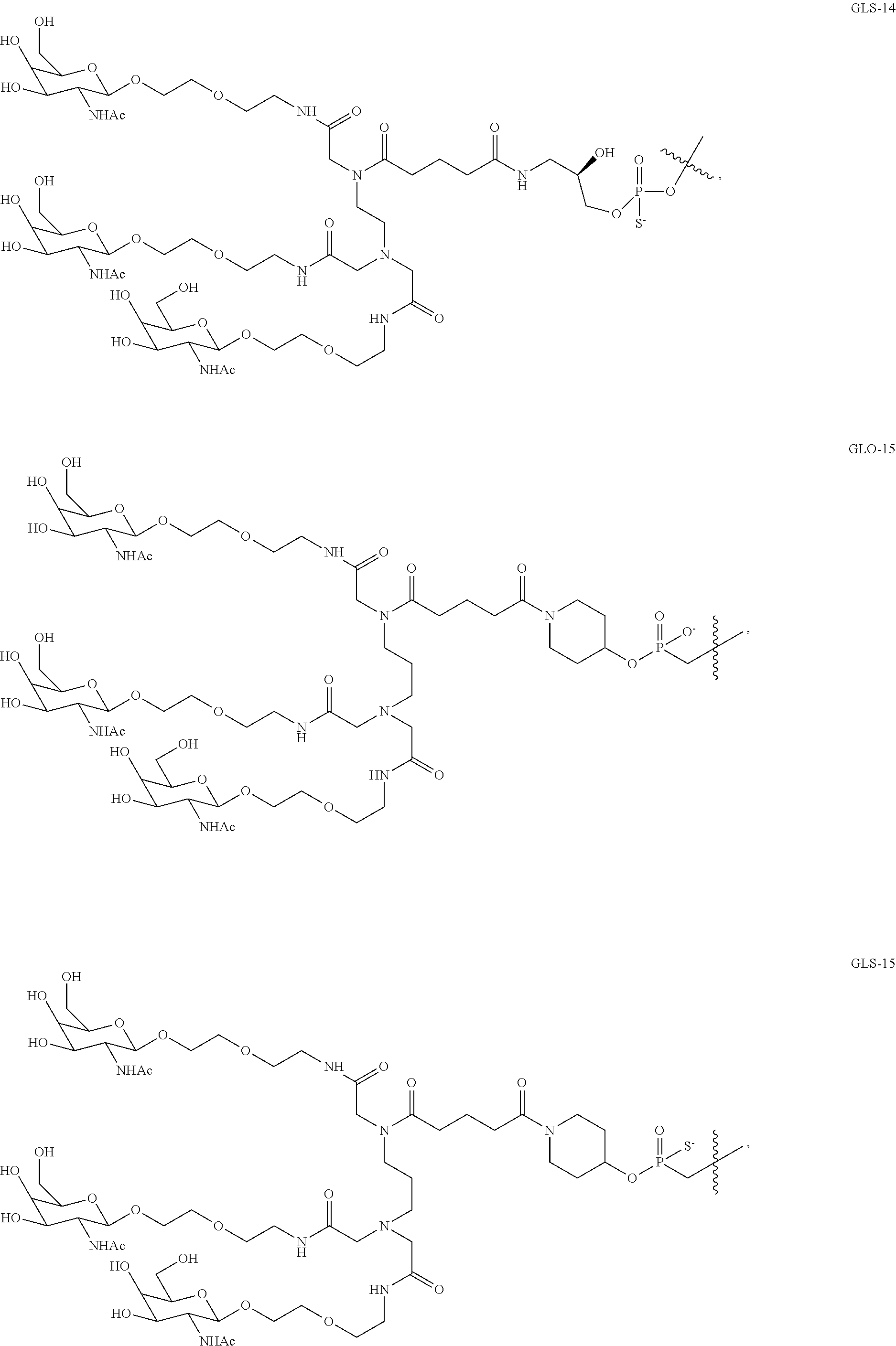 [SMILES: *OP(=O)([S-])OC[C@H](O)CNC(=O)CCCC(=O)N(CCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *CP(=O)([O-])OC1CCN(C(=O)CCCC(=O)N(CCCN(CC(=O)NCCOCCOC2OC(CO)C(O)C(O)C2NC(C)=O)CC(=O)NCCOCCOC2OC(CO)C(O)C(O)C2NC(C)=O)CC(=O)NCCOCCOC2OC(CO)C(O)C(O)C2NC(C)=O)CC1 ; *CP(=O)([S-])OC1CCN(C(=O)CCCC(=O)N(CCCN(CC(=O)NCCOCCOC2OC(CO)C(O)C(O)C2NC(C)=O)CC(=O)NCCOCCOC2OC(CO)C(O)C(O)C2NC(C)=O)CC(=O)NCCOCCOC2OC(CO)C(O)C(O)C2NC(C)=O)CC1]

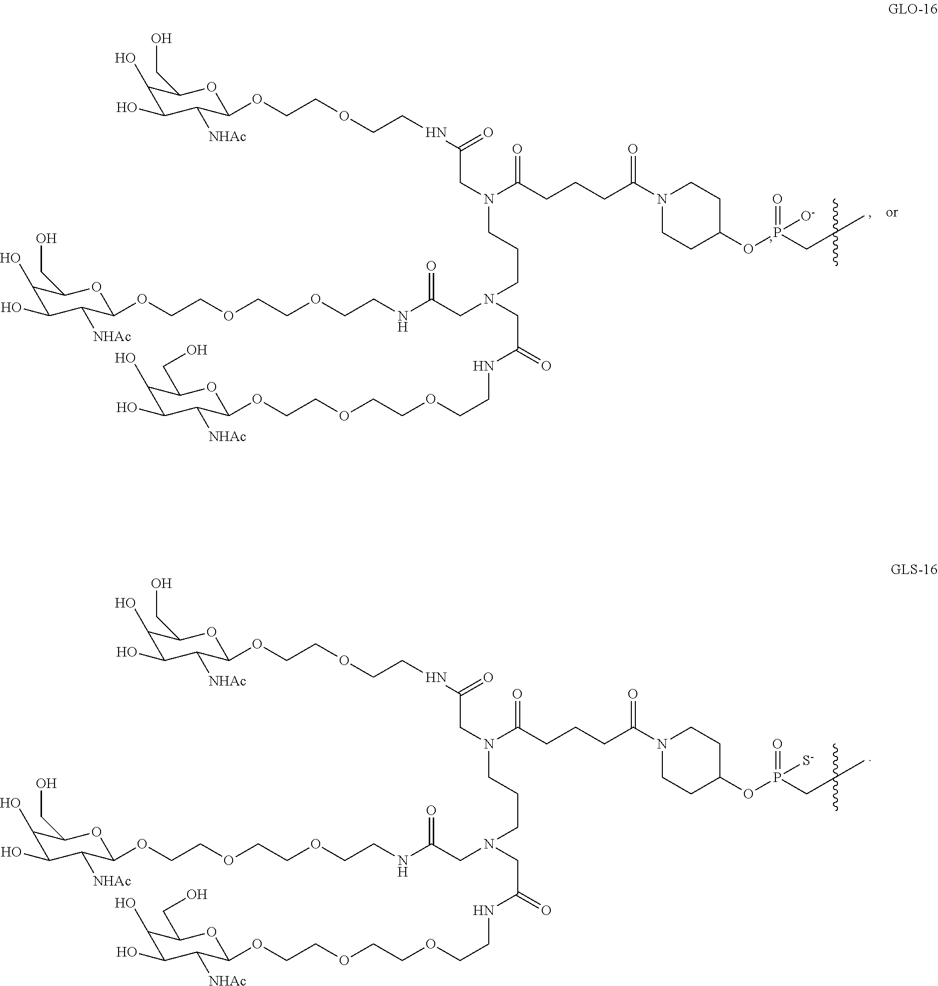 [SMILES: *CP(=O)([O-])OC1CCN(C(=O)CCCC(=O)N(CCCN(CC(=O)NCCOCCOCCOC2OC(CO)C(O)C(O)C2NC(C)=O)CC(=O)NCCOCCOCCOC2OC(CO)C(O)C(O)C2NC(C)=O)CC(=O)NCCOCCOC2OC(CO)C(O)C(O)C2NC(C)=O)CC1 ; *CP(=O)([S-])OC1CCN(C(=O)CCCC(=O)N(CCCN(CC(=O)NCCOCCOCCOC2OC(CO)C(O)C(O)C2NC(C)=O)CC(=O)NCCOCCOCCOC2OC(CO)C(O)C(O)C2NC(C)=O)CC(=O)NCCOCCOC2OC(CO)C(O)C(O)C2NC(C)=O)CC1]

In certain embodiments, the dsRNA agent includes a targeting group that is conjugated to the 5′-terminal end of the sense strand. In some embodiments, the dsRNA agent includes a targeting group that is conjugated to the 3-terminal end of the sense strand. In some embodiments, the antisense strand includes one inverted abasic residue at 3′-terminal end. In certain embodiments, the sense strand includes one or two inverted abasic residues at 3′ or/and 5′ terminal end. In some embodiments, the dsRNA agent has two blunt ends. In some embodiments, at least one strand includes a 3′ overhang of at least 1 nucleotide. In some embodiments, at least one strand includes a 3′ overhang of at least 2 nucleotides. In certain embodiments, the dsRNA comprises a duplex selected from the group consisting of AD00108, AD00108-1, AD00112, AD00112-1, AD00112-2, AD00133, AD00134, AD00135, AD00135-2, AD00136, AD00136-1, AD00142, AD00143, AD00143-2, AD00145 and AD00146. In certain embodiments, the dsRNA comprises a duplex selected from the group consisting of AD00112, AD00112-1, AD00112-2, AD00135, AD00135-2, AD00136 and AD00136-1. In certain embodiments, the dsRNA comprises a duplex selected from the group consisting of AD00112-1, AD00112-2, AD00135-2 and AD00136-1. In certain embodiments, the sense and antisense strands of the dsRNA comprise nucleotide sequences and modification selected from the group consisting of

```
(SEQ ID NO: 343)
5′-g*a*cucucaAgUuUuucaugu*a(GLO-0)-3′
(SEQ ID NO: 370)
5′-u*A*caugAaaaaCuUgAgag*u*c-3′
(SEQ ID NO: 347)
5′-g*a*auggaaGgUuAuacucua*a(GLO-0)-3′
(SEQ ID NO: 374)
5′-u*U*agagUauaaCcUuCcau*u*c-3′
(SEQ ID NO: 349)
5′-g*a*augacaUaUuUcaaaaac*a(GLO-0)-3′
(SEQ ID NO: 376)
5′-u*G*uuuuUgaaaUaUgUcau*u*c-3′
(SEQ ID NO: 350)
5′-g*u*ugaucaGuCuUuuuauga*a (GLO-0)-3′
  (SEQ ID NO: 377)
5′-u*U*cauaAaaagAcUgAuca*a*c-3′
(SEQ ID NO: 351)
5′-g*a*agaacuAcAuAuaaacua*a (GLO-0)-3′
(SEQ ID NO: 378)
5′-u*U*aguuUauauGuAgUucu*u*c-3′
(SEQ ID NO: 352)
5′-g*u*augucaCuUgAacucaac*a (GLO-0)-3′
(SEQ ID NO: 379)
5′-u*G*uugaGuucaAgUgAcau*a*c-3′
(SEQ ID NO: 358)
5′-c*g*aauagaUgGaUcacaaaa*a (GLO-0)-3′
(SEQ ID NO: 385)
5′-u*U*uuugUgaucCaUcUauu*c*g-3′
(SEQ ID NO: 359)
5′-a*u*ggaucaCaAaAcuucaau*a (GLO-0)-3′
(SEQ ID NO: 386)
5′-u*A*uugaAguuuUgUgAucc*a*u-3′
(SEQ ID NO: 361)
5′-g*u*cuuuuuAcUuGggaaauc*a (GLO-0)-3′
(SEQ ID NO: 388)
5′-u*G*auuuCccaaGuAaAaag*a*c-3′
(SEQ ID NO: 362)
5′-c*a*ugaugaGuGuGgagaaaa*a (GLO-0)-3′
(SEQ ID NO: 389)
5′-u*U*uuucUccacAcUcAuca*u*g-3′
(SEQ ID NO: 397)
5′-(GLS-5)*(Invab)*gacucucaAgUuUuucaugua*(Invab)-3′
(SEQ ID NO: 424)
5′-u*A*caugAaaaaCuUgAgag*u*c-3′
(SEQ ID NO: 401)
5′-(GLS-5)*(Invab)*gaauggaaGgUuAuacucuaa*(Invab)-3′
(SEQ ID NO: 428)
5′-u*U*agag UauaaCcUuCcau*u*c-3′
(SEQ ID NO: 403)
5′-(GLS-5)*(Invab)*gaaugacaUaUuUcaaaaaca*(Invab)-3′
(SEQ ID NO: 430)
5′-u*G*uuuuUgaaaUaUgUcau*u*c-3′
(SEQ ID NO: 404)
5′-(GLS-5)*(Invab)*guugaucaGuCuUuuuaugaa*(Invab)-3′
(SEQ ID NO: 431)
5′-u*U*cauaAaaagAcUgAuca*a*c-3′
(SEQ ID NO: 406)
5′-(GLS-5)*(Invab)*guaugucaCuUgAacucaaca*(Invab)-3′
(SEQ ID NO: 433)
5′-u*G*uugaGuucaAgUgAcau*a*c-3′
  (SEQ ID NO: 606)
5′-(GLS-15)*(Invab)*gaauggaaGgUuAuacucuaa*(Invab)-3′
(SEQ ID NO: 611)
5′-u*U*agag UauaaCcUuCcau*u*c-3′
(SEQ ID NO: 607)
5′-(GLS-5)*(Invab)*caagaacuAcAuAuaaacuaa*(Invab)-3′
(SEQ ID NO: 612)
5′-u*U*aguuUauauGuAgUucu*u*g-3′
(SEQ ID NO: 608)
5′-(GLS-15)*(Invab)*caagaacuAcAuAuaaacuaa*(Invab)-3′
(SEQ ID NO: 613)
5′-u*U*aguuUauauGuAgUucu*u*g-3′
(SEQ ID NO: 609)
5′-(GLS-15)*(Invab)*guaugucaCuUgAacucaaca*(Invab)-3′
(SEQ ID NO: 614)
5′-u*G*uugaGuucaAgUgAcau*a*c-3′
(SEQ ID NO: 610)
5′-(GLS-5)*(Invab)*guggaucaCaAaAcuucaaua*(Invab)-3′
(SEQ ID NO: 615)
5′-u*A*uugaAguuuUgUgAucc*a*c-3′
(SEQ ID NO: 616)
5′-(GLS-15)*(Invab)*gacucucaAgUuUuucaugua*(Invab)-3′
(SEQ ID NO: 617)
5′-u*A*caugAaaaaCuUgAgag*u*c-3′
(SEQ ID NO: 618)
5′-(GLS-15)*(Invab)*gaaugacaUaUuUcaaaaaca*(Invab)-3′
(SEQ ID NO: 619)
5′-u*G*uuuuUgaaaUaUgUcau*u*c-3′
  (SEQ ID NO: 620)
5′-(GLS-15)*(Invab)*guugaucaGuCuUuuuaugaa*(Invab)-3′
(SEQ ID NO: 621)
5′-u*U*cauaAaaagAcUgAuca*a*c-3′
(SEQ ID NO: 622)
5′-(GLS-15)*(Invab)*cgaauagaUgGaUcacaaaaa*(Invab)-3′
(SEQ ID NO: 624)
5′-u*U*uuugUgaucCaUcUauu*c*g-3′
(SEQ ID NO: 623)
5′-(GLS-15)*(Invab)*guggaucaCaAaAcuucaaua*(Invab)-3′
(SEQ ID NO: 625)
5′-u*A*uugaAguuuUgUgAucc*a*u-3′
(SEQ ID NO: 626)
5′-(GLS-15)*(Invab)*gucuuuuuAcUuGggaaauca*(Invab)-3′
  (SEQ ID NO: 627)
5′-u*G*auuuCccaaGuAaAaag*a*c-3′
(SEQ ID NO: 628)
5′-(GLS-15)*(Invab)*caugaugaGuGuGgagaaaaa*(Invab)-3′
(SEQ ID NO: 629)
5′-u*U*uuucUccacAcUcAuca*u*g-3′.
```

According to an aspect of the invention, a composition is provided that includes any embodiment of the aforementioned dsRNA agent aspect of the invention. In certain embodiments, the composition also includes a pharmaceutically acceptable carrier. In some embodiments, the composition also includes one or more additional therapeutic agents. In certain embodiments, the composition is packaged in a kit, container, pack, dispenser, pre-filled syringe, or vial. In some embodiments, the composition is formulated for subcutaneous administration or is formulated for intravenous (IV) administration.

According to another aspect of the invention a cell is provided that includes any embodiment of an aforementioned dsRNA agent aspect of the invention. In some embodiments, the cell is a mammalian cell, optionally a human cell.

According to another aspect of the invention, a method of inhibiting the expression of an ANGPTL3 gene in a cell, is provided, the method including: (i) preparing a cell including an effective amount of any embodiment of the aforementioned dsRNA agent aspect of the invention or any embodiment of an aforementioned composition of the invention. In certain embodiments, the method also includes: (ii) maintaining the prepared cell for a time sufficient to obtain degradation of the mRNA transcript of an ANGPTL3 gene, thereby inhibiting expression of the ANGPTL3 gene in the cell. In some embodiments, the cell is in a subject and the dsRNA agent is administered to the subject subcutaneously. In some embodiments, the cell is in a subject and the dsRNA agent is administered to the subject by IV administration. In certain embodiments, the method also includes assessing inhibition of the ANGPTL3 gene, following the administration of the dsRNA agent to the subject, wherein a means for the assessing comprises: (i) determining one or more physiological characteristics of an ANGPTL3-associated disease or condition in the subject and (ii) comparing the determined physiological characteristic(s) to a baseline pre-treatment physiological characteristic of the ANGPTL3-associated disease or condition and/or to a control physiological characteristic of the ANGPTL3-associated disease or condition, wherein the comparison indicates one or more of a presence or absence of inhibition of expression of the ANGPTL3 gene in the subject. In some embodiments, the determined physiological characteristic is one or more of: the subject's serum lipid level, the subject's serum HDL level, the subject's HDL:LDL ratio, the subject's serum triglyceride level, and the amount of fat in the subject's liver. In some embodiments, a reduction in one or more of the subject's serum lipid level, the subject's serum HDL level, the subject's serum triglyceride level, and the amount of fat in the subject's liver indicates reduction of AGNPTL3 gene expression in the subject.

According to another aspect of the invention, a method of inhibiting expression of an ANGPTL3 gene in a subject, is provided, the method including administering to the subject an effective amount of an embodiment of the aforementioned dsRNA agent aspect of the invention or an embodiment of an aforementioned composition of the invention. In some embodiments, the dsRNA agent is administered to the subject subcutaneously. In certain embodiments, the dsRNA agent is administered to the subject by IV administration. In some embodiments, the method also includes: assessing inhibition of the ANGPTL3 gene, following the administration of the dsRNA agent, wherein a means for the assessing comprises: (i) determining one or more physiological characteristics of an ANGPTL3-associated disease or condition in the subject and (ii) comparing the determined physiological characteristic(s) to a baseline pre-treatment physiological characteristic of the ANGPTL3-associated disease or condition and/or to a control physiological characteristic of the ANGPTL3-associated disease or condition, wherein the comparison indicates one or more of a presence or absence of inhibition of expression of the ANGPTL3 gene in the subject. In some embodiments, the determined physiological characteristic is one or more of: the subject's serum lipid level, the subject's serum HDL level, the subject's HDL:LDL ratio, the subject's serum triglyceride level, and the amount of fat in the subject's liver. In certain embodiments, a reduction in one or more of the subject's serum lipid level, the subject's serum HDL level, the subject's serum triglyceride level, and the amount of fat in the subject's liver indicates reduction of AGNPTL3 gene expression in the subject.

According to another aspect of the invention, a method of treating a disease or condition associated with the presence of ANGPTL3 protein is provided, the method including: administering to a subject an effective amount of an embodiment of any aforementioned dsRNA agent aspect of the invention or an embodiment of any aforementioned composition of the invention., to inhibit ANGPTL3 gene expression. In some embodiments, the disease or condition is one or more of: hyperlipidemia, hypertriglyceridemia, abnormal lipid and/or cholesterol metabolism, homozygous and heterozygous familial hypercholesterolemia, statin resistant hypercholesterolemia, cardiometabolic disease, obesity, atherosclerosis, type II diabetes mellitus, cardiovascular disease, coronary artery disease, non-alcoholic steatohepatitis, non-alcoholic fatty liver disease, pancreatitis caused by hypertriglyceridemia. In some embodiments, the method also includes: administering an additional therapeutic regimen to the subject. In some embodiments, the additional therapeutic regimen includes a treatment for the ANGPTL3-associated disease or condition. In certain embodiments, the additional therapeutic regimen comprises: administering to the subject one or more ANGPTL3 antisense polynucleotides of the invention, administering to the subject a non-ANGPTL3 dsRNA therapeutic agent, and a behavioral modification in the subject. In some embodiments, the non-ANGPTL3 dsRNA therapeutic agent is one of more of: (i) a statin; (ii) one or more of an antibody, antisense oligonucleotide (ASO), and a PCSK9 siRNA molecule capable of reducing PCSK9 expression; (iii) a therapeutic agent capable of reducing lipid accumulation in a subject, and (iv) a therapeutic agent capable of reducing cholesterol levels and/or accumulation in a subject. In some embodiments, the dsRNA agent is administered to the subject subcutaneously. In certain embodiments, the dsRNA agent is administered to the subject by IV administration. In some embodiments, the method also includes determining an efficacy of the administered double-stranded ribonucleic acid (dsRNA) agent in the subject. In some embodiments, a means of determining an efficacy of the treatment in the subject comprises: (i) determining one or more physiological characteristics of the ANGPTL3-associated disease or condition in the subject and (ii) comparing the determined physiological characteristic(s) to a baseline pre-treatment physiological characteristic of the ANGPTL3-associated disease or condition wherein the comparison indicates one or more of a presence, absence, and level of efficacy of the administration of the double-stranded ribonucleic acid (dsRNA) agent to the subject. In some embodiments, the determined physiological characteristic is: the subject's serum lipid level, the subject's HDL level, the subjects HDL:LDL ratio, the subject's serum triglyceride level, and the amount of fat in the subject's liver. In certain embodiments, a reduction in one or more of the subject's serum lipid level, the subject's serum HDL level, the subject's serum triglyceride level, and the amount of fat in the subject's liver indicates the presence of efficacy of the administration of the double-stranded ribonucleic acid (dsRNA) agent to the subject.

According to another aspect of the invention, a method of decreasing a level of ANGPTL3 protein in a subject compared to a baseline pre-treatment level of ANGPTL3 protein in the subject, is provided, the method including administering to the subject an effective amount of an embodiment of any aforementioned dsRNA agent aspect of the invention or an embodiment of any aforementioned composition of the invention, to decrease the level of ANGPTL3 gene expression. In some embodiments, the dsRNA agent is administered to the subject subcutaneously or is administered to the subject by IV administration.

According to another aspect of the invention, a method of altering a physiological characteristic of an ANGPTL3-associated disease or condition in a subject compared to a baseline pre-treatment physiological characteristic of the ANGPTL3-associated disease or condition in the subject is provided, the method including administering to the subject an effective amount of an embodiment of any aforementioned dsRNA agent aspect of the invention or an embodiment of any aforementioned composition of the invention, to alter the physiological characteristic of the ANGPTL3-associated disease or condition in the subject. IN some embodiments, the dsRNA agent is administered to the subject subcutaneously or is administered to the subject by IV administration. In certain embodiments, the physiological characteristic is one or more of: the subject's serum lipid level, the subject's HDL level, the subjects HDL:LDL ratio, the subject's serum triglyceride level, and the amount of fat in the subject's liver.

According to another aspect of the invention, the aforementioned dsRNA agent for use in a method of treating a disease or condition associated with the presence of ANGPTL3 protein is provided. In some embodiments, the disease or condition is one or more of: hyperlipidemia, hypertriglyceridemia, abnormal lipid and/or cholesterol metabolism, homozygous and heterozygous familial hypercholesterolemia, statin resistant hypercholesterolemia, cardiometabolic disease, obesity, atherosclerosis, type II diabetes mellitus, cardiovascular disease, coronary artery disease, non-alcoholic steatohepatitis, non-alcoholic fatty liver disease, pancreatitis caused by hypertriglyceridemia.

According to another aspect of the invention, an antisense polynucleotide agent for inhibiting expression of ANGPTL3 protein is provided, the agent including from 10 to 30 contiguous nucleotides, wherein at least one of the contiguous nucleotides is a modified nucleotide, and wherein the nucleotide sequence of the agent is about 80% complementary over its entire length to the equivalent region of the nucleotide sequence of SEQ ID NO: 235. In some embodiments, the equivalent region is any one of the target regions of SEQ ID NO: 235 and the complementary sequence is one provided in one of Tables 1-5. In certain embodiments, the antisense polynucleotide agent includes one of the antisense sequences provided in one of Tables 1-5.

According to another aspect of the invention, a composition including an embodiment of any aforementioned antisense polynucleotide agents is provided. In some embodiments, the composition also includes a pharmaceutically acceptable carrier. In some embodiments, the composition also includes one or more additional therapeutic agents for treatment of an ANGPTL3-associated disease or condition. In certain embodiments, the composition is packaged in a kit, container, pack, dispenser, pre-filled syringe, or vial. In certain embodiments, the composition is formulated for subcutaneous or IV administration.

According to another aspect of the invention a cell that includes an embodiment of any of the aforementioned antisense polynucleotide agents is provided. In some embodiments, the cell is a mammalian cell, optionally a human cell.

According to another aspect of the invention, a method of inhibiting the expression of an ANGPTL3 gene in a cell is provided, the method including: (i) preparing a cell including an effective amount of an embodiment of any aforementioned antisense polynucleotide agents. In some embodiments, the method also includes (ii) maintaining the cell prepared in (i) for a time sufficient to obtain degradation of the mRNA transcript of an ANGPTL3 gene, thereby inhibiting expression of the ANGPTL3 gene in the cell.

According to another aspect of the invention, a method of inhibiting expression of an ANGPTL3 gene in a subject is provided, the method including administering to the subject an effective amount of an embodiment of any of the aforementioned antisense polynucleotide agent.

According to another aspect of the invention, a method of treating a disease or condition associated with the presence of ANGPTL3 protein, the method including administering to a subject an effective amount of an embodiment of any of the aforementioned antisense polynucleotide agents or an embodiment of any aforementioned composition of the invention, to inhibit ANGPTL3 gene expression. In certain embodiments, the disease or condition is one or more of: hyperlipidemia, hypertriglyceridemia, abnormal lipid and/or cholesterol metabolism, homozygous and heterozygous familial hypercholesterolemia, statin resistant hypercholesterolemia, cardiometabolic disease, obesity, atherosclerosis, type II diabetes mellitus, cardiovascular disease, coronary artery disease, non-alcoholic steatohepatitis, non-alcoholic fatty liver disease, pancreatitis caused by hypertriglyceridemia.

According to another aspect of the invention, a method of decreasing a level of ANGPTL3 protein in a subject compared to a baseline pre-treatment level of ANGPTL3 protein in the subject is provided, the method including administering to the subject an effective amount of an embodiment of any of the aforementioned antisense polynucleotide agents or an embodiment of any aforementioned composition of the invention, to decrease the level of ANGPTL3 gene expression. In certain embodiments, the antisense polynucleotide agent is administered to the subject subcutaneously or by IV administration.

According to another aspect of the invention, an antisense polynucleotide agent for inhibiting expression of ANGPTL3 gene, is provided, the agent including from 10 to 30 contiguous nucleotides, wherein at least one of the contiguous nucleotides is a modified nucleotide, and wherein the nucleotide sequence of the agent is about 80% or about 85% complementary over its entire length to the equivalent region of the nucleotide sequence of SEQ ID NO: 235.

According to another aspect of the invention, a method of altering a physiological characteristic of an ANGPTL3-associated disease or condition in a subject compared to a baseline pre-treatment physiological characteristic of the ANGPTL3-associated disease or condition in the subject is provided, the method including administering to the subject an effective amount of an embodiment of any of the aforementioned antisense polynucleotide agents or an embodiment of any aforementioned composition of the invention, to alter the physiological characteristic of the ANGPTL-3 disease or condition in the subject. In some embodiments, the antisense polynucleotide agent is administered to the subject subcutaneously or by IV administration. In some embodiments, the physiological characteristic is one or more of: the subject's serum lipid level, the subject's HDL level, the subjects HDL:LDL ratio, the subject's serum triglyceride level, and the amount of fat in the subject's liver.

### BRIEF DESCRIPTION OF THE SEQUENCES

SEQ ID NOs: 1-117, 484-514 are shown in Table 1 and are sense strand sequences.

SEQ ID NOs: 118-234, 515-545 are shown in Table 1 and are antisense strand sequences.

SEQ ID NO: 235 is Homo sapiens angiopoietin like 3 (ANGPTL3) mRNA [NCBI Reference
Sequence: NM_014495.4]:
agaagaaaacagttccacgttgcttgaaattgaaaatcaagataaaaatgttcacaattaagctccttctttttattgttcctctagttatttcctc
cagaattgatcaagacaattcatcatttgattctctatctccagagccaaaatcaagatttgctatgttagacgatgtaaaaattttagccaatg
gcctccttcagttgggacatggtcttaaagactttgtccataagacgaagggccaaattaatgacatatttcaaaaactcaacatatttgatca
gtctttttatgatctatcgctgcaaaccagtgaaatcaaagaagaagaaaaggaactgagaagaactacatataaactacaagtcaaaaat
gaagaggtaaagaatatgtcacttgaactcaactcaaaacttgaaagcctcctagaagaaaaaattctacttcaacaaaaagtgaaatattt
agaagagcaactaactaacttaattcaaaatcaacctgaaactccagaacacccagaagtaacttcacttaaaacttttgtagaaaaacaa
gataatagcatcaaagaccttctccagaccgtggaagaccaatataaacaattaaaccaacagcatagtcaaataaaagaaatagaaaat
cagctcagaaggactagtattcaagaacccacagaaatttctctatcttccaagccaagagcaccaagaactactccctttcttcagttgaat
gaaataagaaatgtaaaacatgatggcattcctgctgaatgtaccaccatttataacagaggtgaacatacaagtggcatgtatgccatca
gacccagcaactctcaagtttttcatgtctactgtgatgttatatcaggtagtccatggacattaattcaacatcgaatagatggatcacaaaa
cttcaatgaaacgtgggagaactacaaatatggttttgggaggcttgatggagaattttggttgggcctagagaagatatactccatagtga
agcaatctaattatgttttacgaattgagttggaagactggaaagacaacaaacattatattgaatattctttttacttgggaaatcacgaaacc
aactatacgctacatctagttgcgattactggcaatgtccccaatgcaatcccggaaaacaaagatttggtgttttctacttgggatcacaaa
gcaaaaggacacttcaactgtccagagggttattcaggaggctggtggtggcatgatgagtgtggagaaaacaacctaaatggtaaatat
aacaaaccaagagcaaaatctaagccagagaggagaagaggattatcttggaagtctcaaaatggaaggttatactctataaaatcaacc
aaaatgttgatccatccaacagattcagaaagctttgaatgaactgaggcaaatttaaaaggcaataatttaaacattaacctcattccaagtt
aatgtggtctaataatctggtattaaatccttaagagaaagcttgagaaatagattttttttatcttaaagtcactgtctatttaagattaaacatac
aatcacataaccttaaagaataccgtttacatttctcaatcaaaattcttataatactatttgttttaaattttgtgatgtgggaatcaattttagatg
gtcacaatctagattataatcaataggtgaacttattaaataacttttctaaataaaaaatttagagacttttattttaaaaggcatcatatgagct
aatatcacaactttcccagtttaaaaaactagtactcttgttaaaactctaaacttgactaaatacagaggactggtaattgtacagttcttaaat
gttgtagtattaatttcaaaactaaaaatcgtcagcacagagtatgtgtaaaaatctgtaatacaaatttttaaactgatgcttcattttgctacaa
aataatttggagtaaatgtttgatatgatttatttatgaaacctaatgaagcagaattaaatactgtattaaaataagttcgctgtctttaaacaaa
tggagatgactactaagtcacattgactttaacatgaggtatcactataccttatttgttaaaatatatactgtatacattttatatattttaacactt
aatactatgaaaacaaataattgtaaaggaatcttgtcagattacagtaagaatgaacatatttgtggcatcgagttaaagtttatatttcccct
aaatatgctgtgattctaatacattcgtgtaggttttcaagtagaaataaacctcgtaacaagttactgaacgtttaaacagcctgacaagcat
gtatatatgtttaaaattcaataaacaaagacccagtccctaaattatagaaatttaaattattcttgcatgtttatcgacatcacaacagatccc
taaatccctaaatccctaaagattagatacaaattttttaccacagtatcacttgtcagaatttatttttaaatatgattttttaaaactgccagtaa
gaaattttaaattaaacccatttgttaaaggatatagtgcccaagttatatggtgacctacctttgtcaatacttagcattatgtatttcaaattatc
caatatacatgtcatatatatttttatatgtcacatatataaaagatatgtatgatctatgtgaatcctaagtaaatattttgttccagaaaagtaca
aaataataaaggtaaaaataatctataattttcaggaccacagactaagctgtcgaaattaacgctgatttttttagggccagaataccaaaat
ggctcctctcttcccccaaaattggacaatttcaaatgcaaaataattcattatttaatatatgagttgcttcctctatttggtttcc.
SEQ ID NO: 236 Mus musculus angiopoietin-like 3 (Angpt13), mRNA [NCBI Reference
Sequence: NM_013913.4]
acaggagggagaagttccaaattgcttaaaattgaataattgagacaaaaaatgcacacaattaaattattcctttttgttgttcctttagtaatt
gcatccagagtggatccagacctttcatcatttgattctgcaccttcagagccaaaatcaagatttgctatgttggatgatgtcaaaattttagc
gaatggcctcctgcagctgggtcatggacttaaagattttgtccataagactaagggacaaattaacgacatatttcagaagctcaacatatt
tgatcagtctttttatgacctatcacttcgaaccaatgaaatcaaagaagaggaaaaggagctaagaagaactacatctacactacaagtta
aaaacgaggaggtgaagaacatgtcagtagaactgaactcaaagcttgagagtctgctggaagagaagacagcccttcaacacaaggt
cagggctttggaggagcagctaaccaacttaattctaagcccagctggggctcaggagcacccagaagtaacatcactcaaaagttttgt
agaacagcaagacaacagcataagagaactcctccagagtgtggaagaacagtataaacaattaagtcaacagcacatgcagataaaa
gaaatagaaaagcagctcagaaagactggtattcaagaaccctcagaaaattctctttcttctaaatcaagagcaccaagaactactcccc
ctcttcaactgaacgaaacagaaaatacagaacaagatgaccttcctgccgactgctctgccgtttataacagaggcgaacatacaagtg
gcgtgtacactattaaaccaagaaactcccaagggtttaatgtctactgtgatacccaatcaggcagtccatggacattaattcaacaccgg
aaagatggctcacaggacttcaacgaaacatgggaaaactacgaaaagggctttgggaggctcgatggagaattttggttgggcctaga
gaagatctatgctatagtccaacagtctaactacattttacgactcgagctacaagactggaaagacagcaagcactacgttgaatactcct
ttcacctgggcagtcacgaaaccaactacacgctacatgtggctgagattgctggcaatatccctggggccctcccagagcacacagac
ctgatgttttctacatggaatcacagagcaaagggacagctctactgtccagaaagttactcaggtggctggtggtggaatgacatatgtg
gagaaaacaacctaaatggaaaatacaacaaacccagaaccaaatccagaccagagagaagaagagggatctactggagacctcag
agcagaaagctctatgctatcaaatcatccaaaatgatgctccagcccaccacctaagaagcttcaactgaactgagacaaaataaaaga
tcaataaattaaatattaaagtcctcccgatcactgtagtaatctggtattaaaattttaatggaaagcttgagaattgaatttcaattaggtttaa
actcattgttaagatcagatatcaccgaatcaacgtaaacaaaatttatctttttcaatc.

SEQ ID NOs: 237-336, 546-605 are shown in Table 2 with chemical modifications indicated by upper case: 2′-Fluoro; lower case: 2′-OMe; and thiophosphate: *.

SEQ ID NOs: 337-390 are shown in Table 3. A delivery molecule is indicated as “GLX-_” at the 3′ end of each sense strand. Chemical modifications are indicated as: upper case: 2′-Fluoro; lower case: 2′-OMe; and thiophosphate: *

SEQ ID NOs: 391-444, 606-629 are shown in Table 4. Chemical modifications are indicated as: upper case: 2′-Fluoro; lower case: 2′-OMe; thiophosphate: *; and Invab=inverted abasic.

SEQ ID NO: 445-482 are shown in Table 5. Chemical modifications are indicated with: upper case: 2′-Fluoro; lower case: 2′-OMe; thiophosphate: *; and Invab=inverted abasic.

SEQ ID NO: 483 is Predicted Macaca fascicularis angiopoietin like 3 (ANGPTL3), mRNA
[NCBI Reference Sequence: XM_005543185.2]:
tacaatttcaaattacctattaagttagttgctcatttctttgatttcatttagcattgatgtaactcaatgtggaagaaggttacattcgtgcaagtt
aacatggcttaatgattaactatattcacctgccaaccttgccttttctgtggcaaatattggtatatatagagttaagaagtctaggtctgcttc
cagaagaacacagttccacgctgcttgaaattgaaaatcaggataaaaatgttcacaattaagctccttctttttattgttcctctagttatttcct
ccagaattgaccaagacaattcatcatttgattctgtatctccagagccaaaatcaagatttgctatgttagacgatgtaaaaattttagccaat
ggcctccttcagttgggacatggtcttaaagactttgtccataagactaagggccaaattaatgacatatttcaaaaactcaacatatttgatc
agtctttttatgatctatcactgcaaaccagtgaaatcaaagaagaagaaaaggaactgagaagaactacatataaactacaagtcaaaaa
tgaagaggtaaagaatatgtcacttgaactcaactcaaaacttgaaagcctcctagaagaaaaaattctacttcaacaaaaagtgaaatattt
agaagagcaactaactaacttaattcaaaatcaacctgcaactccagaacatccagaagtaacttcacttaaaagttttgtagaaaaacaag
ataatagcatcaaagaccttctccagactgtggaagaacaatataagcaattaaaccaacagcatagtcaaataaaagaaatagaaaatc
agctcagaatgactaatattcaagaacccacagaaatttctctatcttccaagccaagagcaccaagaactactccctttcttcagctgaatg
aaataagaaatgtaaaacatgatggcattcctgctgattgtaccaccatttacaatagaggtgaacatataagtggcacgtatgccatcaga
cccagcaactctcaagtttttcatgtctactgtgatgttgtatcaggtagtccatggacattaattcaacatcgaatagatggatcacaaaactt
caatgaaacgtgggagaactacaaatatggtttcgggaggcttgatggagaattctggttgggcctagagaagatatactccatagtgaa
gcaatctaattacgttttacgaattgagttggaagactggaaagacaacaaacattatattgaatattctttttacttgggaaatcacgaaacc
aactatacgctacatgtagttaagattactggcaatgtccccaatgcaatcccggaaaacaaagatttggtgttttctacttgggatcacaaa
gcaaaaggacacttcagctgtccagagagttattcaggaggctggtggtggcatgatgagtgtggagaaaacaacctaaatggtaaatat
aacaaaccaagaacaaaatctaagccagagcggagaagaggattatcctggaagtctcaaaatggaaggttatactctataaaatcaac
caaaatgttgatccatccaacagattcagaaagctttgaatgaactgaggcaaatttaaaaggcaataaattaaacattaaactcattccaag
ttaatgtggtttaataatctggtattaaatccttaagagaaggcttgagaaatagatttttttatcttaaagtcactgtcaatttaagattaaacata
caatcacataaccttaaagaataccatttacatttctcaatcaaaattcttacaacactatttgttttatattttgtgatgtgggaatcaattttagat
ggtcgcaatctaaattataatcaacaggtgaacttactaaataacttttctaaataaaaaacttagagactttaattttaaaagtcatcatatgag
ctaatatcacaattttcccagtttaaaaaactagttttcttgttaaaactctaaacttgactaaataaagaggactgataattatacagttcttaaat
ttgttgtaatattaatttcaaaactaaaaattgtcagcacagagtatgtgtaaaaatctgtaatataaatttttaaactgatgcctcattttgctaca
aaataatctggagtaaatttttgataggatttatttatgaaacctaatgaagcaggattaaatactgtattaaaataggttcgctgtcttttaaaca
aatggagatgatgattactaagtcacattgactttaatatgaggtatcactataccttaacatatttgttaaaacgtatactgtatacattttgtgta
ttttaatacttaatactatgaaaacaagtaattgtaaacgtatcttgtcagattacaataggaatgaacatattggtgacatcgagttaaagttta
tatttcccctaaatatgctgcgattccaatatattcatgtaggttttcaagcagaaataaaccttgtaacaagttactgactaaacagcctgaca
agtatgtatatatgtttaaaattcaataaataaagacccagtcttctaaattataaaaatttaaattagtcttgcacaaattaaattattcatcacaa
aagatgtattgttatttttaagtcatttaagccctaaatccctaaagattagatataaattttttttgccagagtataaattgtcagaatttatttttaa
atatattttttaaaactaccagtaagaaattttaaattaaacccatttgttaaaggatatagtgcccaagttatacggtgacctacctttgtcaata
tttagcattatgtatttcaaattatccaatatacatgtcatatatatttttatatgttgcatatataaaagatatacacgatttatgtgaatcctatgta
aatattttgttccagaaaagtacaaaataataaaggtaaaaataatcca.

### BRIEF DESCRIPTION OF THE DRAWINGS

FIG. 1 is a graph showing the percent change of ANG3 in monkey plasma normalized to day 1 (before siRNA dosing).

FIG. 2 is a graph showing the percent change of HDL in monkey plasma normalized to day 1 (before siRNA dosing).

FIG. 3 is a graph showing the percent change of LDL in monkey plasma normalized to day 1 (before siRNA dosing).

FIG. 4 is a graph showing the percent change of total cholesterol (TC) in monkey plasma normalized to day 1 (before siRNA dosing).

FIG. 5 is a graph showing the percent change of triglyceride (TG) in monkey plasma normalized to day 1 (before siRNA dosing).

FIG. 6 is a graph showing the percent change of ANG3 in monkey plasma normalized to baseline.

FIG. 7 is a graph showing the percent change of HDL in monkey plasma normalized to baseline (before siRNA dosing).

FIG. 8 is a graph showing the percent change of LDL in monkey plasma normalized to baseline (before siRNA dosing).

FIG. 9 is a graph showing the percent change of total cholesterol (TC) in monkey plasma normalized to baseline (before siRNA dosing).

FIG. 10 is a graph showing the percent change of triglyceride (TG) in monkey plasma normalized to baseline (before siRNA dosing).

### DETAILED DESCRIPTION

The invention in part, includes RNAi agents, for example, though not limited to double stranded (ds) RNAi agents, which are capable of inhibiting Angiopoietin-like 3 (ANGPTL3) gene expression. The invention, in part also includes compositions comprising ANGPTL3 RNAi agents and methods of use of the compositions. ANGPTL3 RNAi agents disclosed herein may be attached to delivery compounds for delivery to cells, including to hepatocytes. Pharmaceutical compositions of the invention may include at least one ds ANGPTL3 agent and a delivery compound. In some embodiments of compositions and methods of the invention, the delivery compound is a GalNAc-containing delivery compound. ANGPTL3 RNAi agents delivered to cells are capable of inhibiting ANGPTL3 gene expression, thereby reducing activity in the cell of the ANGPTL3 protein product of the gene. dsRNAi agents of the invention can be used to treat ANGPTL3-associated diseases and conditions.

In some embodiments of the invention reducing ANGPTL3 expression in a cell or subject treats a disease or condition associated with ANGPTL3 expression in the cell or subject, respectively. Non-limiting examples of diseases and conditions that may be treated by reducing ANGPTL3 activity are: hyperlipidemia, hypertriglyceridemia, abnormal lipid and/or cholesterol metabolism, homozygous and heterozygous familial hypercholesterolemia, statin resistant hypercholesterolemia, cardiometabolic disease, obesity, atherosclerosis, type II diabetes mellitus, cardiovascular disease, coronary artery disease, non-alcoholic steatohepatitis, non-alcoholic fatty liver disease, pancreatitis caused by hypertriglyceridemia, or other diseases for which reducing a level and activity of ANGPTL3 protein is medically beneficial.

The following describes how to make and use compositions comprising ANGPTL3 single-stranded (ssRNA) and dsRNA agents to inhibit ANGPTL3 gene expression, as well as compositions and methods for treating diseases and conditions caused by or modulated by ANGPTL3 gene expression. The term “RNAi” is also known in the art, and may be referred to as “siRNA”.

As used herein, the term “RNAi” refers to an agent that comprises RNA and mediates targeted cleavage of an RNA transcript via an RNA-induced silencing complex (RISC) pathway. As is known in the art, an RNAi a target region refers to a contiguous portion of the nucleotide sequence of an mRNA molecule formed during the transcription of a gene, including messenger RNA (mRNA) that is a product of RNA processing of a primary transcription product. The target portion of the sequence will be at least long enough to serve as a substrate for RNAi-directed cleavage at or near that portion. A target sequence may be from 8-30 nucleotides long (inclusive), from 10-30 nucleotides long (inclusive), from 12-25 nucleotides long (inclusive), from 15-23 nucleotides long (inclusive), from 16-23 nucleotides long (inclusive), or from 18-23 nucleotides long (inclusive), including all shorter lengths within each stated range. In some embodiments of the invention, a target sequence is 9, 10, 11, 12, 13, 14, 15, 16, 17, 18, 19, 20, 21, 22, 23, 24, 25, or 26 nucleotides long. In certain embodiment a target sequence is between 9 and 26 nucleotides long (inclusive), including all sub-ranges and integers there between. For example, though not intended to be limiting, in certain embodiments of the invention a target sequence is 8, 9, 10, 11, 12, 13, 14, 15, 16, 17, 18, 19, 20, 21, 22, 23, 24, 25, 26, 27, 28, 29, or 30 nucleotides long, with the sequence fully or at least substantially complementary to at least part of an RNA transcript of an ANGPTL3 gene. Some aspects of the invention include pharmaceutical compositions comprising one or more ANGPTL3 dsRNA agents and a pharmaceutically acceptable carrier. In certain embodiments of the invention, an ANGPTL3 RNAi as described herein inhibits expression of ANGPTL3 protein.

As used herein, a “dsRNA agent” means a composition that contains an RNA or RNA-like (e.g., chemically modified RNA) oligonucleotide molecule that is capable of degrading or inhibiting translation of messenger RNA (mRNA) transcripts of a target mRNA in a sequence specific manner. Although not wishing to be limited to a particular theory, dsRNA agents of the invention may operate through the RNA interference mechanism (i.e., inducing RNA interference through interaction with the RNA interference pathway machinery (RNA-induced silencing complex or RISC) of mammalian cells), or by any alternative mechanism(s) or pathway(s). Methods for silencing genes in plant, invertebrate, and vertebrate cells are well known in the art [see, for example, (Sharp et al., Genes Dev. 2001, 15:485; Bernstein, et al., (2001) Nature 409:363; Nykanen, et al., (2001) Cell 107:309; and Elbashir, et al., (2001) Genes Dev. 15:188)], the disclosure of each of which is incorporated herein by reference in its entirety.]. Art-known gene silencing procedures can be used in conjunction with the disclosure provided herein to inhibit expression of ANGPTL3.

dsRNA agents disclosed herein are comprised of a sense strand and an antisense strand, and include, but are not limited to: short interfering RNAs (siRNAs), RNAi agents, micro RNAs (miRNAs), short hairpin RNAs (shRNA), and dicer substrates. The antisense strand of the dsRNA agents described herein is at least partially complementary to the mRNA being targeted. It is understood in the art that different lengths of dsRNA duplex structure can be used to inhibit target gene expression. For example, dsRNAs having a duplex structure of 19, 20, 21, 22, and 23 base pairs are known to be effective to induce RNA interference (Elbashir et al., EMBO 2001, 20:6877-6888). It is also known in the art that shorter or longer RNA duplex structures are also effective to induce RNA interference. ANGPTL3 dsRNAs in certain embodiments of the invention can include at least one strand of a length of minimally 21 nt or may have shorter duplexes based on one of the sequences set forth in any one of Tables 1-5 minus 1, 2, 3, or 4 nucleotides on one or both ends may also be effective as compared to the dsRNAs set forth in Tables 1-5, respectively. In some embodiments of the invention, ANGPTL3 dsRNA agents may have a partial sequence of at least 15, 16, 17, 18, 19, 20, or more contiguous nucleotides from one or more sequences of Tables 1-5, and differ in their ability to inhibit the expression of an ANGPTL3 gene by not more than 5, 10, 15, 20, 25, or 30% from the level of inhibition resulting from a dsRNA comprising the full sequence, which is also referred to herein as the “parent” sequence.

Certain embodiments of compositions and methods of the invention comprise a single-strand RNA in a composition and/or administered to a subject. For example, an antisense strand such as one listed in any one of Tables 1-5 may be a composition or in a composition administered to a subject to reduce ANGPTL3 polypeptide activity and/or expression of ANGPTL3 gene in the subject. Tables 1-5 show certain ANGPTL3 dsRNA agent antisense strand and sense strand core stretch base sequences. A single-strand antisense molecule that may be included in certain compositions and/or administered in certain methods of the invention are referred to herein as a “single-strand antisense agent” or an “antisense polynucleotide agent”. A single-strand sense molecule that may be included in certain compositions and/or administered in certain methods of the invention are referred to herein as a “single-strand sense agent” or a “sense polynucleotide agent”. The term “base sequence” is used herein in reference to a polynucleotide sequence without chemical modifications or delivery compounds. For example, the sense strand gaaagacuuuguccauaagaa (SEQ ID NO: 2) shown in Table 1 is the base sequence for SEQ ID NO: 337 in Table 3 and for SEQ ID NO: 391 in Table 4, with SEQ ID NO: 337 and SEQ ID NO: 391 shown with their chemical modifications and a delivery compound. Sequences disclosed herein may be assigned identifiers. For example, a single-stranded sense sequence may be identified with a “Sense strand SS #”; a single stranded antisense sequence may be identified with an “Antisense strand AS #” and a duplex that includes a sense strand and an antisense strand may be identified with a “Duplex AD #/AV #”.

Table 1 includes sense and antisense strands and provides the identification number of duplexes formed from the sense and antisense strand on the same line in Table 1. The sense strands SEQ ID Nos: 69-117 include a random nucleobase (n) at positions 1, 2, 3 and 21. The antisense strands SEQ ID Nos: 186-234 include a random nucleobase (n) at positions at positions 1, 19, 20, and 21. In certain embodiments of the invention an antisense sequence includes nucleobase u or nucleobase a in position 1 of the antisense sequence. In certain embodiments of the invention an antisense sequence includes nucleobase u in position 1 of the antisense sequence. In the sequences shown in Table 1 “n” can be any one of nucleobases a, u, c, g, and t and can be independently selected for the sense and antisense strand. As used in the context of “n” in sense and antisense strands, it will be understood that the nucleobase “n” selected and included in a position in a sense strand is not the same nucleobase as “n” in the antisense strand with which the sense strand pairs, but rather is generally complementary to the nucleobase “n” at the matching position in the opposite strand. As used herein, the term “matching position” in a sense and an antisense strands are the positions in each strand that “pair” when the two strands are duplexed strands. For example, in a 21 nucleobase sense strand and a 21 nucleobase antisense strand, nucleobase in position 1 of the sense strand and position 21 in the antisense strand are in “matching positions”. In yet another non-limiting example in a 23 nucleobase sense strand and a 23 nucleobase antisense strand, nucleobase 2 of the sense strand and position 22 of the antisense strand are in matching positions. In another non-limiting example, in an 18 nucleobase sense strand and an 18 nucleobase antisense strand, nucleobase in position 1 of the sense strand and nucleobase 18 in the antisense strand are in matching positions, and nucleobase 4 in the sense strand and nucleobase 15 in the antisense strand are in matching positions. A skilled artisan will understand how to identify matching positions in sense and antisense strands that are or will be duplexed strands and paired strands.

Although (n) can be any one of a, u, c, g or t, an “n” at position 1 of sense strand is generally complementary to (n) at position 21 of antisense strand. In two non-limiting examples, (1) if position 1 of sense strand is “g” then position 21 of antisense strand is “c”; and (2) if position 1 of sense strand is “a” then position 21 of antisense strand is “u” or “t”. This type of complimentary matching pairing applies to (n) at position 2 of sense strand and position 20 of antisense strand; (n) at position 21 of sense strand and position 1 of antisense strand. It will be understood that even though n can be any nucleotide at these positions, the nucleotides of sense and antisense strand are generally still complementary (match), however, in certain embodiments, they may have mismatch. For example, though not intended to be limiting, in some embodiments “n” can be “random”, meaning might but need not be complementary. In certain embodiments “n” is complementary. As a non-limiting example, “n” in position of 1 of antisense is “u” and “n” in position of 21 of sense strand is “a”.

The final column in Table 1 indicates a Duplex AD #/AV # for a duplex that includes the sense and antisense sequences in the same table row. For example, Table 1 discloses the duplex assigned Duplex AD #AD00007, which includes sense strand SEQ ID NO: 6 and antisense strand SEQ ID NO: 123. Thus, each row in Table 1 identifies a duplex of the invention, each comprising the sense and antisense sequences shown in the same row, with the assigned identifier for each duplex shown in the final column in the row.

In some embodiments of methods of the invention, an RNAi agent comprising a polynucleotide sequence shown in Table 1 is administered to a subject. In some embodiments of the invention an RNAi agent administered to a subject comprises is a duplex comprising at least one of the base sequences set forth in Table 1, including 0, 1, 2, 3, 4, 5, 6, 7, 8, 9, 10, 11, 12, 13, 14, 15, 16, 17, 18, 19, 20, 21, 22, 23, or 24 sequence modifications. In some embodiments of methods of the invention an RNAi agent comprising a polynucleotide sequence shown in Table 1 is attached to a delivery molecule, a non-limiting example of which is a delivery compound comprising a GalNAc compound.

TABLE 1
Unmodified ANGPTL3 RNAi agent antisense strand and sense strand sequences.

| | SEQ | | SEQ | |
|---|---|---|---|---|
| | ID | | ID | Duplex |
| Sense strand | NO | Antisense strand | NO | AD#/AV# |
| guuaaagacuuuguccauaaa | 1 | uuuauggacaaagucuuuaac | 118 | AD00001 |
| gaaagacuuuguccauaagaa | 2 | uucuuauggacaaagucuuuc | 119 | AD00002 |
| gaaugacauauuucaaaaaca | 3 | uguuuuugaaauaugucauuc | 120 | AD00004 |
| gaaacucaacauauuugauca | 4 | ugaucaaauauguugaguuuc | 121 | AD00005 |
| gaacucaacauauuugaucaa | 5 | uugaucaaauauguugaguuc | 122 | AD00006 |
| guugaucagucuuuuuaugaa | 6 | uucauaaaaagacugaucaac | 123 | AD00007 |
| ggaucagucuuuuuaugauca | 7 | ugaucauaaaaagacugaucc | 124 | AD00008 |
| gaagaacuacauauaaacuaa | 8 | uuaguuuauauguaguucuuc | 125 | AD00009 |
| gcuacauauaaacuacaagua | 9 | uacuuguaguuuauauguagc | 126 | AD00010 |
| gaagagguaaagaauauguca | 10 | ugacauauucuuuaccucuuc | 127 | AD00011 |
| gagguaaagaauaugucacua | 11 | uagugacauauucuuuaccuc | 128 | AD00012 |
| guaugucacuugaacucaaca | 12 | uguugaguucaagugacauac | 129 | AD00013 |
| gacucaaaacuugaaagccua | 13 | uaggcuuucaaguuuugaguc | 130 | AD00014 |
| gaacuugaaagccuccuagaa | 14 | uucuaggaggcuuucaaguuc | 131 | AD00015 |
| ggaaaaaauucuacuucaaca | 15 | uguugaaguagaauuuuuucc | 132 | AD00016 |
| ggaagagcaacuaacuaacua | 16 | uaguuaguuaguugcucuucc | 133 | AD00017 |
| guucaagaacccacagaaaua | 17 | uauuucuguggguucuugaac | 134 | AD00018 |
| gucaagaacccacagaaauua | 18 | uaauuucuguggguucuugac | 135 | AD00019 |
| gucucuaucuuccaagccaaa | 19 | uuuggcuuggaagauagagac | 136 | AD00020 |
| gcaagaacuacucccuuucua | 20 | uagaaagggaguaguucuugc | 137 | AD00021 |
| gcaacucucaaguuuuucaua | 21 | uaugaaaaacuugagaguugc | 138 | AD00022 |
| gacucucaaguuuuucaugua | 22 | uacaugaaaaacuugagaguc | 139 | AD00023 |
| gcucucaaguuuuucauguca | 23 | ugacaugaaaaacuugagagc | 140 | AD00024 |
| gcucaaguuuuucaugucuaa | 24 | uuagacaugaaaaacuugagc | 141 | AD00025 |
| gucaaguuuuucaugucuaca | 25 | uguagacaugaaaaacuugac | 142 | AD00026 |
| guuuucaugucuacugugaua | 26 | uaucacaguagacaugaaaac | 143 | AD00027 |
| gucaugucuacugugauguua | 27 | uaacaucacaguagacaugac | 144 | AD00028 |
| guggacauuaauucaacauca | 28 | ugauguugaauuaauguccac | 145 | AD00029 |
| gaacaucgaauagauggauca | 29 | ugauccaucuauucgauguuc | 146 | AD00030 |
| ggaauagauggaucacaaaaa | 30 | uuuuugugauccaucuauucc | 147 | AD00031 |
| gauagauggaucacaaaacua | 31 | uaguuuugugauccaucuauc | 148 | AD00032 |
| guggaucacaaaacuucaaua | 32 | uauugaaguuuugugauccac | 149 | AD00033 |
| gcaaaacuucaaugaaacgua | 33 | uacguuucauugaaguuuugc | 150 | AD00034 |
| ggggagaacuacaaauaugga | 34 | uccauauuuguaguucucccc | 151 | AD00035 |
| gagagaagauauacuccauaa | 35 | uuauggaguauaucuucucuc | 152 | AD00036 |
| ggagaagauauacuccauaga | 36 | ucuauggaguauaucuucucc | 153 | AD00037 |
| gagauauacuccauagugaaa | 37 | uuucacuauggaguauaucuc | 154 | AD00038 |
| gucuuuuuacuugggaaauca | 38 | ugauuucccaaguaaaaagac | 155 | AD00039 |
| gaugaugaguguggagaaaaa | 39 | uuuuucuccacacucaucauc | 156 | AD00040 |
| ggaaaacaaccuaaaugguaa | 40 | uuaccauuuagguuguuuucc | 157 | AD00041 |
| gaauggaagguuauacucuaa | 41 | uuagaguauaaccuuccauuc | 158 | AD00042 |
| ggguuauacucuauaaaauca | 42 | ugauuuuauagaguauaaccc | 159 | AD00043 |
| gucacaaaacuucaaugaaaa | 43 | uuuucauugaaguuuugugac | 160 | AD00044 |
| gcuugaaagccuccuagaaga | 44 | ucuucuaggaggcuuucaagc | 161 | AD00045 |
| gugaacucaacucaaaacuua | 45 | uaaguuuugaguugaguucac | 162 | AD00046 |
| gggacauuaauucaacaucga | 46 | ucgauguugaauuaauguccc | 163 | AD00047 |
| ggacauuaauucaacaucgaa | 47 | uucgauguugaauuaaugucc | 164 | AD00048 |
| guaauucaacaucgaauagaa | 48 | uucuauucgauguugaauuac | 165 | AD00049 |
| gaagggccaaauuaaugacaa | 49 | uugucauuaauuuggcccuuc | 166 | AD00050 |
| aagggccaaauuaaugacaua | 50 | uaugucauuaauuuggcccuu | 167 | AD00132 |
| gaaagauuuuguccauaagaa | 51 | uucuuauggacaaaaucuuuc | 168 | AD00178 |
| gcaaaaucaagauuugcuaua | 52 | uauagcaaaucuugauuuugc | 169 | AD00179 |
| guuaaagauuuuguccauaaa | 53 | uuuauggacaaaaucuuuaac | 170 | AD00180 |
| gaaaccaacuacacgcuacaa | 54 | uuguageguguaguugguuuc | 171 | AD00181 |
| gaagcucaacauauuugauca | 55 | ugaucaaauauguugagcuuc | 172 | AD00182 |
| gagcucaacauauuugaucaa | 56 | uugaucaaauauguugagcuc | 173 | AD00183 |
| guauuugaucagucuuuuuaa | 57 | uuaaaaagacugaucaaauac | 174 | AD00184 |
| gcacccagaaguaacaucaca | 58 | ugugauguuacuucugggugc | 175 | AD00185 |
| guaaagauuuuguccauaaga | 59 | ucuuauggacaaaaucuuuac | 176 | AD00186 |
| gcaguccauggacauuaauua | 60 | uaauuaauguccauggacugc | 177 | AD00187 |
| gaaagacuuuguccauaagaa | 61 | uucuuauggacaaagucuuuc | 178 | AD00102 |
| cuuaaagacuuuguccauaaa | 62 | uuuauggacaaagucuuuaag | 179 | AD00180-1 |
| gaaaccaacuauacgcuacaa | 63 | uuguagcguauaguugguuuc | 180 | AD00181-1 |
| gaaacucaacauauuugauca | 64 | ugaucaaauauguugaguuuc | 181 | AD00103 |
| gaacucaacauauuugaucaa | 65 | uugaucaaauauguugaguuc | 182 | AD00183-1 |
| acacccagaaguaacuucaca | 66 | ugugaaguuacuucugggugu | 183 | AD00185-1 |
| guaaagacuuuguccauaaga | 67 | ucuuauggacaaagucuuuac | 184 | AD00186-1 |
| guaguccauggacauuaauua | 68 | uaauuaauguccauggacuac | 185 | AD00187-1 |
| nnnaaagacuuuguccauaan | 69 | nuuauggacaaagucuuunnn | 186 | AD00001-1 |
| nnnagacuuuguccauaagan | 70 | nucuuauggacaaagucunnn | 187 | AD00002-1 |
| nnnugacauauuucaaaaacn | 71 | nguuuuugaaauaugucannn | 188 | AD00004-1 |
| nnnacucaacauauuugaucn | 72 | ngaucaaauauguugagunnn | 189 | AD00005-1 |
| nnncucaacauauuugaucan | 73 | nugaucaaauauguugagnnn | 190 | AD00006-1 |
| nnngaucagucuuuuuaugan | 74 | nucauaaaaagacugaucnnn | 191 | AD00007-1 |
| nnnucagucuuuuuaugaucn | 75 | ngaucauaaaaagacugannn | 192 | AD00008-1 |
| nnngaacuacauauaaacuan | 76 | nuaguuuauauguaguucnnn | 193 | AD00009-1 |
| nnnacauauaaacuacaagun | 77 | nacuuguaguuuauaugunnn | 194 | AD00010-1 |
| nnngagguaaagaauaugucn | 78 | ngacauauucuuuaccucnnn | 195 | AD00011-1 |
| nnnguaaagaauaugucacun | 79 | nagugacauauucuuuacnnn | 196 | AD00012-1 |
| nnnugucacuugaacucaacn | 80 | nguugaguucaagugacannn | 197 | AD00013-1 |
| nnnucaaaacuugaaagccun | 81 | naggcuuucaaguuuugannn | 198 | AD00014-1 |
| nnncuugaaagccuccuagan | 82 | nucuaggaggcuuucaagnnn | 199 | AD00015-1 |
| nnnaaaaauucuacuucaacn | 83 | nguugaaguagaauuuuunnn | 200 | AD00016-1 |
| nnnagagcaacuaacuaacun | 84 | naguuaguuaguugcucunnn | 201 | AD00017-1 |
| nnncaagaacccacagaaaun | 85 | nauuucuguggguucuugnnn | 202 | AD00018-1 |
| nnnaagaacccacagaaauun | 86 | naauuucuguggguucuunnn | 203 | AD00019-1 |
| nnnucuaucuuccaagccaan | 87 | nuuggcuuggaagauagannn | 204 | AD00020-1 |
| nnnagaacuacucccuuucun | 88 | nagaaagggaguaguucunnn | 205 | AD00021-1 |
| nnnacucucaaguuuuucaun | 89 | naugaaaaacuugagagunnn | 206 | AD00022-1 |
| nnnucucaaguuuuucaugun | 90 | nacaugaaaaacuugagannn | 207 | AD00023-1 |
| nnncucaaguuuuucauguen | 91 | ngacaugaaaaacuugagnnn | 208 | AD00024-1 |
| nnncaaguuuuucaugucuan | 92 | nuagacaugaaaaacuugnnn | 209 | AD00025-1 |
| nnnaaguuuuucaugucuacn | 93 | nguagacaugaaaaacuunnn | 210 | AD00026-1 |
| nnnuucaugucuacugugaun | 94 | naucacaguagacaugaannn | 211 | AD00027-1 |
| nnnaugucuacugugauguun | 95 | naacaucacaguagacaunnn | 212 | AD00028-1 |
| nnngacauuaauucaacaucn | 96 | ngauguugaauuaaugucnnn | 213 | AD00029-1 |
| nnncaucgaauagauggaucn | 97 | ngauccaucuauucgaugnnn | 214 | AD00030-1 |
| nnnauagauggaucacaaaan | 98 | nuuuugugauccaucuaunnn | 215 | AD00031-1 |
| nnnagauggaucacaaaacun | 99 | naguuuugugauccaucunnn | 216 | AD00032-1 |
| nnngaucacaaaacuucaaun | 100 | nauugaaguuuugugaucnnn | 217 | AD00033-1 |
| nnnaaacuucaaugaaacgun | 101 | nacguuucauugaaguuunnn | 218 | AD00034-1 |
| nnngagaacuacaaauauggn | 102 | nccauauuuguaguucucnnn | 219 | AD00035-1 |
| nnnagaagauauacuccauan | 103 | nuauggaguauaucuucunnn | 220 | AD00036-1 |
| nnngaagauauacuccauagn | 104 | ncuauggaguauaucuucnnn | 221 | AD00037-1 |
| nnnauauacuccauagugaan | 105 | nuucacuauggaguauaunnn | 222 | AD00038-1 |
| nnnuuuuuacuugggaaaucn | 106 | ngauuucccaaguaaaaannn | 223 | AD00039-1 |
| nnngaugaguguggagaaaan | 107 | nuuuucuccacacucaucnnn | 224 | AD00040-1 |
| nnnaaacaaccuaaaugguan | 108 | nuaccauuuagguuguuunnn | 225 | AD00041-1 |
| nnnuggaagguuauacucuan | 109 | nuagaguauaaccuuccannn | 226 | AD00042-1 |
| nnnuuauacucuauaaaaucn | 110 | ngauuuuauagaguauaannn | 227 | AD00043-1 |
| nnnacaaaacuucaaugaaan | 111 | nuuucauugaaguuuugunnn | 228 | AD00044-1 |
| nnnugaaagccuccuagaagn | 112 | ncuucuaggaggcuuucannn | 229 | AD00045-1 |
| nnnaacucaacucaaaacuun | 113 | naaguuuugaguugaguunnn | 230 | AD00046-1 |
| nnnacauuaauucaacaucgn | 114 | ncgauguugaauuaaugunnn | 231 | AD00047-1 |
| nnncauuaauucaacaucgan | 115 | nucgauguugaauuaaugnnn | 232 | AD00048-1 |
| nnnauucaacaucgaauagan | 116 | nucuauucgauguugaaunnn | 233 | AD00049-1 |
| nnngggccaaauuaaugacan | 117 | nugucauuaauuuggcccnnn | 234 | AD00050-1 |
| gaauggaagguuauacucuau | 484 | auagaguauaaccuuccauuc | 515 | AV01087 |
| gaauggaagguuauacucuac | 485 | guagaguauaaccuuccauuc | 516 | AV01088 |
| gaauggaagguuauacucuag | 486 | cuagaguauaaccuuccauuc | 517 | AV01089 |
| ggaagguuauacucuaa | 487 | uuagaguauaaccuucc | 518 | AV01090 |
| uggaagguuauacucua | 488 | uagaguauaaccuucca | 519 | AV01320 |
| uggaagguuauacucuaa | 489 | uuagaguauaaccuucca | 520 | AV01091 |
| auggaagguuauacucuaa | 490 | uuagaguauaaccuuccau | 521 | AV01092 |
| uuggaagguuauacucuaa | 491 | uuagaguauaaccuuccaa | 522 | AV01093 |
| guggaagguuauacucuaa | 492 | uuagaguauaaccuuccac | 523 | AV01094 |
| cuggaagguuauacucuaa | 493 | uuagaguauaaccuuccag | 524 | AV01095 |
| aauggaagguuauacucuaa | 494 | uuagaguauaaccuuccauu | 525 | AV01096 |
| uauggaagguuauacucuaa | 495 | uuagaguauaaccuuccaua | 526 | AV01097 |
| gauggaagguuauacucuaa | 496 | uuagaguauaaccuuccauc | 527 | AV01098 |
| cauggaagguuauacucuaa | 497 | uuagaguauaaccuuccaug | 528 | AV01099 |
| caauggaagguuauacucuaa | 498 | uuagaguauaaccuuccauug | 529 | AV01100 |
| aaauggaagguuauacucuaa | 499 | uuagaguauaaccuuccauuu | 530 | AV01101 |
| uaauggaagguuauacucuaa | 500 | uuagaguauaaccuuccauua | 531 | AV01102 |
| aaaauggaagguuauacucuaa | 501 | uuagaguauaaccuuccauuuu | 532 | AV01103 |
| caaaauggaagguuauacucuaa | 502 | uuagaguauaaccuuccauuuug | 533 | AV01104 |
| ucaaaauggaagguuauacucuaa | 503 | uuagaguauaaccuuccauuuuga | 534 | AV01105 |
| cucaaaauggaagguuauacucuaa | 504 | uuagaguauaaccuuccauuuugag | 535 | AV01106 |
| ucucaaaauggaagguuauacucuaa | 505 | uuagaguauaaccuuccauuuugaga | 536 | AV01107 |
| uggaagucucaaaauggaagguuauacu | 506 | uuagaguauaaccuuccauuuuga | 537 | AV01108 |
| cuaa | | gacuucca | | |
| gaauggaagguuauacucuaa | 507 | uuagaguauaaccuuccauucuu | 538 | AV01109 |
| gaauggaagguuauacucuaa | 508 | uuagaguauaaccuuccauucga | 539 | AV01110 |
| gaauggaagguuauacucuaa | 509 | uuagaguauaaccuuccauuc | 540 | AV01111 |
| gaauggaagguuauacucuaa | 510 | uuagaguauaaccuuccauuc | 541 | AV01112 |
| gaauggaagguuauacucuaa | 511 | uuagaguauaaccuuccauuc | 542 | AV01113 |
| gaauggaagguuauauucuaa | 512 | uuagaguauaaccuuccauuc | 543 | AV01114 |
| gaauggaagguuauacucuga | 513 | ucagaguauaaccuuccauuc | 544 | AV01115 |
| gaauggaagguuauacucuaa | 514 | uuagaguauaaccuuccauuc | 545 | AV01116 |

All sequences shown 5′ to 3′ direction.
Duplex AD#s and AV#s are the number assigned to the duplex of the two strands in the same row in the table.

Table 2 shows certain chemically modified ANGPTL3 RNAi agent antisense strand and sense strand sequences of the invention. In some embodiments of methods of the invention, an RNAi agent with a polynucleotide sequence shown in Table 2 is administered to a cell and/or subject. In some embodiments of methods of the invention, an RNAi agent with a polynucleotide sequence shown in Table 2 is administered to a subject. In some embodiments of the invention an RNAi agent administered to a subject comprises is a duplex identified in a row in Table 2, column one and includes the sequence modifications show in the sense and antisense strand sequences in the same row in Table 2, columns three and six, respectively. In some embodiments of methods of the invention, a sequence shown in Table 2 may be attached to (also referred to herein as “conjugated to”) a compound capable of delivering the RNAi agent to a cell and/or tissue in a subject. A non-limiting example of a delivery compound that may be used in certain embodiments of the invention is a GalNAc-containing compound. In Table 2, the first column indicates the Duplex AD #/AV # of the base sequences as shown in Table 1. The Duplex AD #/AV #identifies the base sequences and the sense and antisense strands shown include the base sequence but with the indicated chemical modifications shown in the same row of Table 2. For example, Table 1 shows base single-strand sequences SEQ ID NO: 1 (sense) and SEQ ID NO: 118 (antisense), which together are the double-stranded duplex identified as: Duplex AD #AD00001 and Table 2 lists Duplex AD #AD00001, which indicates that the duplex of SEQ ID NO: 237 and SEQ ID NO: 287 includes base sequences of SEQ ID NO: 1 and SEQ ID NO: 118, respectively, but with the chemical modifications shown in the sense and antisense sequences shown in columns three and six, respectively. The “Sense strand SS #” in Table 2 column two is the assigned identifier for the Sense Sequence (including modifications) shown column 3 in the same row. The “Antisense strand AS #” in Table 2 column five is the assigned identifier for the Antisense sequence (including modifications) shown in column six.

TABLE 2
provides chemically modified ANGPTL3 RNAi agent antisense strand and sense strand sequences.

| | | | SEQ | | | SEQ |
|---|---|---|---|---|---|---|
| Duplex | Sense strand | | ID | Antisense | Antisense Sequence | ID |
| AD#/AV# | SS# | Sense Sequence | NO | strand AS# | | NO |
| AD00001 | AD00001-SS | g*u*uaaagaCuUuGuccauaa*a | 237 | AD00001-AS | u*U*uaugGacaaAgUcUuua*a*c | 287 |
| AD00002 | AD00002-SS | g*a*aagacuUuGuCcauaaga*a | 238 | AD00002-AS | u*U*cuuaUggacAaAgUcuu*u*c | 288 |
| AD00003 | AD00003-SS | g*c*ucaacaUaUuUgaucagu*a | 239 | AD00003-AS | u*A*cugaUcaaaUaUgUuga*g*c | 289 |
| AD00004 | AD00004-SS | g*a*augacaUaUuUcaaaaac*a | 240 | AD00004-AS | u*G*uuuuUgaaaUaUgUcau*u*c | 290 |
| AD00005 | AD00005-SS | g*a*aacucaAcAuAuuugauc*a | 241 | AD00005-AS | u*G*aucaAauauGuUgAguu*u*c | 291 |
| AD00006 | AD00006-SS | g*a*acucaaCaUaUuugauca*a | 242 | AD00006-AS | u*U*gaucAaauaUgUuGagu*u*c | 292 |
| AD00007 | AD00007-SS | g*u*ugaucaGuCuUuuuauga*a | 243 | AD00007-AS | u*U*cauaAaaagAcUgAuca*a*c | 293 |
| AD00008 | AD00008-SS | g*g*aucaguCuUuUuaugauc*a | 244 | AD00008-AS | u*G*aucaUaaaaAgAcUgau*c*c | 294 |
| AD00009 | AD00009-SS | g*a*agaacuAcAuAuaaacua*a | 245 | AD00009-AS | u*U*aguuUauauGuAgUucu*u*c | 295 |
| AD00010 | AD00010-SS | g*c*uacauaUaAaCuacaagu*a | 246 | AD00010-AS | u*A*cuugUaguuUaUaUgua*g*c | 296 |
| AD00011 | AD00011-SS | g*a*agagguAaAgAauauguc*a | 247 | AD00011-AS | u*G*acauAuucuUuAcCucu*u*c | 297 |
| AD00012 | AD00012-SS | g*a*gguaaaGaAuAugucacu*a | 248 | AD00012-AS | u*A*gugaCauauUcUuUacc*u*c | 298 |
| AD00013 | AD00013-SS | g*u*augucaCuUgAacucaac*a | 249 | AD00013-AS | u*G*uugaGuucaAgUgAcau*a*c | 299 |
| AD00014 | AD00014-SS | g*a*cucaaaAcUuGaaagccu*a | 250 | AD00014-AS | u*A*ggcuUucaaGuUuUgag*u*c | 300 |
| AD00015 | AD00015-SS | g*a*acuugaAaGcCuccuaga*a | 251 | AD00015-AS | u*U*cuagGaggcUuUcAagu*u*c | 301 |
| AD00016 | AD00016-SS | g*g*aaaaaaUuCuAcuucaac*a | 252 | AD00016-AS | u*G*uugaAguagAaUuUuuu*c*c | 302 |
| AD00017 | AD00017-SS | g*g*aagagcAaCuAacuaacu*a | 253 | AD00017-AS | u*A*guuaGuuagUuGcUcuu*c*c | 303 |
| AD00018 | AD00018-SS | g*u*ucaagaAcCcAcagaaau*a | 254 | AD00018-AS | u*A*uuucUguggGuUcUuga*a*c | 304 |
| AD00019 | AD00019-SS | g*u*caagaaCcCaCagaaauu*a | 255 | AD00019-AS | u*A*auuuCugugGgUuCuug*a*c | 305 |
| AD00020 | AD00020-SS | g*u*cucuauCuUcCaagccaa*a | 256 | AD00020-AS | u*U*uggcUuggaAgAuAgag*a*c | 306 |
| AD00021 | AD00021-SS | g*c*aagaacUaCuCccuuucu*a | 257 | AD00021-AS | u*A*gaaaGggagUaGuUcuu*g*c | 307 |
| AD00022 | AD00022-SS | g*c*aacucuCaAgUuuuucau*a | 258 | AD00022-AS | u*A*ugaaAaacuUgAgAguu*g*c | 308 |
| AD00023 | AD00023-SS | g*a*cucucaAgUuUuucaugu*a | 259 | AD00023-AS | u*A*caugAaaaaCuUgAgag*u*c | 309 |
| AD00024 | AD00024-SS | g*c*ucucaaGuUuUucauguc*a | 260 | AD00024-AS | u*G*acauGaaaaAcUuGaga*g*c | 310 |
| AD00025 | AD00025-SS | g*c*ucaaguUuUuCaugucua*a | 261 | AD00025-AS | u*U*agacAugaaAaAcUuga*g*c | 311 |
| AD00026 | AD00026-SS | g*u*caaguuUuUcAugucuac*a | 262 | AD00026-AS | u*G*uagaCaugaAaAaCuug*a*c | 312 |
| AD00027 | AD00027-SS | g*u*uuucauGuCuAcugugau*a | 263 | AD00027-AS | u*A*ucacAguagAcAuGaaa*a*c | 313 |
| AD00028 | AD00028-SS | g*u*caugucUaCuGugauguu*a | 264 | AD00028-AS | u*A*acauCacagUaGaCaug*a*c | 314 |
| AD00029 | AD00029-SS | g*u*ggacauUaAuUcaacauc*a | 265 | AD00029-AS | u*G*auguUgaauUaAuGucc*a*c | 315 |
| AD00030 | AD00030-SS | g*a*acaucgAaUaGauggauc*a | 266 | AD00030-AS | u*G*auccAucuaUuCgAugu*u*c | 316 |
| AD00031 | AD00031-SS | g*g*aauagaUgGaUcacaaaa*a | 267 | AD00031-AS | u*U*uuugUgaucCaUcUauu*c*c | 317 |
| AD00032 | AD00032-SS | g*a*uagaugGaUcAcaaaacu*a | 268 | AD00032-AS | u*A*guuuUgugaUcCaUcua*u*c | 318 |
| AD00033 | AD00033-SS | g*u*ggaucaCaAaAcuucaau*a | 269 | AD00033-AS | u*A*uugaAguuuUgUgAucc*a*c | 319 |
| AD00034 | AD00034-SS | g*c*aaaacuUcAaUgaaacgu*a | 270 | AD00034-AS | u*A*cguuUcauuGaAgUuuu*g*c | 320 |
| AD00035 | AD00035-SS | g*g*ggagaaCuAcAaauaugg*a | 271 | AD00035-AS | u*C*cauaUuuguAgUuCucc*c*c | 321 |
| AD00036 | AD00036-SS | g*a*gagaagAuAuAcuccaua*a | 272 | AD00036-AS | u*U*auggAguauAuCuUcuc*u*c | 322 |
| AD00037 | AD00037-SS | g*g*agaagaUaUaCuccauag*a | 273 | AD00037-AS | u*C*uaugGaguaUaUcUucu*c*c | 323 |
| AD00038 | AD00038-SS | g*a*gauauaCuCcAuagugaa*a | 274 | AD00038-AS | u*U*ucacUauggAgUaUauc*u*c | 324 |
| AD00039 | AD00039-SS | g*u*cuuuuuAcUuGggaaauc*a | 275 | AD00039-AS | u*G*auuuCccaaGuAaAaag*a*c | 325 |
| AD00040 | AD00040-SS | g*a*ugaugaGuGuGgagaaaa*a | 276 | AD00040-AS | u*U*uuucUccacAcUcAuca*u*c | 326 |
| AD00041 | AD00041-SS | g*g*aaaacaAcCuAaauggua*a | 277 | AD00041-AS | u*U*accaUuuagGuUgUuuu*c*c | 327 |
| AD00042 | AD00042-SS | g*a*auggaaGgUuAuacucua*a | 278 | AD00042-AS | u*U*agagUauaaCcUuCcau*u*c | 328 |
| AD00043 | AD00043-SS | g*g*guuauaCuCuAuaaaauc*a | 279 | AD00043-AS | u*G*auuuUauagAgUaUaac*c*c | 329 |
| AD00044 | AD00044-SS | g*u*cacaaaAcUuCaaugaaa*a | 280 | AD00044-AS | u*U*uucaUugaaGuUuUgug*a*c | 330 |
| AD00045 | AD00045-SS | g*c*uugaaaGcCuCcuagaag*a | 281 | AD00045-AS | u*C*uucuAggagGcUuUcaa*g*c | 331 |
| AD00046 | AD00046-SS | g*u*gaacucAaCuCaaaacuu*a | 282 | AD00046-AS | u*A*aguuUugagUuGaGuuc*a*c | 332 |
| AD00047 | AD00047-SS | g*g*gacauuAaUuCaacaucg*a | 283 | AD00047-AS | u*C*gaugUugaaUuAaUguc*c*c | 333 |
| AD00048 | AD00048-SS | g*g*acauuaAuUcAacaucga*a | 284 | AD00048-AS | u*U*cgauGuugaAuUaAugu*c*c | 334 |
| AD00049 | AD00049-SS | g*u*aauucaAcAuCgaauaga*a | 285 | AD00049-AS | u*U*cuauUcgauGuUgAauu*a*c | 335 |
| AD00050 | AD00050-SS | g*a*agggccAaAuUaaugaca*a | 286 | AD00050-AS | u*U*gucaUuaauUuGgCccu*u*c | 336 |
| AV01087 | AV01087-SS | g*a*auggaaGgUuAuacucua*u | 546 | AV01087-AS | a*U*agagUauaaCcUuCcau*u*c | 576 |
| AV01088 | AV01088-SS | g*a*auggaaGgUuAuacucua*c | 547 | AV01088-AS | g*U*agagUauaaCcUuCcau*u*c | 577 |
| AV01089 | AV01089-SS | g*a*auggaaGgUuAuacucua*g | 548 | AV01089-AS | c*U*agagUauaaCcUuCcau*u*c | 578 |
| AV01090 | AV01090-SS | g*g*aaGgUuAuacucua*a | 549 | AV01090-AS | u*U*agagUauaaCcUu*C*c | 579 |
| AV01091 | AV01091-SS | u*g*gaaGgUuAuacucua*a | 550 | AV01091-AS | u*U*agagUauaaCcUuC*c*a | 580 |
| AV01092 | AV01092-SS | a*u*ggaaGgUuAuacucua*a | 551 | AV01092-AS | u*U*agagUauaaCcUuCc*a*u | 581 |
| AV01093 | AV01093-SS | u*u*ggaaGgUuAuacucua*a | 552 | AV01093-AS | u*U*agagUauaaCcUuCc*a*a | 582 |
| AV01094 | AV01094-SS | g*u*ggaaGgUuAuacucua*a | 553 | AV01094-AS | u*U*agagUauaaCcUuCc*a*c | 583 |
| AV01095 | AV01095-SS | c*u*ggaaGgUuAuacucua*a | 554 | AV01095-AS | u*U*agagUauaaCcUuCc*a*g | 584 |
| AV01096 | AV01096-SS | a*a*uggaaGgUuAuacucua*a | 555 | AV01096-AS | u*U*agagUauaaCcUuCca*u*u | 585 |
| AV01097 | AV01097-SS | u*a*uggaaGgUuAuacucua*a | 556 | AV01097-AS | u*U*agagUauaaCcUuCca*u*a | 586 |
| AV01098 | AV01098-SS | g*a*uggaaGgUuAuacucua*a | 557 | AV01098-AS | u*U*agagUauaaCcUuCca*u*c | 587 |
| AV01099 | AV01099-SS | c*a*uggaaGgUuAuacucua*a | 558 | AV01099-AS | u*U*agagUauaaCcUuCca*u*g | 588 |
| AV01100 | AV01100-SS | c*a*auggaaGgUuAuacucua*a | 559 | AV01100-AS | u*U*agagUauaaCcUuCcau*u*g | 589 |
| AV01101 | AV01101-SS | a*a*auggaaGgUuAuacucua*a | 560 | AV01101-AS | u*U*agagUauaaCcUuCcau*u*u | 590 |
| AV01102 | AV01102-SS | u*a*auggaaGgUuAuacucua*a | 561 | AV01102-AS | u*U*agagUauaaCcUuCcau*u*a | 591 |
| AV01103 | AV01103-SS | a*a*aauggaaGgUuAuacucua*a | 562 | AV01103-AS | u*U*agagUauaaCcUuCcauu*u*u | 592 |
| AV01104 | AV01104-SS | c*a*aaauggaaGgUuAuacucua*a | 563 | AV01104-AS | u*U*agagUauaaCcUuCcauuu*u*g | 593 |
| AV01105 | AV01105-SS | u*c*aaaauggaaGgUuAuacucua*a | 564 | AV01105-AS | u*U*agagUauaaCcUuCcauuuu*g*a | 594 |
| AV01106 | AV01106-SS | c*u*caaaauggaaGgUuAuacucua*a | 565 | AV01106-AS | u*U*agagUauaaCcUuCcauuuug*a*g | 595 |
| AV01107 | AV01107-SS | u*c*ucaaaauggaaGgUuAuacucua*a | 566 | AV01107-AS | u*U*agagUauaaCcUuCcauuuuga*g*a | 596 |
| AV01108 | AV01108-SS | u*g*gaagucucaaaauggaaGgUuAuac | 567 | AV01108-AS | u*U*agagUauaaCcUuCcauuuugagacu | 597 |
| | | ucua*a | | | uc*c*a | |
| AV01109 | AV01109-SS | g*a*auggaaGgUuAuacucua*a | 568 | AV01109-AS | u*U*agagUauaaCcUuCcauuc*u*u | 598 |
| AV01110 | AV01110-SS | g*a*auggaaGgUuAuacucua*a | 569 | AV01110-AS | u*U*agagUauaaCcUuCcauuc*g*a | 599 |
| AV01111 | AV01111-SS | g*a*auggaaGgUuAuacucua*a | 570 | AV01111-AS | u*U*aga(gUNA)UauaaCcUuCcau*u*c | 600 |
| AV01112 | AV01112-SS | g*a*auggaaGgUuAuacucua*a | 571 | AV01112-AS | u*U*agag(uUNA)auaaCcUuCcau*u*c | 601 |
| AV01113 | AV01113-SS | g*a*auggaaGGUuauacucua*a | 572 | AV01113-AS | u*U*agaGuauaaccUuCcau*u*c | 602 |
| AV01114 | AV01114-SS | g*a*auggaaGgUuAuauucua*a | 573 | AV01114-AS | u*U*agagUauaaCcUuCcau*u*c | 603 |
| AV01115 | AV01115-SS | g*a*auggaaGgUuAuacucug*a | 574 | AV01115-AS | u*C*agagUauaaCcUuCcau*u*c | 604 |
| AV01116 | AV01116-SS | g*a*auggaaGgUuAuacucu*a*a | 575 | AV01116-AS | u*U*agagUauaaCcUuCcau*u*c | 605 |

All sequences shown 5′ to 3′.
These sequences were used in certain in vitro testing studies described herein.
The chemical modifications are indicated by:
Upper case: 2′-Fluoro;
lower case: 2′-OMe;
thiophosphate: *

Table 3 shows certain chemically modified ANGPTL3 RNAi agent antisense strand and sense strand sequences of the invention. In some embodiments of methods of the invention, RNAi agents shown in Table 3 are administered to a cell and/or subject. In some embodiments of methods of the invention, an RNAi agent with a polynucleotide sequence shown in Table 3 is administered to a subject. In some embodiments of the invention an RNAi agent administered to a subject comprises is a duplex identified in a row in Table 3, column one and includes the sequence modifications and/or delivery compound show in the sense and antisense strand sequences in the same row in Table 3, columns three and six, respectively. The sequences were used in certain in vivo testing studies described elsewhere herein. In some embodiments of methods of the invention, a sequence shown in Table 3 may be attached to (also referred to herein as “conjugated to”) a compound for delivery, a non-limiting example of which is a GalNAc-containing compound, with a delivery compound identified in Table 3 as “GLX-n” on sense strands in column three. As used herein and shown in Table 3, “GLX-n” is used to indicate the attached GalNAc-containing compound is any one of compounds GLS-1, GLS-2, GLS-3, GLS-4, GLS-5, GLS-6, GLS-7, GLS-8, GLS-9, GLS-10, GLS-11, GLS-12, GLS-13, GLS-14, GLS-15, GLS-16, GLO-1, GLO-2, GLO-3, GLO-4, GLO-5, GLO-6, GLO-7, GLO-8, GLO-9, GLO-10, GLO-11, GLO-12, GLO-13, GLO-14, GLO-15, and GLO-16, The structure of each of which is provided elsewhere herein. Column one of Table 3 provides a Duplex AD #assigned to the duplex of the sense and antisense sequences in that row of the table. For example, Duplex AD #AD00102 is the duplex of sense strand SEQ ID NO: 337 and antisense strand SEQ ID NO: 364. Each line in Table 3 provides a sense strand and an antisense strand, and discloses the duplex of the sense and antisense strands shown. The “Sense strand SS #” in Table 3 column two is the assigned identifier for the Sense Sequence (including modifications) shown column 3 in the same row. The “Antisense strand AS #” in Table 3 column five is the assigned identifier for the Antisense sequence (including modifications) shown in column six. A identifier for certain attached GalNAc-containing GLO compounds is shown as GLO-0, and it will be understood that another of the GLO-n or GLS-n compounds may be substituted for the compound shown as GLO-0, with the resulting compound included in an embodiment of a method and/or a composition of the invention.

TABLE 3
provides chemically modified ANGPTL3 RNAi agent antisense strand and sense strand sequences.

| | | | SEQ | | | SEQ |
|---|---|---|---|---|---|---|
| | | | ID | | | ID |
| [symbol] AD# | [symbol] SS# | [symbol] | NO | [symbol] AS# | [symbol] | NO |
| AD00102 | AD00102-SS | g*a*aagacuUuGuCcauaaga*a(GLO-0) | 337 | AD00102-AS | u*U*cuuaUggacAaAgUcuu*u*c | 364 |
| AD00103 | AD00103-SS | g*a*aacucaAcAuAuuugauc*a(GLO-0) | 338 | AD00103-AS | u*G*aucaAauauGuUgAguu*u*c | 365 |
| AD00104 | AD00104-SS | g*g*aucaguCuUuUuaugauc*a(GLO-0) | 339 | AD00104-AS | u*G*aucaUaaaaAgAcUgau*c*c | 366 |
| AD00105 | AD00105-SS | g*a*gguaaaGaAuAugucacu*a(GLO-0) | 340 | AD00105-AS | u*A*gugaCauauUcUuUacc*u*c | 367 |
| AD00106 | AD00106-SS | g*g*aagagcAaCuAacuaacu*a(GLO-0) | 341 | AD00106-AS | u*A*guuaGuuagUuGcUcuu*c*c | 368 |
| AD00107 | AD00107-SS | g*u*caagaaCcCaCagaaauu*a(GLO-0) | 342 | AD00107-AS | u*A*auuuCugugGgUuCuug*a*c | 369 |
| AD00108 | AD00108-SS | g*a*cucucaAgUuUuucaugu*a(GLO-0) | 343 | AD00108-AS | u*A*caugAaaaaCuUgAgag*u*c | 370 |
| AD00109 | AD00109-SS | g*c*ucucaaGuUuUucauguc*a(GLO-0) | 344 | AD00109-AS | u*G*acauGaaaaAcUuGaga*g*c | 371 |
| AD00110 | AD00110-SS | g*c*ucaaguUuUuCaugucua*a(GLO-0) | 345 | AD00110-AS | u*U*agacAugaaAaAcUuga*g*c | 372 |
| AD00111 | AD00111-SS | g*u*ggacauUaAuUcaacauc*a(GLO-0) | 346 | AD00111-AS | u*G*auguUgaauUaAuGucc*a*c | 373 |
| AD00112 | AD00112-SS | g*a*auggaaGgUuAuacucua*a(GLO-0) | 347 | AD00112-AS | u*U*agagUauaaCcUuCcau*u*c | 374 |
| AD00132 | AD00132-SS | a*a*gggccaAaUuAaugacau*a(GLO-0) | 348 | AD00132-AS | u*A*ugucAuuaaUuUgGccc*u*u | 375 |
| AD00133 | AD00133-SS | g*a*augacaUaUuUcaaaaac*a(GLO-0) | 349 | AD00133-AS | u*G*uuuuUgaaaUaUgUcau*u*c | 376 |
| AD00134 | AD00134-SS | g*u*ugaucaGuCuUuuuauga*a(GLO-0) | 350 | AD00134-AS | u*U*cauaAaaagAcUgAuca*a*c | 377 |
| AD00135 | AD00135-SS | g*a*agaacuAcAuAuaaacua*a(GLO-0) | 351 | AD00135-AS | u*U*aguuUauauGuAgUucu*u*c | 378 |
| AD00136 | AD00136-SS | g*u*augucaCuUgAacucaac*a(GLO-0) | 352 | AD00136-AS | u*G*uugaGuucaAgUgAcau*a*c | 379 |
| AD00137 | AD00137-SS | g*a*cucaaaAcUuGaaagccu*a(GLO-0) | 353 | AD00137-AS | u*A*ggcuUucaaGuUuUgag*u*c | 380 |
| AD00138 | AD00138-SS | g*c*uugaaaGcCuCcuagaag*a(GLO-0) | 354 | AD00138-AS | u*C*uucuAggagGcUuUcaa*g*c | 381 |
| AD00139 | AD00139-SS | c*u*caaguuUuUcAugucuac*a(GLO-0) | 355 | AD00139-AS | u*G*uagaCaugaAaAaCuug*a*g | 382 |
| AD00140 | AD00140-SS | g*u*uuucauGuCuAcugugau*a(GLO-0) | 356 | AD00140-AS | u*A*ucacAguagAcAuGaaa*a*c | 383 |
| AD00141 | AD00141-SS | c*a*acaucgAaUaGauggauc*a(GLO-0) | 357 | AD00141-AS | u*G*auccAucuaUuCgAugu*u*g | 384 |
| AD00142 | AD00142-SS | c*g*aauagaUgGaUcacaaaa*a(GLO-0) | 358 | AD00142-AS | u*U*uuugUgaucCaUcUauu*c*g | 385 |
| AD00143 | AD00143-SS | a*u*ggaucaCaAaAcuucaau*a(GLO-0) | 359 | AD00143-AS | u*A*uugaAguuuUgUgAucc*a*u | 386 |
| AD00144 | AD00144-SS | g*c*aaaacuUcAaUgaaacgu*a(GLO-0) | 360 | AD00144-AS | u*A*cguuUcauuGaAgUuuu*g*c | 387 |
| AD00145 | AD00145-SS | g*u*cuuuuuAcUuGggaaauc*a(GLO-0) | 361 | AD00145-AS | u*G*auuuCccaaGuAaAaag*a*c | 388 |
| AD00146 | AD00146-SS | c*a*ugaugaGuGuGgagaaaa*a(GLO-0) | 362 | AD00146-AS | u*U*uuucUccacAcUcAuca*u*g | 389 |
| AD00147 | AD00147-SS | a*g*guuauaCuCuAuaaaauc*a(GLO-0) | 363 | AD00147-AS | u*G*auuuUauagAgUaUaac*c*u | 390 |

All sequences shown 5′ to 3′.
The sequences were used in certain in vivo testing studies described elsewhere herein.
A delivery molecule used in the in vivo studies is indicated as “GLO-0” at the 3′ end of each sense strand.
Chemical modifications are indicated as:
upper case: 2′-Fluoro;
lower case: 2′-OMe;
thiophosphate: *

Table 4 shows certain chemically modified ANGPTL3 RNAi agent antisense-strand and sense-strand sequences of the invention. In some embodiments of methods of the invention, an RNAi agent with a polynucleotide sequence shown in Table 4 is administered to a subject. In some embodiments of the invention an RNAi agent administered to a subject comprises is a duplex identified in a row in Table 4, column one and includes the sequence modifications and/or delivery compound show in the sense and antisense strand sequences in the same row in Table 4, columns three and six, respectively. In some embodiments of methods of the invention, a sequence shown in Table 4 may be attached to a compound capable of delivering the RNAi agent to a cell and/or tissue in a subject. A non-limiting example of a delivery compound that may be used in certain embodiments of the invention is a GalNAc-containing compound. In Table 4, the term “GLX-n” indicates a GalNAc-containing compound in the sense strand as shown. “In Table 4, GLX-n is used to indicate the attached GalNAC-containing compound is any one of compound GLS-1, GLS-2, GLS-3, GLS-4, GLS-5, GLS-6, GLS-7, GLS-8, GLS-9, GLS-10, GLS-11, GLS-12, GLS-13, GLS-14, GLS-15, GLS-16, GLO-1, GLO-2, GLO-3, GLO-4, GLO-5, GLO-6, GLO-7, GLO-8, GLO-9, GLO-10, GLO-11, GLO-12, GLO-13, GLO-14, GLO-15, and GLO-16, the structure of each of which is provided elsewhere herein. The first column of Table 4 indicates the Duplex AD #corresponding to the duplexes shown in Table 3. The Duplex AD #identifies the duplex sequences of Table 3, indicating the sense, antisense, and duplex sequences in Table 4 have the same base sequence as those with the same Duplex AD # in Table 3, but the sequences and duplexes in Table 4 have different chemical modifications and/or delivery compounds compared to the corresponding sequences and duplexes shown in Table 3. For example, SEQ ID NO: 337 (sense), SEQ ID NO: 364 (antisense), and their double-stranded duplex as Duplex AD #AD00102 shown in Table 3 have the same base sequences as SEQ ID NO: 391, SEQ ID NO: 418, and AD #AD00102-1, respectively, as shown in Table 4, with chemical modifications and/or delivery compounds as indicated in each table.

TABLE 4
Chemically modified ANGPTL3 RNAi agent antisense strand and sense strand sequences.

| | | | SEQ | | | SEQ |
|---|---|---|---|---|---|---|
| [symbol] | [symbol] | | ID | [symbol] | | ID |
| AD# | SS# | [symbol] 5′->3′ | NO | AS# | [symbol] 5′->3′ | NO |
| AD00102- | AD00102- | (GLS-5)*(Invab)*gaaagacuUuGuCcauaagaa*(Invab) | 391 | AD00102-1- | u*U*cuuaUggacAaAgUcuu*u*c | 418 |
| 1 | 1-SS | | | AS | | |
| AD00103- | AD00103- | (GLS-5)*(Invab)*gaaacucaAcAuAuuugauca*(Invab) | 392 | AD00103-1- | u*G*aucaAauauGuUgAguu*u*c | 419 |
| 1 | 1-SS | | | AS | | |
| AD00104- | AD00104- | (GLS-5)*(Invab)*ggaucaguCuUuUuaugauca*(Invab) | 393 | AD00104-1- | u*G*aucaUaaaaAgAcUgau*c*c | 420 |
| 1 | 1-SS | | | AS | | |
| AD00105- | AD00105- | (GLS-5)*(Invab)*gagguaaaGaAuAugucacua*(Invab) | 394 | AD00105-1- | u*A*gugaCauauUcUuUacc*u*c | 421 |
| 1 | 1-SS | | | AS | | |
| AD00106- | AD00106- | (GLS-5)*(Invab)*ggaagagcAaCuAacuaacua*(Invab) | 395 | AD00106-1- | u*A*guuaGuuagUuGcUcuu*c*c | 422 |
| 1 | 1-SS | | | AS | | |
| AD00107- | AD00107- | (GLS-5)*(Invab)*gucaagaaCcCaCagaaauua*(Invab) | 396 | AD00107-1- | u*A*auuuCugugGgUuCuug*a*c | 423 |
| 1 | 1-SS | | | AS | | |
| AD00108- | AD00108- | (GLS-5)*(Invab)*gacucucaAgUuUuucaugua*(Invab) | 397 | AD00108-1- | u*A*caugAaaaaCuUgAgag*u*c | 424 |
| 1 | 1-SS | | | AS | | |
| AD00109- | AD00109- | (GLS-5)*(Invab)*gcucucaaGuUuUucauguca*(Invab) | 398 | AD00109-1- | u*G*acauGaaaaAcUuGaga*g*c | 425 |
| 1 | 1-SS | | | AS | | |
| AD00110- | AD00110- | (GLS-5)*(Invab)*gcucaaguUuUuCaugucuaa*(Invab) | 399 | AD00110-1- | u*U*agacAugaaAaAcUuga*g*c | 426 |
| 1 | 1-SS | | | AS | | |
| AD00111- | AD00111- | (GLS-5)*(Invab)*guggacauUaAuUcaacauca*(Invab) | 400 | AD00111-1- | u*G*auguUgaauUaAuGucc*a*c | 427 |
| 1 | 1-SS | | | AS | | |
| AD00112- | AD00112- | (GLS-5)*(Invab)*gaauggaaGgUuAuacucuaa*(Invab) | 401 | AD00112-1- | u*U*agagUauaaCcUuCcau*u*c | 428 |
| 1 | 1-SS | | | AS | | |
| AD00132- | AD00132- | (GLS-5)*(Invab)*aagggccaAaUuAaugacaua*(Invab) | 402 | AD00132-1- | u*A*ugucAuuaaUuUgGccc*u*u | 429 |
| 1 | 1-SS | | | AS | | |
| AD00133- | AD00133- | (GLS-5)*(Invab)*gaaugacaUaUuUcaaaaaca*(Invab) | 403 | AD00133-1- | u*G*uuuuUgaaaUaUgUcau*u*c | 430 |
| 1 | 1-SS | | | AS | | |
| AD00134- | AD00134- | (GLS-5)*(Invab)*guugaucaGuCuUuuuaugaa*(Invab) | 404 | AD00134-1- | u*U*cauaAaaagAcUgAuca*a*c | 431 |
| 1 | 1-SS | | | AS | | |
| AD00135- | AD00135- | (GLS-5)*(Invab)*gaagaacuAcAuAuaaacuaa*(Invab) | 405 | AD00135-1- | u*U*aguuUauauGuAgUucu*u*c | 432 |
| 1 | 1-SS | | | AS | | |
| AD00136- | AD00136- | (GLS-5)*(Invab)*guaugucaCuUgAacucaaca*(Invab) | 406 | AD00136-1- | u*G*uugaGuucaAgUgAcau*a*c | 433 |
| 1 | 1-SS | | | AS | | |
| AD00137- | AD00137- | (GLS-5)*(Invab)*gacucaaaAcUuGaaagccua*(Invab) | 407 | AD00137-1- | u*A*ggcuUucaaGuUuUgag*u*c | 434 |
| 1 | 1-SS | | | AS | | |
| AD00138- | AD00138- | (GLS-5)*(Invab)*gcuugaaaGcCuCcuagaaga*(Invab) | 408 | AD00138-1- | u*C*uucuAggagGcUuUcaa*g*c | 435 |
| 1 | 1-SS | | | AS | | |
| AD00139- | AD00139- | (GLS-5)*(Invab)*cucaaguuUuUcAugucuaca*(Invab) | 409 | AD00139-1- | u*G*uagaCaugaAaAaCuug*a*g | 436 |
| 1 | 1-SS | | | AS | | |
| AD00140- | AD00140- | (GLS-5)*(Invab)*guuuucauGuCuAcugugaua*(Invab) | 410 | AD00140-1- | u*A*ucacAguagAcAuGaaa*a*c | 437 |
| 1 | 1-SS | | | AS | | |
| AD00141- | AD00141- | (GLS-5)*(Invab)*caacaucgAaUaGauggauca*(Invab) | 411 | AD00141-1- | u*G*auccAucuaUuCgAugu*u*g | 438 |
| 1 | 1-SS | | | AS | | |
| AD00142- | AD00142- | (GLS-5)*(Invab)*cgaauagaUgGaUcacaaaaa*(Invab) | 412 | AD00142-1- | u*U*uuugUgaucCaUcUauu*c*g | 439 |
| 1 | 1-SS | | | AS | | |
| AD00143- | AD00143- | (GLS-5)*(Invab)*auggaucaCaAaAcuucaaua*(Invab) | 413 | AD00143-1- | u*A*uugaAguuuUgUgAucc*a*u | 440 |
| 1 | 1-SS | | | AS | | |
| AD00144- | AD00144- | (GLS-5)*(Invab)*gcaaaacuUcAaUgaaacgua*(Invab) | 414 | AD00144-1- | u*A*cguuUcauuGaAgUuuu*g*c | 441 |
| 1 | 1-SS | | | AS | | |
| AD00145- | AD00145- | (GLS-5)*(Invab)*gucuuuuuAcUuGggaaauca*(Invab) | 415 | AD00145-1- | u*G*auuuCccaaGuAaAaag*a*c | 442 |
| 1 | 1-SS | | | AS | | |
| AD00146- | AD00146- | (GLS-5)*(Invab)*caugaugaGuGuGgagaaaaa*(Invab) | 416 | AD00146-1- | u*U*uuucUccacAcUcAuca*u*g | 443 |
| 1 | 1-SS | | | AS | | |
| AD00147- | AD00147- | (GLS-5)*(Invab)*agguuauaCuCuAuaaaauca*(Invab) | 417 | AD00147-1- | u*G*auuuUauagAgUaUaac*c*u | 444 |
| 1 | 1-SS | | | AS | | |
| AD00112- | AD00112- | (GLS-15)*(Invab)*gaauggaaGgUuAuacucuaa* | 606 | AD00112-2- | u*U*agagUauaaCcUuCcau*u*c | 611 |
| 2 | 2-SS | (Invab) | | AS | | |
| AD00135- | AD00135- | (GLS-5)*(Invab)*caagaacuAcAuAuaaacuaa*(Invab) | 607 | AD00135-2- | u*U*aguuUauauGuAgUucu*u*g | 612 |
| 2 | 2-SS | | | AS | | |
| AD00135- | AD00135- | (GLS-15)*(Invab)*caagaacuAcAuAuaaacuaa* | 608 | AD00135-3- | u*U*aguuUauauGuAgUucu*u*g | 613 |
| 3 | 3-SS | (Invab) | | AS | | |
| AD00136- | AD00136- | (GLS-15)*(Invab)*guaugucaCuUgAacucaaca* | 609 | AD00136-2- | u*G*uugaGuucaAgUgAcau*a*c | 614 |
| 2 | 2-SS | (Invab) | | AS | | |
| AD00143- | AD00143- | (GLS-5)*(Invab)*guggaucaCaAaAcuucaaua*(Invab) | 610 | AD00143-2- | u*A*uugaAguuuUgUgAucc*a*c | 615 |
| 2 | 2-SS | | | AS | | |
| AD00108- | AD00108- | (GLS-15)*(Invab)*gacucucaAgUuUuucaugua* | 616 | AD00108-2- | u*A*caugAaaaaCuUgAgag*u*c | 617 |
| 2 | 2-SS | (Invab) | | AS | | |
| AD00133- | AD00133- | (GLS-15)*(Invab)*gaaugacaUaUuUcaaaaaca* | 618 | AD00133-2- | u*G*uuuuUgaaaUaUgUcau*u*c | 619 |
| 2 | 2-SS | (Invab) | | AS | | |
| AD00134- | AD00134- | (GLS-15)*(Invab)*guugaucaGuCuUuuuaugaa* | 620 | AD00134-2- | u*U*cauaAaaagAcUgAuca*a*c | 621 |
| 2 | 2-SS | (Invab) | | AS | | |
| AD00142- | AD00142- | (GLS-15)*(Invab)*cgaauagaUgGaUcacaaaaa* | 622 | AD00142-2- | u*U*uuugUgaucCaUcUauu*c*g | 623 |
| 2 | 2-SS | (Invab) | | AS | | |
| AD00143- | AD00143- | (GLS-15)*(Invab)*guggaucaCaAaAcuucaaua* | 624 | AD00143-3- | u*A*uugaAguuuUgUgAucc*a*u | 625 |
| 3 | 3-SS | (Invab) | | AS | | |
| AD00145- | AD00145- | (GLS-15)*(Invab)*gucuuuuuAcUuGggaaauca* | 626 | AD00145-2- | u*G*auuuCccaaGuAaAaag*a*c | 627 |
| 2 | 2-SS | (Invab) | | AS | | |
| AD00146- | AD00146- | (GLS-15)*(Invab)*caugaugaGuGuGgagaaaaa* | 628 | AD00146-2- | u*U*uuucUccacAcUcAuca*u*g | 629 |
| 2 | 2-SS | (Invab) | | AS | | |

Sequences were used in certain in vivo studies described elsewhere herein.
All sequences are shown 5′ to 3′.
Chemical modifications are indicated as:
upper case: 2′-Fluoro;
lower case: 2′-OMe;
thiophosphate: *;
Invab = inverted abasic.

Table 5 shows certain chemically modified ANGPTL3 RNAi agent antisense-strand and sense-strand sequences of the invention. In some embodiments of methods of the invention, an RNAi agent with a polynucleotide sequence shown in Table 5 is administered to a subject. In some embodiments of the invention an RNAi agent administered to a subject comprises is a duplex identified in a row in Table 5, column one and includes the sequence modifications and/or delivery compound show in the sense and antisense strand sequences in the same row in Table 5, columns three and six, respectively. In some embodiments of methods of the invention, a sequence shown in Table 5 may be attached to a compound capable of delivering the RNAi agent to a cell and/or tissue in a subject. A non-limiting example of a delivery compound that may be used in certain embodiments of the invention is a GalNAc-containing compound. In Table 5, the terms “GLO-0” and “GLS-5” each indicates a different GalNAc-containing compound attached to the sense strand as shown. It will be understood that another of the GLO-n or GLS-n compounds may be substituted for the compound shown as GLO-0, with the resulting compound included in an embodiment of a method and/or a composition of the invention. Similarly, another of the GLS-n or GLO-n compounds may be substituted for the compound shown as GLS-5, with the resulting compound included in an embodiment of a method and/or a composition of the invention It will be understood that certain embodiments of the invention include an RNAi agent of the invention with a sequence shown in Table 5, but that is attached to any one of the GalNAc-containing compounds: GLS-1, GLS-2, GLS-3, GLS-4, GLS-5, GLS-6, GLS-7, GLS-8, GLS-9, GLS-10, GLS-11, GLS-12, GLS-13, GLS-14, GLS-15, GLS-16, GLO-1, GLO-2, GLO-3, GLO-4, GLO-5, GLO-6, GLO-7, GLO-8, GLO-9, GLO-10, GLO-11, GLO-12, GLO-13, GLO-14, GLO-15, and GLO-16, the structure of each of which is provided elsewhere herein. The first column of Table 5 identifies Duplex AD #numbers: AD00178 through AD #AD00187, with the number in each row identifying a duplex comprising the sense and antisense strands shown in the same row in columns three and six, respectively, and including the modifications and with an attached 3′ GLO- or GLS-delivery compound on the sense strand. Duplex AD #s AD00178 through AD00187 are fully complementary to mouse ANGPTL3 mRNA sequence but with 0 or 1 mismatch to human ANGPTL3 mRNA sequence.

The first column of Table 5 identifies Duplex AD #numbers: AD00179-1, AD00180-1, AD00181-1, AD00103-1, AD00183-1, AD00184-1, AD00185-1, AD00186-1, and AD00187-1, with the number in each row identifying a duplex comprising the sense and antisense strands shown in the same row in columns three and six, respectively, and including the modifications and with an attached 5′ or 3′ GLS-n or GLO-n delivery compound on the sense strand. Modifications are indicated with: upper case: 2′-Fluoro; lower case: 2′-OMe; thiophosphate: *; Invab=inverted abasic. Duplexes AD00179-1, AD00180-1, AD00181-1, AD00103-1, AD00183-1, AD00184-1, AD00185-1, AD00186-1, and AD00187-1 are fully complementary to human ANGPTL3 mRNA sequence but with 0 or 1 mismatch to mouse ANGPTL3 mRNA sequence.

TABLE 5
provides chemically modified ANGPTL3 RNAi agent antisense strand and sense strand sequences.

| | | | | | | |
|---|---|---|---|---|---|---|
| | | | SEQ | | | SEQ |
| [symbol] | [symbol] | | ID | | | ID |
| AD# | SS# | [symbol] | NO | [symbol] AS# | [symbol] | NO |

[symbol]

1 mm (3′ GLO-)

| | | | | | | |
|---|---|---|---|---|---|---|
| AD00178 | AD00178- | g*a*aagauuUuGuCcauaaga*a(GLO-0) | 445 | AD00178-AS | u*U*cuuaUggacAaAaUcuu*u* | 464 |
| | SS | | | | c | |
| AD00179 | AD00179- | g*c*aaaaucAaGaUuugcuau*a(GLO-0) | 446 | AD00179-AS | u*A*uagcAaaucUuGaUuuu*g* | 465 |
| | SS | | | | C | |
| AD00180 | AD00180- | g*u*uaaagaUuUuGuccauaa*a(GLO-0) | 447 | AD00180-AS | u*U*uaugGacaaAaUcUuua*a* | 466 |
| | SS | | | | c | |
| AD00181 | AD00181- | g*a*aaccaaCuAcAcgcuaca*a(GLO-0) | 448 | AD00181-AS | u*U*guagCguguAgUuGguu*u | 467 |
| | SS | | | | *c | |
| AD00182 | AD00182- | g*a*agcucaAcAuAuuugauc*a(GLO-0) | 449 | AD00182-AS | u*G*aucaAauauGuUgAgcu*u* | 468 |
| | SS | | | | c | |
| AD00183 | AD00183- | g*a*gcucaaCaUaUuugauca*a(GLO-0) | 450 | AD00183-AS | u*U*gaucAaauaUgUuGagc*u* | 469 |
| | SS | | | | C | |
| AD00184 | AD00184- | g*u*auuugaUcAgUcuuuuua*a(GLO-0) | 451 | AD00184-AS | u*U*aaaaAgacuGaUcAaau*a* | 470 |
| | SS | | | | c | |
| AD00185 | AD00185- | g*c*acccagAaGuAacaucac*a(GLO-0) | 452 | AD00185-AS | u*G*ugauGuuacUuCuGggu*g* | 471 |
| | SS | | | | c | |
| AD00186 | AD00186- | g*u*aaagauUuUgUccauaag*a(GLO-0) | 453 | AD00186-AS | u*C*uuauGgacaAaAuCuuu*a* | 472 |
| | SS | | | | c | |
| AD00187 | AD00187- | g*c*aguccaUgGaCauuaauu*a(GLO-0) | 454 | AD00187-AS | u*A*auuaAugucCaUgGacu*g* | 473 |
| | SS | | | | C | |

[symbol]

1 mm- [symbol] & [symbol] (5′ GLS-5)

| | | | | | | |
|---|---|---|---|---|---|---|
| AD00179- | AD00179- | (GLS-5)* | 455 | AD00179-1- | u*A*uagcAaaucUuGaUuuu*g* | 474 |
| 1 | 1-SS | (Invab)*ccaaaaucAaGaUuugcuaua*(Invab) | | AS | g | |
| AD00180- | AD00180- | (GLS-5)* | 456 | AD00180-1- | u*U*uaugGacaaAgUcUuua*a* | 475 |
| 1 | 1-SS | (Invab)*cuuaaagaCuUuGuccauaaa*(Invab) | | AS | g | |
| AD00181- | AD00181- | (GLS-5)* | 457 | AD00181-1- | u*U*guagCguauAgUuGguu*u | 476 |
| 1 | 1-SS | (Invab)*gaaaccaaCuAuAcgcuacaa*(Invab) | | AS | *c | |
| AD00103- | AD00103- | (GLS-5)* | 458 | AD00103-1- | u*G*aucaAauauGuUgAguu*u* | 477 |
| 1 | 1-SS | (Invab)*gaaacucaAcAuAuuugauca*(Invab) | | AS | c | |
| AD00183- | AD00183- | (GLS-5)* | 459 | AD00183-1- | u*U*gaucAaauaUgUuGagu*u* | 478 |
| 1 | 1-SS | (Invab)*gaacucaaCaUaUuugaucaa*(Invab) | | AS | C | |
| AD00184- | AD00184- | (GLS-5)* | 460 | AD00184-1- | u*U*aaaaAgacuGaUcAaau*a* | 479 |
| 1 | 1-SS | (Invab)*guauuugaUcAgUcuuuuuaa*(Invab) | | AS | c | |
| AD00185- | AD00185- | (GLS-5)* | 461 | AD00185-1- | u*G*ugaaGuuacUuCuGggu*g* | 480 |
| 1 | 1-SS | (Invab)*acacccagAaGuAacuucaca*(Invab) | | AS | u | |
| AD00186- | AD00186- | (GLS-5)* | 462 | AD00186-1- | u*C*uuauGgacaAaGuCuuu*a* | 481 |
| 1 | 1-SS | (Invab)*guaaagacUuUgUccauaaga*(Invab) | | AS | c | |
| AD00187- | AD00187- | (GLS-5)* | 463 | AD00187-1- | u*A*auuaAugucCaUgGacu*a* | 482 |
| 1 | 1-SS | (Invab)*guaguccaUgGaCauuaauua*(Invab) | | AS | c | |

The sequences were used in some in vivo studies described elsewhere herein.
All sequences shown 5′ to 3′.
Modifications are indicated with:
upper case: 2′-Fluoro;
lower case: 2′-OMe;
thiophosphate: *;
Invab = inverted abasic

### Mismatches

It is known to skilled in art, mismatches are tolerated for efficacy in dsRNA, especially the mismatches are within terminal region of dsRNA. Certain mismatches tolerate better, for example mismatches with wobble base pairs G:U and A:C are tolerated better for efficacy (Du et el, A systematic analysis of the silencing effects of an active siRNA at all single-nucleotide mismatched target sites. Nucleic Acids Res. 2005 Mar. 21; 33(5):1671-7. Doi: 10.1093/nar/gki312. Nucleic Acids Res. 2005; 33(11):3698). Some embodiments of methods and compounds of the invention an ANGPTL3 dsRNA agent may contain one or more mismatches to the ANGPTL3 target sequence. In some embodiments, ANGPTL3 dsRNA agent of the invention includes no mismatches. In certain embodiments, ANGPTL3 dsRNA agent of the invention includes no more than 1 mismatch. In some embodiments, ANGPTL3 dsRNA agent of the invention includes no more than 2 mismatches. In certain embodiments, ANGPTL3 dsRNA agent of the invention includes no more than 3 mismatches. In some embodiments of the invention, an antisense strand of an ANGPTL3 dsRNA agent contains mismatches to an ANGPTL3 target sequence that are not located in the center of the region of complementarity. In some embodiments, the antisense strand of the ANGPTL3 dsRNA agent includes 1, 2, 3, 4, or more mismatches that are within the last 5, 4, 3, 2, or 1 nucleotides from one or both of the 5′ or 3′ end of the region of complementarity. Methods described herein and/or methods known in the art can be used to determine whether an ANGPTL3 dsRNA agent containing a mismatch to an ANGPTL3 target sequence is effective in inhibiting the expression of the ANGPTL3 gene.

### Complementarity

As used herein, unless otherwise indicated, the term “complementary,” when used to describe a first nucleotide sequence (e.g., ANGPTL3 dsRNA agent sense strand or targeted ANGPTL3 mRNA) in relation to a second nucleotide sequence (e.g., ANGPTL3 dsRNA agent antisense strand or a single-stranded antisense polynucleotide), means the ability of an oligonucleotide or polynucleotide including the first nucleotide sequence to hybridize [form base pair hydrogen bonds under mammalian physiological conditions (or similar conditions in vitro)] and form a duplex or double helical structure under certain conditions with an oligonucleotide or polynucleotide including the second nucleotide sequence. Other conditions, such as physiologically relevant conditions as can be encountered inside an organism, can apply. A skilled artisan will be able to determine the set of conditions most appropriate for a test of complementarity of two sequences in accordance with the ultimate application of the hybridized nucleotides. Complementary sequences include Watson-Crick base pairs or non-Watson-Crick base pairs and include natural or modified nucleotides or nucleotide mimics, at least to the extent that the above hybridization requirements are fulfilled. Sequence identity or complementarity is independent of modification.

Complementary sequences, for example, within an ANGPTL3 dsRNA as described herein, include base-pairing of the oligonucleotide or polynucleotide comprising a first nucleotide sequence to an oligonucleotide or polynucleotide comprising a second nucleotide sequence over the entire length of one or both nucleotide sequences. Such sequences can be referred to as “fully complementary” with respect to each other herein. It will be understood that in embodiments when two oligonucleotides are designed to form, upon hybridization, one or more single stranded overhangs, such overhangs are not regarded herein as mismatches with regard to the determination of complementarity. For example, an ANGPTL3 dsRNA agent comprising one oligonucleotide 19 nucleotides in length and another oligonucleotide 20 nucleotides in length, wherein the longer oligonucleotide comprises a sequence of 19 nucleotides that is fully complementary to the shorter oligonucleotide, can yet be referred to as “fully complementary” for the purposes described herein. Thus, as used herein, “fully complementary” means that all (100%) of the bases in a contiguous sequence of a first polynucleotide will hybridize with the same number of bases in a contiguous sequence of a second polynucleotide. The contiguous sequence may comprise all or a part of a first or second nucleotide sequence.

The term “substantially complementary” as used herein means that in a hybridized pair of nucleobase sequences, at least about 85%, but not all, of the bases in a contiguous sequence of a first polynucleotide will hybridize with the same number of bases in a contiguous sequence of a second polynucleotide. The term “substantially complementary” can be used in reference to a first sequence with respect to a second sequence if the two sequences include one or more, for example at least 1, 2, 3, 4, or 5 mismatched base pairs upon hybridization for a duplex up to 15, 16, 17, 18, 19, 20, 21, 22, 23, 24, 25, 26, 27, 28, 29 or 30 base pairs (bp), while retaining the ability to hybridize under the conditions most relevant to their ultimate application, e.g., inhibition of ANGPTL3 gene expression via a RISC pathway. The term, “partially complementary” may be used herein in reference to a hybridized pair of nucleobase sequences, in which at least 75%, but not all, of the bases in a contiguous sequence of a first polynucleotide will hybridize with the same number of bases in a contiguous sequence of a second polynucleotide. In some embodiments, “partially complementary” means at least 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of the bases in a contiguous sequence of a first polynucleotide will hybridize with the same number of bases in a contiguous sequence of a second polynucleotide

The terms “complementary,” “fully complementary,” “substantially complementary,” and “partially complimentary” are used herein in reference to the base matching between the sense strand and the antisense strand of an ANGPTL3 dsRNA agent, between the antisense strand of an ANGPTL3 dsRNA agent and a sequence of a target ANGPTL3 mRNA, or between a single-stranded antisense oligonucleotide and a sequence of a target ANGPTL3 mRNA. It will be understood that the term “antisense strand of an ANGPTL3 dsRNA agent” may refer to the same sequence of an “ANGPTL3 antisense polynucleotide agent”.

As used herein, the term “substantially identical” or “substantial identity” used in reference to a nucleic acid sequence means a nucleic acid sequence comprising a sequence with at least about 85% sequence identity or more, preferably at least 90%, at least 91%, at least 92%, at least 93%, at least 94%, at least 95%, at least 96%, at least 97%, at least 98%, or at least 99%, compared to a reference sequence. Percentage of sequence identity is determined by comparing two optimally aligned sequences over a comparison window. The percentage is calculated by determining the number of positions at which the identical nucleic acid base occurs in both sequences to yield the number of matched positions, dividing the number of matched positions by the total number of positions in the window of comparison and multiplying the result by 100 to yield the percentage of sequence identity. The inventions disclosed herein encompasses nucleotide sequences substantially identical to those disclosed herein. e.g., in Tables 1-5. In some embodiments, the sequences disclosed herein are exactly identical, or at least about 85%, 86%, 87%, 88%, 89%, 90%, 91%, 92%, 93%, 94%, 95%, 96%, 97%, 98%, or 99% percent identical to those disclosed herein, e.g., in Tables 1-5.

As used herein, the term “strand comprising a sequence” means an oligonucleotide comprising a chain of nucleotides that is described by the sequence referred to using the standard nucleotide nomenclature. The term “double-stranded RNA” or “dsRNA,” as used herein, refers to an RNAi that includes an RNA molecule or complex of molecules having a hybridized duplex region comprising two anti-parallel and substantially or fully complementary nucleic acid strands, which are referred to as having “sense” and “antisense” orientations with respect to a target ANGPTL3 RNA. The duplex region can be of any length that permits specific degradation of a desired target ANGPTL3 RNA through a RISC pathway, but will typically range from 9 to 30 base pairs in length, e.g., 15-30 base pairs in length. Considering a duplex between 9 and 30 base pairs, the duplex can be any length in this range, for example, 9, 10, 11, 12, 13, 14, 15, 16, 17, 18, 19, 20, 21, 22, 23, 24, 25, 26, 27, 28, 29, or 30, and any sub-range therein between, including, but not limited to 15-30 base pairs, 15-26 base pairs, 15-23 base pairs, 15-22 base pairs, 15-21 base pairs, 15-20 base pairs, 15-19 base pairs, 15-18 base pairs, 15-17 base pairs, 18-30 base pairs, 18-26 base pairs, 18-23 base pairs, 18-22 base pairs, 18-21 base pairs, 18-20 base pairs, 19-30 base pairs, 19-26 base pairs, 19-23 base pairs, 19-22 base pairs, 19-21 base pairs, 19-20 base pairs, 20-30 base pairs, 20-26 base pairs, 20-25 base pairs, 20-24 base pairs, 20-23 base pairs, 20-22 base pairs, 20-21 base pairs, 21-30 base pairs, 21-26 base pairs, 21-25 base pairs, 21-24 base pairs, 21-23 base pairs, or 21-22 base pairs. ANGPTL3 dsRNA agents generated in the cell by processing with Dicer and similar enzymes are generally in the range of 19-22 base pairs in length. One strand of the duplex region of an ANGPTL3 dsDNA agent comprises a sequence that is substantially complementary to a region of a target ANGPTL3 RNA. The two strands forming the duplex structure can be from a single RNA molecule having at least one self-complementary region, or can be formed from two or more separate RNA molecules. Where the duplex region is formed from two strands of a single molecule, the molecule can have a duplex region separated by a single stranded chain of nucleotides (herein referred to as a “hairpin loop”) between the 3′-end of one strand and the 5′-end of the respective other strand forming the duplex structure.

In some embodiments of the invention, a hairpin look comprises at least 1, 2, 3, 4, 5, 6, 7, 8, 9, 10, 11, 12, 13, 14, 15, 16, 17, 18, 19, 20, or more unpaired nucleotides. Where the two substantially complementary strands of an ANGPTL3 dsRNA agent are comprised by separate RNA molecules, those molecules need not, but can be covalently connected. Where the two strands are connected covalently by means other than a hairpin loop, the connecting structure is referred to as a “linker.” The term “siRNA” is also used herein to refer to a dsRNA agent as described herein.

In some embodiments of the invention an ANGPTL3 dsRNA agent may include a sense and antisense sequence that have no-unpaired nucleotides or nucleotide analogs at one or both terminal ends of the dsRNA agent. An end with no unpaired nucleotides is referred to as a “blunt end” and as having no nucleotide overhang. If both ends of a dsRNA agent are blunt, the dsRNA is referred to as “blunt ended.” In some embodiments of the invention, a first end of a dsRNA agent is blunt, in some embodiments a second end of a dsRNA agent is blunt, and in certain embodiments of the invention, both ends of an ANGPTL3 dsRNA agent are blunt.

In some embodiments of dsRNA agents of the invention, the dsRNA does not have one or two blunt ends. In such instances there is at least one unpaired nucleotide at the end of a strand of a dsRNA agent. For example, when a 3′-end of one strand of a dsRNA extends beyond the 5′-end of the other strand, or vice versa, there is a nucleotide overhang. A dsRNA can comprise an overhang of at least el, 2, 3, 4, 5, 6, or more nucleotides. A nucleotide overhang can comprise or consist of a nucleotide/nucleoside analog, including a deoxynucleotide/nucleoside. It will be understood that in some embodiments a nucleotide overhang is on a sense strand of a dsRNA agent, on an antisense strand of a dsRNA agent, or on both ends of a dsRNA agent and nucleotide(s) of an overhang can be present on the 5′ end, 3′ end or both ends of either an antisense or sense strand of a dsRNA. In certain embodiments of the invention, one or more of the nucleotides in an overhang is replaced with a nucleoside thiophosphate.

As used herein, the term “antisense strand” or “guide strand” refers to the strand of an ANGPTL3 dsRNA agent that includes a region that is substantially complementary to an ANGPTL3 target sequence. As used herein the term “sense strand,” or “passenger strand” refers to the strand of an ANGPTL3 dsRNA agent that includes a region that is substantially complementary to a region of the antisense strand of the ANGPTL3 dsRNA agent.

### Modifications

In some embodiments of the invention the RNA of an ANGPTL3 RNAi agent is chemically modified to enhance stability and/or one or more other beneficial characteristics. Nucleic acids in certain embodiments of the invention may be synthesized and/or modified by methods well established in the art, for example, those described in “Current protocols in Nucleic Acid Chemistry,” Beaucage, S. L. et al. (Eds.), John Wiley & Sons, Inc., New York, N.Y., USA, which is incorporated herein by reference. Modifications that can be present in certain embodiments of ANGPTL3 dsRNA agents of the invention include, for example, (a) end modifications, e.g., 5′ end modifications (phosphorylation, conjugation, inverted linkages, etc.) 3′ end modifications (conjugation, DNA nucleotides, inverted linkages, etc.), (b) base modifications, e.g., replacement with stabilizing bases, destabilizing bases, or bases that base pair with an expanded repertoire of partners, removal of bases (abasic nucleotides), or conjugated bases, (c) sugar modifications (e.g., at the 2′ position or 4′ position) or replacement of the sugar, as well as (d) backbone modifications, including modification or replacement of the phosphodiester linkages. Specific examples of RNA compounds useful in certain embodiments of ANGPTL3 dsRNA agents, ANGPTL3 antisense polynucleotides, and ANGPTL3 sense polynucleotides of the invention include, but are not limited to RNAs comprising modified backbones or no natural internucleoside linkages. As a non-limiting example, an RNA having a modified backbone may not have a phosphorus atom in the backbone. RNAs that do not have a phosphorus atom in their internucleoside backbone may be referred to as oligonucleosides. In certain embodiments of the invention, a modified RNA has a phosphorus atom in its internucleoside backbone.

It will be understood that the term “RNA molecule” or “RNA” or “ribonucleic acid molecule” encompasses not only RNA molecules as expressed or found in nature, but also analogs and derivatives of RNA comprising one or more ribonucleotide/ribonucleoside analogs or derivatives as described herein or as known in the art. The terms “ribonucleoside” and “ribonucleotide” may be used interchangeably herein. An RNA molecule can be modified in the nucleobase structure or in the ribose-phosphate backbone structure, e.g., as described herein below, and molecules comprising ribonucleoside analogs or derivatives must retain the ability to form a duplex. As non-limiting examples, an RNA molecule can also include at least one modified ribonucleoside including but not limited to a 2′-O-methyl modified nucleoside, a nucleoside comprising a 5′ phosphorothioate group, a terminal nucleoside linked to a cholesteryl derivative or dodecanoic acid bisdecylamide group, a locked nucleoside, an abasic nucleoside, a 2′-deoxy-2′-fluoro modified nucleoside, a 2′-amino-modified nucleoside, 2′-alkyl-modified nucleoside, morpholino nucleoside, a phosphoramidate or a non-natural base comprising nucleoside, or any combination thereof. In some embodiments of the invention, an RNA molecule comprises at least 1, 2, 3, 4, 5, 6, 7, 8, 9, 10, 11, 12, 13, 14, 15, 16, 17, 18, 19, 20, or up to the full length of the ANGPTL3 dsRNA agent molecule's ribonucleosides that are modified ribonucleosides. The modifications need not be the same for each of such a plurality of modified ribonucleosides in an RNA molecule.

dsRNA agents, ANGPTL3 antisense polynucleotides, and/or ANGPTL3 sense polynucleotides of the invention may, in some embodiments comprise one or more independently selected modified nucleotide and/or one or more independently selected non-phosphodiester linkage. As used herein the term “independently selected” used in reference to a selected element, such as a modified nucleotide, non-phosphodiester linkage, etc., means that two or more selected elements can but need not be the same as each other.

As used herein, a “nucleotide base,” “nucleotide,” or “nucleobase” is a heterocyclic pyrimidine or purine compound, which is a standard constituent of all nucleic acids, and includes the bases that form the nucleotides adenine (a), guanine (g), cytosine (c), thymine (t), and uracil (u). A nucleobase may further be modified to include, though not intended to be limiting: universal bases, hydrophobic bases, promiscuous bases, size-expanded bases, and fluorinated bases. The term “ribonucleotide” or “nucleotide” may be used herein to refer to an unmodified nucleotide, a modified nucleotide, or a surrogate replacement moiety. Those in the art will recognize that guanine, cytosine, adenine, and uracil can be replaced by other moieties without substantially altering the base pairing properties of an oligonucleotide comprising a nucleotide bearing such replacement moiety.

In one embodiment, modified RNAs contemplated for use in methods and compositions described herein are peptide nucleic acids (PNAs) that have the ability to form the required duplex structure and that permit or mediate the specific degradation of a target RNA via a RISC pathway. In certain embodiments of the invention, an ANGPTL3 RNA interference agent includes a single stranded RNA that interacts with a target ANGPTL3 RNA sequence to direct the cleavage of the target ANGPTL3 RNA.

Modified RNA backbones can include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those) having inverted polarity wherein the adjacent pairs of nucleoside units are linked 3′-5′ to 5′-3′ or 2′-5′ to 5′-2′. Various salts, mixed salts and free acid forms are also included. Means of preparing phosphorus-containing linkages are routinely practiced in the art and such methods can be used to prepare certain modified ANGPTL3 dsRNA agents, certain modified ANGPTL3 antisense polynucleotides, and/or certain modified ANGPTL3 sense polynucleotides of the invention.

Modified RNA backbones that do not include a phosphorus atom therein have backbones that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. Means of preparing modified RNA backbones that do not include a phosphorus atom are routinely practiced in the art and such methods can be used to prepare certain modified ANGPTL3 dsRNA agents, certain modified ANGPTL3 antisense polynucleotides, and/or certain modified ANGPTL3 sense polynucleotides of the invention.

In certain embodiments of the invention, RNA mimetics are included in ANGPTL3 dsRNAs, ANGPTL3 antisense polynucleotides, and/or ANGPTL3 sense polynucleotides, such as, but not limited to: replacement of the sugar and the internucleoside linkage, i.e., the backbone, of the nucleotide units with novel groups. In such embodiments, base units are maintained for hybridization with an appropriate ANGPTL3 nucleic acid target compound. One such oligomeric compound, an RNA mimetic that has been shown to have excellent hybridization properties, is referred to as a peptide nucleic acid (PNA). In PNA compounds, the sugar backbone of an RNA is replaced with an amide containing backbone, in particular an aminoethylglycine backbone. The nucleobases are retained and are bound directly or indirectly to aza nitrogen atoms of the amide portion of the backbone. Means of preparing RNA mimetics are routinely practiced in the art and such methods can be used to prepare certain modified ANGPTL3 dsRNA agents of the invention.

Some embodiments of the invention include RNAs with phosphorothioate backbones and oligonucleosides with heteroatom backbones, and in particular —CH₂—NH—CH₂—, —CH₂—N(CH₃)—O—CH₂—[known as a methylene (methylimino) or MMI backbone], —CH₂—O—N(CH₃)—CH₂—, —CH₂—N(CH₃)—N(CH₃)—CH₂— and —N(CH₃)—CH₂— [wherein the native phosphodiester backbone is represented as —O—P—O—CH₂—]. Means of preparing RNAs with phosphorothioate backbones and oligonucleosides with heteroatom backbones are routinely practiced in the art and such methods can be used to prepare certain modified ANGPTL3 dsRNA agents, certain ANGPTL3 antisense polynucleotides, and/or certain ANGPTL3 sense polynucleotides of the invention.

Modified RNAs can also contain one or more substituted sugar moieties. ANGPTL3 dsRNAs, ANGPTL3 antisense polynucleotides, and/or ANGPTL3 sense polynucleotides of the invention may comprise one of the following at the 2′ position: OH; F; O—, S—, or N-alkyl; O—, S—, or N-alkenyl; O—, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Exemplary suitable modifications include O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. In other embodiments, dsRNAs include one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an ANGPTL3 dsRNA agent, or a group for improving the pharmacodynamic properties of an ANGPTL3 dsRNA agent, ANGPTL3 antisense polynucleotide, and/or ANGPTL3 sense polynucleotide, and other substituents having similar properties. In some embodiments, the modification includes a 2′-methoxyethoxy (2′-O—CH₂CH₂OCH₃, also known as 2′-O-(2-methoxyethyl) or 2′-MOE) (Martin et al., Helv. Chim. Acta, 1995, 78:486-504) i.e., an alkoxy-alkoxy group. Another exemplary modification is 2′-dimethylaminooxyethoxy, i.e., a O(CH₂)₂ON(CH₃)₂ group, also known as 2′-DMAOE, as described in examples herein below, and 2′-dimethylaminoethoxyethoxy (also known in the art as 2′-O-dimethylaminoethoxyethyl or 2′-DMAEOE), i.e., 2′-O—CH₂—O—CH₂—N(CH₂)₂. Means of preparing modified RNAs such as those described are routinely practiced in the art and such methods can be used to prepare certain modified ANGPTL3 dsRNA agents of the invention.

Other modifications include 2′-methoxy (2′-OCH₃), 2′-aminopropoxy (2′-OCH₂CH₂CH₂NH₂) and 2′-fluoro (2′-F). Similar modifications can also be made at other positions on the RNA of an ANGPTL3 dsRNA agent, ANGPTL3 antisense polynucleotide, and/or ANGPTL3 sense polynucleotide of the invention, particularly the 3′ position of the sugar on the 3′ terminal nucleotide or in 2′-5′ linked ANGPTL3 dsRNAs, ANGPTL3 antisense polynucleotides, or ANGPTL3 sense polynucleotides, and the 5′ position of 5′ terminal nucleotide. ANGPTL3 dsRNA agents, ANGPTL3 antisense polynucleotides, and/or ANGPTL3 sense polynucleotides may also have sugar mimetics such as cyclobutyl moieties in place of the pentofuranosyl sugar. Means of preparing modified RNAs such as those described are routinely practiced in the art and such methods can be used to prepare certain modified ANGPTL3 dsRNA agents, ANGPTL3 antisense polynucleotides, and/or ANGPTL3 sense polynucleotides of the invention.

An ANGPTL3 dsRNA agent, ANGPTL3 antisense polynucleotide, and/or ANGPTL3 sense polynucleotide may, in some embodiments, include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl anal other 8-substituted adenines and guanines, 5-halo, particularly 5-bromo, 5-trifluoromethyl and other 5-substituted uracils and cytosines, 7-methylguanine and 7-methyladenine, 8-azaguanine and 8-azaadenine, 7-deazaguanine and 7-daazaadenine and 3-deazaguanine and 3-deazaadenine. Additional nucleobases that may be included in certain embodiments of ANGPTL3 dsRNA agents of the invention are known in the art, see for example: Modified Nucleosides in Biochemistry, Biotechnology and Medicine, Herdewijn, P. Ed. Wiley-VCH, 2008; The Concise Encyclopedia Of Polymer Science And Engineering, pages 858-859, Kroschwitz, J. L, Ed. John Wiley & Sons, 1990, English et al., Angewandte Chemie, International Edition, 1991, 30, 613, Sanghvi, Y S., Chapter 15, dsRNA Research and Applications, pages 289-302, Crooke, S. T. and Lebleu, B., Ed., CRC Press, 1993. Means of preparing dsRNAs, ANGPTL3 antisense strand polynucleotides and/or ANGPTL3 sense strand polynucleotides that comprise nucleobase modifications and/or substitutions such as those described herein are routinely practiced in the art and such methods can be used to prepare certain modified ANGPTL3 dsRNA agents, ANGPTL3 sense polynucleotides, and/or ANGPTL3 antisense polynucleotides of the invention.

Certain embodiments of ANGPTL3 dsRNA agents, ANGPTL3 antisense polynucleotides, and/or ANGPTL3 sense polynucleotides of the invention include RNA modified to include one or more locked nucleic acids (LNA). A locked nucleic acid is a nucleotide with a modified ribose moiety comprising an extra bridge connecting the 2′ and 4′ carbons. This structure effectively “locks” the ribose in the 3′-endo structural conformation. The addition of locked nucleic acids in an ANGPTL3 dsRNA agent, ANGPTL3 antisense polynucleotides, and/or ANGPTL3 sense polynucleotides of the invention may increase stability in serum, and to reduce off-target effects (Elmen, J. et al., (2005) Nucleic Acids Research 33(1):439-447; Mook, O R. et al., (2007) Mol Canc Ther 6(3):833-843; Grunweller, A. et al., (2003) Nucleic Acids Research 31(12):3185-3193). Means of preparing dsRNA agents, ANGPTL3 antisense polynucleotides, and/or ANGPTL3 sense polynucleotides that comprise locked nucleic acid(s) are routinely practiced in the art and such methods can be used to prepare certain modified ANGPTL3 dsRNA agents of the invention.

Certain embodiments of ANGPTL3 dsRNA compounds, sense polynucleotides, and/or antisense polynucleotides of the invention, include at least one modified nucleotide, wherein the at least one modified nucleotide comprises: a 2′-O-methyl nucleotide,2′-Fluoro nucleotide, 2′-deoxy nucleotide, 2′3′-seco nucleotide mimic, locked nucleotide, 2′-F-Arabino nucleotide, 2′-methoyxyethyl nucleotide, 2′-amino-modified nucleotide, 2′-alkyl-modified nucleotide, mopholino nucleotide, and 3′-Ome nucleotide, a nucleotide comprising a 5′-phosphorothioate group, or a terminal nucleotide linked to a cholesteryl derivative or dodecanoic acid bisdecylamide group, a 2′-amino-modified nucleotide, ′a phosphoramidate, or a non-natural base comprising nucleotide. In some embodiments, an ANGPTL3 dsRNA compound includes an E-vinylphosphonate nucleotide at the 5′ end of the antisense strand, also referred to herein as the guide strand.

Certain embodiments of ANGPTL3 dsRNA compounds, 3′ and 5′ end of sense polynucleotides, and/or 3′ end of antisense polynucleotides of the invention, include at least one modified nucleotide, wherein the at least one modified nucleotide comprises: abasic nucleotide, ribitol, inverted nucleotide, inverted abasic nucleotide, inverted 2′-OMe nucleotide, inverted 2′-deoxy nucleotide. It is known to skilled in art, including an abasic or inverted abasic nucleotide at the end of oligonucleotide enhances stability (Czauderna et al. Structural variations and stabilizing modifications of synthetic siRNAs in mammalian cells. Nucleic Acids Res. 2003; 31(11):2705-2716. doi:10.1093/nar/gkg393).

Certain embodiments of ANGPTL3 dsRNA compounds, antisense polynucleotides of the invention, include at least one modified nucleotide, wherein the at least one modified nucleotide comprises unlocked nucleic acid nucleotide (UNA) or/and glycol nucleic acid nucleotide (GNA). It is known to skilled in art, UNA and GNA are thermally destabilizing chemical modifications, can significantly improves the off-target profile of a siRNA compound (Janas, et al., Selection of GalNAc-conjugated siRNAs with limited off-target-driven rat hepatotoxicity. Nat Commun. 2018; 9(1):723. doi:10.1038/s41467-018-02989-4; Laursen et al., Utilization of unlocked nucleic acid (UNA) to enhance siRNA performance in vitro and in vivo. Mol BioSyst. 2010; 6:862-70).

Another modification that may be included in the RNA of certain embodiments of ANGPTL3 dsRNA agents, ANGPTL3 antisense polynucleotides, and/or ANGPTL3 sense polynucleotides of the invention, comprises chemically linking to the RNA one or more ligands, moieties or conjugates that enhance one or more characteristics of the ANGPTL3 dsRNA agent, ANGPTL3 antisense polynucleotide, and/or ANGPTL3 sense polynucleotide, respectively. Non-limiting examples of characteristics that may be enhanced are: ANGPTL3 dsRNA agent, ANGPTL3 antisense polynucleotide, and/or ANGPTL3 sense polynucleotide activity, cellular distribution, delivery of an ANGPTL3 dsRNA agent, pharmacokinetic properties of an ANGPTL3 dsRNA agent, and cellular uptake of the ANGPTL3 dsRNA agent. In some embodiments of the invention, an ANGPTL3 dsRNA agent comprises one or more targeting groups or linking groups, which in certain embodiments of ANGPTL3 dsRNA agents of the invention are conjugated to the sense strand. A non-limiting example of a targeting group is a compound comprising N-acetyl-galactosamine (GalNAc). The terms “targeting group”, “targeting agent”, “linking agent”, “targeting compound”, and “targeting ligand” may be used interchangeably herein. In certain embodiments of the invention an ANGPTL3 dsRNA agent comprises a targeting compound that is conjugated to the 5′-terminal end of the sense strand. In certain embodiments of the invention an ANGPTL3 dsRNA agent comprises a targeting compound that is conjugated to the 3′-terminal end of the sense strand. In some embodiments of the invention, an ANGPTL3 dsRNA agent comprises a targeting group that comprises GalNAc. In certain embodiments of the invention an ANGPTL3 dsRNA agent does not include a targeting compound conjugated to one or both of the 3′-terminal end and the 5′-terminal end of the sense strand. In certain embodiments of the invention an ANGPTL3 dsRNA agent does not include a GalNAc containing targeting compound conjugated to one or both of the 5′-terminal end and the 3′-terminal end of the sense strand.

Additional targeting and linking agents are well known in the art, for example, targeting and linking agents that may be used in certain embodiments of the invention include but are not limited to lipid moieties such as a cholesterol moiety (Letsinger et al., Proc. Natl. Acid. Sci. USA, 1989, 86: 6553-6556), cholic acid (Manoharan et al., Biorg. Med. Chem. Let., 1994, 4:1053-1060), a thioether, e.g., beryl-S-tritylthiol (Manoharan et al., Ann. N.Y. Acad. Sci., 1992, 660:306-309; Manoharan et al., Biorg. Med. Chem. Let., 1993, 3:2765-2770), a thiocholesterol (Oberhauser et al., Nucl. Acids Res., 1992, 20:533-538), an aliphatic chain, e.g., dodecandiol or undecyl residues (Saison-Behmoaras et al., EMBO J, 1991, 10:1111-1118; Kabanov et al., FEBS Lett., 1990, 259:327-330; Svinarchuk et al., Biochimie, 1993, 75:49-54), a phospholipid, e.g., di-hexadecyl-rac-glycerol or triethyl-ammonium 1,2-di-O-hexadecyl-rac-glycero-3-phosphonate (Manoharan et al., Tetrahedron Lett., 1995, 36:3651-3654; Shea et al., Nucl. Acids Res., 1990, 18:3777-3783), a polyamine or a polyethylene glycol chain (Manoharan et al., Nucleosides & Nucleotides, 1995, 14:969-973), or adamantane acetic acid (Manoharan et al., Tetrahedron Lett., 1995, 36:3651-3654), a palmityl moiety (Mishra et al., Biochim. Biophys. Acta, 1995, 1264:229-237), or an octadecylamine or hexylamino-carbonyloxycholesterol moiety (Crooke et al., J. Pharmacol. Exp. Ther., 1996, 277:923-937).

Certain embodiments of a composition comprising an ANGPTL3 dsRNA agent, ANGPTL3 antisense polynucleotide, and/or ANGPTL3 sense polynucleotide may comprise a ligand that alters distribution, targeting, or etc. of the ANGPTL3 dsRNA agent. In some embodiments of a composition comprising an ANGPTL3 dsRNA agent of the invention, the ligand increases affinity for a selected target, e.g., molecule, cell or cell type, compartment, e.g., a cellular or organ compartment, tissue, organ or region of the body, as, e.g., compared to a species absent such a ligand. A ligand useful in a composition and/or method of the invention may be a naturally occurring substance, such as a protein (e.g., human serum albumin (HSA), low-density lipoprotein (LDL), or globulin); a carbohydrate (e.g., a dextran, pullulan, chitin, chitosan, inulin, cyclodextrin or hyaluronic acid); or a lipid. A ligand may also be a recombinant or synthetic molecule, such as a synthetic polymer, e.g., a synthetic polyamino acid or polyamine. Examples of polyamino acids are a polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacryllic acid), N-isopropylacrylamide polymers, or polyphosphazine. Example of polyamines include: polyethylenimine, polylysine (PLL), spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, or an alpha helical peptide.

A ligand included in a composition and/or method of the invention may comprise a targeting group, non-limiting examples of which are a cell or tissue targeting agent, e.g., a lectin, glycoprotein, lipid or protein, e.g., an antibody that binds to a specified cell type such as a kidney cell or a liver cell. A targeting group can be a thyrotropin, melanotropin, lectin, glycoprotein, surfactant protein A, Mucin carbohydrate, multivalent lactose, multivalent galactose, N-acetyl-galactosamine, N-acetyl-gulucosamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, vitamin A, biotin, or an RGD peptide or RGD peptide mimetic.

Other examples of ligands include dyes, intercalating agents (e.g. acridines), cross-linkers (e.g. psoralene, mitomycin C), porphyrins (TPPC4, texaphyrin, Sapphyrin), polycyclic aromatic hydrocarbons (e.g., phenazine, dihydrophenazine), artificial endonucleases (e.g. EDTA), lipophilic molecules, e.g., cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, or phenoxazine) and peptide conjugates (e.g., antennapedia peptide, Tat peptide), alkylating agents, phosphate, amino, mercapto, PEG (e.g., PEG-40K), MPEG, [MPEG]₂, polyamino, alkyl, substituted alkyl, radiolabeled markers, enzymes, haptens (e.g., biotin), transport/absorption facilitators (e.g., aspirin, vitamin E, folic acid), synthetic ribonucleases (e.g., imidazole, bisimidazole, histamine, imidazole clusters, acridine-imidazole conjugates, Eu3+ complexes of tetraazamacrocycles), dinitrophenyl, HRP, or AP.

A ligand included in a composition and/or method of the invention may be a protein, e.g., glycoprotein, or peptide, for example a molecule with a specific affinity for a co-ligand, or an antibody, for example an antibody, that binds to a specified cell type such as a cancer cell, endothelial cell, cardiac cell, or bone cell. A ligand useful in an embodiment of a composition and/or method of the invention can be a hormone or hormone receptor. A ligand useful in an embodiment of a composition and/or method of the invention can be a lipid, lectin, carbohydrates, vitamin, cofactos, multivalent lactose, multivalent galactose, N-acetyl-galactosamine, N-acetyl-gulucosamine multivalent mannose, or multivalent fucose. A ligand useful in an embodiment of a composition and/or method of the invention can be a substance that can increase uptake of the ANGPTL3 dsRNA agent into the cell, for example, by disrupting the cell's cytoskeleton, e.g., by disrupting the cell's microtubules, microfilaments, and/or intermediate filaments. Non-limiting examples of this type of agent are: taxon, vincristine, vinblastine, cytochalasin, nocodazole, japlakinolide, latrunculin A, phalloidin, swinholide A, indanocine, and myoservin.

In some embodiments, a ligand attached to an ANGPTL3 dsRNA agent of the invention functions as a pharmacokinetic (PK) modulator. An example of a PK modulator that may be used in compositions and methods of the invention includes but is not limited to: a lipophiles, a bile acid, a steroid, a phospholipid analogue, a peptide, a protein binding agent, PEG, a vitamin, cholesterol, a fatty acid, cholic acid, lithocholic acid, dialkylglycerides, diacylglyceride, a phospholipid, a sphingolipid, naproxen, ibuprofen, vitamin E, biotin, an aptamer that binds a serum protein, etc. Oligonucleotides comprising a number of phosphorothioate linkages are also known to bind to serum protein, thus short oligonucleotides, e.g., oligonucleotides of about 5 bases, 10 bases, 15 bases or 20 bases, comprising multiple of phosphorothioate linkages in the backbone may also be used in compositions and/or methods of the invention as ligands.

ANGPTL3 dsRNA Agent Compositions

In some embodiments of the invention, an ANGPTL3 dsRNA agent is in a composition. A composition of the invention may include one or more ANGPTL3 dsRNA agent and optionally one or more of a pharmaceutically acceptable carrier, a delivery agent, a targeting agent, detectable label, etc. A non-limiting example of a targeting agent that may be useful according to some embodiments of methods of the invention is an agent that directs an ANGPTL3 dsRNA agent of the invention to and/or into a cell to be treated. A targeting agent of choice will depend upon such elements as: the nature of the ANGPTL3-associated disease or condition, and on the cell type being targeted. In a non-limiting example, in some embodiments of the invention it may be desirable to target an ANGPTL3 dsRNA agent to and/or into a liver cell. It will be understood that in some embodiments of methods of the invention, a therapeutic agent comprises a ANGPTL3 dsRNA agent with only a delivery agent, such as a delivery agent comprising N-Acetylgalactosamine (GalNAc), without any additional attached elements. For example, in some aspects of the invention an ANGPTL3 dsRNA agent may be attached to a delivery compound comprising GalNAc and included in a composition comprising a pharmaceutically acceptable carrier and administered to a cell or subject without any detectable labels, or targeting agents, etc. attached to the ANGPTL3 dsRNA agent.

In cases where an ANGPTL3 dsRNA agent of the invention is administered with and/or attached to one or more delivery agents, targeting agents, labeling agents, etc. a skilled artisan will be aware of and able to select and use suitable agents for use in methods of the invention. Labeling agents may be used in certain methods of the invention to determine the location of an ANGPTL3 dsRNA agent in cells and tissues and may be used to determine a cell, tissue, or organ location of a treatment composition comprising an ANGPTL3 dsRNA agent that has been administered in methods of the invention. Procedures for attaching and utilizing labeling agents such as enzymatic labels, dyes, radiolabels, etc. are well known in the art. It will be understood that in some embodiments of compositions and methods of the invention, a labeling agent is attached to one or both of a sense polynucleotide and an antisense polynucleotide included in an ANGPTL3 dsRNA agent.

Delivery of ANGPTL3 dsRNA Agents and ANGPTL3 Antisense Polynucleotide Agents

Certain embodiments of methods of the invention, includes delivery of an ANGPTL3 dsRNA agent into a cell. As used herein the term, “delivery” means facilitating or effecting uptake or absorption into the cell. Absorption or uptake of an ANGPTL3 dsRNA agent can occur through unaided diffusive or active cellular processes, or by use of delivery agents, targeting agents, etc. that may be associated with an ANGPTL3 dsRNA agent of the invention. Delivery means that are suitable for use in methods of the invention include, but are not limited to: in vivo delivery, in which an ANGPTL3 dsRNA agent is in injected into a tissue site or administered systemically. In some embodiments of the invention, an ANGPTL3 dsRNA agent is attached to a delivery agent.

Non-limiting examples of methods that can be used to deliver ANGPTL3 dsRNA agents to cells, tissues and/or subjects include: ANGPTL3 dsRNA-GalNAc conjugates, SAMiRNA technology, LNP-based delivery methods, and naked RNA delivery. These and other delivery methods have been used successfully in the art to deliver therapeutic RNAi agents for treatment of various diseases and conditions, such as but not limited to: liver diseases, acute intermittent porphyria (AIP), hemophilia, pulmonary fibrosis, etc. Details of various delivery means are found in publications such as: Nikam, R. R. & K. R. Gore (2018) Nucleic Acid Ther, 28 (4), 209-224 August 2018; Springer A. D. & S. Dowdy (2018) Nucleic Acid Ther. June 1; 28(3): 109-118; Lee, K. et al., (2018) Arch Pharm Res, 41(9), 867-874; and Nair, J. K. et al., (2014) J. Am. Chem. Soc. 136:16958-16961, the content each of which is incorporated by reference herein.

Some embodiments of the invention comprise use of lipid nanoparticles (LNPs) to deliver an ANGPTL3 dsRNA agent of the invention to a cell, tissue, and/or subject. LNPs are routinely used for in vivo delivery of ANGPTL3 dsRNA agents, including therapeutic ANGPTL3 dsRNA agents. One benefit of using an LNP or other delivery agent is an increased stability of the ANGPTL3 RNA agent when it is delivered to a subject using the LNP or other delivery agent. In some embodiments of the invention an LNP comprises a cationic LNP that is loaded with one or more ANGPTL3 RNAi molecules of the invention. The LNP comprising the ANGPTL3 RNAi molecule(s) is administered to a subject, the LNPs and their attached ANGPTL3 RNAi molecules are taken up by cells via endocytosis, their presence results in release of RNAi trigger molecules, which mediate RNAi.

Another non-limiting example of a delivery agent that may be used in embodiments of the invention to delivery an ANGPTL3 dsRNA agent of the invention to a cell, tissue and/or subject is an agent comprising GalNAc that is attached to an ANGPTL3 dsRNA agent of the invention and delivers the ANGPTL3 dsRNA agent to a cell, tissue, and/or subject. Examples of certain additional delivery agents comprising GalNAc that can be used in certain embodiments of methods and composition of the invention are disclosed in PCT Application: WO2020191183A1. A non-limiting example of a GalNAc targeting ligand that can be used in compositions and methods of the invention to deliver an ANGPTL3 dsRNA agent to a cell is a targeting ligand cluster. Examples of targeting ligand clusters that are presented herein are referred to as: GalNAc Ligand with phosphodiester link (GLO) and GalNAc Ligand with phosphorothioate link (GLS). The term “GLX-n” may be used herein to indicate the attached GalNAC-containing compound is any one of compounds GLS-1, GLS-2, GLS-3, GLS-4, GLS-5, GLS-6, GLS-7, GLS-8, GLS-9, GLS-10, GLS-11, GLS-12, GLS-13, GLS-14, GLS-15, GLS-16, GLO-1, GLO-2, GLO-3, GLO-4, GLO-5, GLO-6, GLO-7, GLO-8, GLO-9, GLO-10, GLO-11, GLO-12, GLO-13, GLO-14, GLO-15, and GLO-16, the structure of each of which is shown below, with the below with location of attachment of the GalNAc-targeting ligand to an RNAi agent of the invention at far right of each (shown with “ [symbol] ”). It will be understood that any RNAi and dsRNA molecule of the invention can be attached to the GLS-1, GLS-2, GLS-3, GLS-4, GLS-5, GLS-6, GLS-7, GLS-8, GLS-9, GLS-10, GLS-11, GLS-12, GLS-13, GLS-14, GLS-15, GLS-16, GLO-1, GLO-2, GLO-3, GLO-4, GLO-5, GLO-6, GLO-7, GLO-8, GLO-9, GLO-10, GLO-11, GLO-12, GLO-13, GLO-14, GLO-15, and GLO-16, GLO-1 through GLO-16 and GLS-1 through GLS-16 structures.

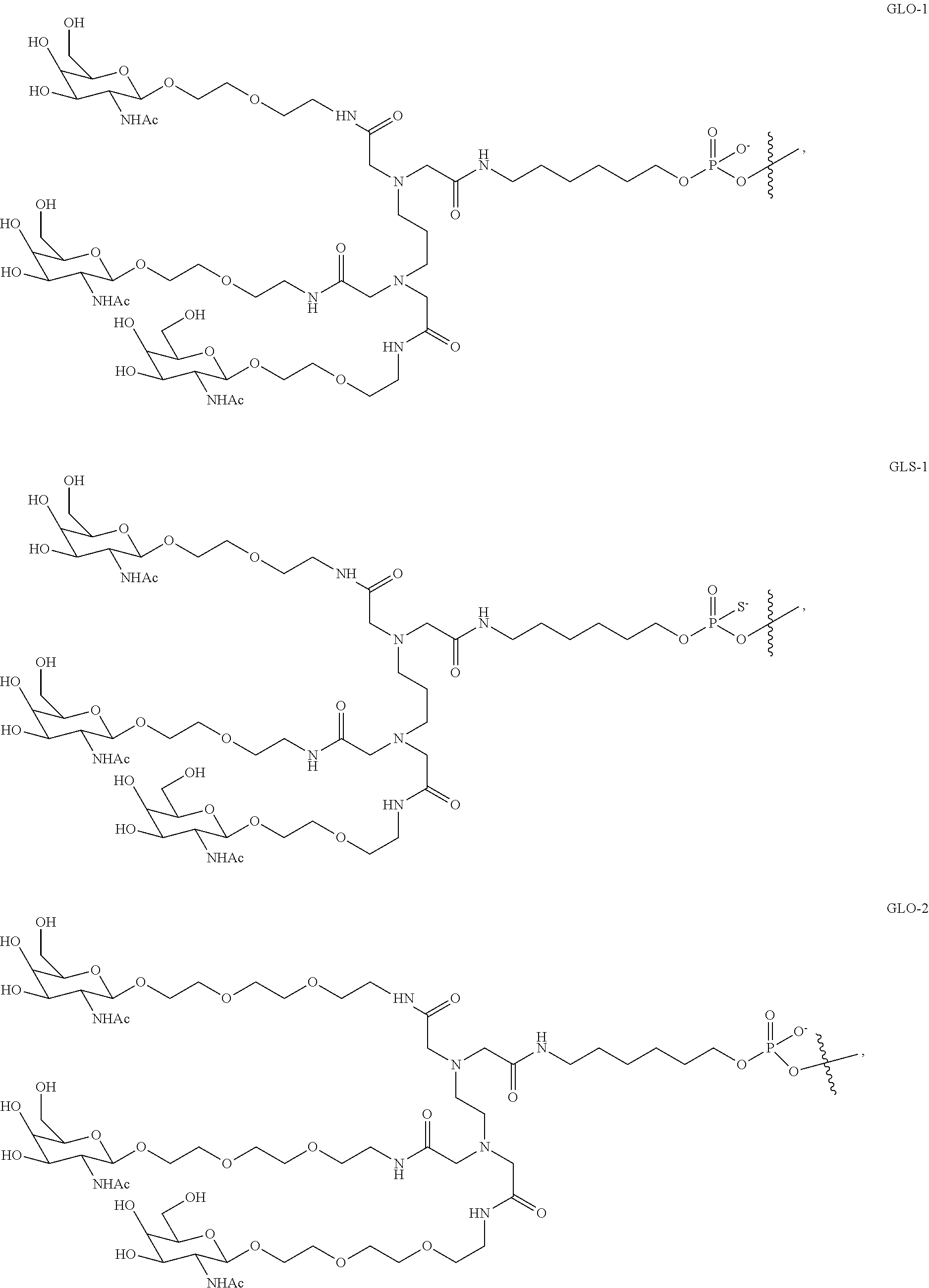 [SMILES: *OP(=O)([O-])OCCCCCCNC(=O)CN(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([S-])OCCCCCCNC(=O)CN(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([O-])OCCCCCCNC(=O)CN(CCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O]

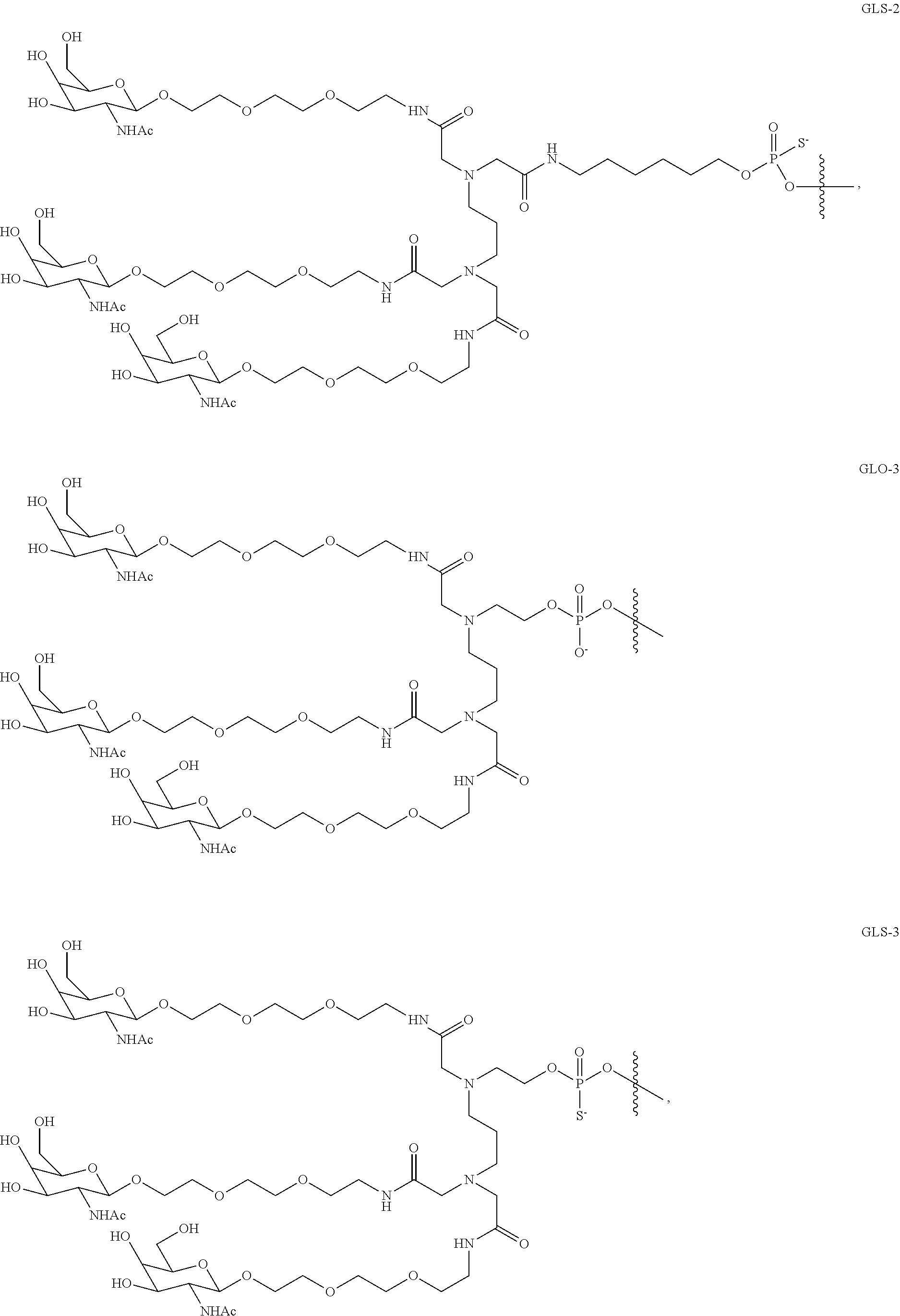 [SMILES: *OP(=O)([S-])OCCCCCCNC(=O)CN(CCCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O ; *OP(=O)([O-])OCCN(CCCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O ; *OP(=O)([S-])OCCN(CCCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O]

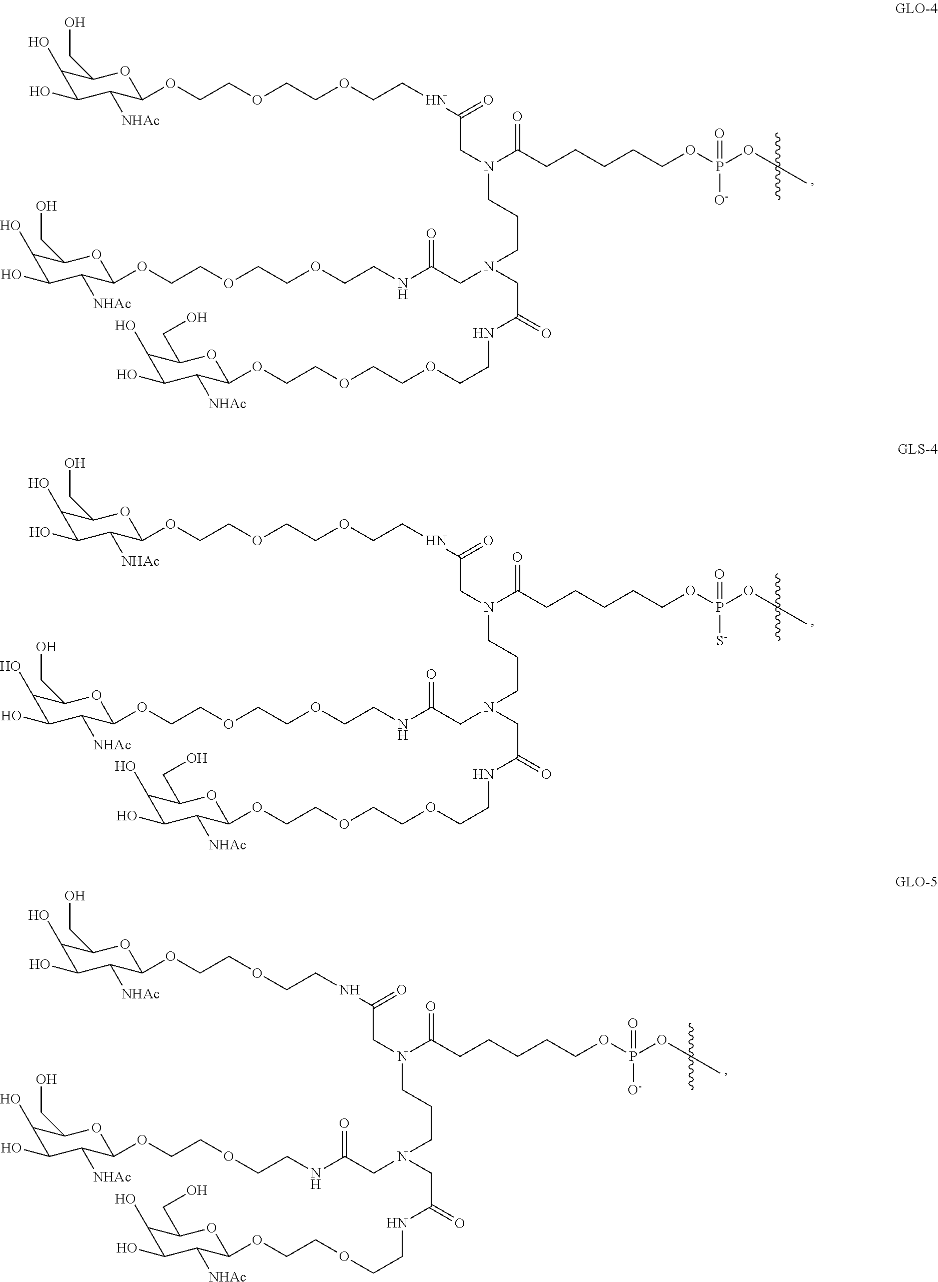 [SMILES: *OP(=O)([O-])OCCCCCC(=O)N(CCCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O ; *OP(=O)([S-])OCCCCCC(=O)N(CCCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O ; *OP(=O)([O-])OCCCCCC(=O)N(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O]

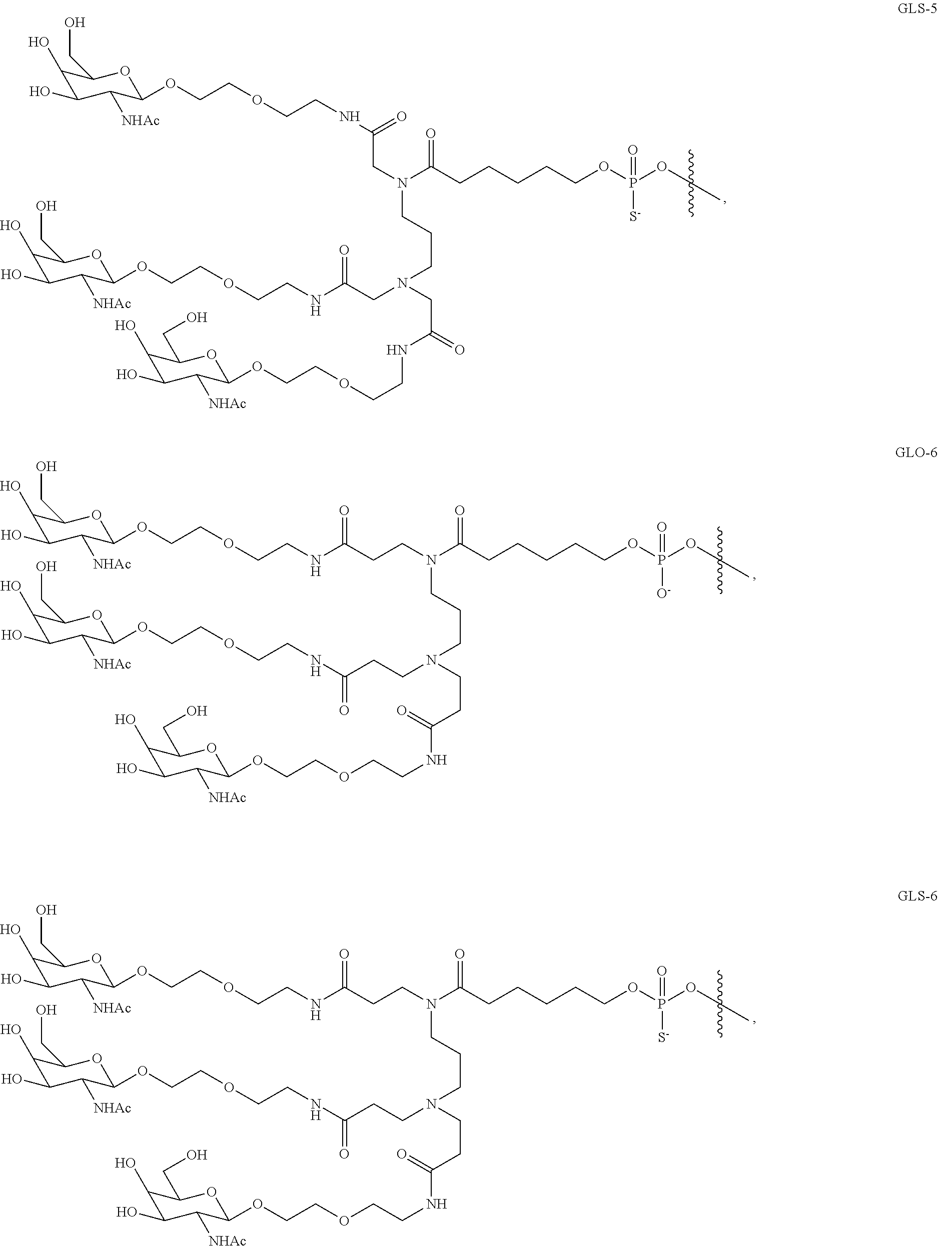 [SMILES: *OP(=O)([S-])OCCCCCC(=O)N(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([O-])OCCCCCC(=O)N(CCCN(CCC(=O)NCCOCCOC1OC(CO)C(O)C(O)C1NC(C)=O)CCC(=O)NCCOCCOC1OC(CO)C(O)C(O)C1NC(C)=O)CCC(=O)NCCOCCOC1OC(CO)C(O)C(O)C1NC(C)=O ; *OP(=O)([S-])OCCCCCC(=O)N(CCCN(CCC(=O)NCCOCCOC1OC(CO)C(O)C(O)C1NC(C)=O)CCC(=O)NCCOCCOC1OC(CO)C(O)C(O)C1NC(C)=O)CCC(=O)NCCOCCOC1OC(CO)C(O)C(O)C1NC(C)=O]

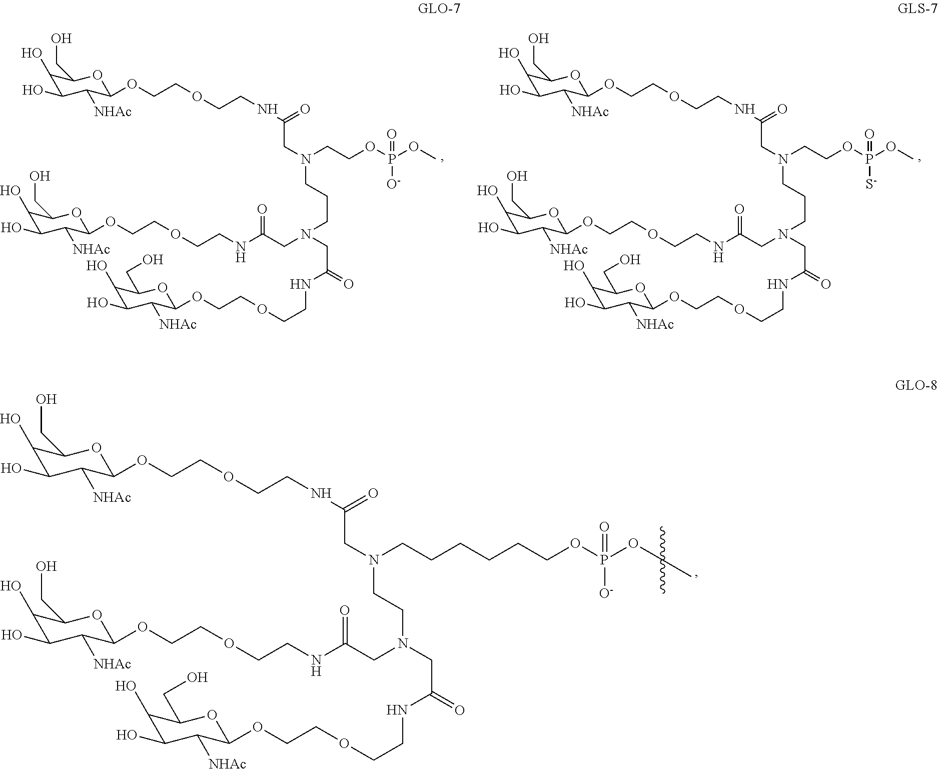 [SMILES: COP(=O)([O-])OCCN(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; COP(=O)([S-])OCCN(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([O-])OCCCCCCN(CCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O]

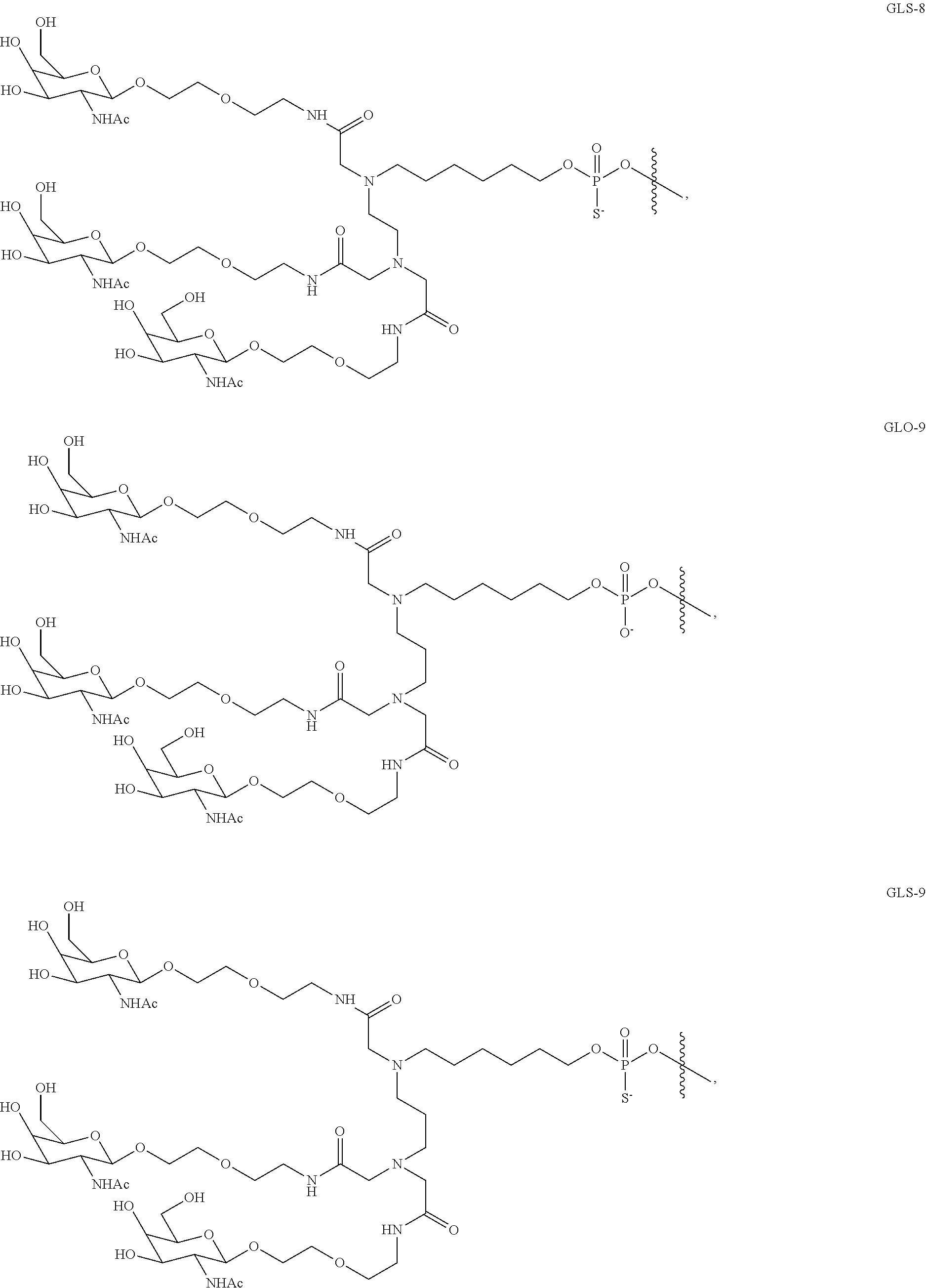 [SMILES: *OP(=O)([S-])OCCCCCCN(CCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([O-])OCCCCCCN(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([S-])OCCCCCCN(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O]

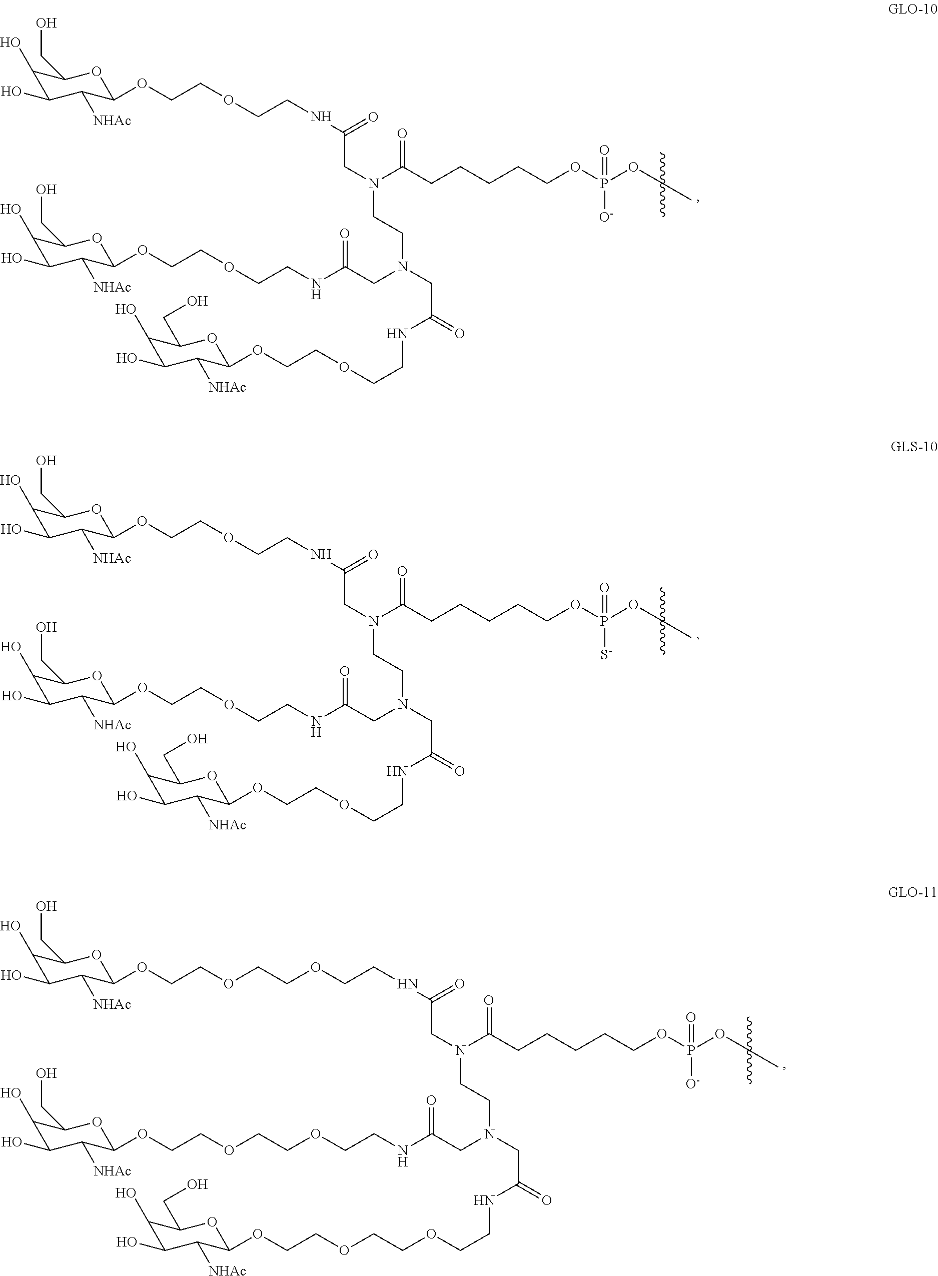 [SMILES: *OP(=O)([O-])OCCCCCC(=O)N(CCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([S-])OCCCCCC(=O)N(CCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([O-])OCCCCCC(=O)N(CCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O]

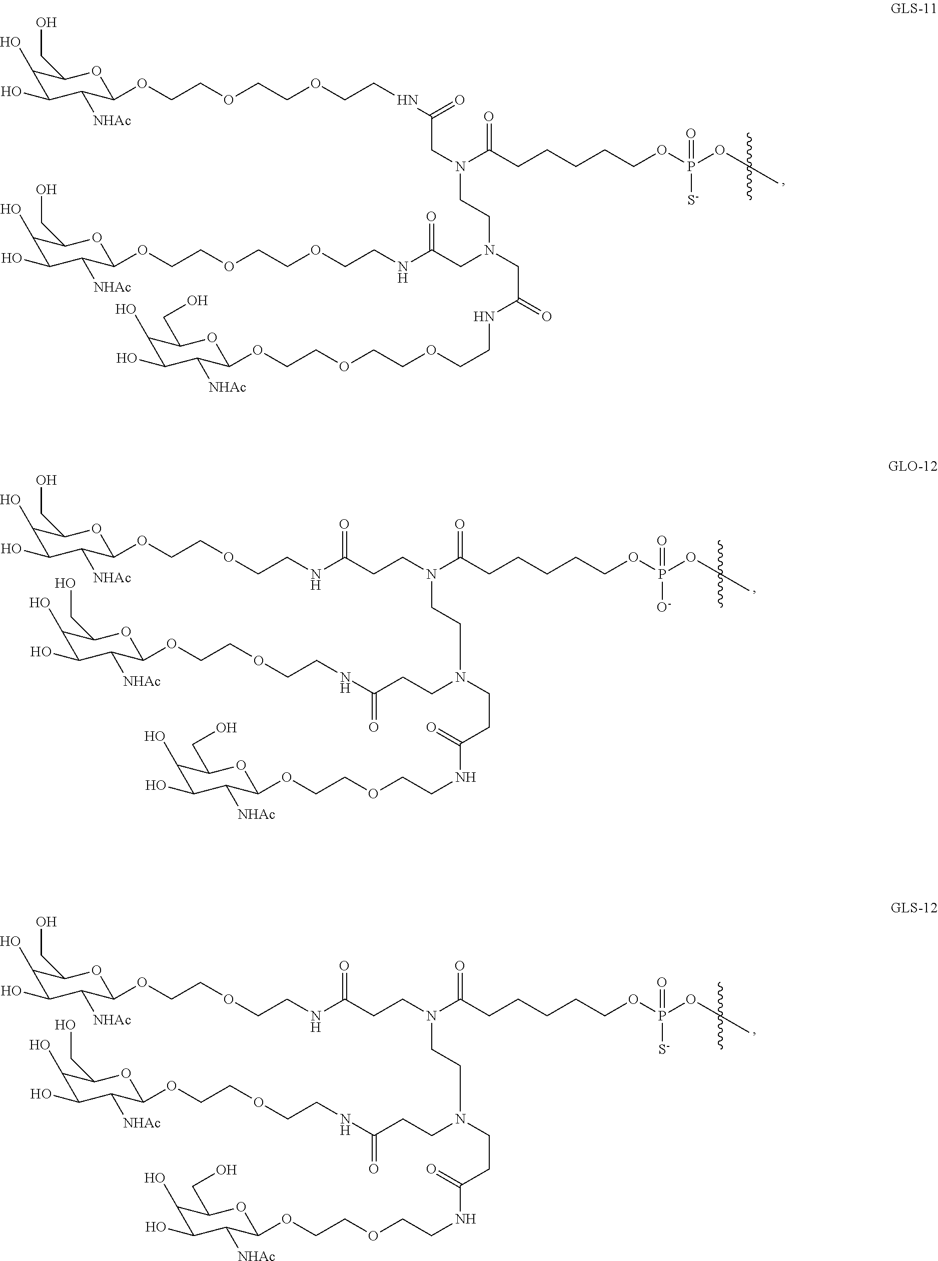 [SMILES: *OP(=O)([S-])OCCCCCC(=O)N(CCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O ; *OP(=O)([O-])OCCCCCC(=O)N(CCC(=O)NCCOCCOC1OC(CO)C(O)C(O)C1NC(C)=O)CCN(CCC(=O)NCCOCCOC1OC(CO)C(O)C(O)C1NC(C)=O)CCC(=O)NCCOCCOC1OC(CO)C(O)C(O)C1NC(C)=O ; *OP(=O)([S-])OCCCCCC(=O)N(CCC(=O)NCCOCCOC1OC(CO)C(O)C(O)C1NC(C)=O)CCN(CCC(=O)NCCOCCOC1OC(CO)C(O)C(O)C1NC(C)=O)CCC(=O)NCCOCCOC1OC(CO)C(O)C(O)C1NC(C)=O]

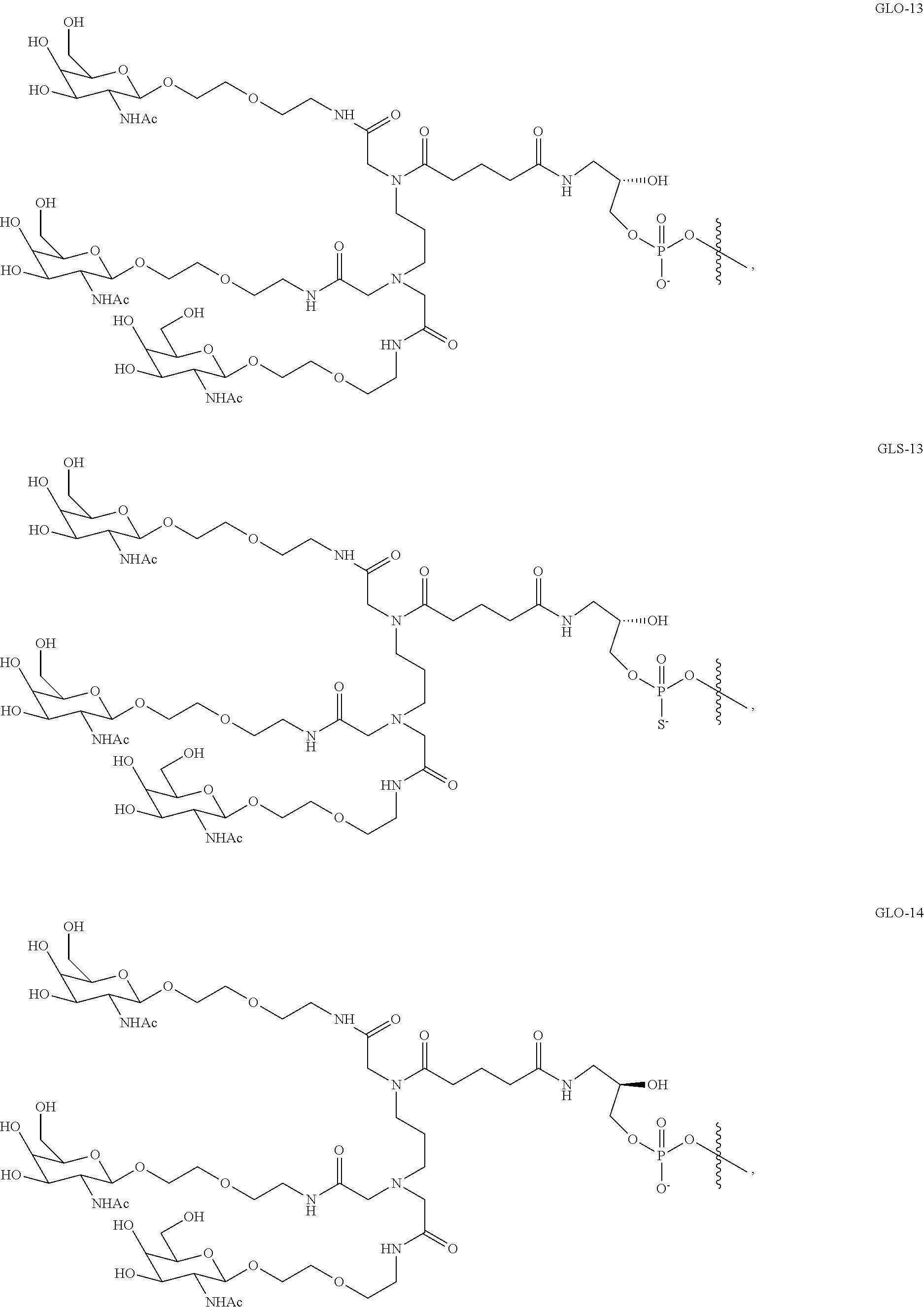 [SMILES: *OP(=O)([O-])OC[C@@H](O)CNC(=O)CCCC(=O)N(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([S-])OC[C@@H](O)CNC(=O)CCCC(=O)N(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([O-])OC[C@H](O)CNC(=O)CCCC(=O)N(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O]

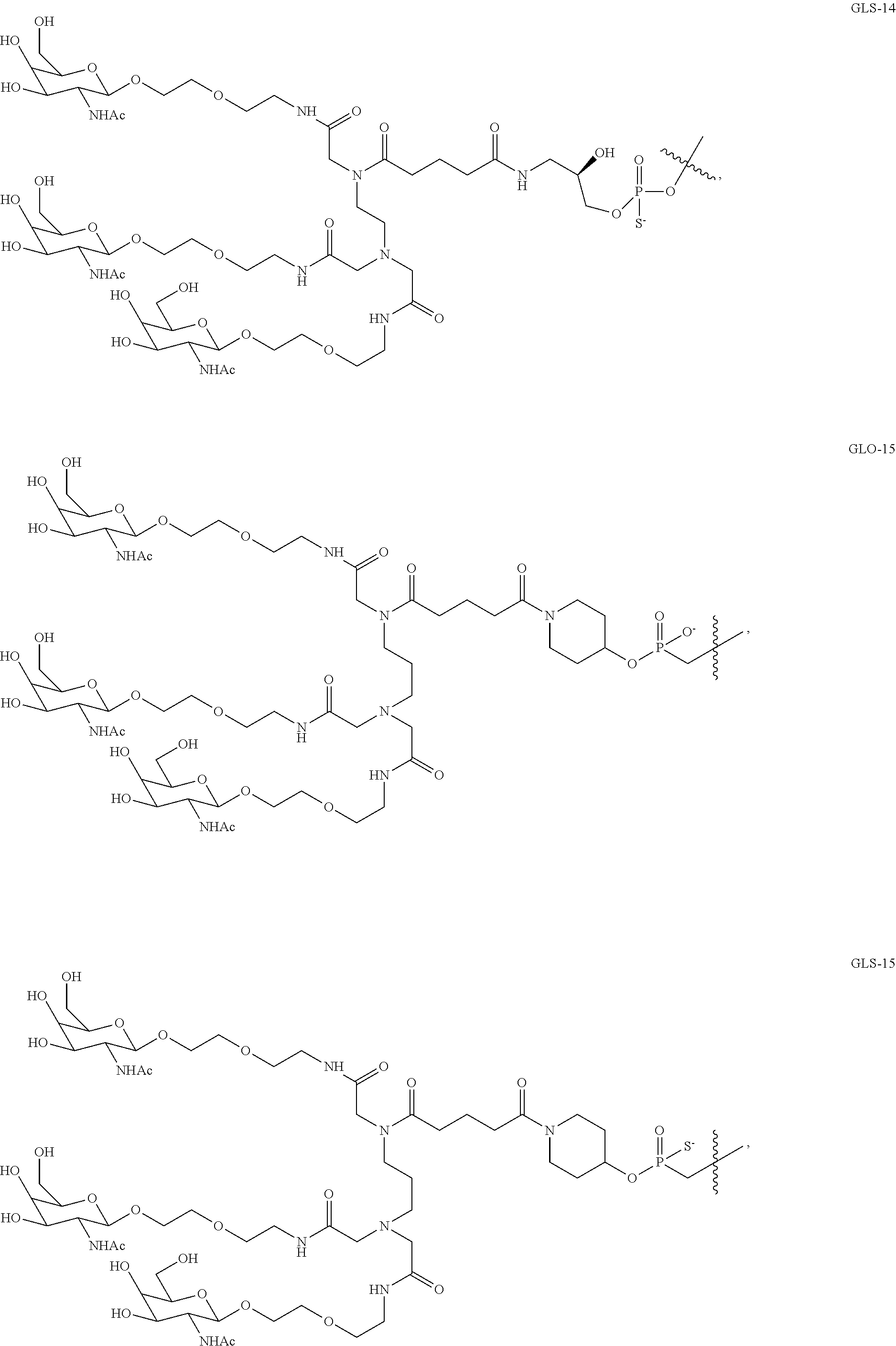 [SMILES: *OP(=O)([S-])OC[C@H](O)CNC(=O)CCCC(=O)N(CCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *CP(=O)([O-])OC1CCN(C(=O)CCCC(=O)N(CCCN(CC(=O)NCCOCCOC2OC(CO)C(O)C(O)C2NC(C)=O)CC(=O)NCCOCCOC2OC(CO)C(O)C(O)C2NC(C)=O)CC(=O)NCCOCCOC2OC(CO)C(O)C(O)C2NC(C)=O)CC1 ; *CP(=O)([S-])OC1CCN(C(=O)CCCC(=O)N(CCCN(CC(=O)NCCOCCOC2OC(CO)C(O)C(O)C2NC(C)=O)CC(=O)NCCOCCOC2OC(CO)C(O)C(O)C2NC(C)=O)CC(=O)NCCOCCOC2OC(CO)C(O)C(O)C2NC(C)=O)CC1]

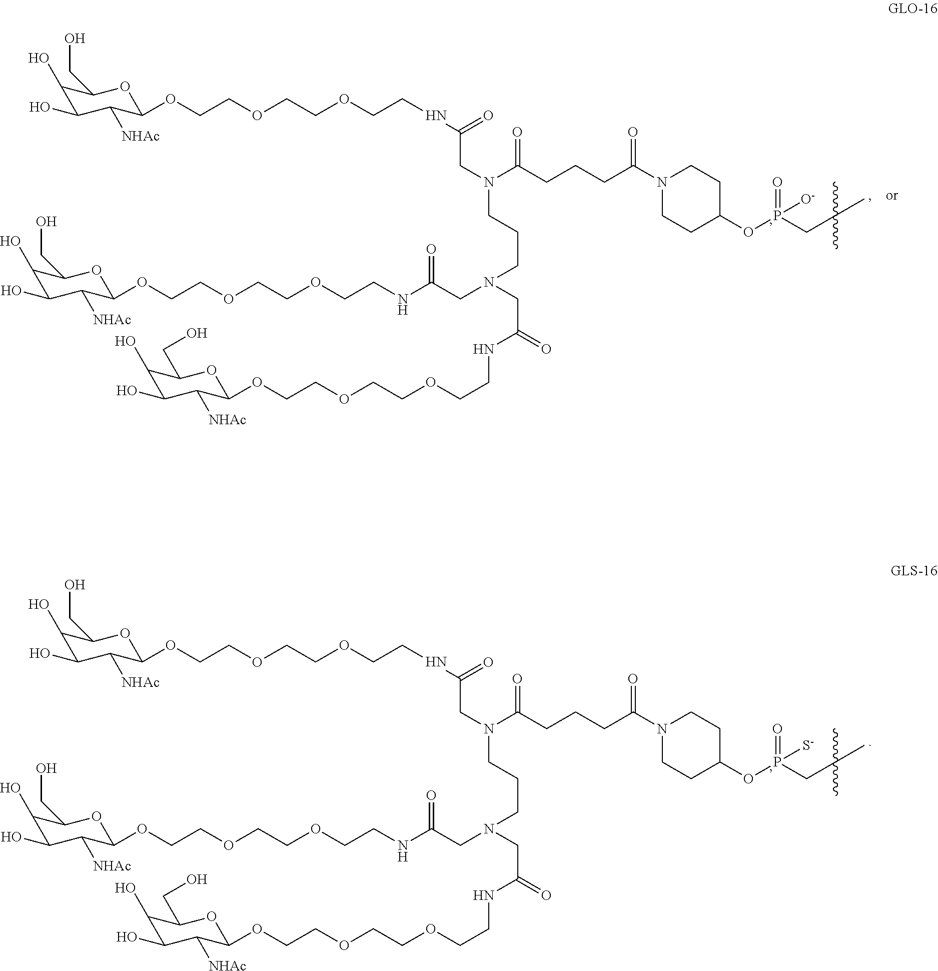 [SMILES: *CP(=O)([O-])OC1CCN(C(=O)CCCC(=O)N(CCCN(CC(=O)NCCOCCOCCOC2OC(CO)C(O)C(O)C2NC(C)=O)CC(=O)NCCOCCOCCOC2OC(CO)C(O)C(O)C2NC(C)=O)CC(=O)NCCOCCOCCOC2OC(CO)C(O)C(O)C2NC(C)=O)CC1 ; *CP(=O)([S-])OC1CCN(C(=O)CCCC(=O)N(CCCN(CC(=O)NCCOCCOCCOC2OC(CO)C(O)C(O)C2NC(C)=O)CC(=O)NCCOCCOCCOC2OC(CO)C(O)C(O)C2NC(C)=O)CC(=O)NCCOCCOCCOC2OC(CO)C(O)C(O)C2NC(C)=O)CC1]

In some embodiments of the invention, in vivo delivery can also be by a beta-glucan delivery system, such as those described in U.S. Pat. Nos. 5,032,401 and 5,607,677, and U.S. Publication No. 2005/0281781, which are hereby incorporated by reference in their entirety. In vitro introduction of an ANGPTL3 RNAi agent into a cell may also be done using art-known methods such as electroporation and lipofection. In certain embodiments of methods of the invention, an ANGPTL3 dsRNA is delivered without a targeting agent. These RNAs may be delivered as “naked” RNA molecules. As a non-limiting example, an ANGPTL3 dsRNA of the invention may be administered to a subject to treat an ANGPTL3-associated disease or condition in the subject, such as a liver disease, in a pharmaceutical composition comprising the RNAi agent, but not including a targeting agent such as a GalNAc targeting compound.

In addition to certain delivery means described herein, it will be understood that RNAi delivery means, such as but not limited to those described herein and those used in the art, can be used in conjunction with embodiments of ANGPTL3 RNAi agents and treatment methods described herein.

ANGPTL3 dsRNA agents of the invention may be administered to a subject in an amount and manner effective to reduce a level and activity of ANGPTL3 polypeptide in a cell and/or subject. In some embodiments of methods of the invention one or more ANGPTL3 dsRNA agents are administered to a cell and/or subject to treat a disease or condition associated with ANGPTL3 expression and activity. Methods of the invention, in some embodiments, include administering one or more ANGPTL3 dsRNA agents to a subject in need of such treatment to reduce a disease or condition associated with ANGPTL3 expression in the subject. ANGPTL3 dsRNA agents or ANGPTL3 antisense polynucleotide agents of the invention can be administered to reduce ANGPTL3 expression and/or activity in one more of in vitro, ex vivo, and in vivo cells.

In some embodiments of the invention, a level, and thus an activity, of ANGPTL3 polypeptide in a cell is reduced by delivering (e.g. introducing) an ANGPTL3 dsRNA agent or ANGPTL3 antisense polynucleotide agent into a cell. Targeting agents and methods may be used to aid in delivery of an ANGPTL3 dsRNA agent or ANGPTL3 antisense polynucleotide agent to a specific cell type, cell subtype, organ, spatial region within a subject, and/or to a sub-cellular region within a cell. An ANGPTL3 dsRNA agent can be administered in certain methods of the invention singly or in combination with one or more additional ANGPTL3 dsRNA agents. In some embodiments 2, 3, 4, or more independently selected ANGPTL3 dsRNA agents are administered to a subject.

In certain embodiments of the invention, an ANGPTL3 dsRNA agent is administered to a subject to treat an ANGPTL3-associated disease or condition in conjunction with one or more additional therapeutic regimens for treating the ANGPTL3-associate disease or condition. Non-limiting examples of additional therapeutic regimens are: administering one or more ANGPTL3 antisense polynucleotides of the invention, administering a non-ANGPTL3 dsRNA therapeutic agent, and a behavioral modification. An additional therapeutic regimen may be administered at a time that is one or more of: prior to, simultaneous with, and following administration of an ANGPTL3 dsRNA agent of the invention. It will be understood that simultaneous with as used herein, within five minutes of time zero, within 10 minutes of time zero, within 30 minutes of time zero, within 45 minutes of time zero, and within 60 minutes of time zero, with “time zero” the time of administration of the ANGPTL3 dsRNA agent of the invention to the subject. Non-limiting examples of non-ANGPTL3 dsRNA therapeutic agents are: one or more statins; one or more of an antibody, or an antisense oligonucleotide (ASO), or a siRNA molecule that are capable of reducing proprotein convertase subtilsin/kexin type 9 (PCSK9) expression (German CA, Shapiro MD. Small Interfering RNA Therapeutic Inclisiran: A New Approach to Targeting PCSK9. BioDrugs. 2020 February; 34(1):1-9. Doi: 10.1007/s40259-019-00399-6. PMID: 31782112); a therapeutic agent capable of reducing lipid accumulation in a subject, and a therapeutic agent capable of reducing cholesterol levels and/or accumulation in a subject. Non-limiting examples of behavioral modifications are: a dietary regimen, counseling, and an exercise regimen. These and other therapeutic agents and behavior modifications are known in the art and used to treat an ANGPTL3 disease or condition in a subject and may be administered to a subject in combination with the administration of one or more ATGPTL3 dsRNA agents of the invention to treat the ANGPTL3 disease or condition. An ANGPTL3 dsRNA agent of the invention administered to a cell or subject to treat an ANGPTL3-associated disease or condition may act in a synergistic manner with one or more other therapeutic agents or activities and increase the effectiveness of the one or more therapeutic agents or activities and/or to increase the effectiveness of the ANGPTL3 dsRNA agent at treating the ANGPTL3-associated disease or condition.

Treatment methods of the invention that include administration of an ANGPTL3 dsRNA agent can be used prior to the onset of an ANGPTL3-associated disease or condition and/or when an ANGPTL3-associated disease or condition is present, including at an early stage, mid-stage, and late stage of the disease or condition and all times before and after any of these stages. Methods of the invention may also be to treat subjects who have previously been treated for an ANGPTL3-associated disease or condition with one or more other therapeutic agents and/or therapeutic activities that were not successful, were minimally successful, and/or are no longer successful at treating the ANGPTL3-associated disease or condition in the subject.

Vector Encoded dsRNAs

In certain embodiments of the invention, an ANGPTL3 dsRNA agent can be delivered into a cell using a vector. ANGPTL3 dsRNA agent transcription units can be included in a DNA or RNA vector. Prepare and use of such vectors encoding transgenes for delivering sequences into a cell and or subject are well known in the art. Vectors can be used in methods of the invention that result in transient expression of ANGPTL3 dsRNA, for example for at least 1, 2, 3, 4, 5, 6, 7, 8, 9, 10 or more hours, 1, 2, 3, 4, 5, 6, 7, 8, 9, 10 or more weeks. The length of the transient expression can be determined using routine methods based on elements such as, but not limited to the specific vector construct selected and the target cell and/or tissue. Such transgenes can be introduced as a linear construct, a circular plasmid, or a viral vector, which can be an integrating or non-integrating vector. The transgene can also be constructed to permit it to be inherited as an extrachromosomal plasmid (Gassmann, et al., Proc. Natl. Acad. Sci. USA (1995) 92:1292).

An individual strand or strands of an ANGPTL3 dsRNA agent can be transcribed from a promoter on an expression vector. Where two separate strands are to be expressed to generate, for example, a dsRNA, two separate expression vectors can be co-introduced to a cell using means such as transfection or infection. In certain embodiments each individual strand of an ANGPTL3 dsRNA agent of the invention can be transcribed by promoters that are both included on the same expression vector. In certain embodiments of the invention an ANGPTL3 dsRNA agent is expressed as inverted repeat polynucleotides joined by a linker polynucleotide sequence such that the ANGPTL3 dsRNA agent has a stem and loop structure.

Non-limiting examples of RNA expression vectors are DNA plasmids or viral vectors. Expression vectors useful in embodiments of the invention can be compatible with eukaryotic cells. Eukaryotic cell expression vectors are routinely used in the art and are available from a number of commercial sources. Delivery of ANGPTL3 dsRNA expressing vectors can be systemic, such as by intravenous or intramuscular administration, by administration to target cells ex-planted from a subject followed by reintroduction into the subject, or by any other means that allows for introduction into a desired target cell.

Viral vector systems that may be included in an embodiment of a method of the include, but are not limited to, (a) adenovirus vectors; (b) retrovirus vectors, including but not limited to lentiviral vectors, moloney murine leukemia virus, etc.; (c) adeno-associated virus vectors; (d) herpes simplex virus vectors; (e) SV 40 vectors; (f) polyoma virus vectors; (g) papilloma virus vectors; (h) picornavirus vectors; (i) pox virus vectors such as an orthopox, e.g., vaccinia virus vectors or avipox, e.g. canary pox or fowl pox; and (j) a helper-dependent or gutless adenovirus. Constructs for the recombinant expression of an ANGPTL3 dsRNA agent may include regulatory elements, such as promoters, enhancers, etc., which may be selected to provide constitutive or regulated/inducible expression. Viral vector systems, and the use of promoters and enhancers, etc. are routine in the art and can be used in conjunction with methods and compositions described herein.

Certain embodiments of the invention include use of viral vectors for delivery of ANGPTL3 dsRNA agents into cells. Numerous adenovirus-based delivery systems are routinely used in the art for deliver to, for example, lung, liver, the central nervous system, endothelial cells, and muscle. Non-limiting examples of viral vectors that may be used in methods of the invention are: AAV vectors, a pox virus such as a vaccinia virus, a Modified Virus Ankara (MVA), NYVAC, an avipox such as fowl pox or canary pox.

Certain embodiments of the invention include methods of delivering ANGPTL3 dsRNA agents into cells using a vector and such vectors may be in a pharmaceutically acceptable carrier that may, but need not, include a slow release matrix in which the gene delivery vehicle is imbedded. In some embodiments, a vector for delivering an ANGPTL3 dsRNA can be produced from a recombinant cell, and a pharmaceutical composition of the invention may include one or more cells that produced the ANGPTL3 dsRNA delivery system.

Pharmaceutical Compositions Containing ANGPTL3 dsRNA or ssRNA Agents

Certain embodiments of the invention include use of pharmaceutical compositions containing an ANGPTL3 dsRNA agent or ANGPTL3 antisense polynucleotide agent and a pharmaceutically acceptable carrier. The pharmaceutical composition containing the ANGPTL3 dsRNA agent or ANGPTL3 antisense polynucleotide agent can be used in methods of the invention to reduce ANGPTL3 gene expression and ANGPTL3 activity in a cell and is useful to treat an ANGPTL3-associated disease or condition. Such pharmaceutical compositions can be formulated based on the mode of delivery. Non-limiting examples of formulations for modes of delivery are: a composition formulated for subcutaneous delivery, a composition formulated for systemic administration via parenteral delivery, a composition formulated for intravenous (IV) delivery, a composition formulated for intrathecal delivery, a composition formulated for direct delivery into brain, etc. Administration of a pharmaceutic composition of the invention to deliver an ANGPTL3 dsRNA agent or ANGPTL3 antisense polynucleotide agent into a cell may be done using one or more means such as: topical (e.g., by a transdermal patch), pulmonary, e.g., by inhalation or insufflation of powders or aerosols, including by nebulizer; intratracheal, intranasal, epidermal and transdermal, oral or parenteral. Parenteral administration includes intravenous, intraarterial, subcutaneous, intraperitoneal or intramuscular injection or infusion; subdermal, e.g., via an implanted device; or intracranial, e.g., by intraparenchymal, intrathecal or intraventricular, administration. An ANGPTL3 dsRNA agent or ANGPTL3 antisense polynucleotide agent can also be delivered directly to a target tissue, for example directly into the liver, directly into a kidney, etc. It will be understood that “delivering an ANGPTL3 dsRNA agent” or “delivering an ANGPTL3 antisense polynucleotide agent” into a cell encompasses delivering an ANGPTL3 dsRNA agent or ANGPTL3 antisense polynucleotide agent, respectively, directly as well as expressing an ANGPTL3 dsRNA agent in a cell from an encoding vector that is delivered into a cell, or by any suitable means with which the ANGPTL3 dsRNA or ANGPTL3 antisense polynucleotide agent becomes present in a cell. Preparation and use of formulations and means for delivering inhibitory RNAs are well known and routinely used in the art.

As used herein, a “pharmaceutical composition” comprises a pharmacologically effective amount of an ANGPTL3 dsRNA agent or ANGPTL3 antisense polynucleotide agent of the invention and a pharmaceutically acceptable carrier. The term “pharmaceutically acceptable carrier” refers to a carrier for administration of a therapeutic agent. Such carriers include, but are not limited to, saline, buffered saline, dextrose, water, glycerol, ethanol, and combinations thereof. The term specifically excludes cell culture medium. For drugs administered orally, pharmaceutically acceptable carriers include, but are not limited to pharmaceutically acceptable excipients such as inert diluents, disintegrating agents, binding agents, lubricating agents, sweetening agents, flavoring agents, coloring agents and preservatives. Suitable inert diluents include sodium and calcium carbonate, sodium and calcium phosphate, and lactose, while corn starch and alginic acid are suitable disintegrating agents. Binding agents may include starch and gelatin, while the lubricating agent, if present, will generally be magnesium stearate, stearic acid or talc. If desired, the tablets may be coated with a material such as glyceryl monostearate or glyceryl distearate, to delay absorption in the gastrointestinal tract. Agents included in drug formulations are described further herein below.

As used herein terms such as: “pharmacologically effective amount,” “therapeutically effective amount” and “effective amount” refers to that amount of an ANGPTL3 dsRNA agent or ANGPTL3 antisense polynucleotide agent of the invention to produce the intended pharmacological, therapeutic or preventive result. For example, if a given clinical treatment is considered effective when there is at least a 10% reduction in a measurable parameter associated with a disease or disorder, a therapeutically effective amount of a drug for the treatment of that disease or disorder is the amount necessary to effect at least a 10% reduction in that parameter. For example, a therapeutically effective amount of an ANGPTL3 dsRNA agent or ANGPTL3 antisense polynucleotide agent can reduce ANGPTL3 polypeptide levels by at least 10%.

### Effective Amounts

Methods of the invention, in some aspects comprise contacting a cell with an ANGPTL3 dsRNA agent or ANGPTL3 antisense polynucleotide agent in an effective amount to reduce ANGPTL3 gene expression in the contacted cell. Certain embodiments of methods of the invention comprise administering an ANGPTL3 dsRNA agent or an ANGPTL3 antisense polynucleotide agent to a subject in an amount effective to reduce ANGPTL3 gene expression and treat an ANGPTL3-associated disease or condition in the subject. An “effective amount” used in terms of reducing expression of ANGPTL3 and/or for treating an ANGPTL3-associated disease or condition, is an amount necessary or sufficient to realize a desired biologic effect. For example, an effective amount of an ANGPTL3 dsRNA agent or ANGPTL3 antisense polynucleotide agent to treat an ANGPTL3-associated disease or condition could be that amount necessary to (i) slow or halt progression of the disease or condition; or (ii) reverse, reduce, or eliminate one or more symptoms of the disease or condition. In some aspects of the invention, an effective amount is that amount of an ANGPTL3 dsRNA agent or ANGPTL3 antisense polynucleotide agent that when administered to a subject in need of a treatment of an ANGPTL3-associated disease or condition, results in a therapeutic response that prevents and/or treats the disease or condition. According to some aspects of the invention, an effective amount is that amount of an ANGPTL3 dsRNA agent or ANGPTL3 antisense polynucleotide agent of the invention that when combined or co-administered with another therapeutic treatment for an ANGPTL3-associated disease or condition, results in a therapeutic response that prevents and/or treats the disease or condition. In some embodiments of the invention, a biologic effect of treating a subject with an ANGPTL3 dsRNA agent or ANGPTL3 antisense polynucleotide agent of the invention may be the amelioration and or absolute elimination of symptoms resulting from the ANGPTL3-associated disease or condition. In some embodiments of the invention, a biologic effect is the complete abrogation of the ANGPTL3-associated disease or condition, as evidenced for example, by a diagnostic test that indicates the subject is free of the ANGPTL3-associated disease or condition. A non-limiting example of a physiological symptom that may be detected includes a reduction in lipid accumulation in liver of a subject following administration of an agent of the invention. Additional art-known means of assessing the status of an ANGPTL3-associated disease or condition can be used to determine an effect of an agent and/or methods of the invention on an ANGPTL3-associated disease or condition.

Typically an effective amount of an ANGPTL3 dsRNA agent or ANGPTL3 antisense polynucleotide agent to decrease ANGPTL3 polypeptide activity to a level to treat an ANGPTL3-associated disease or condition will be determined in clinical trials, establishing an effective dose for a test population versus a control population in a blind study. In some embodiments, an effective amount will be that results in a desired response, e.g., an amount that diminishes an ANGPTL3-associated disease or condition in cells, tissues, and/or subjects with the disease or condition. Thus, an effective amount of an ANGPTL3 dsRNA agent or ANGPTL3 antisense polynucleotide agent to treat an ANGPTL3-associated disease or condition that can be treated by reducing ANGPTL3 polypeptide activity may be the amount that when administered decreases the amount of ANGPTL3 polypeptide activity in the subject to an amount that is less than the amount that would be present in the cell, tissue, and/or subject without the administration of the ANGPTL3 dsRNA agent or ANGPTL3 antisense polynucleotide agent. In certain aspects of the invention the level of ANGPTL3 polypeptide activity, and/or ANGPTL3 gene expression present in a cell, tissue, and/or subject that has not been contacted with or administered an ANGPTL3 dsRNA agent or ANGPTL3 antisense polynucleotide agent of the invention is referred to as a “control” amount. In some embodiments of methods of the invention a control amount for a subject is a pre-treatment amount for the subject, in other words, a level in a subject before administration of an ANGPTL3 agent can be a control level for that subject and compared to a level of ANGPTL3 polypeptide activity and/or ANGPTL3 gene expression in the subject following siRNA administered to the subject. In the case of treating an ANGPTL3-associated disease or condition the desired response may be reducing or eliminating one or more symptoms of the disease or condition in the cell, tissue, and/or subject. The reduction or elimination may be temporary or may be permanent. It will be understood that the status of an ANGPTL3-associated disease or condition can be monitored using methods of determining ANGPTL3 polypeptide activity, ANGPTL3 gene expression, symptom evaluation, clinical testing, etc. In some aspects of the invention, a desired response to treatment of an ANGPTL3-associated disease or condition is delaying the onset or even preventing the onset of the disease or condition.

An effective amount of a compound that decreases ANGPTL3 polypeptide activity may also be determined by assessing physiological effects of administration of an ANGPTL3 dsRNA agent or ANGPTL3 antisense polynucleotide agent on a cell or subject, such as a decrease of an ANGPTL3-associated disease or condition following administration. Assays and/or symptomatic monitoring of a subject can be used to determine efficacy of an ANGPTL3 dsRNA agent or ANGPTL3 antisense polynucleotide agent of the invention, which may be administered in a pharmaceutical compound of the invention, and to determine the presence or absence of a response to the treatment. A non-limiting example, is that one or more art-known tests of serum lipid profile. Another non-limiting example, is that one or more art-known tests of liver function can be used to determine the status of the ANGPTL3-associated liver disease or condition in a subject before and after treatment of the subject with an ANGPTL3 dsRNA agent of the invention. In another non-limiting example, one or more art-known tests of cholesterol accumulation in liver are used to determine the status of an ANGPTL3-associated disease in a subject. In this example the disease includes cholesterol accumulation and the tests are used to determine cholesterol levels in the subject before and after treatment of the subject with an ANGPTL3 dsRNA agent of the invention.

Some embodiments of the invention include methods of determining an efficacy of an dsRNA agent or ANGPTL3 antisense polynucleotide agent of the invention administered to a subject, to treat an ANGPTL3-associated disease or condition by assessing and/or monitoring one or more “physiological characteristics” of the ANGPTL3-associated disease or condition in the subject. Non-limiting examples of physiological characteristics of an ANGPTL3-associated disease or condition are a subject's serum lipid level, a subject's LDL level, a subject's HDL level, a subject's LDL. HDL ratio, a subject's triglyceride level, fat present in a subject's liver, physical symptoms, etc. Standard means of determining such physiological characteristic are known in the art and include, but are not limited to, blood tests, imaging studies, physical examination, etc.

It will be understood that the amount of an ANGPTL3 dsRNA agent or ANGPTL3 antisense polynucleotide agent administered to a subject can be modified based, at least in part, on such determinations of disease and/or condition status and/or physiological characteristics determined for a subject. The amount of a treatment may be varied for example by increasing or decreasing the amount of an ANGPTL3-dsRNA agent or ANGPTL3 antisense polynucleotide agent, by changing the composition in which the ANGPTL3 dsRNA agent or ANGPTL3 antisense polynucleotide agent, respectively, is administered, by changing the route of administration, by changing the dosage timing and so on. The effective amount of an ANGPTL3 dsRNA agent or ANGPTL3 antisense polynucleotide agent will vary with the particular condition being treated, the age and physical condition of the subject being treated; the severity of the condition, the duration of the treatment, the nature of the concurrent therapy (if any), the specific route of administration, and additional factors within the knowledge and expertise of the health practitioner. For example, an effective amount may depend upon the desired level of ANGPTL3 polypeptide activity and or ANGPTL3 gene expression that is effective to treat the ANGPTL3-associated disease or condition. A skilled artisan can empirically determine an effective amount of a particular ANGPTL3 dsRNA agent or ANGPTL3 antisense polynucleotide agent of the invention for use in methods of the invention without necessitating undue experimentation. Combined with the teachings provided herein, by selecting from among various ANGPTL3 dsRNA agents or ANGPTL3 antisense polynucleotide agents of the invention, and weighing factors such as potency, relative bioavailability, patient body weight, severity of adverse side-effects and preferred mode of administration, an effective prophylactic or therapeutic treatment regimen can be planned that is effective to treat the particular subject. As used in embodiments of the invention, an effective amount of an ANGPTL3 dsRNA agent or ANGPTL3 antisense polynucleotide agent of the invention can be that amount that when contacted with a cell results in a desired biological effect in the cell.

It will be recognized that ANGPTL3 gene silencing may be determined in any cell expressing ANGPTL3, either constitutively or by genomic engineering, and by any appropriate assay. In some embodiments of the invention, ANGPTL3 gene expression is reduced by at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 100% by administration of an ANGPTL3 dsRNA agent of the invention. In some embodiments of the invention, ANGPTL3 gene expression is reduced by at between 5% and 10%, 5% and 25%, 10% and 50%, 10% and 75%, 25% and 75%, 25% and 100%, or 50% and 100% by administration of an ANGPTL3 dsRNA agent of the invention.

### Dosing

ANGPTL3 dsRNA agents and ANGPTL3 antisense polynucleotide agents are delivered in pharmaceutical compositions in dosages sufficient to inhibit expression of ANGPTL3 genes. In certain embodiments of the invention, a dose of ANGPTL3 dsRNA agent or ANGPTL3 antisense polynucleotide agent is in a range of 0.01 to 200.0 milligrams per kilogram body weight of the recipient per day, generally in the range of 1 to 50 mg per kilogram body weight, 5 to 40 mg/kg body weight, 10 to 30 mg/kg body weight, 1 to 20 mg/kg body weight, 1 to 10 mg/kg body weight, 4 to 15 mg/kg body weight per day, inclusive. For example, the ANGPTL3 dsRNA agent or ANGPTL3 antisense polynucleotide agent can be administered in an amount that is from about 0.01 mg/kg, 0.05 mg/kg, 0.1 mg/kg, 0.2 mg/kg, 0.3 mg/kg, 0.4 mg/kg, 0.5 mg/kg, 1 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg/kg, 3.0 mg/kg, 3.1 mg/kg, 3.2 mg/kg, 3.3 mg/kg, 3.4 mg/kg, 3.5 mg/kg, 3.6 mg/kg, 3.7 mg/kg, 3.8 mg/kg, 3.9 mg/kg, 4 mg/kg, 4.1 mg/kg, 4.2 mg/kg, 4.3 mg/kg, 4.4 mg/kg, 4.5 mg/kg, 4.6 mg/kg, 4.7 mg/kg, 4.8 mg/kg, 4.9 mg/kg, 5 mg/kg, 5.1 mg/kg, 5.2 mg/kg, 5.3 mg/kg, 5.4 mg/kg, 5.5 mg/kg, 5.6 mg/kg, 5.7 mg/kg, 5.8 mg/kg, 5.9 mg/kg, 6 mg/kg, 6.1 mg/kg, 6.2 mg/kg, 6.3 mg/kg, 6.4 mg/kg, 6.5 mg/kg, 6.6 mg/kg, 6.7 mg/kg, 6.8 mg/kg, 6.9 mg/kg, 7 mg/kg, 7.1 mg/kg, 7.2 mg/kg, 7.3 mg/kg, 7.4 mg/kg, 7.5 mg/kg, 7.6 mg/kg, 7.7 mg/kg, 7.8 mg/kg, 7.9 mg/kg, 8 mg/kg, 8.1 mg/kg, 8.2 mg/kg, 8.3 mg/kg, 8.4 mg/kg, 8.5 mg/kg, 8.6 mg/kg, 8.7 mg/kg, 8.8 mg/kg, 8.9 mg/kg, 9 mg/kg, 9.1 mg/kg, 9.2 mg/kg, 9.3 mg/kg, 9.4 mg/kg, 9.5 mg/kg, 9.6 mg/kg, 9.7 mg/kg, 9.8 mg/kg, 9.9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, through 50 mg/kg body per single dose.

Various factors may be considered in the determination of dosage and timing of delivery of an ANGPTL3 dsRNA agent of the invention. The absolute amount of an ANGPTL3 dsRNA agent or ANGPTL3 antisense polynucleotide agent delivered will depend upon a variety of factors including a concurrent treatment, the number of doses and the individual subject parameters including age, physical condition, size and weight. These are factors well known to those of ordinary skill in the art and can be addressed with no more than routine experimentation. In some embodiments, a maximum dose can be used, that is, the highest safe dose according to sound medical judgment.

Methods of the invention may in some embodiments include administering to a subject 1, 2, 3, 4, 5, 6, 7, 8, 9, 10, or more doses of an ANGPTL3 dsRNA agent or ANGPTL3 antisense polynucleotide agent. In some instances, a pharmaceutical compound, (e.g., comprising an ANGPTL3 dsRNA agent or comprising an ANGPTL3 antisense polynucleotide agent) can be administered to a subject at least daily, every other day, weekly, every other week, monthly, etc. Doses may be administered once per day or more than once per day, for example, 2, 3, 4, 5, or more times in one 24 hour period. A pharmaceutical composition of the invention may be administered once daily, or the ANGPTL3 dsRNA agent or ANGPTL3 antisense polynucleotide agent may be administered as two, three, or more sub-doses at appropriate intervals throughout the day or even using continuous infusion or delivery through a controlled release formulation. In some embodiments of methods of the invention, a pharmaceutical composition of the invention is administered to a subject one or more times per day, one or more times per week, one or more times per month, or one or more times per year.

Methods of the invention, in some aspects, include administration of a pharmaceutical compound alone, in combination with one or more other ANGPTL3 dsRNA agents or ANGPTL3 antisense polynucleotide agents, and/or in combination with other drug therapies or treatment activities or regimens that are administered to subjects with an ANGPTL3-associated disease or condition. Pharmaceutical compounds may be administered in pharmaceutical compositions. Pharmaceutical compositions used in methods of the invention may be sterile and contain an amount of an ANGPTL3 dsRNA agent or ANGPTL3 antisense polynucleotide agent that will reduce activity of an ANGPTL3 polypeptide to a level sufficient to produce the desired response in a unit of weight or volume suitable for administration to a subject. A dose administered to a subject of a pharmaceutical composition that includes an ANGPTL3 dsRNA agent or ANGPTL3 antisense polynucleotide agent to reduce ANGPTL3 protein activity can be chosen in accordance with different parameters, in particular in accordance with the mode of administration used and the state of the subject. Other factors include the desired period of treatment. In the event that a response in a subject is insufficient at the initial doses applied, higher doses (or effectively higher doses by a different, more localized delivery route) may be employed to the extent that patient tolerance permits.

### Treatment

ANGPTL3-associated diseases and conditions in which a decrease in a level and/or activity of ANGPTL3 polypeptide is effective to treat the disease or condition, can be treated using methods and ANGPTL3 dsRNA agents of the invention to inhibit ANGPTL3 expression. Examples of diseases and conditions that may be treated with an ANGPTL3 dsRNA agent or ANGPTL3 antisense polynucleotide agent of the invention and a treatment method of the invention, include, but are not limited to: hyperlipidemia, hypertriglyceridemia, abnormal lipid and/or cholesterol metabolism, homozygous and heterozygous familial hypercholesterolemia, statin resistant hypercholesterolemia, cardiometabolic disease, obesity, atherosclerosis, type II diabetes mellitus, cardiovascular disease, coronary artery disease, non-alcoholic steatohepatitis, non-alcoholic fatty liver disease, and pancreatitis caused by hypertriglyceridemia. Such diseases and conditions may be referred to herein as “ANGPTL3-associated diseases and conditions” and “diseases and conditions caused and/or modulated by ANGPTL3.”

In certain aspects of the invention, a subject may be administered an ANGPTL3 dsRNA agent or ANGPTL3 antisense polynucleotide agent of the invention at a time that is one or more of before or after diagnosis of an ANGPTL3-associated disease or condition. In some aspects of the invention, a subject is at risk of having or developing an ANGPTL3-associated disease or condition. A subject at risk of developing an ANGPTL3-associated disease or condition is one who has an increased probability of developing the ANGPTL3-associated disease or condition, compared to a control risk of developing the ANGPTL3-associated disease or condition. In some embodiments of the invention, a level of risk may be statistically significant compared to a control level of risk. A subject at risk may include, for instance, a subject who is, or will be, a subject who has a preexisting disease and/or a genetic abnormality that makes the subject more susceptible to an ANGPTL3-associated disease or condition than a control subject without the preexisting disease or genetic abnormality; a subject having a family and/or personal medical history of the ANGPTL3-associated disease or condition; and a subject who has previously been treated for an ANGPTL3-associated disease or condition. It will be understood that a preexisting disease and/or a genetic abnormality that makes the subject more susceptible to an ANGPTL3-associated disease or condition, may be a disease or genetic abnormality that when present has been previously identified as having a correlative relation to a higher likelihood of developing an ANGPTL3-associated disease or condition.

It will be understood that an ANGPTL3 dsRNA agent or ANGPTL3 antisense polynucleotide agent may be administered to a subject based on a medical status of the individual subject. For example, a health-care provided for a subject may assess a lipid level measured in a sample obtained from a subject and determine it is desirable to reduce the subject's lipid level, by administration of an ANGPTL3 dsRNA agent or ANGPTL3 antisense polynucleotide agent of the invention. In this example, the lipid level may be considered to be a physiological characteristic of an ANGPTL3-associated condition, even if the subject is not diagnosed as having an ANGPTL3-associated disease such as one disclosed herein. A healthcare provider may monitor changes in the subject's lipid level, as a measure of efficacy of the administered ANGPTL3 dsRNA agent or ANGPTL3 antisense polynucleotide agent of the invention. In a non-limiting example, a biological sample, such as a blood or serum sample may be obtained from a subject and a lipid level for the subject determined in the sample. An ANGPTL3 dsRNA agent or ANGPTL3 antisense polynucleotide agent is administered to the subject and a blood or serum sample is obtained from the subject following the administration and the lipid level determined using the sample and the results compared to the results determined in the subject's pre-administration (prior) sample. A reduction in the subject's lipid level in the later sample compared to the pre-administration level indicates the administered ANGPTL3 dsRNA agent or ANGPTL3 antisense polynucleotide agent efficacy in reducing the lipid level in the subject.

Certain embodiments of methods of the invention include adjusting a treatment that includes administering a dsRNA agent or an ANGPTL3 antisense polynucleotide agent of the invention to a subject based at least in part on assessment of a change in one or more of the subject's physiological characteristics of an ANGPTL3-associated disease or condition resulting from the treatment. For example, in some embodiments of the invention, an effect of an administered dsRNA agent or ANGPTL3 antisense polynucleotide agent of the invention may be determined for a subject and used to assist in adjusting an amount of a dsRNA agent or ANGPTL3 antisense polynucleotide agent of the invention subsequently administered to the subject. In a non-limiting example, a subject is administered a dsRNA agent or ANGPTL3 antisense polynucleotide agent of the invention, the subject's lipid level is determined after the administration, and based at least in part on the determined level, a greater amount of the dsRNA agent or ANGPTL3 antisense polynucleotide agent is determined to be desirable in order to increase the physiological effect of the administered agent, for example to reduce or further reduce the subject's lipid level. In another non-limiting example, a subject is administered a dsRNA agent or ANGPTL3 antisense polynucleotide agent of the invention, the subject's lipid level is determined after the administration and based at least in part on the determined level, a lower amount of the dsRNA agent or ANGPTL3 antisense polynucleotide agent is desirable to administer to the subject.

Thus, some embodiments of the invention include assessing a change in one or more physiological characteristics of resulting from a subject's previous treatment to adjust an amount of a dsRNA agent or ANGPTL3 antisense polynucleotide agent of the invention subsequently administered to the subject. Some embodiments of methods of the invention include 1, 2, 3, 4, 5, 6, or more determinations of a physiological characteristic of an ANGPTL3-associated disease or condition to assess and/or monitor the efficacy of an administered ANGPTL3 dsRNA agent or ANGPTL3 antisense polynucleotide agent of the invention, and optionally using the determinations to adjust one or more of: a dose, administration regimen, and or administration frequency of a dsRNA agent or ANGPTL3 antisense polynucleotide agent of the invention to treat an ANGPTL3-associated disease or condition in a subject. In some embodiments of methods of the invention, a desired result of administering an effective amount of a dsRNA agent or ANGPTL3 antisense polynucleotide agent of the invention to a subject is a reduction of the subject's lipid level, serum lipid level, LDL level, LDL:HDL ratio, triglyceride level, fat present in a subject's liver, etc., as compared to a prior level determined for the subject, or to a control level.

As used herein, the terms “treat”, “treated”, or “treating” when used with respect to an ANGPTL3-associated disease or condition may refer to a prophylactic treatment that decreases the likelihood of a subject developing the ANGPTL3-associated disease or condition, and also may refer to a treatment after the subject has developed an ANGPTL3-associated disease or condition in order to eliminate or reduce the level of the ANGPTL3-associated disease or condition, prevent the ANGPTL3-associated disease or condition from becoming more advanced (e.g., more severe), and/or slow the progression of the ANGPTL3-associated disease or condition in a subject compared to the subject in the absence of the therapy to reduce activity in the subject of ANGPTL3 polypeptide.

Certain embodiments of agents, compositions, and methods of the invention can be used to inhibit ANGPTL3 gene expression. As used herein in reference to expression of an ANGPTL3 gene, the terms “inhibit,” “silence,” “reduce,” “down-regulate,” and “knockdown” mean the expression of the ANGPTL3 gene, as measured by one or more of: a level of RNA transcribed from the gene, a level of activity of ANGPTL3 expressed, and a level of ANGPTL3 polypeptide, protein or protein subunit translated from the mRNA in a cell, group of cells, tissue, organ, or subject in which the ANGPTL3 gene is transcribed, is reduced when the cell, group of cells, tissue, organ, or subject is contacted with (e.g., treated with) an ANGPTL3 dsRNA agent or ANGPTL3 antisense polynucleotide agent of the invention, compared to a control level of RNA transcribed from the ANGPTL3 gene, a level of activity of expressed ANGPTL3, or a level of ANGPTL3 translated from the MRNA, respectively. In some embodiments, a control level is a level in a cell, tissue, organ or subject that has not been contacted with (e.g. treated with) the ANGPTL3 dsRNA agent or ANGPTL3 antisense polynucleotide agent.

### Administration Methods

A variety of administration routes for an ANGPTL3 dsRNA agent or ANGPTL3 antisense polynucleotide agent are available for use in methods of the invention. The particular delivery mode selected will depend at least in part, upon the particular condition being treated and the dosage required for therapeutic efficacy. Methods of this invention, generally speaking, may be practiced using any mode of administration that is medically acceptable, meaning any mode that produces effective levels of treatment of an ANGPTL3-associated disease or condition without causing clinically unacceptable adverse effects. In some embodiments of the invention, an ANGPTL3 dsRNA agent or ANGPTL3 antisense polynucleotide agent may be administered via an oral, enteral, mucosal, subcutaneous, and/or parenteral route. The term “parenteral” includes subcutaneous, intravenous, intrathecal, intramuscular, intraperitoneal, and intrasternal injection, or infusion techniques. Other routes include but are not limited to nasal (e.g., via a gastro-nasal tube), dermal, vaginal, rectal, sublingual, and inhalation. Delivery routes of the invention may include intrathecal, intraventricular, or intracranial. In some embodiments of the invention, an ANGPTL3 dsRNA agent or ANGPTL3 antisense polynucleotide agent may be placed within a slow release matrix and administered by placement of the matrix in the subject. In some aspects of the invention, an ANGPTL3 dsRNA agent or ANGPTL3 antisense polynucleotide agent may be delivered to a subject cell using nanoparticles coated with a delivery agent that targets a specific cell or organelle. Various delivery means, methods, agents are known in the art. Non-limiting examples of delivery methods and delivery agents are additionally provided elsewhere herein. In some aspects of the invention, the term “delivering” in reference to an ANGPTL3 dsRNA agent or ANGPTL3 antisense polynucleotide agent may mean administration to a cell or subject of one or more “naked” ANGPTL3 dsRNA agent or ANGPTL3 antisense polynucleotide agent sequences and in certain aspects of the invention “delivering” means administration to a cell or subject via transfection means, delivering a cell comprising an ANGPTL3 dsRNA agent or ANGPTL3 antisense polynucleotide agent to a subject, delivering a vector encoding an ANGPTL3 dsRNA agent or ANGPTL3 antisense polynucleotide agent into a cell and/or subject, etc. Delivery of an ANGPTL3 dsRNA agent or ANGPTL3 antisense polynucleotide agent using a transfection means may include administration of a vector to a cell and/or subject.

In some methods of the invention one or more ANGPTL3 dsRNA agents or ANGPTL3 antisense polynucleotide agents may be administered in formulations, which may be administered in pharmaceutically acceptable solutions, which may routinely contain pharmaceutically acceptable concentrations of salt, buffering agents, preservatives, compatible carriers, adjuvants, and optionally other therapeutic ingredients. In some embodiments of the invention an ANGPTL3 dsRNA agent or ANGPTL3 antisense polynucleotide agent may be formulated with another therapeutic agent for simultaneous administration. According to methods of the invention, an ANGPTL3 dsRNA agent or ANGPTL3 antisense polynucleotide agent may be administered in a pharmaceutical composition. In general, a pharmaceutical composition comprises an ANGPTL3 dsRNA agent or ANGPTL3 antisense polynucleotide agent and optionally, a pharmaceutically-acceptable carrier. Pharmaceutically-acceptable carriers are well-known to those of ordinary skill in the art. As used herein, a pharmaceutically-acceptable carrier means a non-toxic material that does not interfere with the effectiveness of the biological activity of the active ingredients, e.g., the ability of the ANGPTL3 dsRNA agent or ANGPTL3 antisense polynucleotide agent to inhibit ANGPTL3 gene expression in a cell or subject. Numerous methods to administer and deliver dsRNA agents or ANGPTL3 antisense polynucleotide agents for therapeutic use are known in the art and may be utilized in methods of the invention.

Pharmaceutically acceptable carriers include diluents, fillers, salts, buffers, stabilizers, solubilizers and other materials that are well-known in the art. Exemplary pharmaceutically acceptable carriers are described in U.S. Pat. No. 5,211,657 and others are known by those skilled in the art. Such preparations may routinely contain salt, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

Some embodiments of methods of the invention include administering one or more ANGPTL3 dsRNA agents or ANGPTL3 antisense polynucleotide agents directly to a tissue. In some embodiments, the tissue to which the compound is administered is a tissue in which the ANGPTL3-associated disease or condition is present or is likely to arise, non-limiting examples of which are the liver or kidney. Direct tissue administration may be achieved by direct injection or other means. Many orally delivered compounds naturally travel to and through the liver and kidneys and some embodiments of treatment methods of the invention include oral administration of one or more ANGPTL3 dsRNA agents to a subject. ANGPTL3 dsRNA agents or ANGPTL3 antisense polynucleotide agents, either alone or in conjunction with other therapeutic agents, may be administered once, or alternatively they may be administered in a plurality of administrations. If administered multiple times, the ANGPTL3 dsRNA agent or ANGPTL3 antisense polynucleotide agent may be administered via different routes. For example, though not intended to be limiting, a first (or first several) administrations may be made via subcutaneous means and one or more additional administrations may be oral and/or systemic administrations.

For embodiments of the invention in which it is desirable to administer an ANGPTL3 dsRNA agent or ANGPTL3 antisense polynucleotide agent systemically, the ANGPTL3 dsRNA agent or ANGPTL3 antisense polynucleotide agent may be formulated for parenteral administration by injection, e.g., by bolus injection or continuous infusion. Formulations for injection may be presented in unit dosage form, e.g., in ampoules or in multi-dose containers, with or without an added preservative. ANGPTL3 dsRNA agent formulations (also referred to as pharmaceutical compositions) may take such forms as suspensions, solutions or emulsions in oily or aqueous vehicles, and may contain formulatory agents such as suspending, stabilizing and/or dispersing agents.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Lower doses will result from other forms of administration, such as intravenous administration. In the event that a response in a subject is insufficient at the initial doses applied, higher doses (or effectively higher doses by a different, more localized delivery route) may be employed to the extent that patient tolerance permits. Multiple doses per day may be used as needed to achieve appropriate systemic or local levels of one or more ANGPTL3 dsRNA agents or ANGPTL3 antisense polynucleotide agents and to achieve appropriate reduction in ANGPTL3 protein activity.

In yet other embodiments, methods of the invention include use of a delivery vehicle such as biocompatible microparticle, nanoparticle, or implant suitable for implantation into a recipient, e.g., a subject. Exemplary bioerodible implants that may be useful in accordance with this method are described in PCT Publication No. WO 95/24929 (incorporated by reference herein), which describes a biocompatible, biodegradable polymeric matrix for containing a biological macromolecule.

Both non-biodegradable and biodegradable polymeric matrices can be used in methods of the invention to deliver one or more ANGPTL3 dsRNA agents or ANGPTL3 antisense polynucleotide agents to a subject. In some embodiments, a matrix may be biodegradable. Matrix polymers may be natural or synthetic polymers. A polymer can be selected based on the period of time over which release is desired, generally in the order of a few hours to a year or longer. Typically, release over a period ranging from between a few hours and three to twelve months can be used. The polymer optionally is in the form of a hydrogel that can absorb up to about 90% of its weight in water and further, optionally is cross-linked with multivalent ions or other polymers.

In general, ANGPTL3 dsRNA agents or ANGPTL3 antisense polynucleotide agents may be delivered in some embodiments of the invention using the bioerodible implant by way of diffusion, or by degradation of the polymeric matrix. Exemplary synthetic polymers for such use are well known in the art. Biodegradable polymers and non-biodegradable polymers can be used for delivery of ANGPTL3 dsRNA agents or ANGPTL3 antisense polynucleotide agents using art-known methods. Bioadhesive polymers such as bioerodible hydrogels (see H. S. Sawhney, C. P. Pathak and J. A. Hubell in Macromolecules, 1993, 26, 581-587, the teachings of which are incorporated by reference herein) may also be used to deliver ANGPTL3 dsRNA agents or ANGPTL3 antisense polynucleotide agents for treatment of an ANGPTL3-associated disease or condition. Additional suitable delivery systems can include time-release, delayed release or sustained release delivery systems. Such systems can avoid repeated administrations of an ANGPTL3 dsRNA agent or ANGPTL3 antisense polynucleotide agent, increasing convenience to the subject and the medical care professional. Many types of release delivery systems are available and known to those of ordinary skill in the art. (See for example: U.S. Pat. Nos. 5,075,109; 4,452,775; 4,675,189; 5,736,152; 3,854,480; 5,133,974; and 5,407,686 (the teaching of each of which is incorporated herein by reference). In addition, pump-based hardware delivery systems can be used, some of which are adapted for implantation.

Use of a long-term sustained release implant may be suitable for prophylactic treatment of subjects and for subjects at risk of developing a recurrent ANGPTL3-associated disease or condition. Long-term release, as used herein, means that the implant is constructed and arranged to deliver a therapeutic level of an ANGPTL3 dsRNA agent or ANGPTL3 antisense polynucleotide agent for at least up to 10 days, 20 days, 30 days, 60 days, 90 days, six months, a year, or longer. Long-term sustained release implants are well-known to those of ordinary skill in the art and include some of the release systems described above.

Therapeutic formulations of ANGPTL3 dsRNA agents or ANGPTL3 antisense polynucleotide agents may be prepared for storage by mixing the molecule or compound having the desired degree of purity with optional pharmaceutically acceptable carriers, excipients or stabilizers [Remington's Pharmaceutical Sciences 21^{st} edition, (2006)], in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN©, PLURONICS© or polyethylene glycol (PEG).

### Cells, Subjects, and Controls

Methods of the invention may be used in conjunction with cells, tissues, organs and/or subjects. In some aspects of the invention a subject is a human or vertebrate mammal including but not limited to a dog, cat, horse, cow, goat, mouse, rat, and primate, e.g., monkey. Thus, the invention can be used to treat ANGPTL3-associated diseases or conditions in human and non-human subjects. In some aspects of the invention a subject may be a farm animal, a zoo animal, a domesticated animal or non-domesticated animal and methods of the invention can be used in veterinary prevention and treatment regimens. In some embodiments of the invention, the subject is a human and methods of the invention can be used in human prevention and treatment regimens.

Non-limiting examples of subjects to which the present invention can be applied are subjects who are diagnosed with, suspected of having, or at risk of having a disease or condition associated with a higher than desirable ANGPTL3 expression and/or activity, also referred to as “elevated levels of ANGPTL3 expression”. Non-limiting examples of diseases and conditions associated with a higher than desirable levels of ANGPTL3 expression and/or activity are described elsewhere herein. Methods of the invention may be applied to a subject who, at the time of treatment, has been diagnosed as having the disease or condition associated with a higher than desirable ANGPTL3 expression and/or activity, or a subject who is considered to be at risk for having or developing a disease or condition associated with a higher than desirable ANGPTL3 expression and/or activity. In some aspects of the invention a disease or condition associated with a higher than desirable ANGPTL3 level of expression and/or activity is an acute disease or condition, and in certain aspects of the invention a disease or condition associated with a higher than desirable ANGPTL3 level of expression and/or activity is a chronic disease or condition.

In a non-limiting example, an ANGPTL3 dsRNA agent of the invention is administered to a subject diagnosed with, suspected of having, or at risk of having, statin resistant hypercholesterolemia, which is a disease in which it is desirable to reduce ANGPTL3 expression. Methods of the invention may be applied to the subject who, at the time of treatment, has been diagnosed as having the disease or condition, or a subject who is considered to be at risk for having or developing the disease or condition.

In another non-limiting example, an ANGPTL3 dsRNA agent of the invention is administered to a subject diagnosed with, suspected of having, or at risk of having, hyperlipidemia, which is a disease in which it is desirable to reduce ANGPTL3 expression. Methods of the invention may be applied to the subject who, at the time of treatment, has been diagnosed as having the disease or condition, or a subject who is considered to be at risk for having or developing the disease or condition.

A cell to which methods of the invention may be applied include cells that are in vitro, in vivo, ex vivo cells. Cells may be in a subject, in culture, and/or in suspension, or in any other suitable state or condition. A cell to which a method of the invention may be applied can be a liver cell, a hepatocyte, a cardiac cell, a pancreatic cell, a cardiovascular cell, kidney cell or other type of vertebrate cell, including human and non-human mammalian cells. In certain aspects of the invention, a cell to which methods of the invention may be applied is a healthy, normal cell that is not known to be a disease cell. In certain embodiments of the invention a cell to which methods and compositions of the invention are applied to a liver cell, a hepatocyte, a cardiac cell, a pancreatic cell, a cardiovascular cell, and/or a kidney cell. In certain aspects of the invention, a control cell is a normal cell, but it will be understood that a cell having a disease or condition may also serve as a control cell in particular circumstances for example to compare results in a treated cell having a disease or condition versus an untreated cell having the disease or condition, etc.

A level of ANGPTL3 polypeptide activity can be determined and compared to control level of ANGPTL3 polypeptide activity, according to methods of the invention. A control may be a predetermined value, which can take a variety of forms. It can be a single cut-off value, such as a median or mean. It can be established based upon comparative groups, such as in groups having normal levels of ANGPTL3 polypeptide and/or ANGPTL3 polypeptide activity and groups having increased levels of ANGPTL3 polypeptide and/or ANGPTL3 polypeptide activity. Another non-limiting example of comparative groups may be groups having one or more symptoms of or a diagnosis of an ANGPTL3-associated disease or condition; groups without having one or more symptoms of or a diagnosis of the disease or condition; groups of subjects to whom an siRNA treatment of the invention has been administered; groups of subjects to whom an siRNA treatment of the invention has not been administered. Typically, a control may be based on apparently healthy normal individuals in an appropriate age bracket or apparently healthy cells. It will be understood that controls according to the invention may be, in addition to predetermined values, samples of materials tested in parallel with the experimental materials. Examples include samples from control populations or control samples generated through manufacture to be tested in parallel with the experimental samples. In some embodiments of the invention, a control may include a cell or subject not contacted or treated with an ANGPTL3 dsRNA agent of the invention and in such instances, a control level of ANGPTL3 polypeptide and/or ANGPTL3 polypeptide activity can be compared to a level of ANGPTL3 polypeptide and/or ANGPTL3 polypeptide activity in a cell or subject contacted with an ANGPTL3 dsRNA agent or ANGPTL3 antisense polynucleotide agent of the invention.

In some embodiments of the invention a level of ANGPTL3 polypeptide determined for a subject can be a control level against which a level of ANGPTL3 polypeptide determined for the same subject at a different time is compared. In a non-limiting example, a level of ANGPTL3 is determined in a biological sample obtained from a subject who has not been administered an ANGPTL3 treatment of the invention. In some embodiments, the biological sample is a serum sample. The level of ANGPTL3 polypeptide determined in the sample obtained from the subject can serve as a baseline or control value for the subject. After one or more administrations of an ANGPTL3 dsRNA agent to the subject in a treatment method of the invention, one or more additional serum samples can be obtained from the subject and the level of ANGPTL3 polypeptide in the subsequent sample or samples can be compared to the control/baseline level for the subject. Such comparisons can be used to assess onset, progression, or recession of an ANGPTL3 associated disease or condition in the subject. For example, a level of ANGPTL3 polypeptide in the baseline sample obtained from the subject that is higher than a level obtained from the same subject after the subject has been administered an ANGPTL3 dsRNA agent or ANGPTL3 antisense polynucleotide agent of the invention indicates regression of the ANGPTL3-associated disease or condition and indicates efficacy of the administered ANGPTL3 dsRNA agent of the invention for treatment of the ANGPTL3-associated disease or condition.

In some aspects of the invention, values of one or more of a level of ANGPTL3 polypeptide and/or ANGPTL3 polypeptide activity determined for a subject may serve as control values for later comparison of levels of ANGPTL3 polypeptide and/or ANGPTL3 activity, in that same subject, thus permitting assessment of changes from a “baseline” ANGPTL3 polypeptide activity in a subject. Thus, an initial ANGPTL3 polypeptide level and/or initial ANGPTL3 polypeptide activity level may be present and/or determined in a subject and methods and compounds of the invention may be used to decrease the level of ANGPTL3 polypeptide and/or ANGPTL3 polypeptide activity in the subject, with the initial level serving as a control level for that subject.

Using methods of the invention, ANGPTL3 dsRNA agents and/or ANGPTL3 antisense polynucleotide agents of the invention may be administered to a subject. Efficacy of the administration and treatment of the invention can be assessed when a level of ANGPTL3 polypeptide in a serum sample obtained from a subject is decreased by at least 0.5%, 1%, 5%, 10%, 20%, 30%, 40%, 50%, 60%, 70%, 80%, 90%, 95%, or more compared to a pre-administration level of ANGPTL3 polypeptide in a serum sample obtained from the subject at a prior time point, or compared to a non-contacted control level, for example a level of ANGPTL3 polypeptide in a control serum sample. It will be understood that a level of ANGPTL3 polypeptide and a level of ANGPTL3 polypeptide activity both correlate with a level of ANGPTL3 gene expression. Certain embodiments of methods of the invention comprise administering an ANGPTL3 dsRNA and/or ANGPTL3 antisense agent of the invention to a subject in an amount effective to inhibit ANGPTL3 gene expression and thereby reduce a level of ANGPTL3 polypeptide and reduce a level of ANGPTL3 polypeptide activity in the subject.

Some embodiments of the invention, include determining presence, absence, and/or an amount (also referred to herein as a level) of ANGPTL3 polypeptide in one or more biological samples obtained from one or more subjects. The determination can be used to assess efficacy of a treatment method of the invention. For example, methods and compositions of the invention can be used to determine a level of ANGPTL3 polypeptide in a biological sample obtained from a subject previously treated with administration of an ANGPTL3 dsRNA agent and/or an ANGPTL3 antisense agent of the invention. A level of ANGPTL3 polypeptide determined in a serum sample obtained from the treated subject that is lower by at least 0.5%, 1%, 5%, 10%, 20%, 30%, 40%, 50%, 60%, 70%, 80%, 90%, 95%, or more compared to a pretreatment level of ANGPTL3 polypeptide determined for the subject, or compared to a non-contacted control biological sample level, indicates a level of efficacy of the treatment administered to the subject.

In some embodiments of the invention a physiological characteristic of an ANGPTL3-associated disease or condition determined for a subject can be a control determination against which a determination of the physiological characteristic in the same subject at a different time is compared. In a non-limiting example, a physiological characteristic such as a lipid level and/or an HDL:LDL ratio is determined in a biological sample, such as a serum sample, obtained from a subject who has not been administered an ANGPTL3 treatment of the invention. The lipid level and/or HDL:LDL ratio (and/or other physiological characteristic of an ANGPTL3 disease or condition) determined in the sample obtained from the subject can serve as a baseline or control value for the subject. After one or more administrations of an ANGPTL3 dsRNA agent to the subject in a treatment method of the invention, one or more additional serum samples can be obtained from the subject and the lipid level and/or HDL LDL ratio in the subsequent sample or samples are compared to the control/baseline level and/or ratio, respectively, for the subject. Such comparisons can be used to assess onset, progression, or recession of an ANGPTL3 associated disease or condition in the subject. For example, a lipid level in the baseline sample obtained from the subject that is higher than a lipid level determined in a sample obtained from the same subject after the subject has been administered an ANGPTL3 dsRNA agent or ANGPTL3 antisense polynucleotide agent of the invention indicates regression of the ANGPTL3-associated disease or condition and indicates efficacy of the administered ANGPTL3 dsRNA agent of the invention for treatment of the ANGPTL3-associated disease or condition.

In some aspects of the invention, values of one or more of a physiological characteristic of an ANGPTL3-associcated disease or condition determined for a subject may serve as control values for later comparison of the physiological characteristics in that same subject, thus permitting assessment of changes from a “baseline” physiological characteristic in a subject. Thus, an initial physiological characteristic may be present and/or determined in a subject and methods and compounds of the invention may be used to decrease the level of ANGPTL3 polypeptide and/or ANGPTL3 polypeptide activity in the subject, with the initial physiological characteristic determination serving as a control for that subject.

Using methods of the invention, ANGPTL3 dsRNA agents and/or ANGPTL3 antisense polynucleotide agents of the invention may be administered to a subject in an effective amount to treat an ANGPTL3 disease or condition. Efficacy of the administration and treatment of the invention can be assessed by determining a change in one or more physiological characteristics of the ANGPTL3 disease or condition. In a non-limiting example, a lipid level in a serum sample obtained from a subject is decreased by at least 0.5%, 1%, 5%, 10%, 20%, 30%, 40%, 50%, 60%, 70%, 80%, 90%, 95%, or more compared to a pre-administration lipid in a serum sample obtained from the subject at a prior time point, or compared to a non-contacted control level, for example a lipid level in a control serum sample. It will be understood that a lipid level, HDL level, HDL:LDL ratio, triglyceride level, amount of fat in a subject's liver each correlates with a level of ANGPTL3 gene expression. Certain embodiments of methods of the invention comprise administering an ANGPTL3 dsRNA and/or ANGPTL3 antisense agent of the invention to a subject in an amount effective to inhibit ANGPTL3 gene expression and thereby reduce a lipid level, HDL level, HDL:LDL ratio, triglyceride level, amount of fat in a subject's liver, or otherwise positively impact a physiological characteristic of an ANGPTL3-associated disease or condition in the subject.

Some embodiments of the invention, include determining presence, absence, and/or a change in a physiological characteristic of an ANGPTL3-associated disease or condition using methods such as but not limited to: (1) assessing one or more biological samples obtained from one or more subjects for the physiological characteristic; (2) imaging a subject (for example but not limited to obtaining a liver image); and (3) or physical examination of the subject. The determination can be used to assess efficacy of a treatment method of the invention.

### Kits

Also within the scope of the invention are kits that comprise one or more ANGPTL3 dsRNA agents and/or ANGPTL3 antisense polynucleotide agents and instructions for its use in methods of the invention. Kits of the invention may include one or more of an ANGPTL3 dsRNA agent, ANGPTL3 sense polynucleotide, and ANGPTL3 antisense polynucleotide agent that may be used to treat an ANGPTL3-associated disease or condition. Kits containing one or more ANGPTL3 dsRNA agents, ANGPTL3 sense polynucleotides, and ANGPTL3 antisense polynucleotide agents can be prepared for use in treatment methods of the invention. Components of kits of the invention may be packaged either in aqueous medium or in lyophilized form. A kit of the invention may comprise a carrier being compartmentalized to receive in close confinement therein one or more container means or series of container means such as test tubes, vials, flasks, bottles, syringes, or the like. A first container means or series of container means may contain one or more compounds such as an ANGPTL3 dsRNA agent and/or ANGPTL3 sense or antisense polynucleotide agent. A second container means or series of container means may contain a targeting agent, a labelling agent, a delivery agent, etc. that may be included as a portion of an ANGPTL3 dsRNA agent and/or ANGPTL3 antisense polynucleotide to be administered in an embodiment of a treatment method of the invention.

A kit of the invention may also include instructions. Instructions typically will be in written form and will provide guidance for carrying-out a treatment embodied by the kit and for making a determination based upon that treatment.

The following examples are provided to illustrate specific instances of the practice of the present invention and are not intended to limit the scope of the invention. As will be apparent to one of ordinary skill in the art, the present invention will find application in a variety of compositions and methods.

### EXAMPLES

### Example 1

### Synthesis of ANGPTL3 RNAi Agents

ANGPTL3 RNAi agent duplexes shown in Table 2-5, above, were synthesized in accordance with the following general procedures:

Sense and antisense strand sequences of siRNA were synthesized on oligonucleotide synthesizers using a well-established solid phase synthesis method based on phosphoramidite chemistry. Oligonucleotide chain propagation is achieved through 4-step cycles: a deprotection, a condensation, a capping and an oxidation or a sulfurization step for addition of each nucleotide. Syntheses were performed on a solid support made of controlled pore glass (CPG, 1000 Å). Monomer phosphoramidites were purchased from commercial sources. Phosphoramidites with GalNAc ligand cluster (GLPA1 and GLPA2 as non-limiting examples) were synthesized according to the procedures of Examples 2-3 herein. For siRNAs used for in vitro screening (Table 2.), syntheses were carried out at 2 μmol scale, and for siRNAs used for in vivo testing (Table 3, 4 and 5), syntheses were carried out at scale of 5 μmol or larger. In the case where the GalNAc ligand (GLO-0 as a non-limiting example) is attached at 3′-end of sense strand, GalNAc ligand attached CPG solid support was used. In the case where the GalNAc ligand (GLS-1 or GLS-2 as non-limiting example) is attached at 5′-end of sense strand, a GalNAc phosphoramidite (GLPA1 or GLPA2 as a non-limiting example) was used for the last coupling reaction. Trichloroacetic acid (TCA) 3% in dichloromethane was used for deprotection of 4,4′-dimethoxytrityl protecting group (DMT). 5-Ethylthio-1H-tetrazole was used as an activator. I₂ in THF/Py/H₂O and phenylacetyl disulfide (PADS) in pyridine/MeCN was used for oxidation and sulfurization reactions, respectively. After the final solid phase synthesis step, solid support bound oligomer was cleaved and protecting groups were removed by treating with a 1:1 volume solution of 40 wt. % methylamine in water and 28% ammonium hydroxide solution. For the synthesis of siRNAs used for in vitro screening, crude mixture was concentrated. The remaining solid was dissolved in 1.0 M NaOAc, and ice cold EtOH was added to precipitate out the single strand product as the sodium salt, which was used for annealing without further purification. For the synthesis of siRNAs used for in vivo testing, crude single strand product was further purified by ion pairing reversed phase HPLC (IP-RP-HPLC). Purified single strand oligonucleotide product from IP-RP-HPLC was converted to sodium salt by dissolving in 1.0 M NaOAc and precipitation by addition of ice cold EtOH. Annealing of equimolar complementary sense stand and antisense strand oligonucleotide in water was performed to form the double strand siRNA product, which was lyophilized to afford a fluffy white solid.

TABLE 6
Mass and purity information of siRNAs provided
in Table 2 - see Duplex ID Nos.

| [symbol] | [symbol] | | | [symbol] | | |
|---|---|---|---|---|---|---|
| ID | [symbol] | [symbol] | HPLC | [symbol] | [symbol] | HPLC |
| AD# | [symbol] | [symbol] | [symbol] | [symbol] | [symbol] | [symbol] |
| AD00001 | 6972.77 | 6973.28 | 90% | 6941.70 | 6941.84 | >80% |
| AD00002 | 7034.85 | 7035.29 | 92% | 6894.64 | 6895.1 | >80% |
| AD00003 | 6948.74 | 6949.23 | 86% | 6980.75 | 6980.99 | 84% |
| AD00004 | 7002.85 | 7003.42 | 85% | 6896.61 | 6897.16 | >80% |
| AD00005 | 6955.78 | 6956.4 | 86% | 6958.68 | 6959.34 | >80% |
| AD00006 | 6955.79 | 6956.27 | 85% | 6958.69 | 6959.2 | >80% |
| AD00007 | 6943.68 | 6944.1 | 87% | 6970.80 | 6971.41 | >80% |
| AD00008 | 6942.70 | 6943.41 | 86% | 6986.79 | 6986.93 | >80% |
| AD00009 | 7025.89 | 7026.56 | 89% | 6873.57 | 6874.09 | 82% |
| AD00010 | 6978.83 | 6979.2 | 85% | 6935.66 | 6936.29 | 89% |
| AD00011 | 7137.95 | 7138.63 | 86% | 6791.54 | 6792.11 | 88% |
| AD00012 | 7074.87 | 7075.56 | 87% | 6854.62 | 6855.26 | >80% |
| AD00013 | 6947.76 | 6948.47 | 86% | 6996.73 | 6997.31 | 85% |
| AD00014 | 6993.82 | 6994.51 | 85% | 6950.66 | 6950.84 | 85% |
| AD00015 | 7009.83 | 7010.53 | 84% | 6949.67 | 6950.27 | 84% |
| AD00016 | 6978.83 | 6979.56 | 86% | 6935.65 | 6936.25 | >80% |
| AD00017 | 7056.91 | 7057.52 | 87% | 6887.58 | 6888.14 | 85% |
| AD00018 | 7016.88 | 7017.58 | 86% | 6927.62 | 6928.25 | 83% |
| AD00019 | 7016.90 | 7017.34 | 87% | 6927.64 | 6928.46 | 85% |
| AD00020 | 6883.71 | 6884.18 | 85% | 7075.82 | 7076.53 | >80% |
| AD00021 | 6883.71 | 6884.16 | 88% | 7075.80 | 7076.35 | 89% |
| AD00022 | 6885.68 | 6886.17 | 86% | 7043.82 | 7044.36 | 89% |
| AD00023 | 6902.66 | 6903.06 | 89% | 7026.84 | 7027.34 | 88% |
| AD00024 | 6878.62 | 6879.13 | 85% | 7065.84 | 7066.38 | 88% |
| AD00025 | 6902.67 | 6903.06 | 87% | 7026.83 | 7027.36 | 89% |
| AD00026 | 6902.66 | 6903.09 | 87% | 7026.84 | 7027.36 | 87% |
| AD00027 | 6919.65 | 6920.09 | 89% | 7009.83 | 7010.01 | 81% |
| AD00028 | 6958.69 | 6959.22 | 90% | 6985.82 | 6986.45 | 88% |
| AD00029 | 6971.78 | 6972.48 | 88% | 6957.69 | 6958.29 | 86% |
| AD00030 | 7073.88 | 7074.55 | 87% | 6870.61 | 6871.25 | 86% |
| AD00031 | 7120.95 | 7121.52 | 86% | 6808.54 | 6809.2 | 85% |
| AD00032 | 7057.88 | 7058.26 | 87% | 6871.61 | 6872.22 | 88% |
| AD00033 | 6994.83 | 6995.26 | 85% | 6934.67 | 6935.23 | 89% |
| AD00034 | 7017.86 | 7018.53 | 86% | 6911.62 | 6912.2 | 87% |
| AD00035 | 7152.97 | 7153.65 | 88% | 6806.57 | 6807.27 | 86% |
| AD00036 | 7057.89 | 7058.52 | 86% | 6871.61 | 6872.78 | >80% |
| AD00037 | 7073.90 | 7074.24 | 84% | 6870.61 | 6871.2 | 88% |
| AD00038 | 7034.87 | 7035.46 | 86% | 6894.64 | 6895.21 | 82% |
| AD00039 | 6942.68 | 6943.32 | 86% | 6986.79 | 6987.38 | 86% |
| AD00040 | 7193.95 | 7194.55 | 85% | 6750.52 | 6751 | 85% |
| AD00041 | 7080.94 | 7081.51 | 87% | 6848.55 | 6849.29 | 81% |
| AD00042 | 7051.83 | 7052.46 | 86% | 6877.67 | 6878.18 | 83% |
| AD00043 | 6972.79 | 6973.59 | 89% | 6941.70 | 6942.23 | 81% |
| AD00044 | 7001.87 | 7002.43 | 89% | 6912.61 | 6913.22 | 82% |
| AD00045 | 7025.84 | 7026.44 | 91% | 6948.69 | 6949.15 | 82% |
| AD00046 | 6954.81 | 6955.27 | 86% | 6974.68 | 6975.21 | 83% |
| AD00047 | 7010.83 | 7011.44 | 87% | 6933.69 | 6934.26 | 85% |
| AD00048 | 6994.82 | 6995.43 | 88% | 6934.66 | 6935.26 | 83% |
| AD00049 | 7018.86 | 7019.6 | >80% | 6895.62 | 6896.25 | >80% |
| AD00050 | 7096.93 | 7096.96 | >80% | 6847.57 | 6848.13 | >80% |

### Example 2. Preparation of Intermediate-A and Intermediate-B

As shown in Scheme 1 below, Intermediate-A was synthesized by treating commercially available galactosamine pentaacetate with trimethylsilyl trifluoromethanesulfonate (TMSOTf) in dichloromethane (DCM). This was followed by glycosylation with Cbz protected 2-(2-aminoethoxy)ethan-1-ol to give Compound IL. The Cbz protecting group was removed by hydrogenation to afford Intermediate-A as a trifluoroacetate (TFA) salt. Intermediate B was synthesized based on the same scheme except Cbz protected 2-(2-(2-aminoethoxy)ethoxy)ethan-1-ol was used as the starting material.

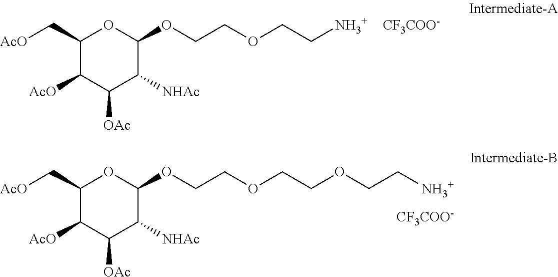

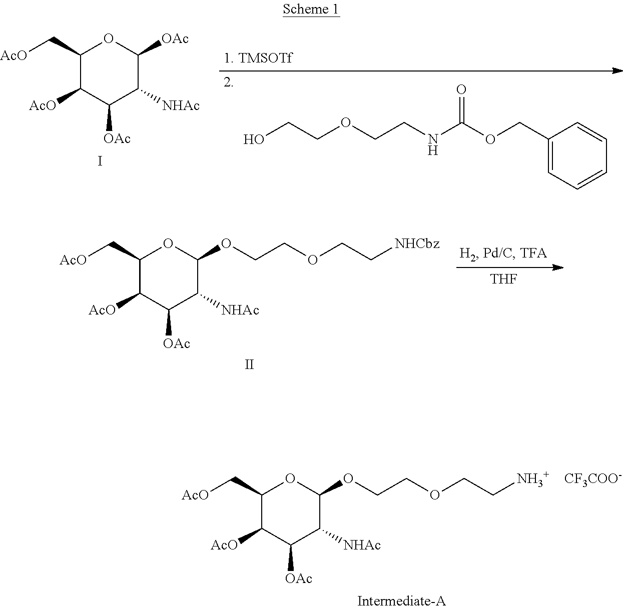

To a solution of Compound I (20.0 g, 51.4 mmol) in 100 mL 1,2-dichloroethane (DCE) was added TMSOTf (17.1 g, 77.2 mmol). The resulting reaction solution was stirred at 60° C. for 2 hrs, and then at 25° C. for 1 hr. Cbz protected 2-(2-aminoethoxy)ethan-1-ol (13.5 g, 56.5 mmol) in DCE (100 mL) dried over 4 Å powder molecular sieves (10 g) was added dropwise to the above mentioned reaction solution at 0° C. under N₂ atmosphere. The resulting reaction mixture was stirred at 25° C. for 16 hrs under N₂ atmosphere. The reaction mixture was filtered and washed with sat. NaHCO₃ (200 mL), water (200 mL) and sat. brine (200 mL). The organic layer was dried over anhydrous Na₂SO₄, filtered and concentrated under reduced pressure to give a crude product, which was triturated with 2-Methyltetrahydrofuran/heptane (5/3, v/v, 1.80 L) for 2 hrs. Resulting mixture was filtered and dried to give Compound II (15.0 g, 50.3% yield) as a white solid.

To a dried and argon purged hydrogenation bottle was carefully added 10% Pd/C (1.50 g), followed by 10 mL tetrahydrofuran (THF) and then a solution of Compound II (15.0 g, 26.4 mmol) in THE (300 mL) and TFA (trifluoroacetic acid, 3.00 g, 26.4 mmol). The resulting mixture was degassed and purged with H₂ three times and stirred at 25° C. for 3 hrs under H₂ (45 psi) atmosphere. Thin-layer chromatography (TLC, solvent: DCM:MeOH=10:1) indicated Compound II was consumed completely. The reaction mixture was filtered and concentrated under reduced pressure. Residue was dissolved in anhydrous DCM (500 mL) and concentrated. This process was repeated 3 times to give Intermediate-A (14.0 g, 96.5% yield) as a foamy white solid. ¹H NMR (400 MHz DMSO-d₆): δ ppm 7.90 (d, J=9.29 Hz, 1H), 7.78 (br s, 3H), 5.23 (d, J=3.26 Hz, 1H), 4.98 (dd, J=11.29, 3.26 Hz, 1H), 4.56 (d, J=8.53 Hz, 1H), 3.98-4.07 (m, 3H), 3.79-3.93 (m, 2H), 3.55-3.66 (m, 5H), 2.98 (br d, J=4.77 Hz, 2 H), 2.11 (s, 3H), 2.00 (s, 3H), 1.90 (s, 3H), 1.76 (s, 3H).

Intermediate-B was synthesized using similar procedures for synthesis of Intermediate-A. ¹H NMR (400 MHz DMSO-d₆): δ ppm 7.90 (br d, J=9.03 Hz, 4H), 5.21 (d, J=3.51 Hz, 1H), 4.97 (dd, J=11.1 Hz, 1H), 4.54 (d, J=8.53 Hz, 1H), 3.98-4.06 (m, 3H), 3.88 (dt, J=10.9 Hz, 1H), 3.76-3.83 (m, 1H), 3.49-3.61 (m, 9H), 2.97 (br s, 2H), 2.10 (s, 3H), 1.99 (s, 3H), 1.88 (s, 3H), 1.78 (s, 3H). Mass calc. for C₂₀H₃₄N₂O₁₁: 478.22; found: 479.3 (M+H⁺).

### Example 3. Synthesis of GalNAc Ligand Cluster Phosphoramidite GLPA1, GLPA2 and GLPA15

Scheme 2 below was followed to prepare GLPA1 and GLPA2. Starting from benzyl protected propane-1,3-diamine, it was alkylated with tert-butyl 2-bromoacetate to afford triester Compound I. The benzyl protecting group was removed by hydrogenation to afford secondary amine Compound II. Amide coupling with 6-hydroxyhexanoic acid afforded Compound III. tert-Butyl protecting groups were then removed upon treatment of HCl in dioxane to generate triacid Compound IV. Amide coupling between triacid compound IV and Intermediate-A or Intermediate-B was performed to afford Compound Va or Vb. Phosphoramidite GLPA1 or GLPA2 was synthesized by phosphitylation of Compound Va or Vb with 2-Cyanoethyl N,N-diisopropylchlorophosphoramidite and a catalytic amount of 1H-tetrazole.

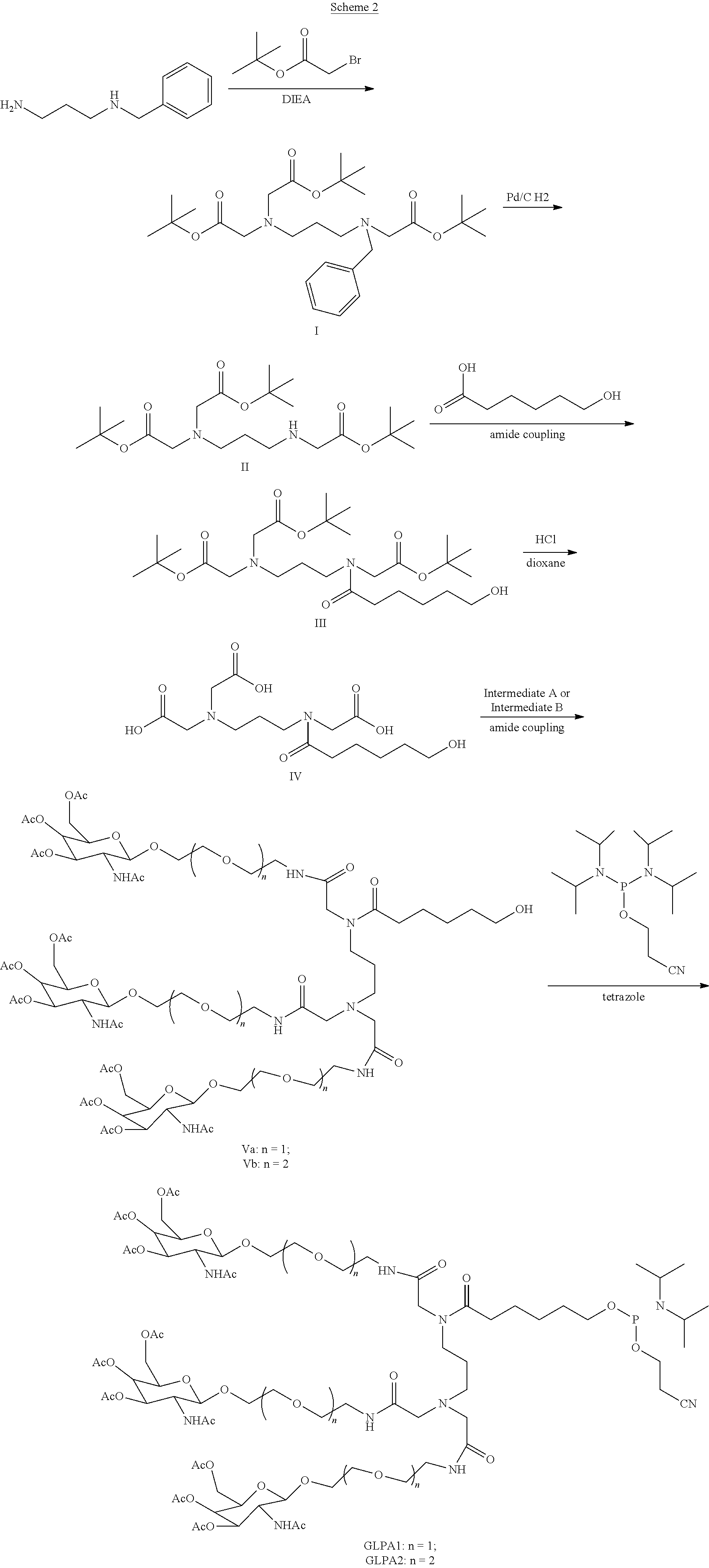

To a solution of N-Benzyl-1,3-propanediamine (5.00 g, 30.4 mmol) in dimethylformamide (DMF, 100 mL) was added tert-butyl 2-bromoacetate (23.7 g, 121 mmol), followed by addition of diisopropylethylamine (DIEA, 23.61 g, 182 mmol) dropwise. The resulting reaction mixture was stirred at 25-30° C. for 16 hrs. LCMS showed N-Benzyl-1,3-propanediamine was consumed completely. Reaction mixture was diluted with H₂O (500 mL) and extracted with EtOAc (500 mL×2). The combined organics were washed with sat. brine (1 L), dried over anhydrous Na₂SO₄, filtered, and concentrated under reduced pressure to give crude product, which was purified by silica gel column chromatography (gradient: petroleum ether:ethyl acetate from 20:1 to 5:1). Compound I (12.1 g, 78.4% yield) was obtained as a colorless oil. ¹H NMR (400 MHz, CDCl₃): δ ppm 7.26-7.40 (m, 5H), 3.79 (s, 2H), 3.43 (s, 4H), 3.21 (s, 2H), 2.72 (dt, J=16.9, 7.34 Hz, 4H), 1.70 (quin, J=7.2 Hz, 2H), 1.44-1.50 (m, 27H).

A dried hydrogenation bottle was purged with Argon three times. Pd/C (200 mg, 10%) was added, followed by MeOH (5 mL) and then a solution of Compound I (1.00 g, 1.97 mmol) in MeOH (5 mL). The reaction mixture was degassed under vacuum and refilled with H₂. This process was repeated three times. The mixture was stirred at 25° C. for 12 hrs under H₂ (15 psi) atmosphere. LCMS showed Compound I was consumed completely. The reaction mixture was filtered under reduced pressure under N₂ atmosphere. Filtrate was concentrated under reduced pressure to give Compound II (655 mg, 79.7% yield) as yellow oil, which was used for the next step without further purification. ¹H NMR (400 MHz, CDCl₃): δ ppm 3.44 (s, 4H), 3.31 (s, 2H), 2.78 (t, J=7.1 Hz, 2H), 2.68 (t, J=6.9 Hz, 2H), 1.88 (br s, 1H), 1.69 (quin, J=7.03 Hz, 2H), 1.44-1.50 (s, 27H).

A mixture of Compound II (655 mg, 1.57 mmol), 6-hydroxyhexanoic acid (249 mg, 1.89 mmol), DIEA (1.02 g, 7.86 mmol), 1-ethyl-3-(3-dimethylaminopropyl)carbodiimide hydrochloride (EDCI, 904 mg, 4.72 mmol), and 1-Hydroxybenzotriazole (HOBt, 637 mg, 4.72 mmol) in DMF (6 mL) was degassed and purged with N₂ three times, and then was stirred at 25° C. for 3 hrs under N₂ atmosphere. LCMS indicated desired product. The reaction mixture was diluted with H₂O (10 mL) and extracted with EtOAc 20 mL (10 mL×2). Organics were combined and washed with sat. brine (20 mL), dried over anhydrous Na₂SO₄, filtered, and concentrated to give crude product, which was purified by silica gel column chromatography (gradient: petroleum ether:ethyl acetate from 5:1 to 1:1) to afford Compound III (650 mg, 77.8% yield) as a yellow oil. ¹H NMR (400 MHz, CDCl₃): δ ppm 3.90-3.95 (s, 2H), 3.63 (t, J=6.40 Hz, 2H), 3.38-3.45 (m, 6H), 2.72 (t, J=6.65 Hz, 2H), 2.40 (t, J=7.28 Hz, 2H), 1.55-1.75 (m, 8H), 1.44 (s, 27H). Mass calc. for C₂₇H₅₀N₂O₈: 530.36; found: 531.3 (M+H⁺).

A mixture of Compound III (5.5 g, 10.3 mmol) in HCl/dioxane (2M, 55 mL) was stirred at 25° C. for 3 hrs. LCMS showed complete consumption of Compound III. Reaction mixture was filtered, washed with EtOAc (50 mL), and dried under reduced pressure to give crude product. It was dissolved in CH₃CN (50 mL), volatiles were removed under vacuum. This process was repeated three times to give Compound IV (2.05 g, 54.5% yield) as a white solid. ¹H NMR (400 MHz, D₂O): δ ppm 4.21 (s, 1H), 4.07 (d, J=4.5 Hz, 4H), 3.99 (s, 1H), 3.45-3.52 (m, 3H), 3.42 (t, J=6.5 Hz, 1H), 3.32-3.38 (m, 1H), 3.24-3.31 (m, 1H), 2.37 (t, J=7.4 Hz, 1H), 2.24 (t, J=7.4 Hz, 1H), 1.99 (dt, J=15.5, 7.53 Hz, 1H), 1.85-1.94 (m, 1H), 1.85-1.94 (m, 1H), 1.39-1.56 (m, 4H), 1.19-1.31 (m, 2H).

A mixture of Compound IV (500 mg, 1.05 mmol), Intermediate-A (2.02 g, 3.67 mmol), DIEA (813 mg, 6.30 mmol), EDCI (704 mg, 3.67 mmol) and HOBt (496 mg, 3.67 mmol) in DMF (10 mL) was degassed and purged with N₂ for 3 times, and then the mixture was stirred at 25° C. for 3 hrs under N2 atmosphere. LCMS indicated desired product. The reaction mixture was quenched by addition of H₂O (10 mL), extracted with DCM (10 mL×2). The combined organics were extracted with 10% citric acid (20 mL). The aqueous phase was neutralized with saturated NaHCO₃ solution and re-extracted with DCM (10 mL×2). Organics were dried over sodium sulfate, filtered and concentrated under reduced pressure to give Compound Va (570 mg, 0.281 mmol, 26.8% yield) as a white solid. ¹H NMR: (400 MHz, CDCl₃) ppm δ 7.84-8.12 (m, 3H), 6.85-7.15 (m, 2H), 6.66-6.81 (m, 1H), 5.36 (brd, J=2.7 Hz, 3H), 5.11-5.27 (m, 3H), 4.63-4.85 (m, 3H), 3.90-4.25 (m, 18H), 3.37-3.75 (m, 28H), 3.15-3.28 (m, 4H), 2.64 (br d, J=6.53 Hz, 2H), 2.30-2.46 (m, 2H), 2.13-2.18 (m, 9H), 2.05 (s, 9H), 1.94-2.03 (m, 18H), 1.68 (br s, 2H), 1.45 (br s, 2H), 1.12 (br t, J=7.0 Hz, 2H).

To a solution of Compound Va (260 mg, 0.161 mmol) in anhydrous DCM (5 mL) was added diisopropylammonium tetrazolide (30.3 mg, 0.177 mmol), followed by dropwise addition of 3-bis(diisopropylamino)phosphanyloxypropanenitrile (194 mg, 0.645 mmol) at ambient temperature under N₂. The reaction mixture was stirred at 20˜ 25° C. for 2 hrs. LCMS indicated Compound Va was consumed completely. After cooling to −20° C., the reaction mixture was added to a stirred solution of brine/saturated aq. NaHCO₃ (1:1, 5 mL) at 0° C. After stirring for 1 min, DCM (5 mL) was added. Layers were separated. Organics were washed with brine/saturated aq. NaHCO₃ solution (1:1, 5 mL), dried over Na₂SO₄, filtered, and concentrated to ˜1 mL of volume. The residue solution was added dropwise to 20 mL methyl tert-butyl ether (MTBE) with stirring. This resulted in precipitation of white solid. The mixture was centrifuged, and solid was collected. The solid was redissolved in 1 mL of DCM and precipitated by addition of MTBE (20 mL). Solid was again isolated by centrifuge. The solid collected was dissolved in anhydrous CH₃CN. Volatiles were removed. This process was repeated two more times to afford GalNAc ligand phosphoramidite compound GLPA1 (153 mg, 84.4 μmol) as a white solid. ¹H NMR (400 MHz, CDCl₃): ppm δ 7.71-8.06 (m, 2H), 6.60-7.06 (m, 3H), 5.37 (br d, J=3.0 Hz, 3H), 5.18-5.32 (m, 3H), 4.70-4.86 (m, 3H), 3.92-4.25 (m, 18H), 3.42-3.85 (m, 30H), 3.25 (m, 4H), 2.59-2.75 (m, 4H), 2.27-2.44 (m, 2H), 2.15-2.20 (s, 9H) 2.07 (s, 9H), 1.96-2.03 (m, 18H), 1.65 (br s, 4H), 1.44 (br d, J=7.28 Hz, 2H), 1.14-1.24 (m, 12H). ³¹P NMR (CDCl₃): ppm δ 147.15.

GalNAc ligand phosphoramidite compound GLPA2 was synthesized using the same procedure except Intermediate-B was used. ¹H NMR (400 MHz, CDCl₃): ppm δ 7.94-8.18 (m, 1H), 7.69 (br s, 1H), 6.66-7.10 (m, 3H), 5.35 (d, J=3.5 Hz, 3H), 5.07-5.25 (m, 3H), 4.76-4.86 (m, 3H), 4.01-4.31 (m, 10H), 3.91-4.01 (m, 8H), 3.74-3.86 (m, 4H), 3.52-3.71 (m, 30H), 3.42-3.50 (m, 6H), 3.15-3.25 (m, 4H), 2.52-2.70 (m, 4H), 2.22-2.45 (m, 2H), 2.15-2.22 (s, 9H), 2.06 (s, 9H), 1.95-2.03 (m, 18H), 1.77 (br s, 2H), 1.58-1.66 (m, 4H), 1.40 (m, 2H), 1.08-1.24 (m, 12H). ³¹P NMR (CDCl₃): ppm δ 147.12.

Scheme 3 below was followed to prepare GLPA15.

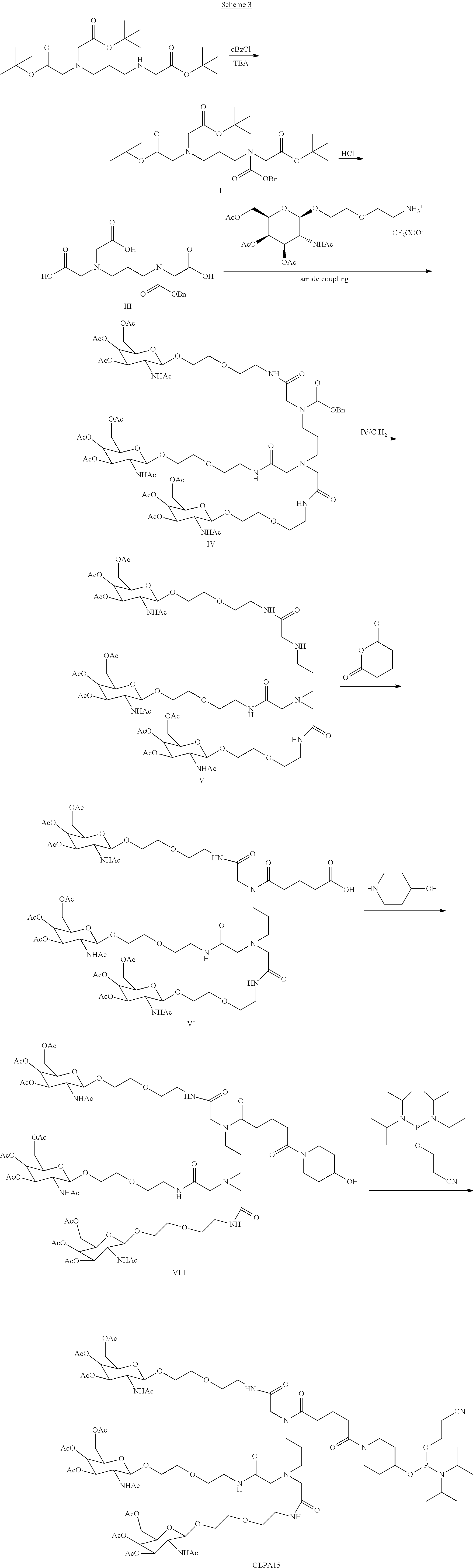

Starting from secondary amine Compound I (Compound II in Scheme 2), Cbz protection was introduced to afford Compound II. The tert-Butyl groups of Compound II were removed by treatment with acid to give triacid Compound III. Amide coupling of Compound III with Intermediate-A afforded Compound IV. The Cbz protecting group of Compound IV was removed by hydrogenation to afford secondary amine Compound V, which was reacted with glutaric anhydride to afford carboxylic Compound VI. Compound VI reacted with piperidin-4-ol under amide coupling reaction condition to affords Compound VII. Phosphoramidite Compound GLPA15 was synthesized by treating Compound VII with 2-Cyanoethyl N,N diisopropylchlorophosphoramidite and a catalytic amount of 1H-tetrazole.

¹H NMR (400 MHz in DMSO-d6): δ ppm 8.05 (br d, J=6.50 Hz, 2H), 7.81 (br d, J=9.01 Hz, 3H), 5.22 (d, J=3.25 Hz, 3H), 4.98 (dd, J=11.26, 3.25 Hz, 3H), 4.55 (br d, J=8.50 Hz, 3H), 4.03 (s, 9H), 3.64-3.97 (m, 12H), 3.55-3.63 (m, 6H), 3.50 (br s, 5H), 3.40 (br d, J=6.13 Hz, 6H), 3.17-3.30 (m, 9H), 3.07 (br d, J=14.26 Hz, 4H), 2.76 (t, J=5.82 Hz, 2H), 2.18-2.47 (m, 6H), 2.10 (s, 9H), 1.99 (s, 9H), 1.89 (s, 9H), 1.78 (s, 9H), 1.52-1.74 (m, 6H), 1.12-1.19 (m, 12H). 31P NMR (DMSO-d6): ppm δ 145.25.

In certain studies, a method used to attach a targeting group comprising GalNAc (also referred to herein as a GalNAc delivery compound) to the 5′-end of a sense strand included use of a GalNAc phosphoramidite (GLPA1) in the last coupling step in the solid phase synthesis, using a synthetic process such as the process used if oligonucleotide chain propagation of adding a nucleotide to the 5′-end of the sense strand is performed.

In some studies a method of attaching a targeting group comprising GalNAc to the 3′-end of a sense strand comprised use of a solid support (CPG) that included a GLO-n. In some studies, a method of attaching a targeting group comprising GalNAc to the 3′-end of a sense strand comprises attaching a GalNAc targeting group to CPG solid support through an ester bond and using the resulting CPG with the attached GalNAc targeting group when synthesizing the sense strand, which results in the GalNAc targeting group attached at the 3′-end of the sense strand.

### Example 4. In Vitro Screening of ANGPTL3 siRNA Duplexes

Hep3B cells were trypsinized and adjusted to appropriate density, and seeded into 96-well plates. Cells were transfected with test siRNAs or a control siRNA using Lipofectamine RNAiMax (Invitrogen-13778-150) at the same time of seeding following the protocol according to manufacturer's recommendation. The siRNAs were tested at two concentrations (0.2 nM and 1.0 nM) in triplicate, while the control siRNA was tested at 8 concentrations with 3-fold dilution from 4.6 pM to 10 nM in triplicate.

Cells were incubated for 24 hours after transfection. The medium was removed, and cells were harvested for RNA extraction. Total RNA was extracted by RNeasy® 96 Kit (QIAGEN-74182) according to the manual.

The cDNA was Synthesized with FastKing RT Kit (With gDNase, Tiangen-KR116-02) according to the manual. The human ANGPTL3 cDNA expression was determined by qPCR with TaqMan Gene Expression Assay (ANGPTL3, Thermo, Assay ID-Hs00205581_m1) normalized to expression of GAPDH (TaqMan Gene Expression Assay, Thermo, Assay ID-Hs02786624_g1). Percent of inhibition was calculated by comparing expression of ANGPTL3 of siRNA to PBS treated samples.

Table 7 provides experimental results of in vitro studies using various ANGPTL3 RNAi agents to inhibit ANGPTL3 expression. The duplex sequences used correspond to those shown in Table 2. Mass and purity information of these siRNA is provided in Table 6.

| | [symbol] | |
|---|---|---|
| [symbol] | 1 nM | 0.2 nM |

| AD#/AV# | [symbol] | SD | [symbol] | SD |
|---|---|---|---|---|
| AD00001 | −5.19 | 5.52 | −0.78 | 10.86 |
| AD00002 | 12.15 | 5.42 | −19.15 | 13.82 |
| AD00003 | 62.73 | 4.90 | 25.33 | 6.36 |
| AD00004 | 57.79 | 4.53 | 27.11 | 2.89 |
| AD00005 | 15.53 | 5.08 | −29.56 | 52.97 |
| AD00006 | 41.97 | 4.52 | 12.94 | 2.66 |
| AD00007 | 69.11 | 2.77 | 51.52 | 7.13 |
| AD00008 | 0.82 | 6.73 | −15.73 | 21.57 |
| AD00009 | 56.00 | 5.01 | 21.08 | 3.43 |
| AD00010 | −3.40 | 10.63 | −16.26 | 1.85 |
| AD00011 | 23.78 | 3.25 | −1.38 | 3.37 |
| AD00012 | −21.96 | 3.21 | −15.78 | 3.86 |
| AD00013 | 70.81 | 4.85 | 46.13 | 6.26 |
| AD00014 | 70.20 | 4.65 | 32.31 | 9.84 |
| AD00015 | 40.80 | 10.10 | 12.68 | 4.57 |
| AD00016 | 6.87 | 9.41 | −9.49 | 4.42 |
| AD00017 | 30.80 | 7.22 | 13.91 | 5.95 |
| AD00018 | 38.10 | 7.57 | 9.06 | 8.13 |
| AD00019 | 19.58 | 9.07 | 3.75 | 8.63 |
| AD00020 | 15.40 | 13.52 | 6.17 | 5.77 |
| AD00021 | 28.15 | 7.00 | 19.27 | 2.01 |
| AD00022 | 37.00 | 9.75 | 11.35 | 10.36 |
| AD00023 | 65.04 | 4.58 | 34.56 | 5.92 |
| AD00024 | 48.97 | 1.68 | 31.66 | 6.61 |
| AD00025 | 61.31 | 1.68 | 19.91 | 2.19 |
| AD00026 | 63.83 | 4.01 | 26.37 | 6.87 |
| AD00027 | 61.27 | 4.59 | 46.87 | 4.54 |
| AD00028 | 33.07 | 5.81 | 8.61 | 13.96 |
| AD00029 | 60.69 | 4.00 | 39.93 | 2.51 |
| AD00030 | 45.51 | 3.70 | 22.33 | 1.27 |
| AD00031 | 54.39 | 3.11 | 25.73 | 3.89 |
| AD00032 | 36.39 | 3.99 | 3.76 | 13.22 |
| AD00033 | 68.59 | 3.52 | 33.45 | 10.79 |
| AD00034 | 75.68 | 1.50 | 34.45 | 5.85 |
| AD00035 | 49.84 | 4.63 | 10.10 | 3.16 |
| AD00036 | 0.77 | 2.56 | −9.31 | 7.84 |
| AD00037 | 36.14 | 2.80 | 11.00 | 1.06 |
| AD00038 | 12.46 | 7.03 | 6.59 | 5.32 |
| AD00039 | 61.61 | 5.84 | 26.26 | 2.53 |
| AD00040 | 79.03 | 1.57 | 48.36 | 2.65 |
| AD00041 | 49.34 | 6.87 | 18.31 | 4.89 |
| AD00042 | 89.60 | 1.39 | 75.58 | 2.05 |
| AD00043 | 49.66 | 6.43 | 18.20 | 1.58 |
| AD00044 | 64.20 | 3.93 | 28.78 | 1.80 |
| AD00045 | 61.35 | 3.18 | 29.41 | 6.91 |
| AD00046 | 58.59 | 2.08 | 13.60 | 1.45 |
| AD00047 | 33.71 | 4.50 | 4.87 | 4.54 |
| AD00048 | 46.07 | 5.28 | 20.77 | 9.12 |
| AD00049 | 35.83 | 3.07 | 12.33 | 4.69 |
| AD00050 | 40.56 | 5.91 | 23.04 | 6.28 |
| AV01087 | 92.70 | 0.21 | 89.84 | 0.24 |
| AV01088 | 89.22 | 0.46 | 80.21 | 3.75 |
| AV01089 | 86.31 | 2.92 | 69.73 | 2.30 |
| AV01091 | 83.55 | 1.69 | 62.86 | 1.78 |
| AV01092 | 88.15 | 0.60 | 77.92 | 1.80 |
| AV01093 | 83.68 | 5.04 | 69.18 | 3.90 |
| AV01094 | 88.17 | 1.24 | 70.78 | 2.59 |
| AV01095 | 86.05 | 1.80 | 65.41 | 2.88 |
| AV01096 | 87.50 | 0.50 | 76.20 | 1.00 |
| AV01097 | 94.68 | 0.31 | 90.56 | 0.91 |
| AV01098 | 94.19 | 0.35 | 87.02 | 2.24 |
| AV01099 | 90.29 | 1.30 | 85.08 | 1.39 |
| AV01100 | 92.59 | 1.52 | 87.37 | 2.42 |
| AV01101 | 93.55 | 0.64 | 89.64 | 0.64 |
| AV01102 | 93.18 | 1.00 | 89.49 | 0.65 |
| AV01103 | 95.83 | 0.46 | 92.55 | 0.38 |
| AV01104 | 94.35 | 0.82 | 91.25 | 0.83 |
| AV01105 | 90.25 | 0.76 | 84.18 | 3.34 |
| AV01106 | 90.51 | 1.29 | 83.34 | 2.67 |
| AV01107 | 89.69 | 1.12 | 79.67 | 0.52 |
| AV01108 | 85.19 | 1.10 | 66.55 | 1.85 |
| AV01109 | 93.62 | 1.44 | 89.83 | 0.67 |
| AV01110 | 92.26 | 1.88 | 84.95 | 1.73 |
| AV01111 | 91.76 | 2.29 | 87.96 | 0.48 |
| AV01112 | 87.14 | 2.11 | 70.92 | 3.59 |
| AV01113 | 92.81 | 0.27 | 82.52 | 0.99 |
| AV01114 | 94.11 | 0.21 | 89.72 | 0.26 |
| AV01115 | 91.31 | 0.64 | 76.63 | 2.43 |
| AV01116 | 93.66 | 1.97 | 84.28 | 5.60 |

### Example 5

In Vivo Testing of ANGPTL3 siRNA Duplexes

To assess in vivo activity of ANGPTL3 siRNAs, mice (4 mice in each group) infected with AAV encoding the human ANGPTL3 gene were used. At 14 days before dosing of siRNAs, female C57BL/6J mice were infected by intravenous administration of 25 μL of stock solution of an adeno-associated virus 8 (AAV8) vector encoding the human ANGPTL3 gene. At day 0, mice were subcutaneously administered a single 3 mg/kg of ANGPTL3 siRNA agents or PBS. Blood samples were collected at day 0, before dosing of siRNA and at day 7, at termination. Human ANGPTL3 protein concentration was measured by ELISA assay per manufacturer's recommended protocol (R&D Systems, Human Angiopoietin-like 3 Quantikine ELISA Kit). Percent of knockdown was calculated by comparing human ANGPTL3 level in day 7 mouse plasma samples of siRNA treated group to PBS treated group. The percent knockdown activity for compound AD00112, AD00135 and AD00143 (Table 3) was 91%, 83% and 84% respectively. In this example, the GLO-0 in said compounds AD00112, AD00135 and AD00143 refers to the compound GalNAc3 in Jayaprakash, et al., (2014) J. Am. Chem.

### Soc., 136, 16958-16961.

### Example 6

In Vivo Testing of ANGPTL3 siRNA Duplexes

At 14 days before dosing of siRNAs, female C57BL/6J mice (4 in each group) were infected by intravenous administration of a solution of adeno-associated virus 8 (AAV8) vector encoding human ANGPTL3 and luciferase gene. At day 0, mice were subcutaneously administered a single 3 mg/kg of ANGPTL3 siRNA agents or PBS. Blood samples were collected at day 0, before dosing of siRNA and at day 7, at termination. Serum samples were isolated and luciferase activity of serum samples was measured per manufacturer's recommended protocol. Since expression of human ANGPTL3 level correlates with expression level of luciferase, measurement of luciferase activity is the surrogate for ANGTPL3 expression. Percent remaining of ANGPTL3 was calculated by comparing luciferase activity in samples from pre- (day 0) and post (day 7) treatment of siRNA for each mouse and normalized by the change of luciferase activity in the samples from the control treated mice during the same period of time. Result is summarized in Table 8. In this example, the GLO-0 in said compounds in Table 3 refers to the compound GalNAc3 in Jayaprakash, et al., (2014) J. Am. Chem. Soc., 136, 16958-16961.

Table 8 provides experimental results of in vivo studies using various ANGPTL3 RNAi agents to inhibit ANGPTL3 expression. The duplex sequences used correspond to those shown in Table 3 and Table 4.

| | | | [symbol] 7 [symbol] remaining of luciferase |
|---|---|---|---|
| | | | activity relative to day 0, |
| | [symbol] 7 [symbol] | | normalized by change in the |
| | PBS | | PBS treated group |
| [symbol] AD# | ( [symbol] ± SD) | [symbol] AD# | (Mean ± SD) |
| PBS | 1 ± 0.21 | AD00135-2 | 0.11 ± 0.04 |
| AD00108 | 0.2 ± 0.06 | AD00136 | 0.09 ± 0.02 |
| AD00108-1 | 0.24 ± 0.03 | AD00136-1 | 0.13 ± 0.06 |
| AD00112 | 0.13 ± 0.04 | AD00142 | 0.16 ± 0.04 |
| AD00112-1 | 0.18 ± 0.02 | AD00143 | 0.15 ± 0.05 |
| AD00133 | 0.13 ± 0.02 | AD00143-2 | 0.22 ± 0.07 |
| AD00134 | 0.17 ± 0.03 | AD00145 | 0.21 ± 0.04 |
| AD00135 | 0.13 ± 0.06 | AD00146 | 0.16 ± 0.05 |

### Example 7

In Vivo Testing of ANGPTL3 siRNA Duplexes

At 14 days before dosing of siRNAs, female C57BL/6J mice were infected by intravenous administration of a solution of adeno-associated virus 8 (AAV8) vector encoding human ANGPTL3 and luciferase gene. At day 0, mice were subcutaneously administered a single dose of AD00112-2 at 1, 3 or 10 mg/kg or PBS. Blood samples were collected at day 0, before dosing of siRNA and at day 7, at termination. Serum samples were isolated and luciferase activity of serum samples was measured per manufacturer's recommended protocol. Since expression of human ANGPTL3 level correlates with expression level of luciferase, measurement of luciferase activity is the surrogate for ANGTPL3 expression. Result is summarized in Table 9.

Table 9 provides experimental results of in vivo studies. The duplex sequences used correspond to those shown in Table 4.

| | | Day 7, remaining of luciferase activity |
|---|---|---|
| | | relative to day 0, normalized by change |
| | Dose | in the PBS treated group |
| Duplex AD# | (mg/kg) | (Mean ± SD) |
| AD00112-2 | 1 | 0.33 ± 0.06 |
| AD00112-2 | 3 | 0.20 ± 0.09 |
| AD00112-2 | 10 | 0.11 ± 0.03 |

### Example 8

In Vivo Testing of ANGPTL3 siRNA Duplexes in NHP PD Model

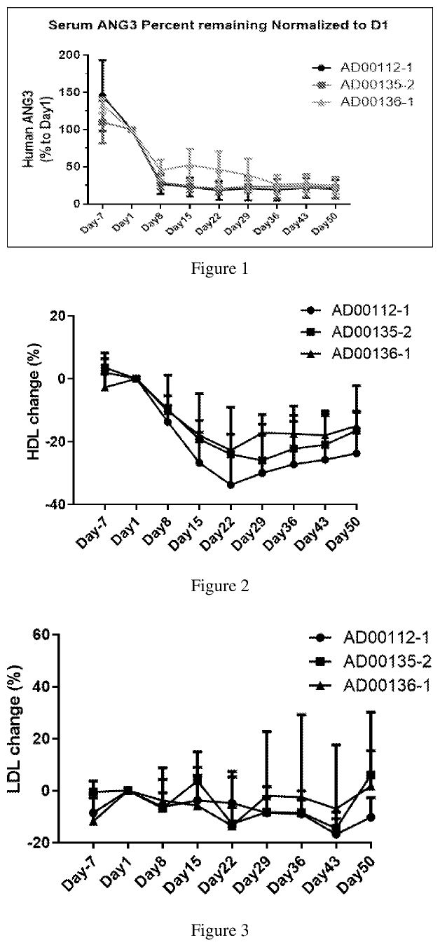

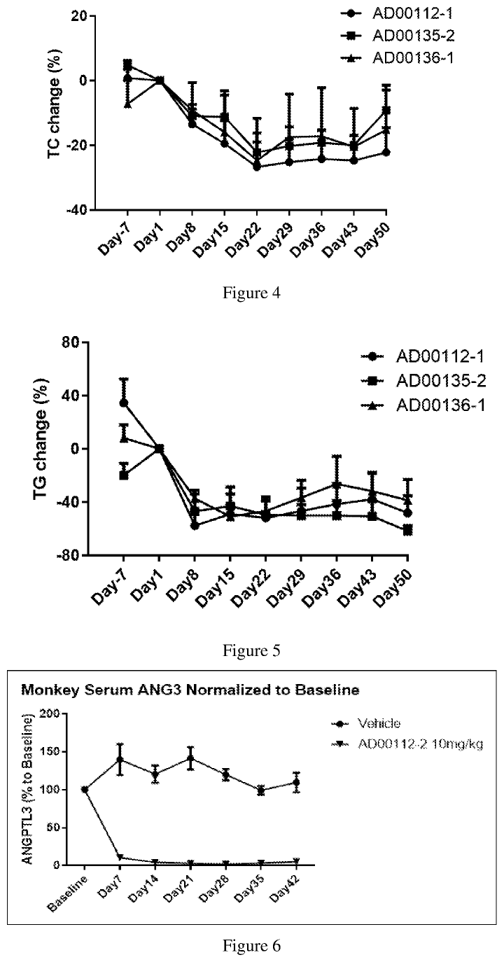

Male cynomolgus monkeys (13-22 years old, 7-9 kg of weights, 4 monkeys in each group) were enrolled in the study. Each monkey received a subcutaneous injection with one of the testing articles formulated in PBS at 4 mg/kg at day 1 (pre-dosing of siRNA). After overnight fast, blood samples were drawn at day −7 (pre-dose), 1 (pre-dose), 8, 15, 22, 29, 43, and 50. ANGPTL3 protein concentration in serum were measured by ELISA method. Percent remaining of ANGPTL3 (normalized to day 1, pre-dosing of siRNA) for groups dosed with compound AD00112, AD00135 and AD00136 is shown in FIG. 1. Lipid profile was also measured. Percent change of HDL, LDL, TC (total cholesterol) and TG (triglyceride) level in serum (normalized to day 1, pre-dosing of siRNA) is shown in FIGS. 2, 3, 4 and 5 respectively. For monkeys in all three groups dosed with siRNA compounds, significant and sustained reduction of ANGPTL3 in serum observed (up to 86%). Significant reduction in TG (up to 60% reduction) and modest reduction in HDL-C and TC was also observed.

### Example 9

In Vivo Testing of ANGPTL3 siRNA Duplexes in NHP Disease Model

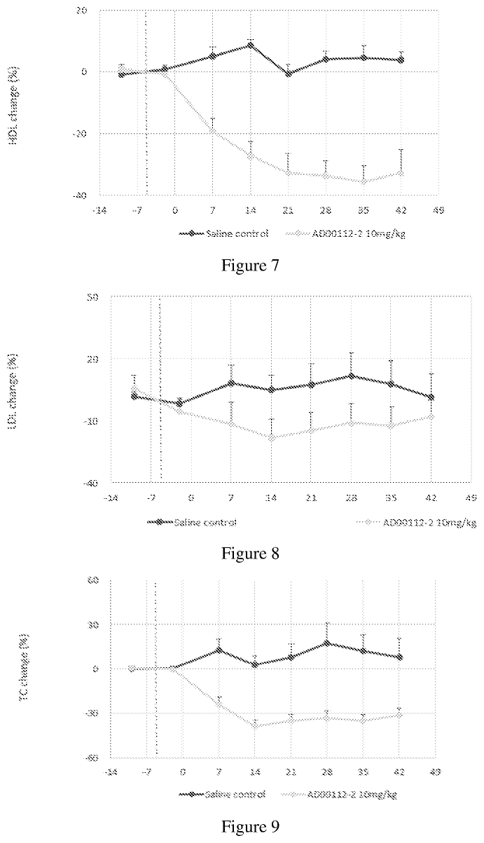

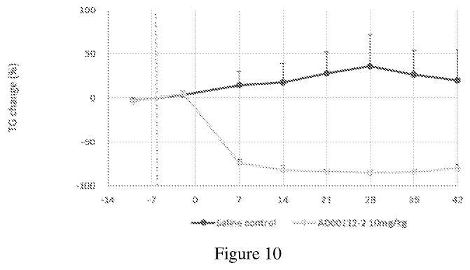

Male cynomolgus monkeys (13-21 years old) were screened for their baseline lipid profiles including HDL, LDL, TC (total cholesterol) and TG (triglyceride). Twenty monkeys with elevated baseline LDL (1.03-2.36 mmol/L) and TG (1.42-6.71 mmol/L) levels were selected and randomized into 2 groups to receive a single subcutaneous injection of saline or AD00112-2 at 10 mg/kg at day 0. After overnight fast, blood samples were drawn at day −10 (pre-dose), −2 (pre-dose), 7, 14, 21, 28, 35, and 42. ANGPTL3 protein concentration in serum were measured by ELISA method. Percent remaining of ANGPTL3 (normalized to baseline, average of day −10 and −2) for each group dosed with saline or compound AD00112-2 at 10 mg/kg is shown in FIG. 6. Lipid profile was also measured. Percent change of HDL, LDL, TC (total cholesterol) and TG (triglyceride) level in serum (normalized to baseline, pre-dosing of siRNA) is shown in FIGS. 7, 8, 9 and 10 respectively. Deep and sustained reduction of ANGPTL3 concentration and TG level was observed. Modest reduction in HDL-C, LDL-C and TC level was also observed.

### EQUIVALENTS

Although several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto; the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified, unless clearly indicated to the contrary.

1. A double-stranded ribonucleic acid (dsRNA) agent for inhibiting expression of Angiopoietin-like 3 (ANGPTL3), wherein the dsRNA agent comprises a sense strand and an antisense strand, nucleotide positions 2 to 18 in the antisense strand comprising a region of complementarity to an ANGPTL3 RNA transcript, wherein the region of complementarity comprises at least 15 contiguous nucleotides that differ by 0, 1, 2, or 3 nucleotides from one of the antisense sequences listed in one of Tables 1-5, and optionally comprising a targeting ligand, optionally, wherein the region of complementarity to an ANGPTL3 RNA transcript comprises at least 15, 16, 17, 18, or 19 contiguous nucleotides that differ by no more than 3 nucleotides from one of the antisense sequences listed in one of Tables 1-5.

2. (canceled)

3. The dsRNA agent of claim 1, wherein the antisense strand of dsRNA is at least substantially complementary to any one of a target region of SEQ ID NO: 235 and is provided in any one of Tables 1-5, and optionally, wherein the antisense strand of dsRNA is fully complementary to any one of a target region of SEQ ID NO: 235 and is provided in any one of Tables 1-5.

4. (canceled)

5. The dsRNA agent of claim 1, wherein the dsRNA agent comprises a sense strand sequence set forth in any one of Tables 1-5, wherein the sense strand sequence is at least substantially complementary to the antisense strand sequence in the dsRNA agent, or wherein the sense strand sequence is fully complementary to the antisense strand sequence in the dsRNA agent.

6. (canceled)

7. The dsRNA agent of claim 1, wherein the dsRNA agent comprises an antisense strand sequence set forth in any one of Tables 1-5, and optionally, wherein the dsRNA agent comprises the sequences set forth as a duplex sequence in any of Tables 1-5.

8. (canceled)

9. The dsRNA of claim 1, wherein the dsRNA agent comprises at least one modified nucleotide, optionally, wherein all or substantially all of the nucleotides of the antisense strand are modified nucleotides, and optionally wherein the at least one modified nucleotide comprises: a 2′-O-methyl nucleotide,2′-Fluoro nucleotide, 2′-deoxy nucleotide, 2′3′-seco nucleotide mimic, locked nucleotide, unlocked nucleic acid nucleotide (UNA), glycol nucleic acid nucleotide (GNA), 2′-F-Arabino nucleotide, 2′-methoyxyethyl nucleotide, abasic nucleotide, ribitol, inverted nucleotide, inverted abasic nucleotide, inverted 2′-Ome nucleotide, inverted 2′-deoxy nucleotide, 2′-amino-modified nucleotide, 2′-alkyl-modified nucleotide, mopholino nucleotide, and 3′-OMe nucleotide, a nucleotide comprising a 5′-phosphorothioate group, or a terminal nucleotide linked to a cholesteryl derivative or dodecanoic acid bisdecylamide group, a 2′-amino-modified nucleotide, ′a phosphoramidate, or a non-natural base comprising nucleotide.

10-11. (canceled)

12. The dsRNA agent of claim 9, comprises an E-vinylphosphonate nucleotide at the 5′ end of the antisense strand, optionally, wherein the dsRNA agent comprises at least one phosphorothioate internucleoside linkage, optionally, wherein the sense strand comprises at least one phosphorothioate internucleoside linkage, optionally, wherein the antisense strand comprises at least one phosphorothioate internucleoside linkage, optionally wherein the sense strand comprises 1, 2, 3, 4, 5, or 6, phosphorothioate internucleoside linkages, optionally wherein the antisense strand comprises 1, 2, 3, 4, 5, or 6, phosphorothioate internucleoside linkages, optionally wherein all or substantially all of the nucleotides of the sense strand and the antisense strand are modified nucleotides, optionally wherein the modified sense strand is a modified sense strand sequence set forth in one of Tables 2-5, and optionally, wherein the modified antisense strand is a modified antisense strand sequence set forth in one of Tables 2-5.

13-20. (canceled)

21. The dsRNA agent of claim 1, wherein the sense strand is complementary or substantially complementary to the antisense strand, and the region of complementarity is between 16 and 23 nucleotides in length, optionally wherein the region of complementarity is 19-21 nucleotides in length, optionally wherein each strand is no more than 30 nucleotides in length, optionally wherein each strand is no more than 25 nucleotides in length, and optionally wherein each strand is no more than 23 nucleotides in length.

22-28. (canceled)

29. The dsRNA agent of claim 1, wherein the dsRNA agent comprises at least one modified nucleotide and further comprises one or more targeting groups or linking groups, optionally wherein the one or more targeting groups or linking groups are conjugated to the sense strand, optionally, wherein the targeting group or linking group comprises N-acetyl-galactosamine (GalNAc), and optionally wherein the targeting group has a structure:

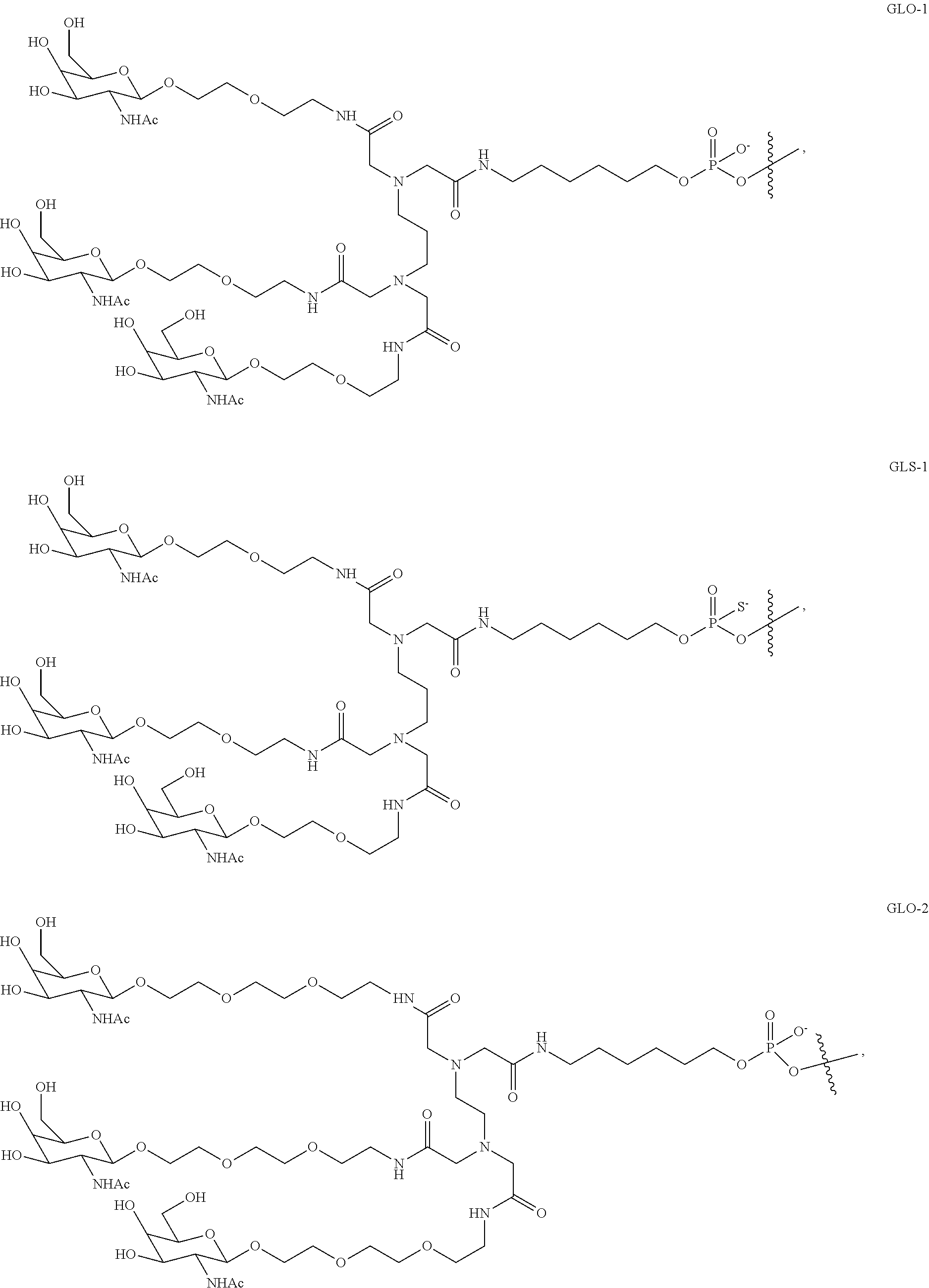 [SMILES: *OP(=O)([O-])OCCCCCCNC(=O)CN(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([S-])OCCCCCCNC(=O)CN(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([O-])OCCCCCCNC(=O)CN(CCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O]

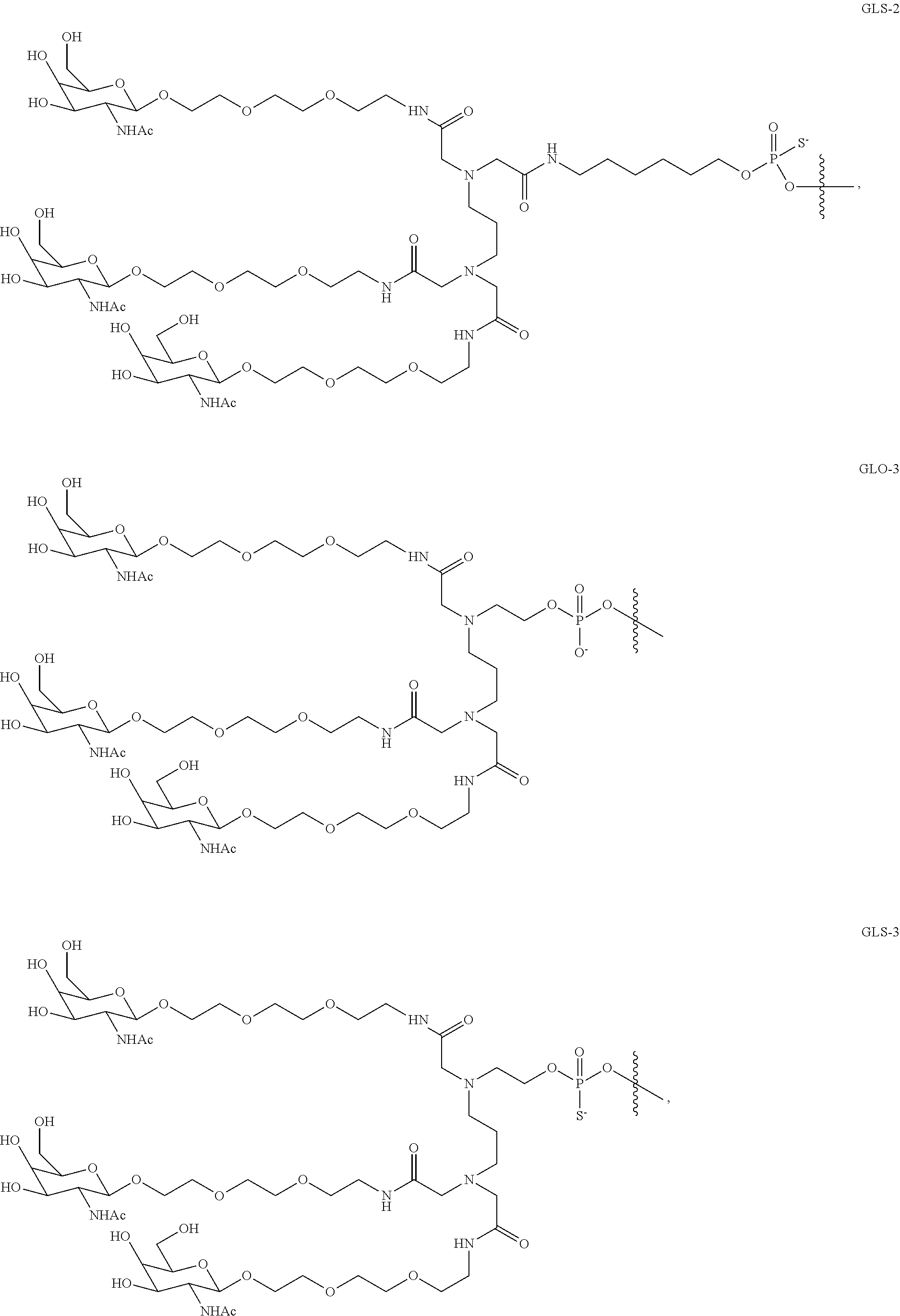 [SMILES: *OP(=O)([S-])OCCCCCCNC(=O)CN(CCCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O ; *OP(=O)([O-])OCCN(CCCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O ; *OP(=O)([S-])OCCN(CCCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O]

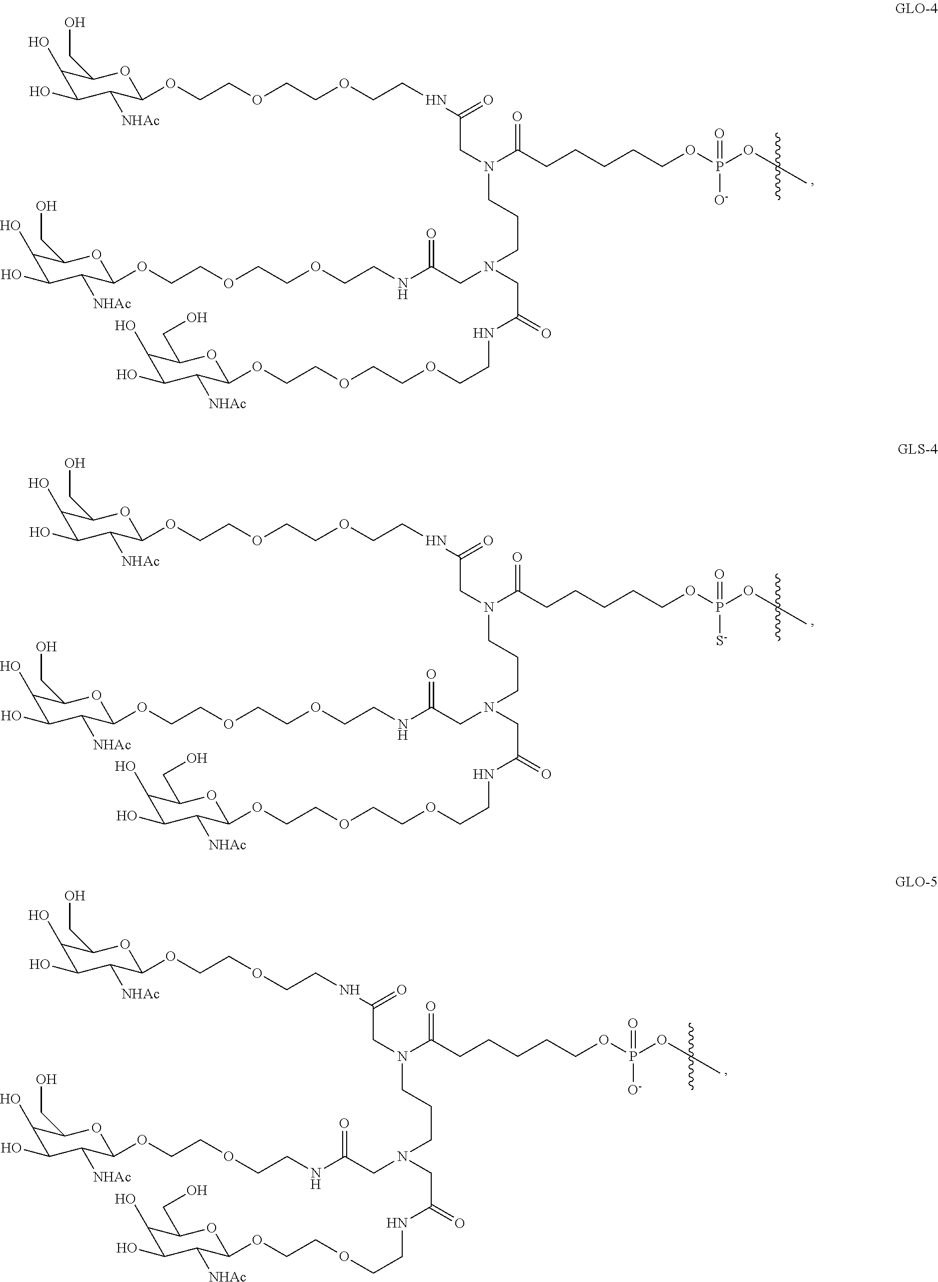 [SMILES: *OP(=O)([O-])OCCCCCC(=O)N(CCCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O ; *OP(=O)([S-])OCCCCCC(=O)N(CCCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O ; *OP(=O)([O-])OCCCCCC(=O)N(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O]

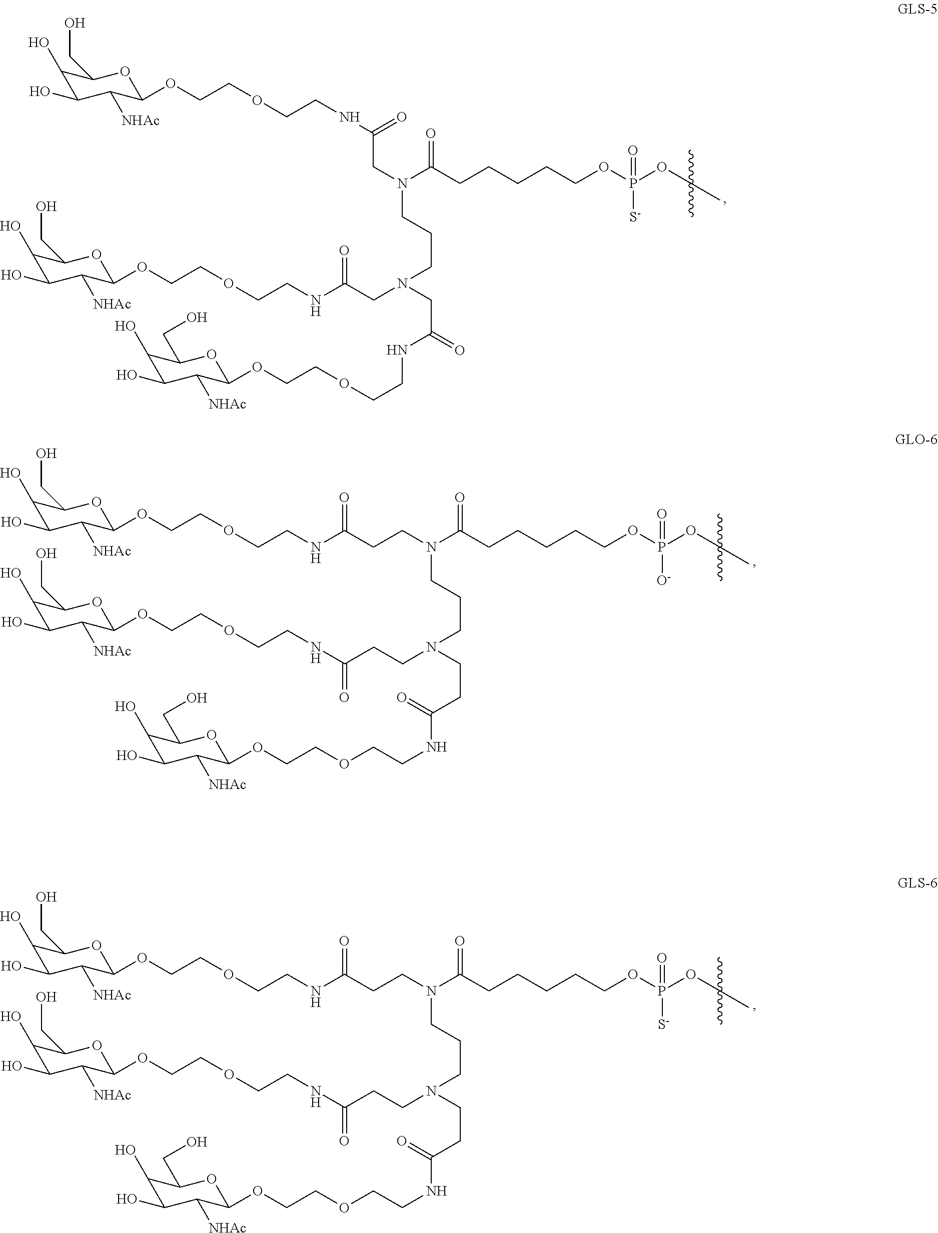 [SMILES: *OP(=O)([S-])OCCCCCC(=O)N(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([O-])OCCCCCC(=O)N(CCCN(CCC(=O)NCCOCCOC1OC(CO)C(O)C(O)C1NC(C)=O)CCC(=O)NCCOCCOC1OC(CO)C(O)C(O)C1NC(C)=O)CCC(=O)NCCOCCOC1OC(CO)C(O)C(O)C1NC(C)=O ; *OP(=O)([S-])OCCCCCC(=O)N(CCCN(CCC(=O)NCCOCCOC1OC(CO)C(O)C(O)C1NC(C)=O)CCC(=O)NCCOCCOC1OC(CO)C(O)C(O)C1NC(C)=O)CCC(=O)NCCOCCOC1OC(CO)C(O)C(O)C1NC(C)=O]

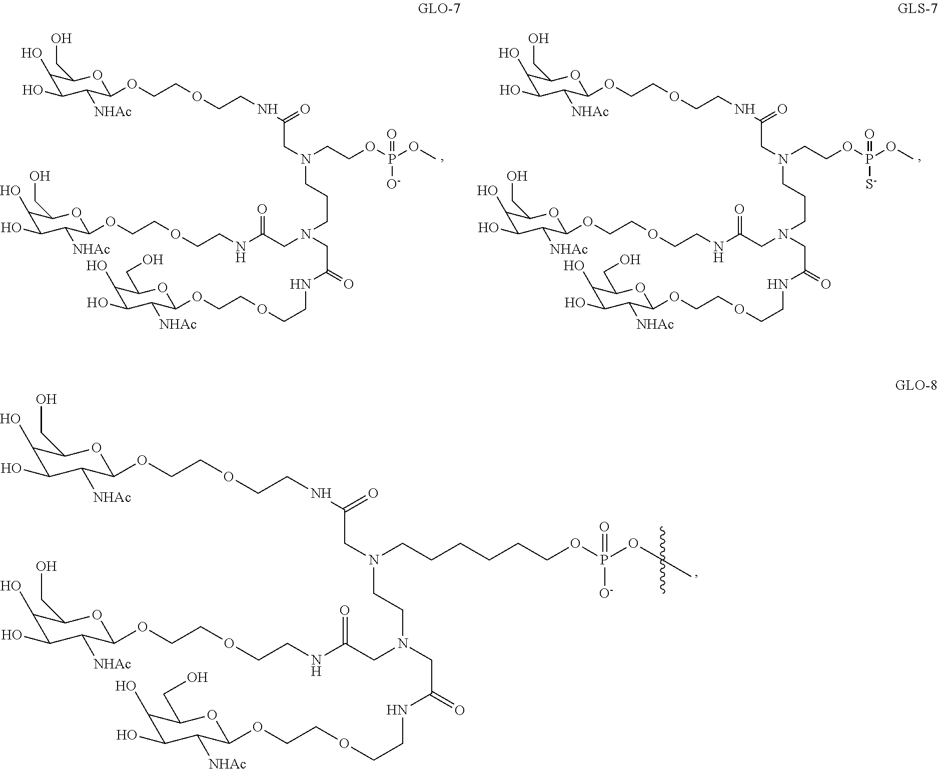 [SMILES: COP(=O)([O-])OCCN(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; COP(=O)([S-])OCCN(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([O-])OCCCCCCN(CCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O]

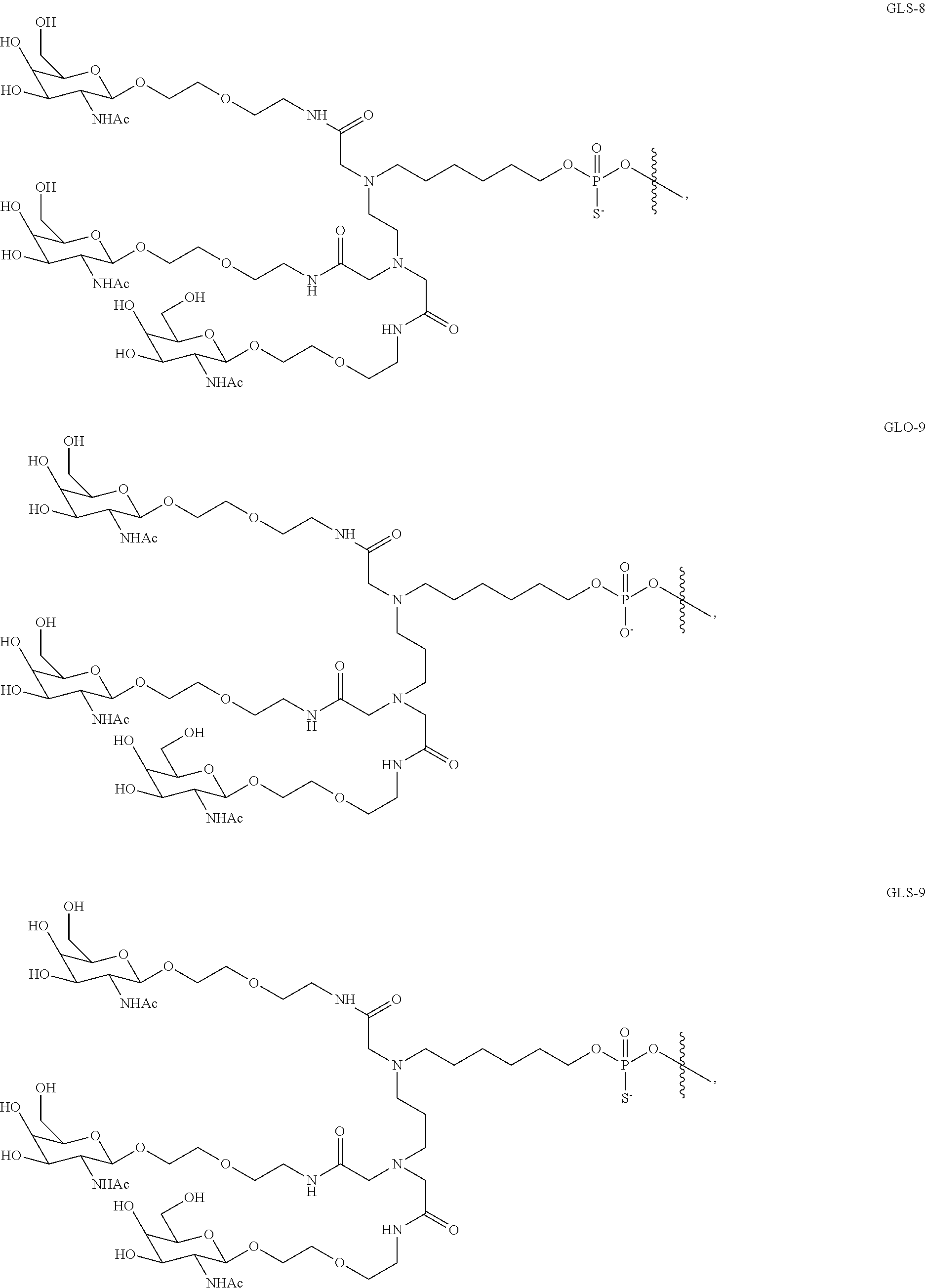 [SMILES: *OP(=O)([S-])OCCCCCCN(CCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([O-])OCCCCCCN(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([S-])OCCCCCCN(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O]

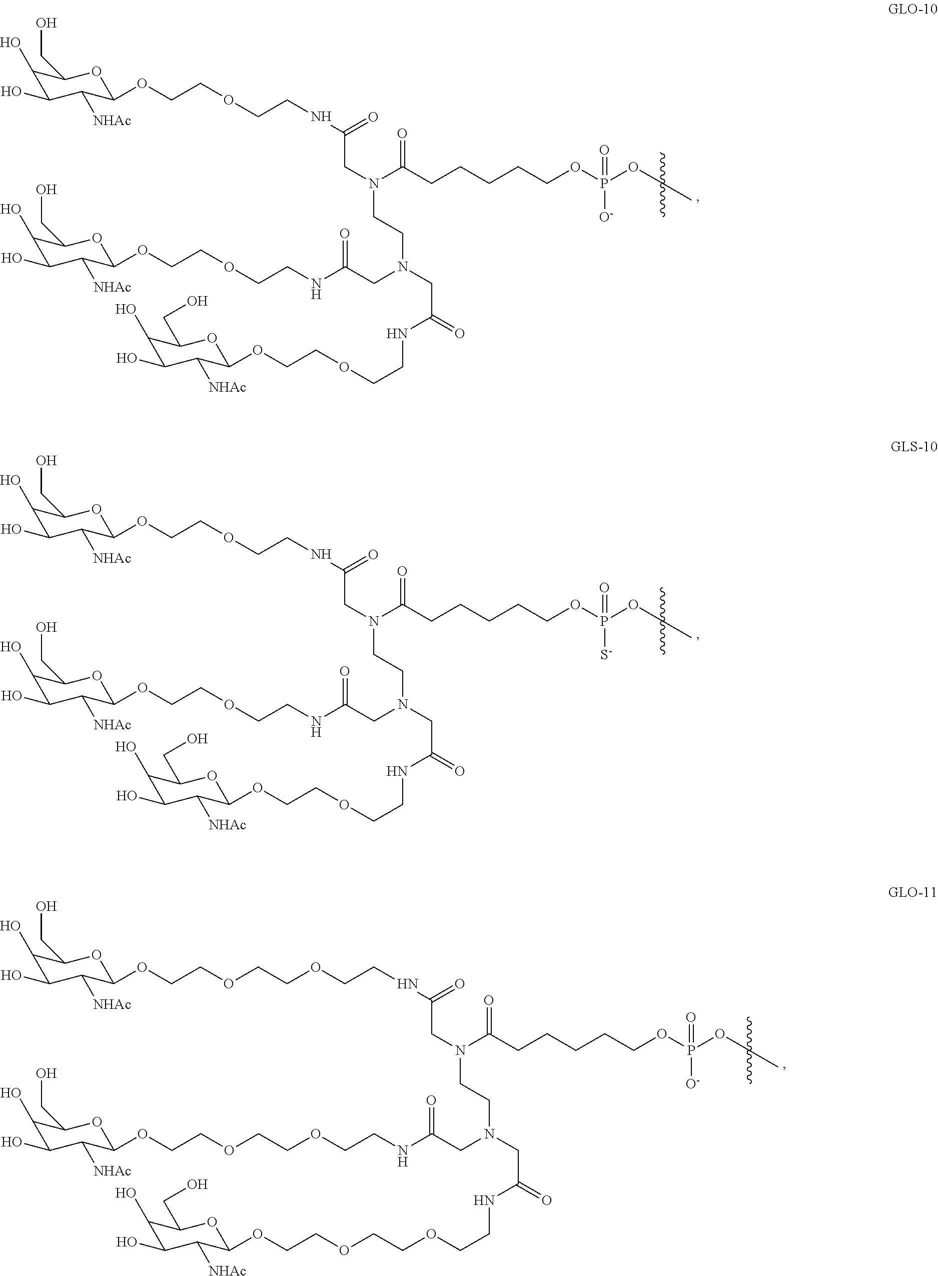 [SMILES: *OP(=O)([O-])OCCCCCC(=O)N(CCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([S-])OCCCCCC(=O)N(CCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([O-])OCCCCCC(=O)N(CCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O]

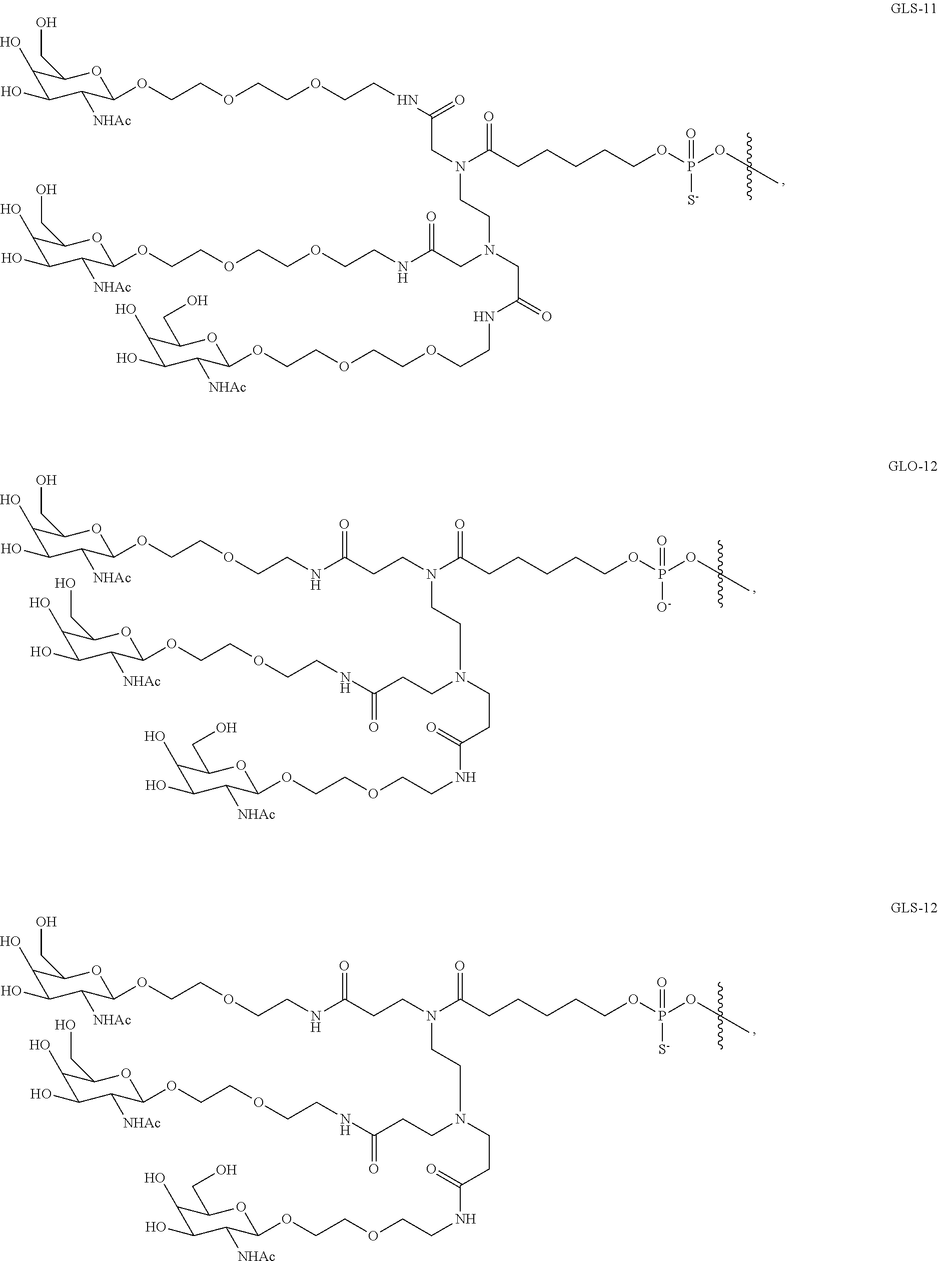 [SMILES: *OP(=O)([S-])OCCCCCC(=O)N(CCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O ; *OP(=O)([O-])OCCCCCC(=O)N(CCC(=O)NCCOCCOC1OC(CO)C(O)C(O)C1NC(C)=O)CCN(CCC(=O)NCCOCCOC1OC(CO)C(O)C(O)C1NC(C)=O)CCC(=O)NCCOCCOC1OC(CO)C(O)C(O)C1NC(C)=O ; *OP(=O)([S-])OCCCCCC(=O)N(CCC(=O)NCCOCCOC1OC(CO)C(O)C(O)C1NC(C)=O)CCN(CCC(=O)NCCOCCOC1OC(CO)C(O)C(O)C1NC(C)=O)CCC(=O)NCCOCCOC1OC(CO)C(O)C(O)C1NC(C)=O]

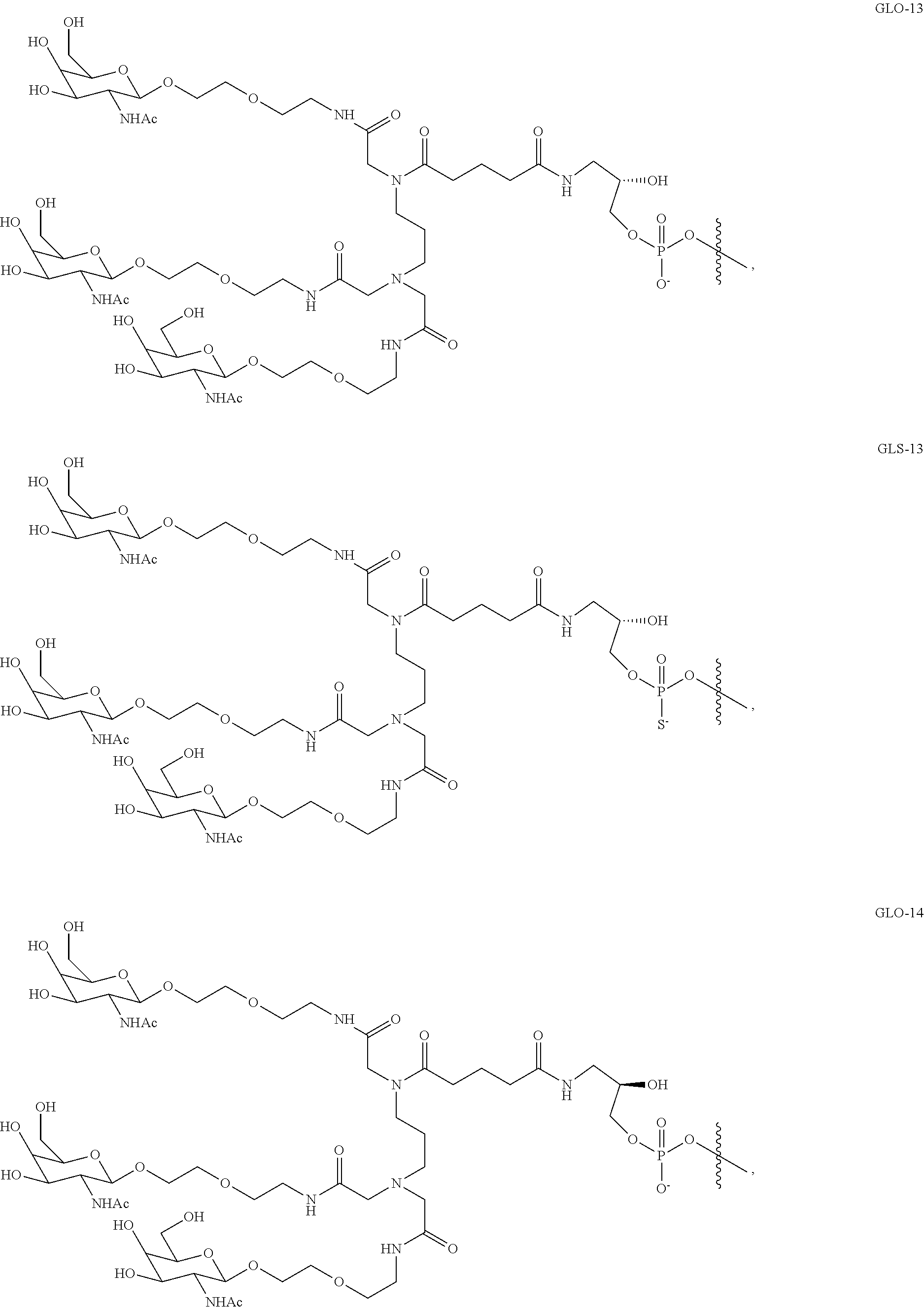 [SMILES: *OP(=O)([O-])OC[C@@H](O)CNC(=O)CCCC(=O)N(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([S-])OC[C@@H](O)CNC(=O)CCCC(=O)N(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([O-])OC[C@H](O)CNC(=O)CCCC(=O)N(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O]

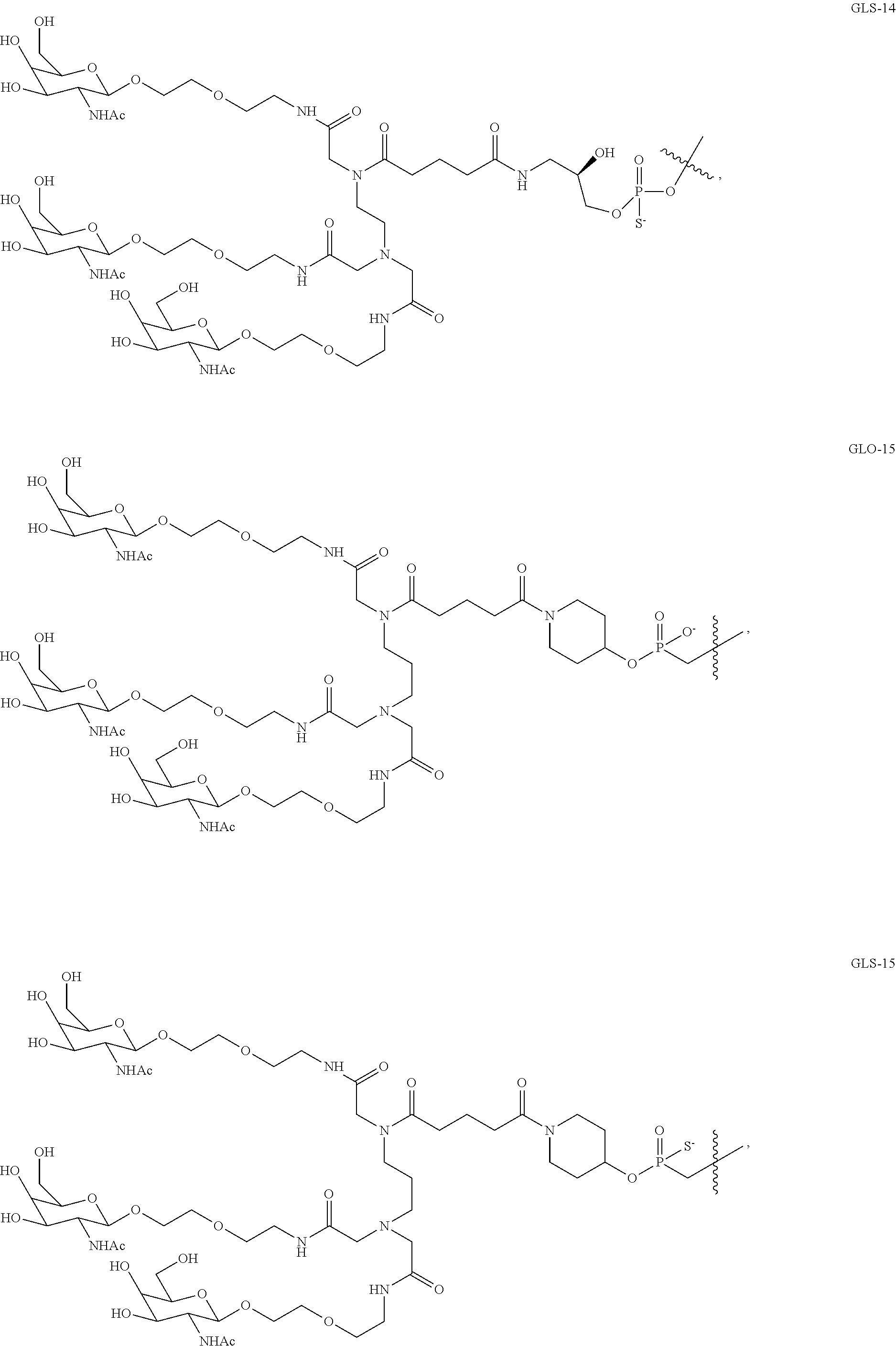 [SMILES: *OP(=O)([S-])OC[C@H](O)CNC(=O)CCCC(=O)N(CCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *CP(=O)([O-])OC1CCN(C(=O)CCCC(=O)N(CCCN(CC(=O)NCCOCCOC2OC(CO)C(O)C(O)C2NC(C)=O)CC(=O)NCCOCCOC2OC(CO)C(O)C(O)C2NC(C)=O)CC(=O)NCCOCCOC2OC(CO)C(O)C(O)C2NC(C)=O)CC1 ; *CP(=O)([S-])OC1CCN(C(=O)CCCC(=O)N(CCCN(CC(=O)NCCOCCOC2OC(CO)C(O)C(O)C2NC(C)=O)CC(=O)NCCOCCOC2OC(CO)C(O)C(O)C2NC(C)=O)CC(=O)NCCOCCOC2OC(CO)C(O)C(O)C2NC(C)=O)CC1]

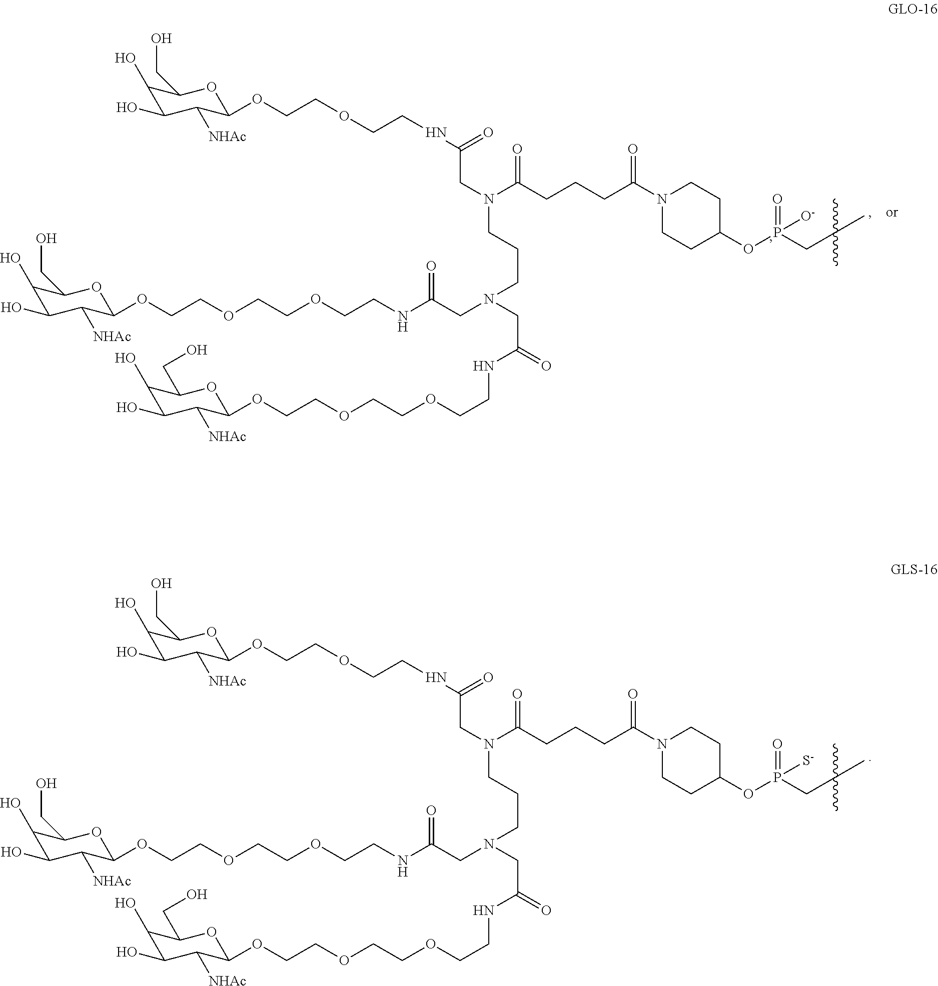 [SMILES: *CP(=O)([O-])OC1CCN(C(=O)CCCC(=O)N(CCCN(CC(=O)NCCOCCOCCOC2OC(CO)C(O)C(O)C2NC(C)=O)CC(=O)NCCOCCOCCOC2OC(CO)C(O)C(O)C2NC(C)=O)CC(=O)NCCOCCOC2OC(CO)C(O)C(O)C2NC(C)=O)CC1 ; *CP(=O)([S-])OC1CCN(C(=O)CCCC(=O)N(CCCN(CC(=O)NCCOCCOCCOC2OC(CO)C(O)C(O)C2NC(C)=O)CC(=O)NCCOCCOCCOC2OC(CO)C(O)C(O)C2NC(C)=O)CC(=O)NCCOCCOC2OC(CO)C(O)C(O)C2NC(C)=O)CC1]

30. The dsRNA agent of claim 1, wherein the dsRNA agent comprises a targeting group that is conjugated to the 5′-terminal end of the sense strand and/or the dsRNA agent comprises a targeting group that is conjugated to the 3-terminal end of the sense strand.

31. (canceled)

32. The dsRNA agent of claim 1, wherein the antisense strand comprises one inverted abasic residue at 3′-terminal end, optionally, wherein the sense strand comprises one or two inverted abasic residues at 3′ or/and 5′ terminal end.

33. (canceled)

34. The dsRNA agent of claim 1, wherein the dsRNA agent has two blunt ends, or wherein at least one strand comprises a 3′ overhang of at least 1 or 2 nucleotides.

35-36. (canceled)

37. A composition comprising a dsRNA agent of claim 1, optionally further comprising a pharmaceutically acceptable carrier, and optionally further comprising one or more additional therapeutic agents, optionally wherein the composition is formulated for subcutaneous administration or is formulated for intravenous (IV) administration.

38-43. (canceled)

44. A method of inhibiting the expression of an ANGPTL3 gene in a cell, the method comprising:
(i) preparing a cell comprising an effective amount of a double-stranded ribonucleic acid (dsRNA) agent of claim 1, optionally, (ii) maintaining the cell prepared in (i) for a time sufficient to obtain degradation of the mRNA transcript of an ANGPTL3 gene, thereby inhibiting expression of the ANGPTL3 gene in the cell.

45. (canceled)

46. The method claim 44, wherein the cell is in a subject and the dsRNA agent is administered to the subject subcutaneously or by IV administration.

47. (canceled)

48. The method of claim 46, further comprising assessing inhibition of the ANGPTL3 gene, following the administration of the dsRNA agent to the subject, wherein a means for the assessing comprises:
(i) determining one or more physiological characteristics of an ANGPTL3-associated disease or condition in the subject and
(ii) comparing the determined physiological characteristic(s) to a baseline pre-treatment physiological characteristic of the ANGPTL3-associated disease or condition and/or to a control physiological characteristic of the ANGPTL3-associated disease or condition, wherein the comparison indicates one or more of a presence or absence of inhibition of expression of the ANGPTL3 gene in the subject, optionally, wherein the determined physiological characteristic is one or more of: the subject's serum lipid level, the subject's serum HDL level, the subject's HDL:LDL ratio, the subject's serum triglyceride level, and the amount of fat in the subject's liver, and optionally wherein a reduction in one or more of the subject's serum lipid level, the subject's serum HDL level, the subject's serum triglyceride level, and the amount of fat in the subject's liver indicates reduction of ANGPTL3 gene expression in the subject.

49-50. (canceled)

51. A method of inhibiting expression of an ANGPTL3 gene in a subject, the method comprising administering to the subject an effective amount of a double-stranded ribonucleic acid (dsRNA) agent of claim 1, optionally, further comprising assessing inhibition of the ANGPTL3 gene, following the administration of the dsRNA agent, wherein a means for the assessing comprises:
(i) determining one or more physiological characteristics of an ANGPTL3-associated disease or condition in the subject and
(ii) comparing the determined physiological characteristic(s) to a baseline pre-treatment physiological characteristic of the ANGPTL3-associated disease or condition and/or to a control physiological characteristic of the ANGPTL3-associated disease or condition, wherein the comparison indicates one or more of a presence or absence of inhibition of expression of the ANGPTL3 gene in the subject, optionally wherein the determined physiological characteristic is one or more of: the subject's serum lipid level, the subject's serum HDL level, the subject's HDL:LDL ratio, the subject's serum triglyceride level, and the amount of fat in the subject's liver, optionally, wherein a reduction in one or more of the subject's serum lipid level, the subject's serum HDL level, the subject's serum triglyceride level, and the amount of fat in the subject's liver indicates reduction of AGNPTL3 gene expression in the subject, and optionally, wherein the dsRNA agent is administered to the subject subcutaneously or by IV administration.

52-56. (canceled)

57. A method of treating a disease or condition associated with the presence of ANGPTL3 protein, the method comprising administering to a subject an effective amount of a double-stranded ribonucleic acid (dsRNA) agent of claim 1, to inhibit ANGPTL3 gene expression, optionally wherein the disease or condition is one or more of: hyperlipidemia, hypertriglyceridemia, abnormal lipid and/or cholesterol metabolism, homozygous and heterozygous familial hypercholesterolemia, statin resistant hypercholesterolemia, cardiometabolic disease, obesity, atherosclerosis, type II diabetes mellitus, cardiovascular disease, coronary artery disease, non-alcoholic steatohepatitis, non-alcoholic fatty liver disease, pancreatitis caused by hypertriglyceridemia.

58. (canceled)

59. The method of claim 57, further comprising administering an additional therapeutic regimen to the subject, optionally, wherein the additional therapeutic regimen comprises: administering to the subject one or more ANGPTL3 antisense polynucleotides of the invention, administering to the subject a non-ANGPTL3 dsRNA therapeutic agent, and a behavioral modification in the subject, and optionally, wherein the non-ANGPTL3 dsRNA therapeutic agent is one of more of: (i) a statin; (ii) one or more of an antibody, antisense oligonucleotide (ASO), and a PCSK9 siRNA molecule capable of reducing PCSK9 expression; (iii) a therapeutic agent capable of reducing lipid accumulation in a subject, and (iv) a therapeutic agent capable of reducing cholesterol levels and/or accumulation in a subject.

60-67. (canceled)

68. A method of decreasing a level of ANGPTL3 protein in a subject compared to a baseline pre-treatment level of ANGPTL3 protein in the subject, the method comprising administering to the subject an effective amount of a double-stranded ribonucleic acid (dsRNA) agent of claim 1, to decrease the level of ANGPTL3 gene expression, optionally wherein the dsRNA agent is administered to the subject subcutaneously or is administered to the subject by IV administration.

69. (canceled)

70. A method of altering a physiological characteristic of an ANGPTL3-associated disease or condition in a subject compared to a baseline pre-treatment physiological characteristic of the ANGPTL3-associated disease or condition in the subject, the method comprising administering to the subject an effective amount of a double-stranded ribonucleic acid (dsRNA) agent of claim 1, to alter the physiological characteristic of the ANGPTL3-associated disease or condition in the subject, optionally, wherein the dsRNA agent is administered to the subject subcutaneously or is administered to the subject by IV administration.

71-93. (canceled)


SEQUENCE LISTING (629 sequences)

SEQ ID NO: 1 - RNA, 21 nt, synthetic construct
  source 1..21: mol_type = other RNA
gttaaagactttgtccataaa

SEQ ID NO: 2 - RNA, 21 nt, synthetic construct
  source 1..21: mol_type = other RNA
gaaagactttgtccataagaa

SEQ ID NO: 3 - RNA, 21 nt, synthetic construct
  source 1..21: mol_type = other RNA
gaatgacatatttcaaaaaca

SEQ ID NO: 4 - RNA, 21 nt, synthetic construct
  source 1..21: mol_type = other RNA
gaaactcaacatatttgatca

SEQ ID NO: 5 - RNA, 21 nt, synthetic construct
  source 1..21: mol_type = other RNA
gaactcaacatatttgatcaa

SEQ ID NO: 6 - RNA, 21 nt, synthetic construct
  source 1..21: mol_type = other RNA
gttgatcagtctttttatgaa

SEQ ID NO: 7 - RNA, 21 nt, synthetic construct
  source 1..21: mol_type = other RNA
ggatcagtctttttatgatca

SEQ ID NO: 8 - RNA, 21 nt, synthetic construct
  source 1..21: mol_type = other RNA
gaagaactacatataaactaa

SEQ ID NO: 9 - RNA, 21 nt, synthetic construct
  source 1..21: mol_type = other RNA
gctacatataaactacaagta

SEQ ID NO: 10 - RNA, 21 nt, synthetic construct
  source 1..21: mol_type = other RNA
gaagaggtaaagaatatgtca

SEQ ID NO: 11 - RNA, 21 nt, synthetic construct
  source 1..21: mol_type = other RNA
gaggtaaagaatatgtcacta

SEQ ID NO: 12 - RNA, 21 nt, synthetic construct
  source 1..21: mol_type = other RNA
gtatgtcacttgaactcaaca

SEQ ID NO: 13 - RNA, 21 nt, synthetic construct
  source 1..21: mol_type = other RNA
gactcaaaacttgaaagccta

SEQ ID NO: 14 - RNA, 21 nt, synthetic construct
  source 1..21: mol_type = other RNA
gaacttgaaagcctcctagaa

SEQ ID NO: 15 - RNA, 21 nt, synthetic construct
  source 1..21: mol_type = other RNA
ggaaaaaattctacttcaaca

SEQ ID NO: 16 - RNA, 21 nt, synthetic construct
  source 1..21: mol_type = other RNA
ggaagagcaactaactaacta

SEQ ID NO: 17 - RNA, 21 nt, synthetic construct
  source 1..21: mol_type = other RNA
gttcaagaacccacagaaata

SEQ ID NO: 18 - RNA, 21 nt, synthetic construct
  source 1..21: mol_type = other RNA
gtcaagaacccacagaaatta

SEQ ID NO: 19 - RNA, 21 nt, synthetic construct
  source 1..21: mol_type = other RNA
gtctctatcttccaagccaaa

SEQ ID NO: 20 - RNA, 21 nt, synthetic construct
  source 1..21: mol_type = other RNA
gcaagaactactccctttcta

SEQ ID NO: 21 - RNA, 21 nt, synthetic construct
  source 1..21: mol_type = other RNA
gcaactctcaagtttttcata

SEQ ID NO: 22 - RNA, 21 nt, synthetic construct
  source 1..21: mol_type = other RNA
gactctcaagtttttcatgta

SEQ ID NO: 23 - RNA, 21 nt, synthetic construct
  source 1..21: mol_type = other RNA
gctctcaagtttttcatgtca

SEQ ID NO: 24 - RNA, 21 nt, synthetic construct
  source 1..21: mol_type = other RNA
gctcaagtttttcatgtctaa

SEQ ID NO: 25 - RNA, 21 nt, synthetic construct
  source 1..21: mol_type = other RNA
gtcaagtttttcatgtctaca

SEQ ID NO: 26 - RNA, 21 nt, synthetic construct
  source 1..21: mol_type = other RNA
gttttcatgtctactgtgata

SEQ ID NO: 27 - RNA, 21 nt, synthetic construct
  source 1..21: mol_type = other RNA
gtcatgtctactgtgatgtta

SEQ ID NO: 28 - RNA, 21 nt, synthetic construct
  source 1..21: mol_type = other RNA
gtggacattaattcaacatca

SEQ ID NO: 29 - RNA, 21 nt, synthetic construct
  source 1..21: mol_type = other RNA
gaacatcgaatagatggatca

SEQ ID NO: 30 - RNA, 21 nt, synthetic construct
  source 1..21: mol_type = other RNA
ggaatagatggatcacaaaaa

SEQ ID NO: 31 - RNA, 21 nt, synthetic construct
  source 1..21: mol_type = other RNA
gatagatggatcacaaaacta

SEQ ID NO: 32 - RNA, 21 nt, synthetic construct
  source 1..21: mol_type = other RNA
gtggatcacaaaacttcaata

SEQ ID NO: 33 - RNA, 21 nt, synthetic construct
  source 1..21: mol_type = other RNA
gcaaaacttcaatgaaacgta

SEQ ID NO: 34 - RNA, 21 nt, synthetic construct
  source 1..21: mol_type = other RNA
ggggagaactacaaatatgga

SEQ ID NO: 35 - RNA, 21 nt, synthetic construct
  source 1..21: mol_type = other RNA
gagagaagatatactccataa

SEQ ID NO: 36 - RNA, 21 nt, synthetic construct
  source 1..21: mol_type = other RNA
ggagaagatatactccataga

SEQ ID NO: 37 - RNA, 21 nt, synthetic construct
  source 1..21: mol_type = other RNA
gagatatactccatagtgaaa

SEQ ID NO: 38 - RNA, 21 nt, synthetic construct
  source 1..21: mol_type = other RNA
gtctttttacttgggaaatca

SEQ ID NO: 39 - RNA, 21 nt, synthetic construct
  source 1..21: mol_type = other RNA
gatgatgagtgtggagaaaaa

SEQ ID NO: 40 - RNA, 21 nt, synthetic construct
  source 1..21: mol_type = other RNA
ggaaaacaacctaaatggtaa

SEQ ID NO: 41 - RNA, 21 nt, synthetic construct
  source 1..21: mol_type = other RNA
gaatggaaggttatactctaa

SEQ ID NO: 42 - RNA, 21 nt, synthetic construct
  source 1..21: mol_type = other RNA
gggttatactctataaaatca

SEQ ID NO: 43 - RNA, 21 nt, synthetic construct
  source 1..21: mol_type = other RNA
gtcacaaaacttcaatgaaaa

SEQ ID NO: 44 - RNA, 21 nt, synthetic construct
  source 1..21: mol_type = other RNA
gcttgaaagcctcctagaaga

SEQ ID NO: 45 - RNA, 21 nt, synthetic construct
  source 1..21: mol_type = other RNA
gtgaactcaactcaaaactta

SEQ ID NO: 46 - RNA, 21 nt, synthetic construct
  source 1..21: mol_type = other RNA
gggacattaattcaacatcga

SEQ ID NO: 47 - RNA, 21 nt, synthetic construct
  source 1..21: mol_type = other RNA
ggacattaattcaacatcgaa

SEQ ID NO: 48 - RNA, 21 nt, synthetic construct
  source 1..21: mol_type = other RNA
gtaattcaacatcgaatagaa

SEQ ID NO: 49 - RNA, 21 nt, synthetic construct
  source 1..21: mol_type = other RNA
gaagggccaaattaatgacaa

SEQ ID NO: 50 - RNA, 21 nt, synthetic construct
  source 1..21: mol_type = other RNA
aagggccaaattaatgacata

SEQ ID NO: 51 - RNA, 21 nt, synthetic construct
  source 1..21: mol_type = other RNA
gaaagattttgtccataagaa

SEQ ID NO: 52 - RNA, 21 nt, synthetic construct
  source 1..21: mol_type = other RNA
gcaaaatcaagatttgctata

SEQ ID NO: 53 - RNA, 21 nt, synthetic construct
  source 1..21: mol_type = other RNA
gttaaagattttgtccataaa

SEQ ID NO: 54 - RNA, 21 nt, synthetic construct
  source 1..21: mol_type = other RNA
gaaaccaactacacgctacaa

SEQ ID NO: 55 - RNA, 21 nt, synthetic construct
  source 1..21: mol_type = other RNA
gaagctcaacatatttgatca

SEQ ID NO: 56 - RNA, 21 nt, synthetic construct
  source 1..21: mol_type = other RNA
gagctcaacatatttgatcaa

SEQ ID NO: 57 - RNA, 21 nt, synthetic construct
  source 1..21: mol_type = other RNA
gtatttgatcagtctttttaa

SEQ ID NO: 58 - RNA, 21 nt, synthetic construct
  source 1..21: mol_type = other RNA
gcacccagaagtaacatcaca

SEQ ID NO: 59 - RNA, 21 nt, synthetic construct
  source 1..21: mol_type = other RNA
gtaaagattttgtccataaga

SEQ ID NO: 60 - RNA, 21 nt, synthetic construct
  source 1..21: mol_type = other RNA
gcagtccatggacattaatta

SEQ ID NO: 61 - RNA, 21 nt, synthetic construct
  source 1..21: mol_type = other RNA
gaaagactttgtccataagaa

SEQ ID NO: 62 - RNA, 21 nt, synthetic construct
  source 1..21: mol_type = other RNA
cttaaagactttgtccataaa

SEQ ID NO: 63 - RNA, 21 nt, synthetic construct
  source 1..21: mol_type = other RNA
gaaaccaactatacgctacaa

SEQ ID NO: 64 - RNA, 21 nt, synthetic construct
  source 1..21: mol_type = other RNA
gaaactcaacatatttgatca

SEQ ID NO: 65 - RNA, 21 nt, synthetic construct
  source 1..21: mol_type = other RNA
gaactcaacatatttgatcaa

SEQ ID NO: 66 - RNA, 21 nt, synthetic construct
  source 1..21: mol_type = other RNA
acacccagaagtaacttcaca

SEQ ID NO: 67 - RNA, 21 nt, synthetic construct
  source 1..21: mol_type = other RNA
gtaaagactttgtccataaga

SEQ ID NO: 68 - RNA, 21 nt, synthetic construct
  source 1..21: mol_type = other RNA
gtagtccatggacattaatta

SEQ ID NO: 69 - RNA, 21 nt, synthetic construct
  source 1..21: mol_type = other RNA
nnnaaagactttgtccataan

SEQ ID NO: 70 - RNA, 21 nt, synthetic construct
  source 1..21: mol_type = other RNA
nnnagactttgtccataagan

SEQ ID NO: 71 - RNA, 21 nt, synthetic construct
  source 1..21: mol_type = other RNA
nnntgacatatttcaaaaacn

SEQ ID NO: 72 - RNA, 21 nt, synthetic construct
  source 1..21: mol_type = other RNA
nnnactcaacatatttgatcn

SEQ ID NO: 73 - RNA, 21 nt, synthetic construct
  source 1..21: mol_type = other RNA
nnnctcaacatatttgatcan

SEQ ID NO: 74 - RNA, 21 nt, synthetic construct
  source 1..21: mol_type = other RNA
nnngatcagtctttttatgan

SEQ ID NO: 75 - RNA, 21 nt, synthetic construct
  source 1..21: mol_type = other RNA
nnntcagtctttttatgatcn

SEQ ID NO: 76 - RNA, 21 nt, synthetic construct
  source 1..21: mol_type = other RNA
nnngaactacatataaactan

SEQ ID NO: 77 - RNA, 21 nt, synthetic construct
  source 1..21: mol_type = other RNA
nnnacatataaactacaagtn

SEQ ID NO: 78 - RNA, 21 nt, synthetic construct
  source 1..21: mol_type = other RNA
nnngaggtaaagaatatgtcn

SEQ ID NO: 79 - RNA, 21 nt, synthetic construct
  source 1..21: mol_type = other RNA
nnngtaaagaatatgtcactn

SEQ ID NO: 80 - RNA, 21 nt, synthetic construct
  source 1..21: mol_type = other RNA
nnntgtcacttgaactcaacn

SEQ ID NO: 81 - RNA, 21 nt, synthetic construct
  source 1..21: mol_type = other RNA
nnntcaaaacttgaaagcctn

SEQ ID NO: 82 - RNA, 21 nt, synthetic construct
  source 1..21: mol_type = other RNA
nnncttgaaagcctcctagan

SEQ ID NO: 83 - RNA, 21 nt, synthetic construct
  source 1..21: mol_type = other RNA
nnnaaaaattctacttcaacn

SEQ ID NO: 84 - RNA, 21 nt, synthetic construct
  source 1..21: mol_type = other RNA
nnnagagcaactaactaactn

SEQ ID NO: 85 - RNA, 21 nt, synthetic construct
  source 1..21: mol_type = other RNA
nnncaagaacccacagaaatn

SEQ ID NO: 86 - RNA, 21 nt, synthetic construct
  source 1..21: mol_type = other RNA
nnnaagaacccacagaaattn

SEQ ID NO: 87 - RNA, 21 nt, synthetic construct
  source 1..21: mol_type = other RNA
nnntctatcttccaagccaan

SEQ ID NO: 88 - RNA, 21 nt, synthetic construct
  source 1..21: mol_type = other RNA
nnnagaactactccctttctn

SEQ ID NO: 89 - RNA, 21 nt, synthetic construct
  source 1..21: mol_type = other RNA
nnnactctcaagtttttcatn

SEQ ID NO: 90 - RNA, 21 nt, synthetic construct
  source 1..21: mol_type = other RNA
nnntctcaagtttttcatgtn

SEQ ID NO: 91 - RNA, 21 nt, synthetic construct
  source 1..21: mol_type = other RNA
nnnctcaagtttttcatgtcn

SEQ ID NO: 92 - RNA, 21 nt, synthetic construct
  source 1..21: mol_type = other RNA
nnncaagtttttcatgtctan

SEQ ID NO: 93 - RNA, 21 nt, synthetic construct
  source 1..21: mol_type = other RNA
nnnaagtttttcatgtctacn

SEQ ID NO: 94 - RNA, 21 nt, synthetic construct
  source 1..21: mol_type = other RNA
nnnttcatgtctactgtgatn

SEQ ID NO: 95 - RNA, 21 nt, synthetic construct
  source 1..21: mol_type = other RNA
nnnatgtctactgtgatgttn

SEQ ID NO: 96 - RNA, 21 nt, synthetic construct
  source 1..21: mol_type = other RNA
nnngacattaattcaacatcn

SEQ ID NO: 97 - RNA, 21 nt, synthetic construct
  source 1..21: mol_type = other RNA
nnncatcgaatagatggatcn

SEQ ID NO: 98 - RNA, 21 nt, synthetic construct
  source 1..21: mol_type = other RNA
nnnatagatggatcacaaaan

SEQ ID NO: 99 - RNA, 21 nt, synthetic construct
  source 1..21: mol_type = other RNA
nnnagatggatcacaaaactn

SEQ ID NO: 100 - RNA, 21 nt, synthetic construct
  source 1..21: mol_type = other RNA
nnngatcacaaaacttcaatn

SEQ ID NO: 101 - RNA, 21 nt, synthetic construct
  source 1..21: mol_type = other RNA
nnnaaacttcaatgaaacgtn

SEQ ID NO: 102 - RNA, 21 nt, synthetic construct
  source 1..21: mol_type = other RNA
nnngagaactacaaatatggn

SEQ ID NO: 103 - RNA, 21 nt, synthetic construct
  source 1..21: mol_type = other RNA
nnnagaagatatactccatan

SEQ ID NO: 104 - RNA, 21 nt, synthetic construct
  source 1..21: mol_type = other RNA
nnngaagatatactccatagn

SEQ ID NO: 105 - RNA, 21 nt, synthetic construct
  source 1..21: mol_type = other RNA
nnnatatactccatagtgaan

SEQ ID NO: 106 - RNA, 21 nt, synthetic construct
  source 1..21: mol_type = other RNA
nnntttttacttgggaaatcn

SEQ ID NO: 107 - RNA, 21 nt, synthetic construct
  source 1..21: mol_type = other RNA
nnngatgagtgtggagaaaan

SEQ ID NO: 108 - RNA, 21 nt, synthetic construct
  source 1..21: mol_type = other RNA
nnnaaacaacctaaatggtan

SEQ ID NO: 109 - RNA, 21 nt, synthetic construct
  source 1..21: mol_type = other RNA
nnntggaaggttatactctan

SEQ ID NO: 110 - RNA, 21 nt, synthetic construct
  source 1..21: mol_type = other RNA
nnnttatactctataaaatcn

SEQ ID NO: 111 - RNA, 21 nt, synthetic construct
  source 1..21: mol_type = other RNA
nnnacaaaacttcaatgaaan

SEQ ID NO: 112 - RNA, 21 nt, synthetic construct
  source 1..21: mol_type = other RNA
nnntgaaagcctcctagaagn

SEQ ID NO: 113 - RNA, 21 nt, synthetic construct
  source 1..21: mol_type = other RNA
nnnaactcaactcaaaacttn

SEQ ID NO: 114 - RNA, 21 nt, synthetic construct
  source 1..21: mol_type = other RNA
nnnacattaattcaacatcgn

SEQ ID NO: 115 - RNA, 21 nt, synthetic construct
  source 1..21: mol_type = other RNA
nnncattaattcaacatcgan

SEQ ID NO: 116 - RNA, 21 nt, synthetic construct
  source 1..21: mol_type = other RNA
nnnattcaacatcgaatagan

SEQ ID NO: 117 - RNA, 21 nt, synthetic construct
  source 1..21: mol_type = other RNA
nnngggccaaattaatgacan

SEQ ID NO: 118 - RNA, 21 nt, synthetic construct
  source 1..21: mol_type = other RNA
tttatggacaaagtctttaac

SEQ ID NO: 119 - RNA, 21 nt, synthetic construct
  source 1..21: mol_type = other RNA
ttcttatggacaaagtctttc

SEQ ID NO: 120 - RNA, 21 nt, synthetic construct
  source 1..21: mol_type = other RNA
tgtttttgaaatatgtcattc

SEQ ID NO: 121 - RNA, 21 nt, synthetic construct
  source 1..21: mol_type = other RNA
tgatcaaatatgttgagtttc

SEQ ID NO: 122 - RNA, 21 nt, synthetic construct
  source 1..21: mol_type = other RNA
ttgatcaaatatgttgagttc

SEQ ID NO: 123 - RNA, 21 nt, synthetic construct
  source 1..21: mol_type = other RNA
ttcataaaaagactgatcaac

SEQ ID NO: 124 - RNA, 21 nt, synthetic construct
  source 1..21: mol_type = other RNA
tgatcataaaaagactgatcc

SEQ ID NO: 125 - RNA, 21 nt, synthetic construct
  source 1..21: mol_type = other RNA
ttagtttatatgtagttcttc

SEQ ID NO: 126 - RNA, 21 nt, synthetic construct
  source 1..21: mol_type = other RNA
tacttgtagtttatatgtagc

SEQ ID NO: 127 - RNA, 21 nt, synthetic construct
  source 1..21: mol_type = other RNA
tgacatattctttacctcttc

SEQ ID NO: 128 - RNA, 21 nt, synthetic construct
  source 1..21: mol_type = other RNA
tagtgacatattctttacctc

SEQ ID NO: 129 - RNA, 21 nt, synthetic construct
  source 1..21: mol_type = other RNA
tgttgagttcaagtgacatac

SEQ ID NO: 130 - RNA, 21 nt, synthetic construct
  source 1..21: mol_type = other RNA
taggctttcaagttttgagtc

SEQ ID NO: 131 - RNA, 21 nt, synthetic construct
  source 1..21: mol_type = other RNA
ttctaggaggctttcaagttc

SEQ ID NO: 132 - RNA, 21 nt, synthetic construct
  source 1..21: mol_type = other RNA
tgttgaagtagaattttttcc

SEQ ID NO: 133 - RNA, 21 nt, synthetic construct
  source 1..21: mol_type = other RNA
tagttagttagttgctcttcc

SEQ ID NO: 134 - RNA, 21 nt, synthetic construct
  source 1..21: mol_type = other RNA
tatttctgtgggttcttgaac

SEQ ID NO: 135 - RNA, 21 nt, synthetic construct
  source 1..21: mol_type = other RNA
taatttctgtgggttcttgac

SEQ ID NO: 136 - RNA, 21 nt, synthetic construct
  source 1..21: mol_type = other RNA
tttggcttggaagatagagac

SEQ ID NO: 137 - RNA, 21 nt, synthetic construct
  source 1..21: mol_type = other RNA
tagaaagggagtagttcttgc

SEQ ID NO: 138 - RNA, 21 nt, synthetic construct
  source 1..21: mol_type = other RNA
tatgaaaaacttgagagttgc

SEQ ID NO: 139 - RNA, 21 nt, synthetic construct
  source 1..21: mol_type = other RNA
tacatgaaaaacttgagagtc

SEQ ID NO: 140 - RNA, 21 nt, synthetic construct
  source 1..21: mol_type = other RNA
tgacatgaaaaacttgagagc

SEQ ID NO: 141 - RNA, 21 nt, synthetic construct
  source 1..21: mol_type = other RNA
ttagacatgaaaaacttgagc

SEQ ID NO: 142 - RNA, 21 nt, synthetic construct
  source 1..21: mol_type = other RNA
tgtagacatgaaaaacttgac

SEQ ID NO: 143 - RNA, 21 nt, synthetic construct
  source 1..21: mol_type = other RNA
tatcacagtagacatgaaaac

SEQ ID NO: 144 - RNA, 21 nt, synthetic construct
  source 1..21: mol_type = other RNA
taacatcacagtagacatgac

SEQ ID NO: 145 - RNA, 21 nt, synthetic construct
  source 1..21: mol_type = other RNA
tgatgttgaattaatgtccac

SEQ ID NO: 146 - RNA, 21 nt, synthetic construct
  source 1..21: mol_type = other RNA
tgatccatctattcgatgttc

SEQ ID NO: 147 - RNA, 21 nt, synthetic construct
  source 1..21: mol_type = other RNA
tttttgtgatccatctattcc

SEQ ID NO: 148 - RNA, 21 nt, synthetic construct
  source 1..21: mol_type = other RNA
tagttttgtgatccatctatc

SEQ ID NO: 149 - RNA, 21 nt, synthetic construct
  source 1..21: mol_type = other RNA
tattgaagttttgtgatccac

SEQ ID NO: 150 - RNA, 21 nt, synthetic construct
  source 1..21: mol_type = other RNA
tacgtttcattgaagttttgc

SEQ ID NO: 151 - RNA, 21 nt, synthetic construct
  source 1..21: mol_type = other RNA
tccatatttgtagttctcccc

SEQ ID NO: 152 - RNA, 21 nt, synthetic construct
  source 1..21: mol_type = other RNA
ttatggagtatatcttctctc

SEQ ID NO: 153 - RNA, 21 nt, synthetic construct
  source 1..21: mol_type = other RNA
tctatggagtatatcttctcc

SEQ ID NO: 154 - RNA, 21 nt, synthetic construct
  source 1..21: mol_type = other RNA
tttcactatggagtatatctc

SEQ ID NO: 155 - RNA, 21 nt, synthetic construct
  source 1..21: mol_type = other RNA
tgatttcccaagtaaaaagac

SEQ ID NO: 156 - RNA, 21 nt, synthetic construct
  source 1..21: mol_type = other RNA
tttttctccacactcatcatc

SEQ ID NO: 157 - RNA, 21 nt, synthetic construct
  source 1..21: mol_type = other RNA
ttaccatttaggttgttttcc

SEQ ID NO: 158 - RNA, 21 nt, synthetic construct
  source 1..21: mol_type = other RNA
ttagagtataaccttccattc

SEQ ID NO: 159 - RNA, 21 nt, synthetic construct
  source 1..21: mol_type = other RNA
tgattttatagagtataaccc

SEQ ID NO: 160 - RNA, 21 nt, synthetic construct
  source 1..21: mol_type = other RNA
ttttcattgaagttttgtgac

SEQ ID NO: 161 - RNA, 21 nt, synthetic construct
  source 1..21: mol_type = other RNA
tcttctaggaggctttcaagc

SEQ ID NO: 162 - RNA, 21 nt, synthetic construct
  source 1..21: mol_type = other RNA
taagttttgagttgagttcac

SEQ ID NO: 163 - RNA, 21 nt, synthetic construct
  source 1..21: mol_type = other RNA
tcgatgttgaattaatgtccc

SEQ ID NO: 164 - RNA, 21 nt, synthetic construct
  source 1..21: mol_type = other RNA
ttcgatgttgaattaatgtcc

SEQ ID NO: 165 - RNA, 21 nt, synthetic construct
  source 1..21: mol_type = other RNA
ttctattcgatgttgaattac

SEQ ID NO: 166 - RNA, 21 nt, synthetic construct
  source 1..21: mol_type = other RNA
ttgtcattaatttggcccttc

SEQ ID NO: 167 - RNA, 21 nt, synthetic construct
  source 1..21: mol_type = other RNA
tatgtcattaatttggccctt

SEQ ID NO: 168 - RNA, 21 nt, synthetic construct
  source 1..21: mol_type = other RNA
ttcttatggacaaaatctttc

SEQ ID NO: 169 - RNA, 21 nt, synthetic construct
  source 1..21: mol_type = other RNA
tatagcaaatcttgattttgc

SEQ ID NO: 170 - RNA, 21 nt, synthetic construct
  source 1..21: mol_type = other RNA
tttatggacaaaatctttaac

SEQ ID NO: 171 - RNA, 21 nt, synthetic construct
  source 1..21: mol_type = other RNA
ttgtagcgtgtagttggtttc

SEQ ID NO: 172 - RNA, 21 nt, synthetic construct
  source 1..21: mol_type = other RNA
tgatcaaatatgttgagcttc

SEQ ID NO: 173 - RNA, 21 nt, synthetic construct
  source 1..21: mol_type = other RNA
ttgatcaaatatgttgagctc

SEQ ID NO: 174 - RNA, 21 nt, synthetic construct
  source 1..21: mol_type = other RNA
ttaaaaagactgatcaaatac

SEQ ID NO: 175 - RNA, 21 nt, synthetic construct
  source 1..21: mol_type = other RNA
tgtgatgttacttctgggtgc

SEQ ID NO: 176 - RNA, 21 nt, synthetic construct
  source 1..21: mol_type = other RNA
tcttatggacaaaatctttac

SEQ ID NO: 177 - RNA, 21 nt, synthetic construct
  source 1..21: mol_type = other RNA
taattaatgtccatggactgc

SEQ ID NO: 178 - RNA, 21 nt, synthetic construct
  source 1..21: mol_type = other RNA
ttcttatggacaaagtctttc

SEQ ID NO: 179 - RNA, 21 nt, synthetic construct
  source 1..21: mol_type = other RNA
tttatggacaaagtctttaag

SEQ ID NO: 180 - RNA, 21 nt, synthetic construct
  source 1..21: mol_type = other RNA
ttgtagcgtatagttggtttc

SEQ ID NO: 181 - RNA, 21 nt, synthetic construct
  source 1..21: mol_type = other RNA
tgatcaaatatgttgagtttc

SEQ ID NO: 182 - RNA, 21 nt, synthetic construct
  source 1..21: mol_type = other RNA
ttgatcaaatatgttgagttc

SEQ ID NO: 183 - RNA, 21 nt, synthetic construct
  source 1..21: mol_type = other RNA
tgtgaagttacttctgggtgt

SEQ ID NO: 184 - RNA, 21 nt, synthetic construct
  source 1..21: mol_type = other RNA
tcttatggacaaagtctttac

SEQ ID NO: 185 - RNA, 21 nt, synthetic construct
  source 1..21: mol_type = other RNA
taattaatgtccatggactac

SEQ ID NO: 186 - RNA, 21 nt, synthetic construct
  source 1..21: mol_type = other RNA
nttatggacaaagtctttnnn

SEQ ID NO: 187 - RNA, 21 nt, synthetic construct
  source 1..21: mol_type = other RNA
ntcttatggacaaagtctnnn

SEQ ID NO: 188 - RNA, 21 nt, synthetic construct
  source 1..21: mol_type = other RNA
ngtttttgaaatatgtcannn

SEQ ID NO: 189 - RNA, 21 nt, synthetic construct
  source 1..21: mol_type = other RNA
ngatcaaatatgttgagtnnn

SEQ ID NO: 190 - RNA, 21 nt, synthetic construct
  source 1..21: mol_type = other RNA
ntgatcaaatatgttgagnnn

SEQ ID NO: 191 - RNA, 21 nt, synthetic construct
  source 1..21: mol_type = other RNA
ntcataaaaagactgatcnnn

SEQ ID NO: 192 - RNA, 21 nt, synthetic construct
  source 1..21: mol_type = other RNA
ngatcataaaaagactgannn

SEQ ID NO: 193 - RNA, 21 nt, synthetic construct
  source 1..21: mol_type = other RNA
ntagtttatatgtagttcnnn

SEQ ID NO: 194 - RNA, 21 nt, synthetic construct
  source 1..21: mol_type = other RNA
nacttgtagtttatatgtnnn

SEQ ID NO: 195 - RNA, 21 nt, synthetic construct
  source 1..21: mol_type = other RNA
ngacatattctttacctcnnn

SEQ ID NO: 196 - RNA, 21 nt, synthetic construct
  source 1..21: mol_type = other RNA
nagtgacatattctttacnnn

SEQ ID NO: 197 - RNA, 21 nt, synthetic construct
  source 1..21: mol_type = other RNA
ngttgagttcaagtgacannn

SEQ ID NO: 198 - RNA, 21 nt, synthetic construct
  source 1..21: mol_type = other RNA
naggctttcaagttttgannn

SEQ ID NO: 199 - RNA, 21 nt, synthetic construct
  source 1..21: mol_type = other RNA
ntctaggaggctttcaagnnn

SEQ ID NO: 200 - RNA, 21 nt, synthetic construct
  source 1..21: mol_type = other RNA
ngttgaagtagaatttttnnn

SEQ ID NO: 201 - RNA, 21 nt, synthetic construct
  source 1..21: mol_type = other RNA
nagttagttagttgctctnnn

SEQ ID NO: 202 - RNA, 21 nt, synthetic construct
  source 1..21: mol_type = other RNA
natttctgtgggttcttgnnn

SEQ ID NO: 203 - RNA, 21 nt, synthetic construct
  source 1..21: mol_type = other RNA
naatttctgtgggttcttnnn

SEQ ID NO: 204 - RNA, 21 nt, synthetic construct
  source 1..21: mol_type = other RNA
nttggcttggaagatagannn

SEQ ID NO: 205 - RNA, 21 nt, synthetic construct
  source 1..21: mol_type = other RNA
nagaaagggagtagttctnnn

SEQ ID NO: 206 - RNA, 21 nt, synthetic construct
  source 1..21: mol_type = other RNA
natgaaaaacttgagagtnnn

SEQ ID NO: 207 - RNA, 21 nt, synthetic construct
  source 1..21: mol_type = other RNA
nacatgaaaaacttgagannn

SEQ ID NO: 208 - RNA, 21 nt, synthetic construct
  source 1..21: mol_type = other RNA
ngacatgaaaaacttgagnnn

SEQ ID NO: 209 - RNA, 21 nt, synthetic construct
  source 1..21: mol_type = other RNA
ntagacatgaaaaacttgnnn

SEQ ID NO: 210 - RNA, 21 nt, synthetic construct
  source 1..21: mol_type = other RNA
ngtagacatgaaaaacttnnn

SEQ ID NO: 211 - RNA, 21 nt, synthetic construct
  source 1..21: mol_type = other RNA
natcacagtagacatgaannn

SEQ ID NO: 212 - RNA, 21 nt, synthetic construct
  source 1..21: mol_type = other RNA
naacatcacagtagacatnnn

SEQ ID NO: 213 - RNA, 21 nt, synthetic construct
  source 1..21: mol_type = other RNA
ngatgttgaattaatgtcnnn

SEQ ID NO: 214 - RNA, 21 nt, synthetic construct
  source 1..21: mol_type = other RNA
ngatccatctattcgatgnnn

SEQ ID NO: 215 - RNA, 21 nt, synthetic construct
  source 1..21: mol_type = other RNA
nttttgtgatccatctatnnn

SEQ ID NO: 216 - RNA, 21 nt, synthetic construct
  source 1..21: mol_type = other RNA
nagttttgtgatccatctnnn

SEQ ID NO: 217 - RNA, 21 nt, synthetic construct
  source 1..21: mol_type = other RNA
nattgaagttttgtgatcnnn

SEQ ID NO: 218 - RNA, 21 nt, synthetic construct
  source 1..21: mol_type = other RNA
nacgtttcattgaagtttnnn

SEQ ID NO: 219 - RNA, 21 nt, synthetic construct
  source 1..21: mol_type = other RNA
nccatatttgtagttctcnnn

SEQ ID NO: 220 - RNA, 21 nt, synthetic construct
  source 1..21: mol_type = other RNA
ntatggagtatatcttctnnn

SEQ ID NO: 221 - RNA, 21 nt, synthetic construct
  source 1..21: mol_type = other RNA
nctatggagtatatcttcnnn

SEQ ID NO: 222 - RNA, 21 nt, synthetic construct
  source 1..21: mol_type = other RNA
nttcactatggagtatatnnn

SEQ ID NO: 223 - RNA, 21 nt, synthetic construct
  source 1..21: mol_type = other RNA
ngatttcccaagtaaaaannn

SEQ ID NO: 224 - RNA, 21 nt, synthetic construct
  source 1..21: mol_type = other RNA
nttttctccacactcatcnnn

SEQ ID NO: 225 - RNA, 21 nt, synthetic construct
  source 1..21: mol_type = other RNA
ntaccatttaggttgtttnnn

SEQ ID NO: 226 - RNA, 21 nt, synthetic construct
  source 1..21: mol_type = other RNA
ntagagtataaccttccannn

SEQ ID NO: 227 - RNA, 21 nt, synthetic construct
  source 1..21: mol_type = other RNA
ngattttatagagtataannn

SEQ ID NO: 228 - RNA, 21 nt, synthetic construct
  source 1..21: mol_type = other RNA
ntttcattgaagttttgtnnn

SEQ ID NO: 229 - RNA, 21 nt, synthetic construct
  source 1..21: mol_type = other RNA
ncttctaggaggctttcannn

SEQ ID NO: 230 - RNA, 21 nt, synthetic construct
  source 1..21: mol_type = other RNA
naagttttgagttgagttnnn

SEQ ID NO: 231 - RNA, 21 nt, synthetic construct
  source 1..21: mol_type = other RNA
ncgatgttgaattaatgtnnn

SEQ ID NO: 232 - RNA, 21 nt, synthetic construct
  source 1..21: mol_type = other RNA
ntcgatgttgaattaatgnnn

SEQ ID NO: 233 - RNA, 21 nt, synthetic construct
  source 1..21: mol_type = other RNA
ntctattcgatgttgaatnnn

SEQ ID NO: 234 - RNA, 21 nt, synthetic construct
  source 1..21: mol_type = other RNA
ntgtcattaatttggcccnnn

SEQ ID NO: 235 - RNA, 2926 nt, Homo sapiens
  source 1..2926: mol_type = mRNA
agaagaaaacagttccacgttgcttgaaattgaaaatcaagataaaaatgttcacaattaagctccttctttttattgttcctctagttatttcctccagaattgatcaagacaattcatcatttgattctctatctccagagccaaaatcaagatttgctatgttagacgatgtaaaaattttagccaatggcctccttcagttgggacatggtcttaaagactttgtccataagacgaagggccaaattaatgacatatttcaaaaactcaacatatttgatcagtctttttatgatctatcgctgcaaaccagtgaaatcaaagaagaagaaaaggaactgagaagaactacatataaactacaagtcaaaaatgaagaggtaaagaatatgtcacttgaactcaactcaaaacttgaaagcctcctagaagaaaaaattctacttcaacaaaaagtgaaatatttagaagagcaactaactaacttaattcaaaatcaacctgaaactccagaacacccagaagtaacttcacttaaaacttttgtagaaaaacaagataatagcatcaaagaccttctccagaccgtggaagaccaatataaacaattaaaccaacagcatagtcaaataaaagaaatagaaaatcagctcagaaggactagtattcaagaacccacagaaatttctctatcttccaagccaagagcaccaagaactactccctttcttcagttgaatgaaataagaaatgtaaaacatgatggcattcctgctgaatgtaccaccatttataacagaggtgaacatacaagtggcatgtatgccatcagacccagcaactctcaagtttttcatgtctactgtgatgttatatcaggtagtccatggacattaattcaacatcgaatagatggatcacaaaacttcaatgaaacgtgggagaactacaaatatggttttgggaggcttgatggagaattttggttgggcctagagaagatatactccatagtgaagcaatctaattatgttttacgaattgagttggaagactggaaagacaacaaacattatattgaatattctttttacttgggaaatcacgaaaccaactatacgctacatctagttgcgattactggcaatgtccccaatgcaatcccggaaaacaaagatttggtgttttctacttgggatcacaaagcaaaaggacacttcaactgtccagagggttattcaggaggctggtggtggcatgatgagtgtggagaaaacaacctaaatggtaaatataacaaaccaagagcaaaatctaagccagagaggagaagaggattatcttggaagtctcaaaatggaaggttatactctataaaatcaaccaaaatgttgatccatccaacagattcagaaagctttgaatgaactgaggcaaatttaaaaggcaataatttaaacattaacctcattccaagttaatgtggtctaataatctggtattaaatccttaagagaaagcttgagaaatagattttttttatcttaaagtcactgtctatttaagattaaacatacaatcacataaccttaaagaataccgtttacatttctcaatcaaaattcttataatactatttgttttaaattttgtgatgtgggaatcaattttagatggtcacaatctagattataatcaataggtgaacttattaaataacttttctaaataaaaaatttagagacttttattttaaaaggcatcatatgagctaatatcacaactttcccagtttaaaaaactagtactcttgttaaaactctaaacttgactaaatacagaggactggtaattgtacagttcttaaatgttgtagtattaatttcaaaactaaaaatcgtcagcacagagtatgtgtaaaaatctgtaatacaaatttttaaactgatgcttcattttgctacaaaataatttggagtaaatgtttgatatgatttatttatgaaacctaatgaagcagaattaaatactgtattaaaataagttcgctgtctttaaacaaatggagatgactactaagtcacattgactttaacatgaggtatcactataccttatttgttaaaatatatactgtatacattttatatattttaacacttaatactatgaaaacaaataattgtaaaggaatcttgtcagattacagtaagaatgaacatatttgtggcatcgagttaaagtttatatttcccctaaatatgctgtgattctaatacattcgtgtaggttttcaagtagaaataaacctcgtaacaagttactgaacgtttaaacagcctgacaagcatgtatatatgtttaaaattcaataaacaaagacccagtccctaaattatagaaatttaaattattcttgcatgtttatcgacatcacaacagatccctaaatccctaaatccctaaagattagatacaaattttttaccacagtatcacttgtcagaatttatttttaaatatgattttttaaaactgccagtaagaaattttaaattaaacccatttgttaaaggatatagtgcccaagttatatggtgacctacctttgtcaatacttagcattatgtatttcaaattatccaatatacatgtcatatatatttttatatgtcacatatataaaagatatgtatgatctatgtgaatcctaagtaaatattttgttccagaaaagtacaaaataataaaggtaaaaataatctataattttcaggaccacagactaagctgtcgaaattaacgctgatttttttagggccagaataccaaaatggctcctctcttcccccaaaattggacaatttcaaatgcaaaataattcattatttaatatatgagttgcttcctctatttggtttcc

SEQ ID NO: 236 - RNA, 1609 nt, Mus musculus
  source 1..1609: mol_type = mRNA
acaggagggagaagttccaaattgcttaaaattgaataattgagacaaaaaatgcacacaattaaattattcctttttgttgttcctttagtaattgcatccagagtggatccagacctttcatcatttgattctgcaccttcagagccaaaatcaagatttgctatgttggatgatgtcaaaattttagcgaatggcctcctgcagctgggtcatggacttaaagattttgtccataagactaagggacaaattaacgacatatttcagaagctcaacatatttgatcagtctttttatgacctatcacttcgaaccaatgaaatcaaagaagaggaaaaggagctaagaagaactacatctacactacaagttaaaaacgaggaggtgaagaacatgtcagtagaactgaactcaaagcttgagagtctgctggaagagaagacagcccttcaacacaaggtcagggctttggaggagcagctaaccaacttaattctaagcccagctggggctcaggagcacccagaagtaacatcactcaaaagttttgtagaacagcaagacaacagcataagagaactcctccagagtgtggaagaacagtataaacaattaagtcaacagcacatgcagataaaagaaatagaaaagcagctcagaaagactggtattcaagaaccctcagaaaattctctttcttctaaatcaagagcaccaagaactactccccctcttcaactgaacgaaacagaaaatacagaacaagatgaccttcctgccgactgctctgccgtttataacagaggcgaacatacaagtggcgtgtacactattaaaccaagaaactcccaagggtttaatgtctactgtgatacccaatcaggcagtccatggacattaattcaacaccggaaagatggctcacaggacttcaacgaaacatgggaaaactacgaaaagggctttgggaggctcgatggagaattttggttgggcctagagaagatctatgctatagtccaacagtctaactacattttacgactcgagctacaagactggaaagacagcaagcactacgttgaatactcctttcacctgggcagtcacgaaaccaactacacgctacatgtggctgagattgctggcaatatccctggggccctcccagagcacacagacctgatgttttctacatggaatcacagagcaaagggacagctctactgtccagaaagttactcaggtggctggtggtggaatgacatatgtggagaaaacaacctaaatggaaaatacaacaaacccagaaccaaatccagaccagagagaagaagagggatctactggagacctcagagcagaaagctctatgctatcaaatcatccaaaatgatgctccagcccaccacctaagaagcttcaactgaactgagacaaaataaaagatcaataaattaaatattaaagtcctcccgatcactgtagtaatctggtattaaaattttaatggaaagcttgagaattgaatttcaattaggtttaaactcattgttaagatcagatatcaccgaatcaacgtaaacaaaatttatctttttcaatc

SEQ ID NO: 237 - RNA, 21 nt, synthetic construct
  source 1..21: mol_type = other RNA
gttaaagactttgtccataaa

SEQ ID NO: 238 - RNA, 21 nt, synthetic construct
  source 1..21: mol_type = other RNA
gaaagactttgtccataagaa

SEQ ID NO: 239 - RNA, 21 nt, synthetic construct
  source 1..21: mol_type = other RNA
gctcaacatatttgatcagta

SEQ ID NO: 240 - RNA, 21 nt, synthetic construct
  source 1..21: mol_type = other RNA
gaatgacatatttcaaaaaca

SEQ ID NO: 241 - RNA, 21 nt, synthetic construct
  source 1..21: mol_type = other RNA
gaaactcaacatatttgatca

SEQ ID NO: 242 - RNA, 21 nt, synthetic construct
  source 1..21: mol_type = other RNA
gaactcaacatatttgatcaa

SEQ ID NO: 243 - RNA, 21 nt, synthetic construct
  source 1..21: mol_type = other RNA
gttgatcagtctttttatgaa

SEQ ID NO: 244 - RNA, 21 nt, synthetic construct
  source 1..21: mol_type = other RNA
ggatcagtctttttatgatca

SEQ ID NO: 245 - RNA, 21 nt, synthetic construct
  source 1..21: mol_type = other RNA
gaagaactacatataaactaa

SEQ ID NO: 246 - RNA, 21 nt, synthetic construct
  source 1..21: mol_type = other RNA
gctacatataaactacaagta

SEQ ID NO: 247 - RNA, 21 nt, synthetic construct
  source 1..21: mol_type = other RNA
gaagaggtaaagaatatgtca

SEQ ID NO: 248 - RNA, 21 nt, synthetic construct
  source 1..21: mol_type = other RNA
gaggtaaagaatatgtcacta

SEQ ID NO: 249 - RNA, 21 nt, synthetic construct
  source 1..21: mol_type = other RNA
gtatgtcacttgaactcaaca

SEQ ID NO: 250 - RNA, 21 nt, synthetic construct
  source 1..21: mol_type = other RNA
gactcaaaacttgaaagccta

SEQ ID NO: 251 - RNA, 21 nt, synthetic construct
  source 1..21: mol_type = other RNA
gaacttgaaagcctcctagaa

SEQ ID NO: 252 - RNA, 21 nt, synthetic construct
  source 1..21: mol_type = other RNA
ggaaaaaattctacttcaaca

SEQ ID NO: 253 - RNA, 21 nt, synthetic construct
  source 1..21: mol_type = other RNA
ggaagagcaactaactaacta

SEQ ID NO: 254 - RNA, 21 nt, synthetic construct
  source 1..21: mol_type = other RNA
gttcaagaacccacagaaata

SEQ ID NO: 255 - RNA, 21 nt, synthetic construct
  source 1..21: mol_type = other RNA
gtcaagaacccacagaaatta

SEQ ID NO: 256 - RNA, 21 nt, synthetic construct
  source 1..21: mol_type = other RNA
gtctctatcttccaagccaaa

SEQ ID NO: 257 - RNA, 21 nt, synthetic construct
  source 1..21: mol_type = other RNA
gcaagaactactccctttcta

SEQ ID NO: 258 - RNA, 21 nt, synthetic construct
  source 1..21: mol_type = other RNA
gcaactctcaagtttttcata

SEQ ID NO: 259 - RNA, 21 nt, synthetic construct
  source 1..21: mol_type = other RNA
gactctcaagtttttcatgta

SEQ ID NO: 260 - RNA, 21 nt, synthetic construct
  source 1..21: mol_type = other RNA
gctctcaagtttttcatgtca

SEQ ID NO: 261 - RNA, 21 nt, synthetic construct
  source 1..21: mol_type = other RNA
gctcaagtttttcatgtctaa

SEQ ID NO: 262 - RNA, 21 nt, synthetic construct
  source 1..21: mol_type = other RNA
gtcaagtttttcatgtctaca

SEQ ID NO: 263 - RNA, 21 nt, synthetic construct
  source 1..21: mol_type = other RNA
gttttcatgtctactgtgata

SEQ ID NO: 264 - RNA, 21 nt, synthetic construct
  source 1..21: mol_type = other RNA
gtcatgtctactgtgatgtta

SEQ ID NO: 265 - RNA, 21 nt, synthetic construct
  source 1..21: mol_type = other RNA
gtggacattaattcaacatca

SEQ ID NO: 266 - RNA, 21 nt, synthetic construct
  source 1..21: mol_type = other RNA
gaacatcgaatagatggatca

SEQ ID NO: 267 - RNA, 21 nt, synthetic construct
  source 1..21: mol_type = other RNA
ggaatagatggatcacaaaaa

SEQ ID NO: 268 - RNA, 21 nt, synthetic construct
  source 1..21: mol_type = other RNA
gatagatggatcacaaaacta

SEQ ID NO: 269 - RNA, 21 nt, synthetic construct
  source 1..21: mol_type = other RNA
gtggatcacaaaacttcaata

SEQ ID NO: 270 - RNA, 21 nt, synthetic construct
  source 1..21: mol_type = other RNA
gcaaaacttcaatgaaacgta

SEQ ID NO: 271 - RNA, 21 nt, synthetic construct
  source 1..21: mol_type = other RNA
ggggagaactacaaatatgga

SEQ ID NO: 272 - RNA, 21 nt, synthetic construct
  source 1..21: mol_type = other RNA
gagagaagatatactccataa

SEQ ID NO: 273 - RNA, 21 nt, synthetic construct
  source 1..21: mol_type = other RNA
ggagaagatatactccataga

SEQ ID NO: 274 - RNA, 21 nt, synthetic construct
  source 1..21: mol_type = other RNA
gagatatactccatagtgaaa

SEQ ID NO: 275 - RNA, 21 nt, synthetic construct
  source 1..21: mol_type = other RNA
gtctttttacttgggaaatca

SEQ ID NO: 276 - RNA, 21 nt, synthetic construct
  source 1..21: mol_type = other RNA
gatgatgagtgtggagaaaaa

SEQ ID NO: 277 - RNA, 21 nt, synthetic construct
  source 1..21: mol_type = other RNA
ggaaaacaacctaaatggtaa

SEQ ID NO: 278 - RNA, 21 nt, synthetic construct
  source 1..21: mol_type = other RNA
gaatggaaggttatactctaa

SEQ ID NO: 279 - RNA, 21 nt, synthetic construct
  source 1..21: mol_type = other RNA
gggttatactctataaaatca

SEQ ID NO: 280 - RNA, 21 nt, synthetic construct
  source 1..21: mol_type = other RNA
gtcacaaaacttcaatgaaaa

SEQ ID NO: 281 - RNA, 21 nt, synthetic construct
  source 1..21: mol_type = other RNA
gcttgaaagcctcctagaaga

SEQ ID NO: 282 - RNA, 21 nt, synthetic construct
  source 1..21: mol_type = other RNA
gtgaactcaactcaaaactta

SEQ ID NO: 283 - RNA, 21 nt, synthetic construct
  source 1..21: mol_type = other RNA
gggacattaattcaacatcga

SEQ ID NO: 284 - RNA, 21 nt, synthetic construct
  source 1..21: mol_type = other RNA
ggacattaattcaacatcgaa

SEQ ID NO: 285 - RNA, 21 nt, synthetic construct
  source 1..21: mol_type = other RNA
gtaattcaacatcgaatagaa

SEQ ID NO: 286 - RNA, 21 nt, synthetic construct
  source 1..21: mol_type = other RNA
gaagggccaaattaatgacaa

SEQ ID NO: 287 - RNA, 21 nt, synthetic construct
  source 1..21: mol_type = other RNA
tttatggacaaagtctttaac

SEQ ID NO: 288 - RNA, 21 nt, synthetic construct
  source 1..21: mol_type = other RNA
ttcttatggacaaagtctttc

SEQ ID NO: 289 - RNA, 21 nt, synthetic construct
  source 1..21: mol_type = other RNA
tactgatcaaatatgttgagc

SEQ ID NO: 290 - RNA, 21 nt, synthetic construct
  source 1..21: mol_type = other RNA
tgtttttgaaatatgtcattc

SEQ ID NO: 291 - RNA, 21 nt, synthetic construct
  source 1..21: mol_type = other RNA
tgatcaaatatgttgagtttc

SEQ ID NO: 292 - RNA, 21 nt, synthetic construct
  source 1..21: mol_type = other RNA
ttgatcaaatatgttgagttc

SEQ ID NO: 293 - RNA, 21 nt, synthetic construct
  source 1..21: mol_type = other RNA
ttcataaaaagactgatcaac

SEQ ID NO: 294 - RNA, 21 nt, synthetic construct
  source 1..21: mol_type = other RNA
tgatcataaaaagactgatcc

SEQ ID NO: 295 - RNA, 21 nt, synthetic construct
  source 1..21: mol_type = other RNA
ttagtttatatgtagttcttc

SEQ ID NO: 296 - RNA, 21 nt, synthetic construct
  source 1..21: mol_type = other RNA
tacttgtagtttatatgtagc

SEQ ID NO: 297 - RNA, 21 nt, synthetic construct
  source 1..21: mol_type = other RNA
tgacatattctttacctcttc

SEQ ID NO: 298 - RNA, 21 nt, synthetic construct
  source 1..21: mol_type = other RNA
tagtgacatattctttacctc

SEQ ID NO: 299 - RNA, 21 nt, synthetic construct
  source 1..21: mol_type = other RNA
tgttgagttcaagtgacatac

SEQ ID NO: 300 - RNA, 21 nt, synthetic construct
  source 1..21: mol_type = other RNA
taggctttcaagttttgagtc

SEQ ID NO: 301 - RNA, 21 nt, synthetic construct
  source 1..21: mol_type = other RNA
ttctaggaggctttcaagttc

SEQ ID NO: 302 - RNA, 21 nt, synthetic construct
  source 1..21: mol_type = other RNA
tgttgaagtagaattttttcc

SEQ ID NO: 303 - RNA, 21 nt, synthetic construct
  source 1..21: mol_type = other RNA
tagttagttagttgctcttcc

SEQ ID NO: 304 - RNA, 21 nt, synthetic construct
  source 1..21: mol_type = other RNA
tatttctgtgggttcttgaac

SEQ ID NO: 305 - RNA, 21 nt, synthetic construct
  source 1..21: mol_type = other RNA
taatttctgtgggttcttgac

SEQ ID NO: 306 - RNA, 21 nt, synthetic construct
  source 1..21: mol_type = other RNA
tttggcttggaagatagagac

SEQ ID NO: 307 - RNA, 21 nt, synthetic construct
  source 1..21: mol_type = other RNA
tagaaagggagtagttcttgc

SEQ ID NO: 308 - RNA, 21 nt, synthetic construct
  source 1..21: mol_type = other RNA
tatgaaaaacttgagagttgc

SEQ ID NO: 309 - RNA, 21 nt, synthetic construct
  source 1..21: mol_type = other RNA
tacatgaaaaacttgagagtc

SEQ ID NO: 310 - RNA, 21 nt, synthetic construct
  source 1..21: mol_type = other RNA
tgacatgaaaaacttgagagc

SEQ ID NO: 311 - RNA, 21 nt, synthetic construct
  source 1..21: mol_type = other RNA
ttagacatgaaaaacttgagc

SEQ ID NO: 312 - RNA, 21 nt, synthetic construct
  source 1..21: mol_type = other RNA
tgtagacatgaaaaacttgac

SEQ ID NO: 313 - RNA, 21 nt, synthetic construct
  source 1..21: mol_type = other RNA
tatcacagtagacatgaaaac

SEQ ID NO: 314 - RNA, 21 nt, synthetic construct
  source 1..21: mol_type = other RNA
taacatcacagtagacatgac

SEQ ID NO: 315 - RNA, 21 nt, synthetic construct
  source 1..21: mol_type = other RNA
tgatgttgaattaatgtccac

SEQ ID NO: 316 - RNA, 21 nt, synthetic construct
  source 1..21: mol_type = other RNA
tgatccatctattcgatgttc

SEQ ID NO: 317 - RNA, 21 nt, synthetic construct
  source 1..21: mol_type = other RNA
tttttgtgatccatctattcc

SEQ ID NO: 318 - RNA, 21 nt, synthetic construct
  source 1..21: mol_type = other RNA
tagttttgtgatccatctatc

SEQ ID NO: 319 - RNA, 21 nt, synthetic construct
  source 1..21: mol_type = other RNA
tattgaagttttgtgatccac

SEQ ID NO: 320 - RNA, 21 nt, synthetic construct
  source 1..21: mol_type = other RNA
tacgtttcattgaagttttgc

SEQ ID NO: 321 - RNA, 21 nt, synthetic construct
  source 1..21: mol_type = other RNA
tccatatttgtagttctcccc

SEQ ID NO: 322 - RNA, 21 nt, synthetic construct
  source 1..21: mol_type = other RNA
ttatggagtatatcttctctc

SEQ ID NO: 323 - RNA, 21 nt, synthetic construct
  source 1..21: mol_type = other RNA
tctatggagtatatcttctcc

SEQ ID NO: 324 - RNA, 21 nt, synthetic construct
  source 1..21: mol_type = other RNA
tttcactatggagtatatctc

SEQ ID NO: 325 - RNA, 21 nt, synthetic construct
  source 1..21: mol_type = other RNA
tgatttcccaagtaaaaagac

SEQ ID NO: 326 - RNA, 21 nt, synthetic construct
  source 1..21: mol_type = other RNA
tttttctccacactcatcatc

SEQ ID NO: 327 - RNA, 21 nt, synthetic construct
  source 1..21: mol_type = other RNA
ttaccatttaggttgttttcc

SEQ ID NO: 328 - RNA, 21 nt, synthetic construct
  source 1..21: mol_type = other RNA
ttagagtataaccttccattc

SEQ ID NO: 329 - RNA, 21 nt, synthetic construct
  source 1..21: mol_type = other RNA
tgattttatagagtataaccc

SEQ ID NO: 330 - RNA, 21 nt, synthetic construct
  source 1..21: mol_type = other RNA
ttttcattgaagttttgtgac

SEQ ID NO: 331 - RNA, 21 nt, synthetic construct
  source 1..21: mol_type = other RNA
tcttctaggaggctttcaagc

SEQ ID NO: 332 - RNA, 21 nt, synthetic construct
  source 1..21: mol_type = other RNA
taagttttgagttgagttcac

SEQ ID NO: 333 - RNA, 21 nt, synthetic construct
  source 1..21: mol_type = other RNA
tcgatgttgaattaatgtccc

SEQ ID NO: 334 - RNA, 21 nt, synthetic construct
  source 1..21: mol_type = other RNA
ttcgatgttgaattaatgtcc

SEQ ID NO: 335 - RNA, 21 nt, synthetic construct
  source 1..21: mol_type = other RNA
ttctattcgatgttgaattac

SEQ ID NO: 336 - RNA, 21 nt, synthetic construct
  source 1..21: mol_type = other RNA
ttgtcattaatttggcccttc

SEQ ID NO: 337 - RNA, 21 nt, synthetic construct
  source 1..21: mol_type = other RNA
gaaagactttgtccataagaa

SEQ ID NO: 338 - RNA, 21 nt, synthetic construct
  source 1..21: mol_type = other RNA
gaaactcaacatatttgatca

SEQ ID NO: 339 - RNA, 21 nt, synthetic construct
  source 1..21: mol_type = other RNA
ggatcagtctttttatgatca

SEQ ID NO: 340 - RNA, 21 nt, synthetic construct
  source 1..21: mol_type = other RNA
gaggtaaagaatatgtcacta

SEQ ID NO: 341 - RNA, 21 nt, synthetic construct
  source 1..21: mol_type = other RNA
ggaagagcaactaactaacta

SEQ ID NO: 342 - RNA, 21 nt, synthetic construct
  source 1..21: mol_type = other RNA
gtcaagaacccacagaaatta

SEQ ID NO: 343 - RNA, 21 nt, synthetic construct
  source 1..21: mol_type = other RNA
gactctcaagtttttcatgta

SEQ ID NO: 344 - RNA, 21 nt, synthetic construct
  source 1..21: mol_type = other RNA
gctctcaagtttttcatgtca

SEQ ID NO: 345 - RNA, 21 nt, synthetic construct
  source 1..21: mol_type = other RNA
gctcaagtttttcatgtctaa

SEQ ID NO: 346 - RNA, 21 nt, synthetic construct
  source 1..21: mol_type = other RNA
gtggacattaattcaacatca

SEQ ID NO: 347 - RNA, 21 nt, synthetic construct
  source 1..21: mol_type = other RNA
gaatggaaggttatactctaa

SEQ ID NO: 348 - RNA, 21 nt, synthetic construct
  source 1..21: mol_type = other RNA
aagggccaaattaatgacata

SEQ ID NO: 349 - RNA, 21 nt, synthetic construct
  source 1..21: mol_type = other RNA
gaatgacatatttcaaaaaca

SEQ ID NO: 350 - RNA, 21 nt, synthetic construct
  source 1..21: mol_type = other RNA
gttgatcagtctttttatgaa

SEQ ID NO: 351 - RNA, 21 nt, synthetic construct
  source 1..21: mol_type = other RNA
gaagaactacatataaactaa

SEQ ID NO: 352 - RNA, 21 nt, synthetic construct
  source 1..21: mol_type = other RNA
gtatgtcacttgaactcaaca

SEQ ID NO: 353 - RNA, 21 nt, synthetic construct
  source 1..21: mol_type = other RNA
gactcaaaacttgaaagccta

SEQ ID NO: 354 - RNA, 21 nt, synthetic construct
  source 1..21: mol_type = other RNA
gcttgaaagcctcctagaaga

SEQ ID NO: 355 - RNA, 21 nt, synthetic construct
  source 1..21: mol_type = other RNA
ctcaagtttttcatgtctaca

SEQ ID NO: 356 - RNA, 21 nt, synthetic construct
  source 1..21: mol_type = other RNA
gttttcatgtctactgtgata

SEQ ID NO: 357 - RNA, 21 nt, synthetic construct
  source 1..21: mol_type = other RNA
caacatcgaatagatggatca

SEQ ID NO: 358 - RNA, 21 nt, synthetic construct
  source 1..21: mol_type = other RNA
cgaatagatggatcacaaaaa

SEQ ID NO: 359 - RNA, 21 nt, synthetic construct
  source 1..21: mol_type = other RNA
atggatcacaaaacttcaata

SEQ ID NO: 360 - RNA, 21 nt, synthetic construct
  source 1..21: mol_type = other RNA
gcaaaacttcaatgaaacgta

SEQ ID NO: 361 - RNA, 21 nt, synthetic construct
  source 1..21: mol_type = other RNA
gtctttttacttgggaaatca

SEQ ID NO: 362 - RNA, 21 nt, synthetic construct
  source 1..21: mol_type = other RNA
catgatgagtgtggagaaaaa

SEQ ID NO: 363 - RNA, 21 nt, synthetic construct
  source 1..21: mol_type = other RNA
aggttatactctataaaatca

SEQ ID NO: 364 - RNA, 21 nt, synthetic construct
  source 1..21: mol_type = other RNA
ttcttatggacaaagtctttc

SEQ ID NO: 365 - RNA, 21 nt, synthetic construct
  source 1..21: mol_type = other RNA
tgatcaaatatgttgagtttc

SEQ ID NO: 366 - RNA, 21 nt, synthetic construct
  source 1..21: mol_type = other RNA
tgatcataaaaagactgatcc

SEQ ID NO: 367 - RNA, 21 nt, synthetic construct
  source 1..21: mol_type = other RNA
tagtgacatattctttacctc

SEQ ID NO: 368 - RNA, 21 nt, synthetic construct
  source 1..21: mol_type = other RNA
tagttagttagttgctcttcc

SEQ ID NO: 369 - RNA, 21 nt, synthetic construct
  source 1..21: mol_type = other RNA
taatttctgtgggttcttgac

SEQ ID NO: 370 - RNA, 21 nt, synthetic construct
  source 1..21: mol_type = other RNA
tacatgaaaaacttgagagtc

SEQ ID NO: 371 - RNA, 21 nt, synthetic construct
  source 1..21: mol_type = other RNA
tgacatgaaaaacttgagagc

SEQ ID NO: 372 - RNA, 21 nt, synthetic construct
  source 1..21: mol_type = other RNA
ttagacatgaaaaacttgagc

SEQ ID NO: 373 - RNA, 21 nt, synthetic construct
  source 1..21: mol_type = other RNA
tgatgttgaattaatgtccac

SEQ ID NO: 374 - RNA, 21 nt, synthetic construct
  source 1..21: mol_type = other RNA
ttagagtataaccttccattc

SEQ ID NO: 375 - RNA, 21 nt, synthetic construct
  source 1..21: mol_type = other RNA
tatgtcattaatttggccctt

SEQ ID NO: 376 - RNA, 21 nt, synthetic construct
  source 1..21: mol_type = other RNA
tgtttttgaaatatgtcattc

SEQ ID NO: 377 - RNA, 21 nt, synthetic construct
  source 1..21: mol_type = other RNA
ttcataaaaagactgatcaac

SEQ ID NO: 378 - RNA, 21 nt, synthetic construct
  source 1..21: mol_type = other RNA
ttagtttatatgtagttcttc

SEQ ID NO: 379 - RNA, 21 nt, synthetic construct
  source 1..21: mol_type = other RNA
tgttgagttcaagtgacatac

SEQ ID NO: 380 - RNA, 21 nt, synthetic construct
  source 1..21: mol_type = other RNA
taggctttcaagttttgagtc

SEQ ID NO: 381 - RNA, 21 nt, synthetic construct
  source 1..21: mol_type = other RNA
tcttctaggaggctttcaagc

SEQ ID NO: 382 - RNA, 21 nt, synthetic construct
  source 1..21: mol_type = other RNA
tgtagacatgaaaaacttgag

SEQ ID NO: 383 - RNA, 21 nt, synthetic construct
  source 1..21: mol_type = other RNA
tatcacagtagacatgaaaac

SEQ ID NO: 384 - RNA, 21 nt, synthetic construct
  source 1..21: mol_type = other RNA
tgatccatctattcgatgttg

SEQ ID NO: 385 - RNA, 21 nt, synthetic construct
  source 1..21: mol_type = other RNA
tttttgtgatccatctattcg

SEQ ID NO: 386 - RNA, 21 nt, synthetic construct
  source 1..21: mol_type = other RNA
tattgaagttttgtgatccat

SEQ ID NO: 387 - RNA, 21 nt, synthetic construct
  source 1..21: mol_type = other RNA
tacgtttcattgaagttttgc

SEQ ID NO: 388 - RNA, 21 nt, synthetic construct
  source 1..21: mol_type = other RNA
tgatttcccaagtaaaaagac

SEQ ID NO: 389 - RNA, 21 nt, synthetic construct
  source 1..21: mol_type = other RNA
tttttctccacactcatcatg

SEQ ID NO: 390 - RNA, 21 nt, synthetic construct
  source 1..21: mol_type = other RNA
tgattttatagagtataacct

SEQ ID NO: 391 - RNA, 21 nt, synthetic construct
  source 1..21: mol_type = other RNA
gaaagactttgtccataagaa

SEQ ID NO: 392 - RNA, 21 nt, synthetic construct
  source 1..21: mol_type = other RNA
gaaactcaacatatttgatca

SEQ ID NO: 393 - RNA, 21 nt, synthetic construct
  source 1..21: mol_type = other RNA
ggatcagtctttttatgatca

SEQ ID NO: 394 - RNA, 21 nt, synthetic construct
  source 1..21: mol_type = other RNA
gaggtaaagaatatgtcacta

SEQ ID NO: 395 - RNA, 21 nt, synthetic construct
  source 1..21: mol_type = other RNA
ggaagagcaactaactaacta

SEQ ID NO: 396 - RNA, 21 nt, synthetic construct
  source 1..21: mol_type = other RNA
gtcaagaacccacagaaatta

SEQ ID NO: 397 - RNA, 21 nt, synthetic construct
  source 1..21: mol_type = other RNA
gactctcaagtttttcatgta

SEQ ID NO: 398 - RNA, 21 nt, synthetic construct
  source 1..21: mol_type = other RNA
gctctcaagtttttcatgtca

SEQ ID NO: 399 - RNA, 21 nt, synthetic construct
  source 1..21: mol_type = other RNA
gctcaagtttttcatgtctaa

SEQ ID NO: 400 - RNA, 21 nt, synthetic construct
  source 1..21: mol_type = other RNA
gtggacattaattcaacatca

SEQ ID NO: 401 - RNA, 21 nt, synthetic construct
  source 1..21: mol_type = other RNA
gaatggaaggttatactctaa

SEQ ID NO: 402 - RNA, 21 nt, synthetic construct
  source 1..21: mol_type = other RNA
aagggccaaattaatgacata

SEQ ID NO: 403 - RNA, 21 nt, synthetic construct
  source 1..21: mol_type = other RNA
gaatgacatatttcaaaaaca

SEQ ID NO: 404 - RNA, 21 nt, synthetic construct
  source 1..21: mol_type = other RNA
gttgatcagtctttttatgaa

SEQ ID NO: 405 - RNA, 21 nt, synthetic construct
  source 1..21: mol_type = other RNA
gaagaactacatataaactaa

SEQ ID NO: 406 - RNA, 21 nt, synthetic construct
  source 1..21: mol_type = other RNA
gtatgtcacttgaactcaaca

SEQ ID NO: 407 - RNA, 21 nt, synthetic construct
  source 1..21: mol_type = other RNA
gactcaaaacttgaaagccta

SEQ ID NO: 408 - RNA, 21 nt, synthetic construct
  source 1..21: mol_type = other RNA
gcttgaaagcctcctagaaga

SEQ ID NO: 409 - RNA, 21 nt, synthetic construct
  source 1..21: mol_type = other RNA
ctcaagtttttcatgtctaca

SEQ ID NO: 410 - RNA, 21 nt, synthetic construct
  source 1..21: mol_type = other RNA
gttttcatgtctactgtgata

SEQ ID NO: 411 - RNA, 21 nt, synthetic construct
  source 1..21: mol_type = other RNA
caacatcgaatagatggatca

SEQ ID NO: 412 - RNA, 21 nt, synthetic construct
  source 1..21: mol_type = other RNA
cgaatagatggatcacaaaaa

SEQ ID NO: 413 - RNA, 21 nt, synthetic construct
  source 1..21: mol_type = other RNA
atggatcacaaaacttcaata

SEQ ID NO: 414 - RNA, 21 nt, synthetic construct
  source 1..21: mol_type = other RNA
gcaaaacttcaatgaaacgta

SEQ ID NO: 415 - RNA, 21 nt, synthetic construct
  source 1..21: mol_type = other RNA
gtctttttacttgggaaatca

SEQ ID NO: 416 - RNA, 21 nt, synthetic construct
  source 1..21: mol_type = other RNA
catgatgagtgtggagaaaaa

SEQ ID NO: 417 - RNA, 21 nt, synthetic construct
  source 1..21: mol_type = other RNA
aggttatactctataaaatca

SEQ ID NO: 418 - RNA, 21 nt, synthetic construct
  source 1..21: mol_type = other RNA
ttcttatggacaaagtctttc

SEQ ID NO: 419 - RNA, 21 nt, synthetic construct
  source 1..21: mol_type = other RNA
tgatcaaatatgttgagtttc

SEQ ID NO: 420 - RNA, 21 nt, synthetic construct
  source 1..21: mol_type = other RNA
tgatcataaaaagactgatcc

SEQ ID NO: 421 - RNA, 21 nt, synthetic construct
  source 1..21: mol_type = other RNA
tagtgacatattctttacctc

SEQ ID NO: 422 - RNA, 21 nt, synthetic construct
  source 1..21: mol_type = other RNA
tagttagttagttgctcttcc

SEQ ID NO: 423 - RNA, 21 nt, synthetic construct
  source 1..21: mol_type = other RNA
taatttctgtgggttcttgac

SEQ ID NO: 424 - RNA, 21 nt, synthetic construct
  source 1..21: mol_type = other RNA
tacatgaaaaacttgagagtc

SEQ ID NO: 425 - RNA, 21 nt, synthetic construct
  source 1..21: mol_type = other RNA
tgacatgaaaaacttgagagc

SEQ ID NO: 426 - RNA, 21 nt, synthetic construct
  source 1..21: mol_type = other RNA
ttagacatgaaaaacttgagc

SEQ ID NO: 427 - RNA, 21 nt, synthetic construct
  source 1..21: mol_type = other RNA
tgatgttgaattaatgtccac

SEQ ID NO: 428 - RNA, 21 nt, synthetic construct
  source 1..21: mol_type = other RNA
ttagagtataaccttccattc

SEQ ID NO: 429 - RNA, 21 nt, synthetic construct
  source 1..21: mol_type = other RNA
tatgtcattaatttggccctt

SEQ ID NO: 430 - RNA, 21 nt, synthetic construct
  source 1..21: mol_type = other RNA
tgtttttgaaatatgtcattc

SEQ ID NO: 431 - RNA, 21 nt, synthetic construct
  source 1..21: mol_type = other RNA
ttcataaaaagactgatcaac

SEQ ID NO: 432 - RNA, 21 nt, synthetic construct
  source 1..21: mol_type = other RNA
ttagtttatatgtagttcttc

SEQ ID NO: 433 - RNA, 21 nt, synthetic construct
  source 1..21: mol_type = other RNA
tgttgagttcaagtgacatac

SEQ ID NO: 434 - RNA, 21 nt, synthetic construct
  source 1..21: mol_type = other RNA
taggctttcaagttttgagtc

SEQ ID NO: 435 - RNA, 21 nt, synthetic construct
  source 1..21: mol_type = other RNA
tcttctaggaggctttcaagc

SEQ ID NO: 436 - RNA, 21 nt, synthetic construct
  source 1..21: mol_type = other RNA
tgtagacatgaaaaacttgag

SEQ ID NO: 437 - RNA, 21 nt, synthetic construct
  source 1..21: mol_type = other RNA
tatcacagtagacatgaaaac

SEQ ID NO: 438 - RNA, 21 nt, synthetic construct
  source 1..21: mol_type = other RNA
tgatccatctattcgatgttg

SEQ ID NO: 439 - RNA, 21 nt, synthetic construct
  source 1..21: mol_type = other RNA
tttttgtgatccatctattcg

SEQ ID NO: 440 - RNA, 21 nt, synthetic construct
  source 1..21: mol_type = other RNA
tattgaagttttgtgatccat

SEQ ID NO: 441 - RNA, 21 nt, synthetic construct
  source 1..21: mol_type = other RNA
tacgtttcattgaagttttgc

SEQ ID NO: 442 - RNA, 21 nt, synthetic construct
  source 1..21: mol_type = other RNA
tgatttcccaagtaaaaagac

SEQ ID NO: 443 - RNA, 21 nt, synthetic construct
  source 1..21: mol_type = other RNA
tttttctccacactcatcatg

SEQ ID NO: 444 - RNA, 21 nt, synthetic construct
  source 1..21: mol_type = other RNA
tgattttatagagtataacct

SEQ ID NO: 445 - RNA, 21 nt, synthetic construct
  source 1..21: mol_type = other RNA
gaaagattttgtccataagaa

SEQ ID NO: 446 - RNA, 21 nt, synthetic construct
  source 1..21: mol_type = other RNA
gcaaaatcaagatttgctata

SEQ ID NO: 447 - RNA, 21 nt, synthetic construct
  source 1..21: mol_type = other RNA
gttaaagattttgtccataaa

SEQ ID NO: 448 - RNA, 21 nt, synthetic construct
  source 1..21: mol_type = other RNA
gaaaccaactacacgctacaa

SEQ ID NO: 449 - RNA, 21 nt, synthetic construct
  source 1..21: mol_type = other RNA
gaagctcaacatatttgatca

SEQ ID NO: 450 - RNA, 21 nt, synthetic construct
  source 1..21: mol_type = other RNA
gagctcaacatatttgatcaa

SEQ ID NO: 451 - RNA, 21 nt, synthetic construct
  source 1..21: mol_type = other RNA
gtatttgatcagtctttttaa

SEQ ID NO: 452 - RNA, 21 nt, synthetic construct
  source 1..21: mol_type = other RNA
gcacccagaagtaacatcaca

SEQ ID NO: 453 - RNA, 21 nt, synthetic construct
  source 1..21: mol_type = other RNA
gtaaagattttgtccataaga

SEQ ID NO: 454 - RNA, 21 nt, synthetic construct
  source 1..21: mol_type = other RNA
gcagtccatggacattaatta

SEQ ID NO: 455 - RNA, 21 nt, synthetic construct
  source 1..21: mol_type = other RNA
ccaaaatcaagatttgctata

SEQ ID NO: 456 - RNA, 21 nt, synthetic construct
  source 1..21: mol_type = other RNA
cttaaagactttgtccataaa

SEQ ID NO: 457 - RNA, 21 nt, synthetic construct
  source 1..21: mol_type = other RNA
gaaaccaactatacgctacaa

SEQ ID NO: 458 - RNA, 21 nt, synthetic construct
  source 1..21: mol_type = other RNA
gaaactcaacatatttgatca

SEQ ID NO: 459 - RNA, 21 nt, synthetic construct
  source 1..21: mol_type = other RNA
gaactcaacatatttgatcaa

SEQ ID NO: 460 - RNA, 21 nt, synthetic construct
  source 1..21: mol_type = other RNA
gtatttgatcagtctttttaa

SEQ ID NO: 461 - RNA, 21 nt, synthetic construct
  source 1..21: mol_type = other RNA
acacccagaagtaacttcaca

SEQ ID NO: 462 - RNA, 21 nt, synthetic construct
  source 1..21: mol_type = other RNA
gtaaagactttgtccataaga

SEQ ID NO: 463 - RNA, 21 nt, synthetic construct
  source 1..21: mol_type = other RNA
gtagtccatggacattaatta

SEQ ID NO: 464 - RNA, 21 nt, synthetic construct
  source 1..21: mol_type = other RNA
ttcttatggacaaaatctttc

SEQ ID NO: 465 - RNA, 21 nt, synthetic construct
  source 1..21: mol_type = other RNA
tatagcaaatcttgattttgc

SEQ ID NO: 466 - RNA, 21 nt, synthetic construct
  source 1..21: mol_type = other RNA
tttatggacaaaatctttaac

SEQ ID NO: 467 - RNA, 21 nt, synthetic construct
  source 1..21: mol_type = other RNA
ttgtagcgtgtagttggtttc

SEQ ID NO: 468 - RNA, 21 nt, synthetic construct
  source 1..21: mol_type = other RNA
tgatcaaatatgttgagcttc

SEQ ID NO: 469 - RNA, 21 nt, synthetic construct
  source 1..21: mol_type = other RNA
ttgatcaaatatgttgagctc

SEQ ID NO: 470 - RNA, 21 nt, synthetic construct
  source 1..21: mol_type = other RNA
ttaaaaagactgatcaaatac

SEQ ID NO: 471 - RNA, 21 nt, synthetic construct
  source 1..21: mol_type = other RNA
tgtgatgttacttctgggtgc

SEQ ID NO: 472 - RNA, 21 nt, synthetic construct
  source 1..21: mol_type = other RNA
tcttatggacaaaatctttac

SEQ ID NO: 473 - RNA, 21 nt, synthetic construct
  source 1..21: mol_type = other RNA
taattaatgtccatggactgc

SEQ ID NO: 474 - RNA, 21 nt, synthetic construct
  source 1..21: mol_type = other RNA
tatagcaaatcttgattttgg

SEQ ID NO: 475 - RNA, 21 nt, synthetic construct
  source 1..21: mol_type = other RNA
tttatggacaaagtctttaag

SEQ ID NO: 476 - RNA, 21 nt, synthetic construct
  source 1..21: mol_type = other RNA
ttgtagcgtatagttggtttc

SEQ ID NO: 477 - RNA, 21 nt, synthetic construct
  source 1..21: mol_type = other RNA
tgatcaaatatgttgagtttc

SEQ ID NO: 478 - RNA, 21 nt, synthetic construct
  source 1..21: mol_type = other RNA
ttgatcaaatatgttgagttc

SEQ ID NO: 479 - RNA, 21 nt, synthetic construct
  source 1..21: mol_type = other RNA
ttaaaaagactgatcaaatac

SEQ ID NO: 480 - RNA, 21 nt, synthetic construct
  source 1..21: mol_type = other RNA
tgtgaagttacttctgggtgt

SEQ ID NO: 481 - RNA, 21 nt, synthetic construct
  source 1..21: mol_type = other RNA
tcttatggacaaagtctttac

SEQ ID NO: 482 - RNA, 21 nt, synthetic construct
  source 1..21: mol_type = other RNA
taattaatgtccatggactac

SEQ ID NO: 483 - RNA, 2995 nt, Macaca fascicularis
  source 1..2995: mol_type = mRNA
tacaatttcaaattacctattaagttagttgctcatttctttgatttcatttagcattgatgtaactcaatgtggaagaaggttacattcgtgcaagttaacatggcttaatgattaactatattcacctgccaaccttgccttttctgtggcaaatattggtatatatagagttaagaagtctaggtctgcttccagaagaacacagttccacgctgcttgaaattgaaaatcaggataaaaatgttcacaattaagctccttctttttattgttcctctagttatttcctccagaattgaccaagacaattcatcatttgattctgtatctccagagccaaaatcaagatttgctatgttagacgatgtaaaaattttagccaatggcctccttcagttgggacatggtcttaaagactttgtccataagactaagggccaaattaatgacatatttcaaaaactcaacatatttgatcagtctttttatgatctatcactgcaaaccagtgaaatcaaagaagaagaaaaggaactgagaagaactacatataaactacaagtcaaaaatgaagaggtaaagaatatgtcacttgaactcaactcaaaacttgaaagcctcctagaagaaaaaattctacttcaacaaaaagtgaaatatttagaagagcaactaactaacttaattcaaaatcaacctgcaactccagaacatccagaagtaacttcacttaaaagttttgtagaaaaacaagataatagcatcaaagaccttctccagactgtggaagaacaatataagcaattaaaccaacagcatagtcaaataaaagaaatagaaaatcagctcagaatgactaatattcaagaacccacagaaatttctctatcttccaagccaagagcaccaagaactactccctttcttcagctgaatgaaataagaaatgtaaaacatgatggcattcctgctgattgtaccaccatttacaatagaggtgaacatataagtggcacgtatgccatcagacccagcaactctcaagtttttcatgtctactgtgatgttgtatcaggtagtccatggacattaattcaacatcgaatagatggatcacaaaacttcaatgaaacgtgggagaactacaaatatggtttcgggaggcttgatggagaattctggttgggcctagagaagatatactccatagtgaagcaatctaattacgttttacgaattgagttggaagactggaaagacaacaaacattatattgaatattctttttacttgggaaatcacgaaaccaactatacgctacatgtagttaagattactggcaatgtccccaatgcaatcccggaaaacaaagatttggtgttttctacttgggatcacaaagcaaaaggacacttcagctgtccagagagttattcaggaggctggtggtggcatgatgagtgtggagaaaacaacctaaatggtaaatataacaaaccaagaacaaaatctaagccagagcggagaagaggattatcctggaagtctcaaaatggaaggttatactctataaaatcaaccaaaatgttgatccatccaacagattcagaaagctttgaatgaactgaggcaaatttaaaaggcaataaattaaacattaaactcattccaagttaatgtggtttaataatctggtattaaatccttaagagaaggcttgagaaatagatttttttatcttaaagtcactgtcaatttaagattaaacatacaatcacataaccttaaagaataccatttacatttctcaatcaaaattcttacaacactatttgttttatattttgtgatgtgggaatcaattttagatggtcgcaatctaaattataatcaacaggtgaacttactaaataacttttctaaataaaaaacttagagactttaattttaaaagtcatcatatgagctaatatcacaattttcccagtttaaaaaactagttttcttgttaaaactctaaacttgactaaataaagaggactgataattatacagttcttaaatttgttgtaatattaatttcaaaactaaaaattgtcagcacagagtatgtgtaaaaatctgtaatataaatttttaaactgatgcctcattttgctacaaaataatctggagtaaatttttgataggatttatttatgaaacctaatgaagcaggattaaatactgtattaaaataggttcgctgtcttttaaacaaatggagatgatgattactaagtcacattgactttaatatgaggtatcactataccttaacatatttgttaaaacgtatactgtatacattttgtgtattttaatacttaatactatgaaaacaagtaattgtaaacgtatcttgtcagattacaataggaatgaacatattggtgacatcgagttaaagtttatatttcccctaaatatgctgcgattccaatatattcatgtaggttttcaagcagaaataaaccttgtaacaagttactgactaaacagcctgacaagtatgtatatatgtttaaaattcaataaataaagacccagtcttctaaattataaaaatttaaattagtcttgcacaaattaaattattcatcacaaaagatgtattgttatttttaagtcatttaagccctaaatccctaaagattagatataaattttttttgccagagtataaattgtcagaatttatttttaaatatattttttaaaactaccagtaagaaattttaaattaaacccatttgttaaaggatatagtgcccaagttatacggtgacctacctttgtcaatatttagcattatgtatttcaaattatccaatatacatgtcatatatatttttatatgttgcatatataaaagatatacacgatttatgtgaatcctatgtaaatattttgttccagaaaagtacaaaataataaaggtaaaaataatcca

SEQ ID NO: 484 - RNA, 21 nt, synthetic construct
  source 1..21: mol_type = other RNA
gaatggaaggttatactctat

SEQ ID NO: 485 - RNA, 21 nt, synthetic construct
  source 1..21: mol_type = other RNA
gaatggaaggttatactctac

SEQ ID NO: 486 - RNA, 21 nt, synthetic construct
  source 1..21: mol_type = other RNA
gaatggaaggttatactctag

SEQ ID NO: 487 - RNA, 17 nt, synthetic construct
  source 1..17: mol_type = other RNA
ggaaggttatactctaa

SEQ ID NO: 488 - RNA, 17 nt, synthetic construct
  source 1..17: mol_type = other RNA
tggaaggttatactcta

SEQ ID NO: 489 - RNA, 18 nt, synthetic construct
  source 1..18: mol_type = other RNA
tggaaggttatactctaa

SEQ ID NO: 490 - RNA, 19 nt, synthetic construct
  source 1..19: mol_type = other RNA
atggaaggttatactctaa

SEQ ID NO: 491 - RNA, 19 nt, synthetic construct
  source 1..19: mol_type = other RNA
ttggaaggttatactctaa

SEQ ID NO: 492 - RNA, 19 nt, synthetic construct
  source 1..19: mol_type = other RNA
gtggaaggttatactctaa

SEQ ID NO: 493 - RNA, 19 nt, synthetic construct
  source 1..19: mol_type = other RNA
ctggaaggttatactctaa

SEQ ID NO: 494 - RNA, 20 nt, synthetic construct
  source 1..20: mol_type = other RNA
aatggaaggttatactctaa

SEQ ID NO: 495 - RNA, 20 nt, synthetic construct
  source 1..20: mol_type = other RNA
tatggaaggttatactctaa

SEQ ID NO: 496 - RNA, 20 nt, synthetic construct
  source 1..20: mol_type = other RNA
gatggaaggttatactctaa

SEQ ID NO: 497 - RNA, 20 nt, synthetic construct
  source 1..20: mol_type = other RNA
catggaaggttatactctaa

SEQ ID NO: 498 - RNA, 21 nt, synthetic construct
  source 1..21: mol_type = other RNA
caatggaaggttatactctaa

SEQ ID NO: 499 - RNA, 21 nt, synthetic construct
  source 1..21: mol_type = other RNA
aaatggaaggttatactctaa

SEQ ID NO: 500 - RNA, 21 nt, synthetic construct
  source 1..21: mol_type = other RNA
taatggaaggttatactctaa

SEQ ID NO: 501 - RNA, 22 nt, synthetic construct
  source 1..22: mol_type = other RNA
aaaatggaaggttatactctaa

SEQ ID NO: 502 - RNA, 23 nt, synthetic construct
  source 1..23: mol_type = other RNA
caaaatggaaggttatactctaa

SEQ ID NO: 503 - RNA, 24 nt, synthetic construct
  source 1..24: mol_type = other RNA
tcaaaatggaaggttatactctaa

SEQ ID NO: 504 - RNA, 25 nt, synthetic construct
  source 1..25: mol_type = other RNA
ctcaaaatggaaggttatactctaa

SEQ ID NO: 505 - RNA, 26 nt, synthetic construct
  source 1..26: mol_type = other RNA
tctcaaaatggaaggttatactctaa

SEQ ID NO: 506 - RNA, 32 nt, synthetic construct
  source 1..32: mol_type = other RNA
tggaagtctcaaaatggaaggttatactctaa

SEQ ID NO: 507 - RNA, 21 nt, synthetic construct
  source 1..21: mol_type = other RNA
gaatggaaggttatactctaa

SEQ ID NO: 508 - RNA, 21 nt, synthetic construct
  source 1..21: mol_type = other RNA
gaatggaaggttatactctaa

SEQ ID NO: 509 - RNA, 21 nt, synthetic construct
  source 1..21: mol_type = other RNA
gaatggaaggttatactctaa

SEQ ID NO: 510 - RNA, 21 nt, synthetic construct
  source 1..21: mol_type = other RNA
gaatggaaggttatactctaa

SEQ ID NO: 511 - RNA, 21 nt, synthetic construct
  source 1..21: mol_type = other RNA
gaatggaaggttatactctaa

SEQ ID NO: 512 - RNA, 21 nt, synthetic construct
  source 1..21: mol_type = other RNA
gaatggaaggttatattctaa

SEQ ID NO: 513 - RNA, 21 nt, synthetic construct
  source 1..21: mol_type = other RNA
gaatggaaggttatactctga

SEQ ID NO: 514 - RNA, 21 nt, synthetic construct
  source 1..21: mol_type = other RNA
gaatggaaggttatactctaa

SEQ ID NO: 515 - RNA, 21 nt, synthetic construct
  source 1..21: mol_type = other RNA
atagagtataaccttccattc

SEQ ID NO: 516 - RNA, 21 nt, synthetic construct
  source 1..21: mol_type = other RNA
gtagagtataaccttccattc

SEQ ID NO: 517 - RNA, 21 nt, synthetic construct
  source 1..21: mol_type = other RNA
ctagagtataaccttccattc

SEQ ID NO: 518 - RNA, 17 nt, synthetic construct
  source 1..17: mol_type = other RNA
ttagagtataaccttcc

SEQ ID NO: 519 - RNA, 17 nt, synthetic construct
  source 1..17: mol_type = other RNA
tagagtataaccttcca

SEQ ID NO: 520 - RNA, 18 nt, synthetic construct
  source 1..18: mol_type = other RNA
ttagagtataaccttcca

SEQ ID NO: 521 - RNA, 19 nt, synthetic construct
  source 1..19: mol_type = other RNA
ttagagtataaccttccat

SEQ ID NO: 522 - RNA, 19 nt, synthetic construct
  source 1..19: mol_type = other RNA
ttagagtataaccttccaa

SEQ ID NO: 523 - RNA, 19 nt, synthetic construct
  source 1..19: mol_type = other RNA
ttagagtataaccttccac

SEQ ID NO: 524 - RNA, 19 nt, synthetic construct
  source 1..19: mol_type = other RNA
ttagagtataaccttccag

SEQ ID NO: 525 - RNA, 20 nt, synthetic construct
  source 1..20: mol_type = other RNA
ttagagtataaccttccatt

SEQ ID NO: 526 - RNA, 20 nt, synthetic construct
  source 1..20: mol_type = other RNA
ttagagtataaccttccata

SEQ ID NO: 527 - RNA, 20 nt, synthetic construct
  source 1..20: mol_type = other RNA
ttagagtataaccttccatc

SEQ ID NO: 528 - RNA, 20 nt, synthetic construct
  source 1..20: mol_type = other RNA
ttagagtataaccttccatg

SEQ ID NO: 529 - RNA, 21 nt, synthetic construct
  source 1..21: mol_type = other RNA
ttagagtataaccttccattg

SEQ ID NO: 530 - RNA, 21 nt, synthetic construct
  source 1..21: mol_type = other RNA
ttagagtataaccttccattt

SEQ ID NO: 531 - RNA, 21 nt, synthetic construct
  source 1..21: mol_type = other RNA
ttagagtataaccttccatta

SEQ ID NO: 532 - RNA, 22 nt, synthetic construct
  source 1..22: mol_type = other RNA
ttagagtataaccttccatttt

SEQ ID NO: 533 - RNA, 23 nt, synthetic construct
  source 1..23: mol_type = other RNA
ttagagtataaccttccattttg

SEQ ID NO: 534 - RNA, 24 nt, synthetic construct
  source 1..24: mol_type = other RNA
ttagagtataaccttccattttga

SEQ ID NO: 535 - RNA, 25 nt, synthetic construct
  source 1..25: mol_type = other RNA
ttagagtataaccttccattttgag

SEQ ID NO: 536 - RNA, 26 nt, synthetic construct
  source 1..26: mol_type = other RNA
ttagagtataaccttccattttgaga

SEQ ID NO: 537 - RNA, 32 nt, synthetic construct
  source 1..32: mol_type = other RNA
ttagagtataaccttccattttgagacttcca

SEQ ID NO: 538 - RNA, 23 nt, synthetic construct
  source 1..23: mol_type = other RNA
ttagagtataaccttccattctt

SEQ ID NO: 539 - RNA, 23 nt, synthetic construct
  source 1..23: mol_type = other RNA
ttagagtataaccttccattcga

SEQ ID NO: 540 - RNA, 21 nt, synthetic construct
  source 1..21: mol_type = other RNA
ttagagtataaccttccattc

SEQ ID NO: 541 - RNA, 21 nt, synthetic construct
  source 1..21: mol_type = other RNA
ttagagtataaccttccattc

SEQ ID NO: 542 - RNA, 21 nt, synthetic construct
  source 1..21: mol_type = other RNA
ttagagtataaccttccattc

SEQ ID NO: 543 - RNA, 21 nt, synthetic construct
  source 1..21: mol_type = other RNA
ttagagtataaccttccattc

SEQ ID NO: 544 - RNA, 21 nt, synthetic construct
  source 1..21: mol_type = other RNA
tcagagtataaccttccattc

SEQ ID NO: 545 - RNA, 21 nt, synthetic construct
  source 1..21: mol_type = other RNA
ttagagtataaccttccattc

SEQ ID NO: 546 - RNA, 21 nt, synthetic construct
  source 1..21: mol_type = other RNA
gaatggaaggttatactctat

SEQ ID NO: 547 - RNA, 21 nt, synthetic construct
  source 1..21: mol_type = other RNA
gaatggaaggttatactctac

SEQ ID NO: 548 - RNA, 21 nt, synthetic construct
  source 1..21: mol_type = other RNA
gaatggaaggttatactctag

SEQ ID NO: 549 - RNA, 17 nt, synthetic construct
  source 1..17: mol_type = other RNA
ggaaggttatactctaa

SEQ ID NO: 550 - RNA, 18 nt, synthetic construct
  source 1..18: mol_type = other RNA
tggaaggttatactctaa

SEQ ID NO: 551 - RNA, 19 nt, synthetic construct
  source 1..19: mol_type = other RNA
atggaaggttatactctaa

SEQ ID NO: 552 - RNA, 19 nt, synthetic construct
  source 1..19: mol_type = other RNA
ttggaaggttatactctaa

SEQ ID NO: 553 - RNA, 19 nt, synthetic construct
  source 1..19: mol_type = other RNA
gtggaaggttatactctaa

SEQ ID NO: 554 - RNA, 19 nt, synthetic construct
  source 1..19: mol_type = other RNA
ctggaaggttatactctaa

SEQ ID NO: 555 - RNA, 20 nt, synthetic construct
  source 1..20: mol_type = other RNA
aatggaaggttatactctaa

SEQ ID NO: 556 - RNA, 20 nt, synthetic construct
  source 1..20: mol_type = other RNA
tatggaaggttatactctaa

SEQ ID NO: 557 - RNA, 20 nt, synthetic construct
  source 1..20: mol_type = other RNA
gatggaaggttatactctaa

SEQ ID NO: 558 - RNA, 20 nt, synthetic construct
  source 1..20: mol_type = other RNA
catggaaggttatactctaa

SEQ ID NO: 559 - RNA, 21 nt, synthetic construct
  source 1..21: mol_type = other RNA
caatggaaggttatactctaa

SEQ ID NO: 560 - RNA, 21 nt, synthetic construct
  source 1..21: mol_type = other RNA
aaatggaaggttatactctaa

SEQ ID NO: 561 - RNA, 21 nt, synthetic construct
  source 1..21: mol_type = other RNA
taatggaaggttatactctaa

SEQ ID NO: 562 - RNA, 22 nt, synthetic construct
  source 1..22: mol_type = other RNA
aaaatggaaggttatactctaa

SEQ ID NO: 563 - RNA, 23 nt, synthetic construct
  source 1..23: mol_type = other RNA
caaaatggaaggttatactctaa

SEQ ID NO: 564 - RNA, 24 nt, synthetic construct
  source 1..24: mol_type = other RNA
tcaaaatggaaggttatactctaa

SEQ ID NO: 565 - RNA, 25 nt, synthetic construct
  source 1..25: mol_type = other RNA
ctcaaaatggaaggttatactctaa

SEQ ID NO: 566 - RNA, 26 nt, synthetic construct
  source 1..26: mol_type = other RNA
tctcaaaatggaaggttatactctaa

SEQ ID NO: 567 - RNA, 32 nt, synthetic construct; identical to SEQ ID NO: 506
  source 1..32: mol_type = other RNA

SEQ ID NO: 568 - RNA, 21 nt, synthetic construct
  source 1..21: mol_type = other RNA
gaatggaaggttatactctaa

SEQ ID NO: 569 - RNA, 21 nt, synthetic construct
  source 1..21: mol_type = other RNA
gaatggaaggttatactctaa

SEQ ID NO: 570 - RNA, 21 nt, synthetic construct
  source 1..21: mol_type = other RNA
gaatggaaggttatactctaa

SEQ ID NO: 571 - RNA, 21 nt, synthetic construct
  source 1..21: mol_type = other RNA
gaatggaaggttatactctaa

SEQ ID NO: 572 - RNA, 21 nt, synthetic construct
  source 1..21: mol_type = other RNA
gaatggaaggttatactctaa

SEQ ID NO: 573 - RNA, 21 nt, synthetic construct
  source 1..21: mol_type = other RNA
gaatggaaggttatattctaa

SEQ ID NO: 574 - RNA, 21 nt, synthetic construct
  source 1..21: mol_type = other RNA
gaatggaaggttatactctga

SEQ ID NO: 575 - RNA, 21 nt, synthetic construct
  source 1..21: mol_type = other RNA
gaatggaaggttatactctaa

SEQ ID NO: 576 - RNA, 21 nt, synthetic construct
  source 1..21: mol_type = other RNA
atagagtataaccttccattc

SEQ ID NO: 577 - RNA, 21 nt, synthetic construct
  source 1..21: mol_type = other RNA
gtagagtataaccttccattc

SEQ ID NO: 578 - RNA, 21 nt, synthetic construct
  source 1..21: mol_type = other RNA
ctagagtataaccttccattc

SEQ ID NO: 579 - RNA, 17 nt, synthetic construct
  source 1..17: mol_type = other RNA
ttagagtataaccttcc

SEQ ID NO: 580 - RNA, 18 nt, synthetic construct
  source 1..18: mol_type = other RNA
ttagagtataaccttcca

SEQ ID NO: 581 - RNA, 19 nt, synthetic construct
  source 1..19: mol_type = other RNA
ttagagtataaccttccat

SEQ ID NO: 582 - RNA, 19 nt, synthetic construct
  source 1..19: mol_type = other RNA
ttagagtataaccttccaa

SEQ ID NO: 583 - RNA, 19 nt, synthetic construct
  source 1..19: mol_type = other RNA
ttagagtataaccttccac

SEQ ID NO: 584 - RNA, 19 nt, synthetic construct
  source 1..19: mol_type = other RNA
ttagagtataaccttccag

SEQ ID NO: 585 - RNA, 20 nt, synthetic construct
  source 1..20: mol_type = other RNA
ttagagtataaccttccatt

SEQ ID NO: 586 - RNA, 20 nt, synthetic construct
  source 1..20: mol_type = other RNA
ttagagtataaccttccata

SEQ ID NO: 587 - RNA, 20 nt, synthetic construct
  source 1..20: mol_type = other RNA
ttagagtataaccttccatc

SEQ ID NO: 588 - RNA, 20 nt, synthetic construct
  source 1..20: mol_type = other RNA
ttagagtataaccttccatg

SEQ ID NO: 589 - RNA, 21 nt, synthetic construct
  source 1..21: mol_type = other RNA
ttagagtataaccttccattg

SEQ ID NO: 590 - RNA, 21 nt, synthetic construct
  source 1..21: mol_type = other RNA
ttagagtataaccttccattt

SEQ ID NO: 591 - RNA, 21 nt, synthetic construct
  source 1..21: mol_type = other RNA
ttagagtataaccttccatta

SEQ ID NO: 592 - RNA, 22 nt, synthetic construct
  source 1..22: mol_type = other RNA
ttagagtataaccttccatttt

SEQ ID NO: 593 - RNA, 23 nt, synthetic construct
  source 1..23: mol_type = other RNA
ttagagtataaccttccattttg

SEQ ID NO: 594 - RNA, 24 nt, synthetic construct
  source 1..24: mol_type = other RNA
ttagagtataaccttccattttga

SEQ ID NO: 595 - RNA, 25 nt, synthetic construct
  source 1..25: mol_type = other RNA
ttagagtataaccttccattttgag

SEQ ID NO: 596 - RNA, 26 nt, synthetic construct
  source 1..26: mol_type = other RNA
ttagagtataaccttccattttgaga

SEQ ID NO: 597 - RNA, 32 nt, synthetic construct; identical to SEQ ID NO: 537
  source 1..32: mol_type = other RNA

SEQ ID NO: 598 - RNA, 23 nt, synthetic construct
  source 1..23: mol_type = other RNA
ttagagtataaccttccattctt

SEQ ID NO: 599 - RNA, 23 nt, synthetic construct
  source 1..23: mol_type = other RNA
ttagagtataaccttccattcga

SEQ ID NO: 600 - RNA, 21 nt, synthetic construct
  source 1..21: mol_type = other RNA
ttagagtataaccttccattc

SEQ ID NO: 601 - RNA, 21 nt, synthetic construct
  source 1..21: mol_type = other RNA
ttagagtataaccttccattc

SEQ ID NO: 602 - RNA, 21 nt, synthetic construct
  source 1..21: mol_type = other RNA
ttagagtataaccttccattc

SEQ ID NO: 603 - RNA, 21 nt, synthetic construct
  source 1..21: mol_type = other RNA
ttagagtataaccttccattc

SEQ ID NO: 604 - RNA, 21 nt, synthetic construct
  source 1..21: mol_type = other RNA
tcagagtataaccttccattc

SEQ ID NO: 605 - RNA, 21 nt, synthetic construct
  source 1..21: mol_type = other RNA
ttagagtataaccttccattc

SEQ ID NO: 606 - RNA, 21 nt, synthetic construct
  source 1..21: mol_type = other RNA
gaatggaaggttatactctaa

SEQ ID NO: 607 - RNA, 21 nt, synthetic construct
  source 1..21: mol_type = other RNA
caagaactacatataaactaa

SEQ ID NO: 608 - RNA, 21 nt, synthetic construct
  source 1..21: mol_type = other RNA
caagaactacatataaactaa

SEQ ID NO: 609 - RNA, 21 nt, synthetic construct
  source 1..21: mol_type = other RNA
gtatgtcacttgaactcaaca

SEQ ID NO: 610 - RNA, 21 nt, synthetic construct
  source 1..21: mol_type = other RNA
gtggatcacaaaacttcaata

SEQ ID NO: 611 - RNA, 21 nt, synthetic construct
  source 1..21: mol_type = other RNA
ttagagtataaccttccattc

SEQ ID NO: 612 - RNA, 21 nt, synthetic construct
  source 1..21: mol_type = other RNA
ttagtttatatgtagttcttg

SEQ ID NO: 613 - RNA, 21 nt, synthetic construct
  source 1..21: mol_type = other RNA
ttagtttatatgtagttcttg

SEQ ID NO: 614 - RNA, 21 nt, synthetic construct
  source 1..21: mol_type = other RNA
tgttgagttcaagtgacatac

SEQ ID NO: 615 - RNA, 21 nt, synthetic construct
  source 1..21: mol_type = other RNA
tattgaagttttgtgatccac

SEQ ID NO: 616 - RNA, 21 nt, synthetic construct
  source 1..21: mol_type = other RNA
gactctcaagtttttcatgta

SEQ ID NO: 617 - RNA, 21 nt, synthetic construct
  source 1..21: mol_type = other RNA
tacatgaaaaacttgagagtc

SEQ ID NO: 618 - RNA, 21 nt, synthetic construct
  source 1..21: mol_type = other RNA
gaatgacatatttcaaaaaca

SEQ ID NO: 619 - RNA, 21 nt, synthetic construct
  source 1..21: mol_type = other RNA
tgtttttgaaatatgtcattc

SEQ ID NO: 620 - RNA, 21 nt, synthetic construct
  source 1..21: mol_type = other RNA
gttgatcagtctttttatgaa

SEQ ID NO: 621 - RNA, 21 nt, synthetic construct
  source 1..21: mol_type = other RNA
ttcataaaaagactgatcaac

SEQ ID NO: 622 - RNA, 21 nt, synthetic construct
  source 1..21: mol_type = other RNA
cgaatagatggatcacaaaaa

SEQ ID NO: 623 - RNA, 21 nt, synthetic construct
  source 1..21: mol_type = other RNA
tttttgtgatccatctattcg

SEQ ID NO: 624 - RNA, 21 nt, synthetic construct
  source 1..21: mol_type = other RNA
gtggatcacaaaacttcaata

SEQ ID NO: 625 - RNA, 21 nt, synthetic construct
  source 1..21: mol_type = other RNA
tattgaagttttgtgatccat

SEQ ID NO: 626 - RNA, 21 nt, synthetic construct
  source 1..21: mol_type = other RNA
gtctttttacttgggaaatca

SEQ ID NO: 627 - RNA, 21 nt, synthetic construct
  source 1..21: mol_type = other RNA
tgatttcccaagtaaaaagac

SEQ ID NO: 628 - RNA, 21 nt, synthetic construct
  source 1..21: mol_type = other RNA
catgatgagtgtggagaaaaa

SEQ ID NO: 629 - RNA, 21 nt, synthetic construct
  source 1..21: mol_type = other RNA
tttttctccacactcatcatg